SEQUENCE LISTING (20 sequences)

SEQ ID NO: 1 - protein, 1368 aa, Streptococcus pyogenes
  MISC_FEATURE (1)..(20): AKP81606.1
  MISC_FEATURE (1)..(1368): AKP81606.1
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 2560 aa, Francisella tularensis
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNSLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 15 aa, Artificial Sequence
  Synthetic
IGLDIGTNSVGWAVI

SEQ ID NO: 4 - protein, 8 aa, Artificial Sequence
  Synthetic Sequence
IVIEMARE

SEQ ID NO: 5 - protein, 27 aa, Artificial Sequence
  Synthetic Sequence
DVDHIVPQSFLKDDSIDNKVLTRSDKN

SEQ ID NO: 6 - protein, 8 aa, Artificial Sequence
  Synthetic Sequence
HHAHDAYL

SEQ ID NO: 7 - protein, 11 aa, Artificial Sequence
  Synthetic Sequence
YGRKKRRQRRR

SEQ ID NO: 8 - protein, 12 aa, Artificial Sequence
  Synthetic Sequence
RRQRRTSKLMKR

SEQ ID NO: 9 - protein, 27 aa, Artificial Sequence
  Synthetic Sequence
GWTLNSAGYLLGKINLKALAALAKKIL

SEQ ID NO: 10 - protein, 33 aa, Artificial Sequence
  Synthetic Sequence
KALAWEAKLAKALAKALAKHLAKALAKALKCEA

SEQ ID NO: 11 - protein, 16 aa, Artificial Sequence
  Synthetic Sequence
RQIKIWFQNRRMKWKK

SEQ ID NO: 12 - protein, 11 aa, Artificial Sequence
  Synthetic Sequence
YGRKKRRQRRR

SEQ ID NO: 13 - protein, 9 aa, Artificial Sequence
  Synthetic Sequence
RKKRRQRRR

SEQ ID NO: 14 - protein, 11 aa, Artificial Sequence
  Synthetic Sequence
YGRKKRRQRRR

SEQ ID NO: 15 - protein, 8 aa, Artificial Sequence
  Synthetic Sequence
RKKRRQRR

SEQ ID NO: 16 - protein, 11 aa, Artificial Sequence
  Synthetic Sequence
YARAAARQARA

SEQ ID NO: 17 - protein, 11 aa, Artificial Sequence
  Synthetic Sequence
THRLPRRRRRR

SEQ ID NO: 18 - protein, 11 aa, Artificial Sequence
  Synthetic Sequence
GGRRARRRRRR

SEQ ID NO: 19 - protein, 1307 aa, Artificial Sequence
  Synthetic Sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1228 aa, Artificial Sequence
  Synthetic
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

(19) United States
(12) Patent Application Publication
(10) Pub. No.: US 2023/0147779 A1
(43) Pub. Date: May 11, 2023

(54) POLYMER WITH CATIONIC AND HYDROPHOBIC SIDE CHAINS

(71) Applicant: GeneEdit, Inc., South San Francisco, CA (US)

(72) Inventors: Kunwoo Lee, South San Francisco, CA (US); Santanu Maity, South San Francisco, CA (US)

(21) Appl. No.: 17/921,016

(22) PCT Filed: Apr. 23, 2021
(86) PCT No.: PCT/US2021/028967

§ 371 Date: Oct. 24, 2022

Related U.S. Application Data
(60) Provisional application No. 63/106,824, filed on Oct. 28, 2020.
(60) Provisional application No. 63/120,681, filed on Dec. 2, 2020.

(51) Int. Cl. A61K 47/34; C08G 69/10; C08G 69/40; C08G 69/48; C08G 73/02; A61K 31/7105; A61K 38/45; A61K 48/00; A61K 9/51

(52) U.S. Cl. CPC A61K 47/34; C08G 69/10; C08G 69/40; C08G 69/48; C08G 73/028; A61K 31/7105; A61K 38/45; C12Y 207/07; A61K 48/0041; A61K 9/5146

(57) ABSTRACT

Provided is a polymer comprising a hydrolysable polymer backbone, the polymer backbone comprising (i) monomer units comprising a hydrophobic side chain; and (ii) monomer units comprising a cationic side chain; wherein the cationic side chain comprises a polyamine with at least one tertiary amine and only a single nucleophilic center, optionally at the terminus of the polyamine, as well as a method of preparing said polymer, and a method of delivering a nucleic acid and/or polypeptide to a cell using the polymer.

### CROSS-REFERENCE TO RELATED APPLICATIONS

This application is the national stage of International Patent Application No. PCT/2020/029660, filed Apr. 23, 2020, which claims the benefit of U.S. Provisional Patent Application No. 63/106,824 filed on Oct. 28, 2020, and U.S. Provisional Patent Application No. 63/120,681 filed on Dec. 2, 2020, the entire disclosures of which are hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted concurrently herewith and identified as follows: One 60,500 Byte ASCII (Text) file named “513448_ST25.TXT,” created on Apr. 23, 2021.

### BACKGROUND OF THE INVENTION

Peptide, protein, and nucleic based technologies have countless applications to prevent, cure and treat diseases. However, the safe and effective delivery of large molecules (e.g., polypeptides and nucleic acids) to their target tissues remains problematic. Accordingly, there continues to be a need for new compositions and methods useful for delivering therapeutic molecules.

### BRIEF SUMMARY OF THE INVENTION

Provided herein is a polymer comprising a hydrolysable polymer backbone, the polymer backbone comprising (i) monomer units comprising a hydrophobic side chain; and (ii) monomer units comprising a cationic side chain; wherein the cationic side chain comprises a polyamine with at least one tertiary amine and only a single nucleophilic center, optionally at the terminus of the polyamine, as well as a method of preparing said polymer, and a method of delivering a nucleic acid and/or polypeptide to a cell using the polymer.

Also provided herein is a polymer comprising a structure of Formula 1:

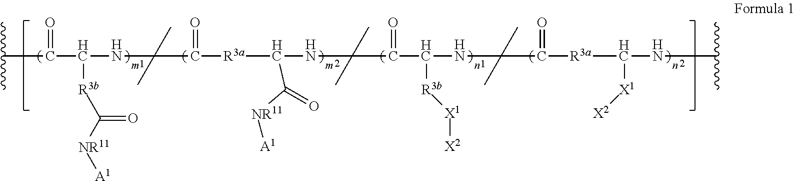

wherein:

each of m¹ and m² is an integer from 0 to 1000, provided that the sum of m¹+m² is greater than 2;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents, optionally hydrogen or a C₁-C₃ alkyl group;

each instance of X¹ independently is —C(O)O—, —C(O)NR¹¹—, —C(O)—, —S(O)(O)—, or a bond;

each instance of X² is independently a hydrophobic group, such as a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents;

and each instance of A¹ is independently a group of formula

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5, optionally wherein p1, p3, or both are greater than p2, and/or p1, p3, or both are integers of 3 to 5; r1 is an integer of 0 to 5; and each instance of R² is independently a C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group.

Also provided is a polymer of Formula 2:

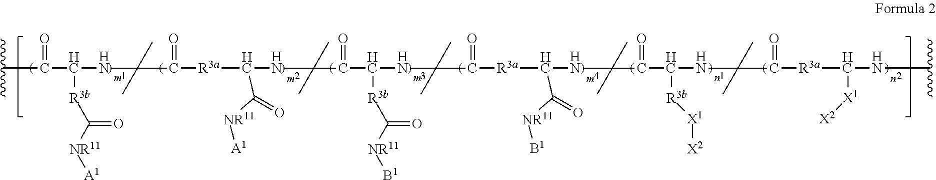

wherein:

each of m¹, m², m³, and m⁴ is an integer from 0 to 1000, provided that the sum of m¹+m²+m³+m⁴ is greater than 2;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each instance of R¹¹ is independently hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group;

each X¹ independently is —C(O)O—, —C(O)NR¹¹—, —C(O)—, —S(O)(O)—, or a bond;

each instance of X² is independently a hydrophobic group, such as a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof; any of which can be substituted with one or more substituents;

each instance of A¹ is independently:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

each instance of B¹ is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴-R⁵,

wherein each of p1 to p3 independently is an integer of 1 to 5, optionally wherein p1, p3, or both are greater than p2 and/or p1, p3, or both are integers of 3 to 5; r1 is an integer of 0 to 5; s1 is an integer from 1 to 5; each instance of R² is independently a C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof.

Additional polymers, compositions comprising same, and methods of preparing and using the polymers also are provided herein.

### BRIEF DESCRIPTION OF THE DRAWINGS

FIG. 1 provides the amino acid sequence of Cas9 from Streptococcus pyogene (SEQ ID NO:1).

FIG. 2 provides the amino acid sequence of Cpf1 from Francisella tularensis subsp. Novicida U112 (SEQ ID NO:2).

FIG. 3 provides the sequence of AsCpf1 (SEQ ID NO: 19).

FIG. 4 provides the sequence of LbCpf1 (SEQ ID NO: 20).

FIG. 5 shows the transfections efficiency of polymers ELN10, GenP9, GenP13, and GenP17 to deliver GFP mRNA to Hep3B cells.

FIG. 6 shows the payload functionality of polymers ELN10, GenP9, GenP13, and GenP17 upon delivery of GFP mRNA to Hep3B cells.

FIG. 7 shows the mouse weight percentage change from baseline after administration of polymer nanoparticles as described in Example 8.

FIG. 8 shows the in vivo bioluminescence body imaging of mice treated with polymer nanoparticles as described in Example 8.

FIG. 9 shows the quantification of the ex vivo bioluminescence intensity from lung tissue, spleen tissue, liver tissue, heart tissue, kidney tissue, thymus tissue, and skeletal muscle tissue of mice treated with polymer nanoparticles as described in Example 8.

FIG. 10 shows the histological analysis of delivery to the lungs and spleen of mice treated with polymer nanoparticles as described in Example 9. Expression of tdTomato RFP is shown in red and CD68+ macrophages are shown in green.

### DETAILED DESCRIPTION OF THE INVENTION

Provided herein is a polymer comprising a hydrolysable polymer backbone, the polymer backbone comprising (i) monomer units comprising a hydrophobic side chain; and (ii) monomer units comprising a cationic side chain; wherein the cationic side chain comprises a polyamine with at least one tertiary amine and only a single nucleophilic center, optionally at the terminus of the polyamine. The polymer can further comprise (iii) monomer units comprising a cationic side chain comprising a polyamine with at least one tertiary amine and a tissue-specific or cell-specific targeting moiety and/or a group comprising polyalkylene oxide, polyglycolic acid, or polylactic acid linked to the terminus of the polyamine. In some embodiments, the ratio of monomer units (iii) (when present) to monomer units (ii) is at least 1:1, optionally at least 2:1 or at least 5:1. For example, the monomer units (ii) comprising a cationic side chain with a polyamine with at least one tertiary amine and only a single nucleophilic center, optionally at the terminus of the polyamine, can be modified to include a tissue-specific or cell-specific targeting moiety and/or a group comprising polyalkylene oxide, polyglycolic acid, or polylactic acid linked to the terminus of a polyamine so as to provide monomer units (iii). In some embodiments, only a portion of the polyamines are so modified; in other embodiments substantially all or all of the polyamines are so modified. The tissue-specific or cell-specific targeting moiety and/or a group comprising polyalkylene oxide, polyglycolic acid, or polylactic acid can be linked to the terminus of the polyamine by any suitable means. Exemplary methods for incorporating the tissue-specific or cell-specific targeting moiety and/or a group comprising polyalkylene oxide, polyglycolic acid, or polylactic acid will be readily apparent from the disclosure provided herein.

As used herein, the term the term “polyamine” encompasses any chemical moiety comprising two or three or more amine groups (e.g., oligoamines) or larger polyamines comprising four or more (e.g., 4, 5, 6, 7, 8, 9, 10, 11, 12, 13, 14, 15, 16, or more) amine groups. Examples of polyamines include, for instance, polyalkylamines. The polyamines can be linear or branched. In some embodiments, the polyamines are linear.

As used herein, the term “polyalkylene oxide” encompasses any chemical moiety comprising two or three or more alkylene oxide groups (e.g., oligoalkylene oxide) or larger polyalkylene oxides comprising four or more (e.g., 4, 5, 6, 7, 8, 9, 10, 11, 12, 13, 14, 15, 16, 20, 25, 50, 75, 100 or more) alkylene oxide groups. Examples of polyalkylene oxide groups include polyethylene oxide (PEG), polypropylene oxide (PPO), or a mixture thereof (EO/PO). It will be understood to a person of ordinary skill in the art that the number of alkylene oxide groups can exist as a range of species having different amounts of alkylene oxide groups such that the reported number of alkylene oxide groups is the average.

As used herein, the terms “polyglycolic acid” encompasses any chemical moiety comprising two or three or more glycolic acid groups (e.g., oligoglycolic acid) or larger polyglycolic acids comprising four or more (e.g., 4, 5, 6, 7, 8, 9, 10, 11, 12, 13, 14, 15, 16, 20, 25, 50, 75, 100 or more) glycolic acid groups. It will be understood to a person of ordinary skill in the art that the number of glycolic acid groups can exist as a range of species having different amounts of glycolic acid groups such that the reported number of glycolic acid groups is the average.

As used herein, the term “polylactic acid” encompasses any chemical moiety comprising two or three or more lactic acid groups (e.g., oligolactic acid) or larger polylactic acids comprising four or more (e.g., 4, 5, 6, 7, 8, 9, 10, 11, 12, 13, 14, 15, 16, 20, 25, 50, 75, 100 or more) lactic acid groups. It will be understood to a person of ordinary skill in the art that the number of lactic acid groups can exist as a range of species having different amounts of lactic acid groups such that the reported number of lactic acid groups is the average.

As used herein, the phrase “hydrolysable polymer backbone” refers to a polymer backbone having bonds that are susceptible to cleavage under physiological conditions (e.g., physiological pH, physiological temperature, or in a given in vivo tissue such as blood, serum, etc. due to naturally occurring factors (e.g., enzymes). Generally, the hydrolysable polymer backbone comprises a polyamide, poly-N-alkylamide, polyester, polycarbonate, polycarbamate, or a combination thereof. In certain embodiments, the hydrolysable polymer backbone comprises a polyamide.

The polymer comprises monomer units with a side chain comprising a hydrophobic group (e.g., a hydrophobic side chain). The monomer can comprise any hydrophobic group. Examples of hydrophobic groups include, for instance, a C₁-C₁₂ (e.g., C₂-C₁₂, C₂-C₁₀, C₂-C₈, C₂-C₆, C₃-C₁₂, C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅-C₁₀,) alkyl or heteroalkyl group, a C₂-C₁₂ (e.g., C₂-C₆, C₃-C₁₂, C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅-C₁₀,) alkenyl group, a C₃-C₁₂ (C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅-C₁₀,) cycloalkyl group or cycloalkenyl group, and an aryl group. In some embodiments, the hydrophobic group is an aliphatic group. In certain embodiments, the hydrophobic group comprises a C₄-C₁₂ alkyl, alkenyl, cycloalkyl, or cycloalkenyl group. In some embodiments, the hydrophobic group comprises fewer than 10 carbons or fewer than 8 carbons. For example, the hydrophobic group can comprise a C₂-C₁₀, C₂-C₈, or C₂-C₆ (e.g., C₃-C₈ or C₃-C₆) alkyl group. The alkyl or alkenyl groups can be branched or straight-chain. In any of the foregoing emodiments, the hydrophobic group can be linked to the polymer backbone direclty or via a linkage comprising, for instance, an ester, an amide, or an ether group, optionally further comprising an alkylene linker (e.g., a methylene or ethylene linker).

The polymer also comprises monomer units with a cationic side chain; wherein the cationic side chain comprises a polyamine with at least one tertiary amine and only a single nucleophilic center, optionally at the terminus of the polyamine. The amine groups can be primary amine groups, secondary amine groups, tertiary amine groups, or any combination thereof, so long as the polyamine has at least one tertiary amine and only a single nucleophilic center, optionally at the terminus of the polyamine. In some embodiments, the polyamine comprises only one primary amine or only one secondary amine as the single nucleophilic center (e.g., at the terminus of the polyamine), and all other amines in the polyamine are tertiary amines. The polyamine can be linked to the polymer backbone (the monomer of the backbone) directly or via a linkage, for instance, an ester, an amide, or an ether group, optionally further comprising an alkylene linker (e.g., a methylene or ethylene linker).

In some embodiments, the polyamine is of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5 (e.g., 1, 2, 3, 4, or 5); r1 is an integer of 0 to 5 (e.g., 1, 2, 3, 4, or 5); and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In some embodiments, p1, p3, or both are greater than p2 and/or p1, p3, or both are integers of 3 to 5 (e.g., 3, 4, or 5) and, optionally, p2 is an integer of 1 or 2. In some embodiments, the polyamine is —(CH₂)ₚ₁—[NR²—(CH₂)ₚ₂—]ᵣ₁NR²—(CH₂)ₚ₃—NH₂, optionally wherein R² is independently a C₁-C₆ alkyl group (e.g., methyl or ethyl). In some embodiments, the poly amine is —(CH₂)ₚ₁—[NR²—(CH₂)ₚ₂—]ᵣ₁NR²—(CH₂)ₚ₃—NHR², optionally wherein R² is independently a C₁-C₆ alkyl group (e.g., methyl or ethyl) group. In further embodiments comprising any of these features, p1, p3, or both can be greater than p2 and/or p1, p3, or both can be integers of 3 to 5 (e.g., 3, 4, or 5).

In some embodiments, the polymer described above having monomer units (i) comprising a hydrophobic side chain, and monomer units (ii) comprising a cationic side chain with a polyamine with at least one tertiary amine and only a single nucleophilic center, optionally at the terminus of the polyamine, can be modified at the terminus of the polyamine to include a group comprising a polyalkylene oxide, a polyglycolic acid, or a polylactic acid (including any combination thereof); a tissue-specific or cell-specific targeting moiety; or both types of groups (e.g., a group comprising a polyalkylene oxide, a polyglycolic acid, or a polylactic acid linking a tissue-specific or cell-specific targeting moiety to the terminus of the polyamine). Some portion or all of the cationic side chances can be so modified. Thus, in some embodiments, there is provided a polymer comprising (i) monomer units comprising a hydrophobic side chain as described above, and (ii) monomer units comprising a cationic side chain of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,

wherein p1 to p3, r1, s1, and R² are as described above; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid or combination thereof; a tissue-specific targeting moiety, a cell-specific targeting moiety, or a combination thereof. In some embodiments, at least a portion (or all) of the monomer units comprise a cationic side chain of with an R⁵ group:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵.

In some embodiments, the monomer units comprise a cationic side chain of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵;

optionally wherein at least some portion (or all) comprise a side chain of the formula:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵.

In some of any of the foregoing embodiments, p1, p3, or both are greater than p2, and/or p1, p3, or both are integers of 3 to 5 (e.g., 3, 4, or 5), optionally wherein p2 is 1 or 2. In some embodiments, R² is a C₁-C₆ alkyl group, such as a C₁-C₃ alkyl group (e.g., methyl or ethyl).

In some embodiments, the polymer further comprises additional monomer units, such as monomer units with a side chain comprising an ionizable group. As used herein, the phrase “ionizable group” refers to any chemical moiety with a substituent that can readily be converted into a charged species. For example, the ionizable group can be a group that is a proton-donor or proton-acceptor. The group can be protonated or deprotonated under physiological conditions. In certain embodiments, the ionizable group has a pKa less than 7 (in water at 25° C.). For example, the ionizable group described herein can have a pKa of less than 6, a pKa of less than 5, a pKa of less than 4, a pKa of less than 3, a pKa of less than 2, or a pKa of less than 1. Alternatively, or additionally, the ionizable group described herein can have a pKa of greater than −2, a pKa of greater than −1, a pKa of greater than 0, a pKa of greater than 1, a pKa of greater than 2, a pKa of greater than 3, a pKa of greater than 4, a pKa of greater than 5, or a pKa of greater than 6. Accordingly, the ionizable group described herein can have a pKa from −2 to 7, for example, a pKa from −1 to 7, a pKa from 0 to 7, a pKa from 1 to 7, a pKa from 2 to 7, a pKa from 3 to 7, a pKa from 4 to 7, a pKa from 5 to 7, a pKa from 6 to 7, a pKa from 0 to 6, a pKa from 2 to 6, a pKa from 4 to 6, a pKa from 0 to 5, a pKa from 2 to 5, or a pKa from 4 to 5. Examples of ionizable groups include, for instance, sulfonic acid, sulfonamide, carboxylic acid, thiol, phenol, amine salt, imide, and amide groups.

The polymer also can, optionally, comprise further monomer units with a side chain comprising a polyalkylene oxide, polyglycolic acid, polylactic acid, or combination thereof linked to the polymer backbone (e.g., without an intervening polyamine group as described herein with respect to other optional monomers). Such an embodiment might be particularly useful when R⁵ is absent or is something other than a polyalkylene oxide, polyglycolic acid, polylactic acid, but can also be employed together with a polymer comprising such groups attached to the polyamine as well. The polyalkylene oxide, polyglycolic acid, polylactic acid, or combination thereof can be linked to the polymer backbone in any suitable manner, such as directly or via a linkage comprising, for instance, an ester, an amide, or an ether group, optionally further comprising an alkylene linker (e.g., a methylene or ethylene linker). In some embodiments, the side chain comprises at least one polyethylene glycol group (a.k.a. polyethylene oxide group) having a sum total of from 2 to 200 ethylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polypropylene oxide group having a sum total of from 2 to 200 propylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polylactic acid and/or polyglycolic acid group having a sum total of from 2 to 200 polylactic acid and/or polyglycolic acid units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units. In certain embodiments, the side chain comprises at least one polyethylene glycol/polypropylene oxide group having a sum total of from 2 to 200 ethylene glycol and propylene oxide units. The side chain can comprise the ethylene glycol, propylene oxide, polylactic acid, and/or polyglycolic acid units in any suitable structure, such as an alternating polymer, random polymer, block polymer, graft polymer, linear polymer, branched polymer, cyclic polymer, or a combination thereof. In certain embodiments, the side chain comprises polyalkylene oxide units (e.g., ethylene oxide units, propylene oxide units, or both) and optionally further comprises polylactic acid or polyglycolic acid units. For example, the side chain of at least some of the monomers can be derived from PLURONIC® F65 or PLURONIC® F127.

By way of further illustration, the polymer can comprise monomer units with a side chain comprising a polyalkylene oxide, polyglycolic acid, polylactic acid, or combination thereof, which comprise a structure of the following formula:

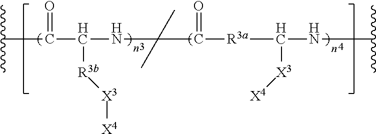

wherein:

each of n³ and n⁴ is an integer from 0 to 1000, provided that the sum of n³+n⁴ is greater than 1;

the symbol “/” indicates that the units separated thereby are linked randomly or in any order;

each instance of R^{3a} is independently a methylene or ethylene group;

each instance of R^{3b} is independently a methylene or ethylene group;

each X³ independently is —C(O)O—, —C(O)NR¹¹—, —C(O)—, —S(O)(O)—, or a bond;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X⁴ comprises polyalkylene oxide, polyglycolic acid, polylactic acid, or a combination thereof; and

each instance of p is independently an integer from 2 to 200.

In some embodiments, the polymer has an overall pKa of less than 7 (in water at 25° C.). For example, the polymer described herein can have a pKa of less than 6, a pKa of less than 5, a pKa of less than 4, a pKa of less than 3, a pKa of less than 2, or a pKa of less than 1. Alternatively, or additionally, the polymer described herein can have a pKa of greater than −2, a pKa of greater than −1, a pKa of greater than 0, a pKa of greater than 1, a pKa of greater than 2, a pKa of greater than 3, a pKa of greater than 4, a pKa of greater than 5, or a pKa of greater than 6. Accordingly, the polymer described herein can have a pKa from −2 to 7, for example, a pKa from −1 to 7, a pKa from 0 to 7, a pKa from 1 to 7, a pKa from 2 to 7, a pKa from 3 to 7, a pKa from 4 to 7, a pKa from 5 to 7, a pKa from 6 to 7, a pKa from 0 to 6, a pKa from 2 to 6, a pKa from 4 to 6, a pKa from 0 to 5, a pKa from 2 to 5, or a pKa from 4 to 5.

The polymer can comprise any suitable number or amount (e.g., weight or number percent composition) of the monomer units with a side chain comprising a hydrophobic group, the monomer units with a side chain comprising a polyamine, and, when present, the monomer units with a side chain comprising an ionizable group. In some embodiments, the polymer comprises about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having a hydrophobic group, about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having a polyamine side chain; and 0 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having an ionizable group and/or monomer units having a polyalkylene oxide, polyglycolic acid, polylactic acid, or combination thereof.

As used herein, “alkyl” or “alkylene” refers to a substituted or unsubstituted hydrocarbon chain. The alkyl group can have any number of carbon atoms (e.g., C₁-C₁₀₀ alkyl, C₁-C₅₀ alkyl, C₁-C₁₂ alkyl, C₁-C₈ alkyl, C₁-C₆ alkyl, C₁-C₄ alkyl, C₁-C₂ alkyl, etc.). The alkyl or alkylene can be saturated, or can be unsaturated (e.g., to provide an alkenyl or alkynyl), and can be linear, branched, straight-chained, cyclic (e.g., cycloalkyl or cycloalkenyl), or a combination thereof. Cyclic groups can be monocyclic, fused to form bicyclic or tricyclic groups, linked by a bond, or spirocyclic. In some embodiments, the alkyl substituent can be interrupted by one or more heteroatoms (e.g., oxygen, nitrogen, and sulfur), thereby providing a heteroalkyl, heteroalkylene, or heterocyclyl (i.e., a heterocyclic group). In some embodiments, the alkyl is substituted with one or more substituents.

The term “aryl” refers to an aromatic ring system having any suitable number of ring atoms and any suitable number of rings. Aryl groups can include, for instance, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. In some embodiments, the aryl group comprises an alkylene linking group so as to form an arylalkyl group (e.g., a benzyl group). Some aryl groups have from 6 to 12 ring members, such as phenyl, naphthyl or biphenyl. Other aryl groups have from 6 to 10 ring members, such as phenyl or naphthyl. In some embodiments, the aryl substituent can be interrupted by one or more heteroatoms (e.g., oxygen, nitrogen, and sulfur), thereby proving a heterocyclyl (i.e., a heterocyclic or heteroaryl group). In some embodiments, the aryl is substituted with one or more substituents.

The term “heterocyclyl,” or “heterocyclic group” refers to a cyclic group, e.g., aromatic (e.g., heteroaryl) or non-aromatic where the cyclic group has one or more heteroatoms (e.g., oxygen, nitrogen, and sulfur). In some embodiments, the heterocyclyl or heterocyclic group (i.e., cyclic group, e.g., aromatic (e.g., heteroaryl) or non-aromatic where the cyclic group has one or more heteroatoms) is substituted with one or more substituents.

As used herein, the term “substituted” can mean that one or more hydrogens on the designated atom or group (e.g., substituted alkyl group) are replaced with another group provided that the designated atom's normal valence is not exceeded. For example, when the substituent is oxo (i.e., ═O), then two hydrogens on the atom are replaced. Substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

In some embodiments, the polymer provided herein is a polymer comprising a structure of Formula 1:

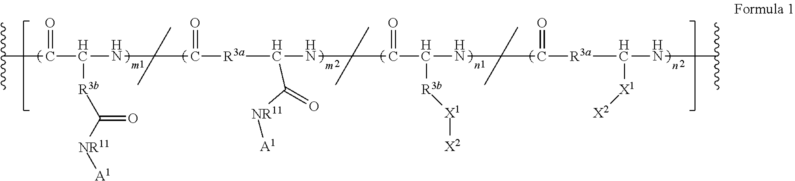

wherein:

each of m¹ and m² is an integer from 0 to 1000, provided that the sum of m¹+m² is greater than 2;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X¹ independently is —C(O)O—, —C(O)NR¹¹—, —C(O)—, —S(O)(O)—, or a bond;

each instance of X² is a hydrophobic side chain;

each instance of A¹ is a polyamine with at least one tertiary amine and only a single nucleophilic center, optionally at the terminus of the polyamine.

In some embodiments of the polymer of Formula 1, each instance of A¹ is independently a group of formula:

—(CH₂)ₚ₁—[NR²—(CH₂)ₚ₂—]ᵣ₁NR²—(CH₂)ₚ₃—NH₂, or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5 (e.g., 1, 2, 3, 4, or 5), optionally wherein p1, p3, or both are optionally greater than p2, and/or wherein p1, p3, or both are integers of 3 to 5 (e.g., 3, 4, or 5); r1 is an integer of 0 to 5 (e.g., 0, 1, 2, 3, 4, or 5); and each instance of R² is independently a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group. In some embodiments, each instance of A¹ is independently:

—(CH₂)ₚ₁—[NR²—(CH₂)ₚ₂—]ᵣ₁NR²—(CH₂)ₚ₃—NH₂,

In other embodiments, each instance of A¹ is independently:

—(CH₂)ₚ₁—[NR²—(CH₂)ₚ₂—]ᵣ₁NR²—(CH₂)ₚ₃—NHR².

Further, each instance of R² can be, in some embodiments, a C₁-C₆ (e.g., C₁-C₆, C₁-C₄, or C₁-C₃) alkyl group, C₂-C₆ (e.g., C₂-C₅, C₂-C₄, or C₂-C₃) alkenyl group, C₃-C₆ (e.g., C₃-C₅ or C₃-C₄) cycloalkyl group, or C₃-C₆ (e.g., C₃-C₅ or C₃-C₄) cycloalkenyl group. In certain embodiments, R² is a C₁-C₆ (e.g., C₁-C₆, C₁-C₄, or C₁-C₃) alkyl group, such as a methyl or ethyl group. In conjunction with any of the foregoing embodiments, p1, p3, or both are optionally greater than p2, and/or p1, p3, or both are integers of 3 to 5 (e.g., 3, 4, or 5).

Specific non-limiting examples of A¹ groups include, for instance, —CH₂—CH₂—CH₂—N(CH₃)—CH₂—CH₂— N(CH₃)—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—CH₂—NH₂; or —CH₂—CH₂—CH₂—N(CH₃)—CH₂—CH₂—CH₂—NHCH₃.

In any of the foregoing embodiments, R^{3a} and R^{3b} are each independently a methylene or ethylene group. In some embodiments, R^{3a} is an ethylene group and R^{3b} is a methylene group; or R^{3a} is a methylene group and R^{3b} is an ethylene group. In certain embodiments, R^{3a} and R^{3b} are both ethylene groups. In some embodiments, R^{3a} and R^{3b} are both methylene groups.

In any of the foregoing embodiments, each X¹ group of Formula 1 is independently-C(O)O—, —C(O)NR¹¹—, —C(O)—, —S(O)(O)—, or a bond. Each X¹ group can be the same or different from one another. In some embodiments, X¹ is —C(O)NR¹¹—. In some embodiments, X¹ is —C(O)O—.

In any of the foregoing embodiments, each instance of R¹¹ is independently hydrogen or a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, aryl group, or heterocyclic group (e.g., 3-12, 3-10, 3-8, or 3-6 membered heterocyclic group comprising one, two, or three heteroatoms), any of which can be substituted with one or more substituents. In some embodiments, R¹¹ is a C₁-C₁₂ alkyl group (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) which can be linear or branched. In certain embodiments, each R¹¹ is hydrogen or a C₁-C₃ alkyl group. In some embodiments, R¹¹ is methyl; in other embodiments, R¹¹ is hydrogen. Each R¹¹ is independently chosen and can be the same or different; however, in some embodiments, each R¹¹ is the same (e.g., all methyl or all hydrogen).

In any of the foregoing embodiments, each instance of X² is a hydrophobic side chain. For example, each instance of X² independently can be a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, aryl group, or heterocyclic group (e.g., 3-12, 3-10, 3-8, or 3-6 membered heterocyclic group comprising one, two, or three heteroatoms) or combination thereof, any of which can be substituted with one or more substituents. In some embodiments, X² is an aliphatic group. In some embodiments, X² optionally can comprise one or more primary, secondary, or tertiary amines. Each X² is independently selected and, therefore, can be the same or different from one another. In certain embodiments, each instance of X² is independently a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, or combination thereof optionally comprising one or more primary, secondary, or tertiary amines. In some embodiments, one or more (or all) X² groups can be independently a C₂-C₁₂ (e.g., C₃-C₁₂, C₃-C₈, C₃-C₆, C₄-C₁₂, C₄-C₆, C₆-C₁₂, or C₅-C₁₂) alkyl group or alkenyl group, or C₃-C₁₂ (e.g., C₃-C₈, C₃-C₆, C₄-C₁₂, C₄-C₆, C₆-C₁₂, or C₅-C₁₂) cycloalkyl group or cycloalkenyl group. In other embodiments, one or more (or all) X² groups can be independently a C₁-C₈ (e.g., C₁-C₆, C₁-C₄, C₁-C₃, C₂-C₈, or C₂-C₆) alkyl groups. Any of the foregoing alkyl or alkenyl groups can be linear or branched.

In some embodiments, the polymer has structure of Formula 1A:

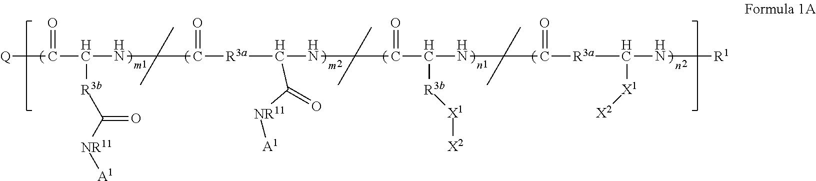

wherein

Q is of formula:

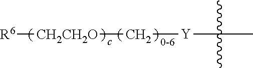

c is an integer from 0 to 50;

Y is optionally present and is a cleavable linker;

R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and

R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety.

All other aspects of Formula 1A are as described with respect to Formula 1, above, including any and all embodiments thereof.

In some embodiments, the polymer has the structure of Formula 1B:

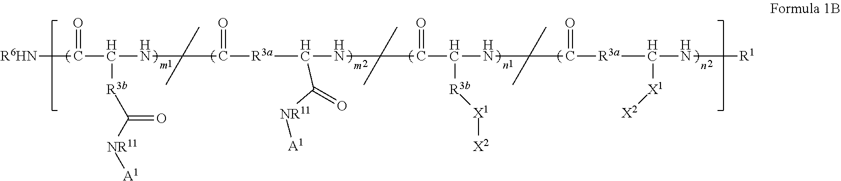

wherein

R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and

R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety.

All other aspects of Formula 1B are as described with respect to Formula 1 and 1A, including any and all embodiments thereof.

In some embodiments, the polymer has the structure of Formula 1C:

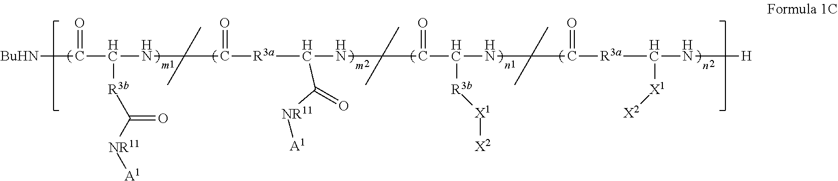

All other aspects of Formula 1C are as described with respect to Formulas, 1, 1A, and 1B, including any and all embodiments thereof.

In some embodiments of the polymer comprising a structure of Formula 1 (or Formulas 1A-1C), each of m¹ and m² is an integer from 0 to 1000 (e.g., 0 to 500, 0 to 200, 0 to 100, or 0 to 50), provided that the sum of m¹+m² is greater than 2 or greater than 5 (e.g., 5-2000, 5-1000, 5-500, 5-100, or 5-50). In some embodiments, the sum of m¹+m² is greater than 10 or greater than 20 (e.g., 10-5000, 10-2000, 10-1000, 10-500, 10-100, or 10-50; or 20-5000, 20-2000, 20-1000, 20-500, 20-100, or 20-50). In some embodiments, m¹+m² is about 20-50 or 20-40. Furthermore, each of n¹ and n² is an integer from 0 to 1000 (e.g., 0 to 500, 0 to 200, 0 to 100, 0 to 50, or 0-25), provided that the sum of n¹+n² is greater than 2 (e.g., 2-2000, 2-1000, 2-500, 2-200, 2-100, 2-50, or 2-25). In some embodiments, the sum of n¹+n² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25). In some embodiments, the sum of n¹+n² is about 20-50 or 20-40, optionally wherein m¹+m² is about 20-50 or 20-40.

The polymer according to Formula 1 can have any suitable degree of polymerization (e.g., sum of m¹+m²+n¹+n²). In some instances, the polymer has a degree of polymerization of about 20 or more (e.g., about 20 to about 1000, or about 20 to about 100, or about 20 to about 65, or about 20 to 64). In some instances, the polymer has a degree of polymerization of about 50 or more (e.g., about 50 to about 1000, or about 50 to about 500, or 66 to about 200).

The polymer can comprise any suitable ratio of monomers comprising A1 groups (“A monomers”) to monomers comprising X² groups (“X monomers”) (e.g., the ratio of (m¹+m²)/(n¹+n²)). In some embodiments, the polymer comprises a ratio of A monomers to X monomers of about 25 or less (e.g., about 10 or less, about 5 or less, about 3 or less, or about 2 or less) and, optionally, about 0.2 or more (e.g., about 0.3 or more, 0.4 or more, 0.5 or more, 0.6 or more, 0.7 or more, or about 1 or more). For example, the ratio of A monomers to X monomers can be about 0.3 to about 25, from about 0.3 to about 20, from about 0.3 to about 10, from about 0.3 to about 5, from about 0.3 to about 3, from about 0.3 to about 2. In some embodiments, the polymer comprises a ratio of A monomers to X monomers of about 0.2 to about 3 (e.g., about 0.3 to about 2), optionally wherein the sum of n¹+n² is about 20-50 or 20-40, and/or wherein the sum of m¹+m² is about 20-50 or 20-40.

Also provided herein is a polymer comprising a structure of Formula 2:

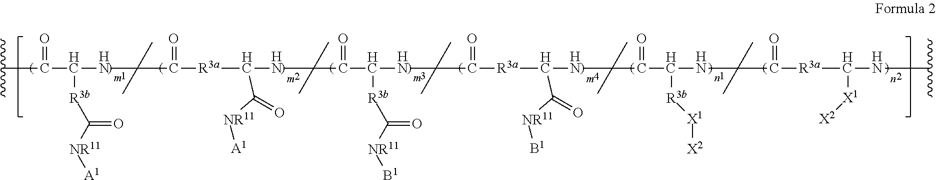

wherein:

each of m¹, m², m³, and m⁴ is an integer from 0 to 1000, provided that the sum of m¹+m²+m³+m⁴ is greater than 2;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each instance of R¹¹ is independently hydrogen, an aryl group, a heterocyclic group, a C₁-C₁₂ alkyl group, alkenyl group, cycloalkyl group, or cycloalkenyl group, any of which can be optionally substituted with one or more substituents;

each X¹ independently is —C(O)O—, —C(O)NR¹—, —C(O)—, —S(O)(O)—, or a bond;

each instance of X² is a hydrophobic side chain (e.g., hydrophobic group);

each instance of A¹ is a polyamine with at least one tertiary amine and only a single nucleophilic center, optionally at the terminus of the polyamine; and each instance of B¹ is a polyamine with at least one tertiary amine and a polyalkylene oxide group, a polyglycolic acid group, a polylactic acid group, a tissue-specific targeting moiety, a cell-specific targeting moiety, or a combination thereof linked to the terminus of the polyamine.

In some embodiments, each instance of A¹ is independently:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

and each instance of B¹ is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵;

or wherein each instance of B¹ is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵;

In some embodiments, each instance of A¹ is independently:

—(CH₂)ₚ₁—[NR²—(CH₂)ₚ₂—]ᵣ₁NR²—(CH₂)ₚ₃—NH₂;

and each instance of B¹ is independently:

—(CH₂)ₚ₁—[NR²—(CH₂)ₚ₂—]ᵣ₁NR²—(CH₂)ₚ₃—NH—(CH₂)ₛ₁—R⁴-R⁵.

In other embodiments, each instance of A¹ is independently:

—(CH₂)ₚ₁—[NR²—(CH₂)ₚ₂—]ᵣ₁NR²—(CH₂)ₚ₃—NHR²;

and each instance of B¹ is independently:

—(CH₂)ₚ₁—[NR²—(CH₂)ₚ₂—]ᵣ₁NR²—(CH₂)ₚ₃—NR²—(CH₂)ₛ₁—R⁴-R⁵.

In groups A¹ and B¹, variables p1 to p3 (i.e., p1, p2, and p3) are each independently an integer of 1 to 5 (e.g., 1, 2, 3, 4, or 5). In some embodiments, p1, p3, or both are integers greater than the integer of p2. In addition, or alternatively, p1 or p3, or both are integers of 3 to 5 (e.g., 3, 4, or 5). In certain embodiment both of p1 and p3 are integers greater than p2, or both are integers of 3 to 5 (e.g., 3, 4, or 5).

In groups A¹ and B¹, variables r1 and s1 are each independently an integer of 1 to 5 (e.g., 1, 2, 3, 4, or 5). In some embodiments, r1 and/or s1 are each independently an integer of 1 to 3 (e.g., 1, 2, or 3).

Each instance of R² can be a C₁-C₁₂ (e.g., C₁-C₆, C₁-C₄, or C₁-C₃) alkyl group, C₂-C₁₂ (e.g., C₂-C₆, C₂-C₄, or C₂-C₃) alkenyl group, C₃-C₁₂ (e.g., C₃-C₈, C₃-C₆ or C₃-C₄) cycloalkyl group, or C₃-C₁₂ (e.g., C₃-C₈, C₃-C₆ or C₃-C₄) cycloalkenyl group, or R² is combined with a second R² so as to form a heterocyclic group. In certain embodiments, R² is a C₁-C₆ (e.g., C₁-C₄ or C₁-C₃) alkyl group, such as an ethyl or methyl group.

Specific non-limiting examples of A¹ groups include, for instance, —CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—N(CH₃)—CH₂—CH₂—CH₂—NH₂; —CH₂—CH₂—CH₂—N(CH₃)—CH₂—CH₂—N(CH₃)—CH₂—CH₂—CH₂—NHCH₃; —CH₂—CH₂—CH₂—N(CH₃)—CH₂—CH₂—CH₂—NH₂; or —CH₂—CH₂—CH₂—N(CH₃)—CH₂—CH₂—CH₂—NHCH₃.

Specific non-limiting examples of B¹ groups include, for instance, —CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂— NR²—(CH₂)ₛ₁—R⁴-R⁵; —CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂— NH—(CH₂)ₛ₁—R⁴-R⁵, —CH₂—CH₂—CH₂—N(CH₃)—CH₂—CH₂—N(CH₃)—CH₂—CH₂—N(CH₃)—CH₂—CH₂—CH₂— NR²—(CH₂)ₛ₁—R⁴-R⁵; —CH₂—CH₂—CH₂—N(CH₃)—CH₂—CH₂—N(CH₃)—CH₂—CH₂—N(CH₃)—CH₂—CH₂—CH₂— NH—(CH₂)ₛ₁—R⁴-R⁵; —CH₂—CH₂—CH₂—N(CH₃)—CH₂—CH₂—N(CH₃)—CH₂—CH₂—CH₂— NR²—(CH₂)ₛ₁—R⁴-R⁵; —CH₂—CH₂—CH₂—N(CH₃)—CH₂—CH₂—N(CH₃)—CH₂—CH₂—CH₂— NH—(CH₂)ₛ₁—R⁴-R⁵; —CH₂—CH₂—CH₂—N(CH₃)—CH₂—CH₂—CH₂— NR²—(CH₂)ₛ₁—R⁴-R⁵; or —CH₂—CH₂—CH₂—N(CH₃)—CH₂—CH₂—CH₂— NH—(CH₂)ₛ₁—R⁴-R⁵.

In accordance with any of the foregoing embodiments, each instance of R⁴ is independently —C(O)O—, —C(O)NH—, —C—O—C(O)—O—C—, —O—, or —S(O)(O)—. In some embodiments, each instance of R⁴ is independently —C(O)O— or —C(O)NH—. In certain embodiments, each instance of R⁴ is —C(O)O—. In certain embodiments, each instance of R⁴ is —C(O)NH—.

In accordance with any of the foregoing embodiments, each instance of R⁵ is a group comprising a polyalkylene oxide, a polyglycolic acid, a polylactic acid, or combination thereof, a tissue-specific targeting moiety; a cell-specific targeting moiety; or a combination thereof, optionally including a linking group. In some embodiments, each instance of R⁵ is a tissue-specific targeting moiety or a cell-specific targeting moiety. In some embodiments, each instance of R⁵ is a group comprising a polyalkylene oxide, a polyglycolic acid, a polylactic acid, or a combination thereof, optionally in combination with a tissue-specific targeting moiety or a cell-specific targeting moiety. For example, R⁵ can be a group comprising a polyalkylene oxide, a polyglycolic acid, a polylactic acid, or a combination thereof, linking a tissue-specific or cell-specific targeting moiety to the polyamine (e.g., a PEG, PGA, or PLA group which has been capped with a tissue-specific targeting moiety or a cell-specific targeting moiety). The polyalkylene oxide group, polyglycolic acid group, polylactic acid group, or combination thereof, can be linked to the polymer (e.g., the terminus of the polyamine side chain) in any suitable manner.

In some embodiments, R⁵ comprises at least one polyethylene glycol group (a.k.a. polyethylene oxide group) having a sum total of from 2 to 200 ethylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, R⁵ comprises at least one polypropylene oxide group having a sum total of from 2 to 200 propylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, R⁵ comprises at least one polylactic acid and/or polyglycolic acid group having a sum total of from 2 to 200 polylactic acid and/or polyglycolic acid units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In certain embodiments, R⁵ comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units. Without wishing to be bound by any particular theory, it is believed that the polyethylene glycol/polypropylene oxide group may enhance biodistribution and/or reduced toxicity when injected in vivo, and the polylactic acid and/or polyglycolic acid group may help to adjust the zeta potential of the positively charged nanoparticles (e.g., to negatively charged or neutral). In embodiments where R⁵ comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units, R⁵ can exist as any suitable structure type. For example, R⁵ can comprise the ethylene glycol and/or propylene oxide units and the polylactic acid and/or polyglycolic acid units as an alternating polymer, random polymer, block polymer, graft polymer, linear polymer, branched polymer, cyclic polymer, or a combination thereof. In certain embodiments, R⁵ comprises polyalkylene oxide units (e.g., ethylene oxide units, propylene oxide units, or both) and optionally further comprises polylactic acid or polyglycolic acid units. For example, R⁵ of at least some of the monomers can be derived from PLURONIC® F65 or PLURONIC® F127.

In some embodiments, each instance of R⁵ is independently of the formula:

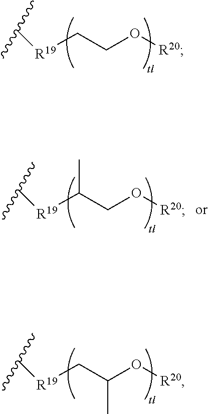

wherein:
R¹⁹ is a bond or a methylene, ethylene, or propylene group; R²⁰ is hydrogen, an aryl group (e.g., a C₆-C₁₂ alkyl group), a heterocyclic group (e.g., a C₂-C₁₂ heterocyclic group), a alkyl group (e.g., a C₁-C₁₂ alkyl group), alkenyl group (e.g., C₂-C₁₂ alkenyl group), cycloalkyl group (e.g., a C₃-C₁₂ cycloalkyl group), or cycloalkenyl group (e.g., a C₃-C₁₂ cycloalkenyl group), any of which can be optionally substituted with one or more substituents, optionally hydrogen or a C₁-C₃ alkyl group; and 11 is an integer from 2 to 200 (e.g., from 2 to 150, from 2 to 100, from 2 to 50, from 10 to 200, from 10 to 150, from 10 to 100, from 10 to 50, from 25 to 200, from 25 to 150, from 25 to 100, from 25 to 50, from 50 to 200, from 50 to 150, or from 50 to 100).

The tissue-specific or cell-specific targeting moiety referred to with respect to any of the foregoing polymers can be any small molecule, protein (e.g., antibody or antigen), amino acid sequence, sugar, oligonucleotide, metal-based nanoparticle, or combination thereof, capable of recognizing (e.g., specifically binding) a given target tissue or cell (e.g., specifically binding a particular ligand, receptor, or other protein or molecule that allows the targeting moiety to discriminate between the target tissue or cell and other non-target tissues or cells). In some embodiments, the tissue-specific or cell-specific targeting moiety is a small molecule (e.g., a small molecule drug or other small-molecule moiety with less than about 900 daltons or less than about 500 daltons, and/or having fewer than about 100 atoms, such as about 10-100 or about 20-100 atoms). In other embodiments, the tissue-specific or cell-specific targeting moiety is a receptor for a ligand, or a ligand for a receptor.

The tissue-specific or cell-specific targeting moiety can be used to target any desired tissue or cell type. In some embodiments, the tissue-specific or cell-specific targeting moiety localizes the polymer to tissues of the peripheral nervous system, the central nervous system, liver, muscle (e.g., cardiac muscle), lung, bone (e.g., hematopoietic cells), or the eye of the subject. In certain embodiments, the tissue-specific or cell-specific targeting moiety localizes the polymer to tumor cells. In a particular embodiment, tissue-specific or cell-specific targeting moiety targets lung tissue. For example, the tissue-specific or cell-specific targeting moiety can be a sugar that binds to a receptor on a specific tissue or cell.

Illustrative, non-limiting examples of tissue-specific or cell-specific targeting moieties include:

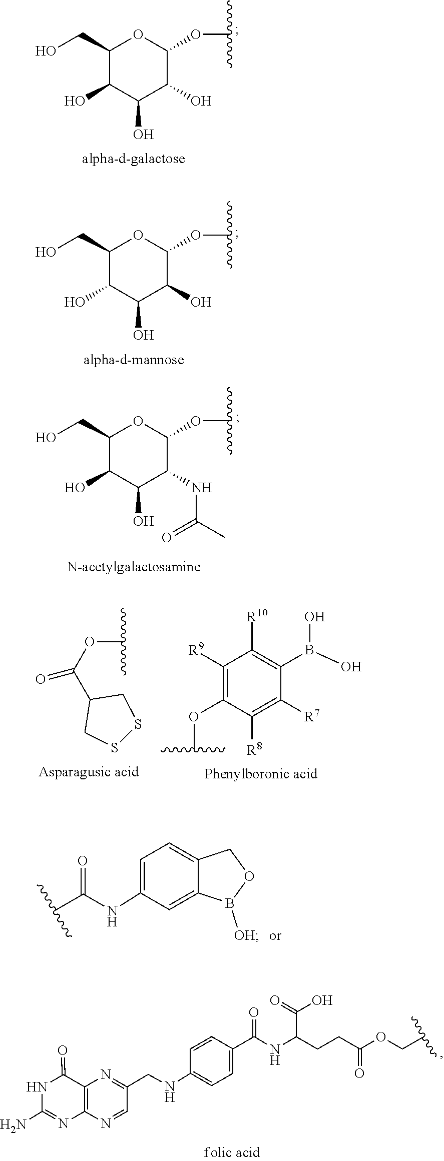

wherein each of R⁷, R⁸, R⁹, and R¹⁰ is independently hydrogen, halogen, C₁-C₄ alkyl, or C₁-C₄ alkoxy, optionally substituted with one or more amino groups. The specified tissue-specific or cell-specific targeting moieties can be chosen to localize the polymer to a tissue described herein. For example, alpha-d-mannose can be used to localize the polymer to the peripheral nervous system, the central nervous system, or immune cells, alpha-d-galactose and N-acetylgalactosamine can be used to localize the polymer to liver hepatocytes, and folic acid can be used to localize the polymer to tumor cells.

All other aspects of the polymer of Formula 2 are as described with respect to Formula 1, including all embodiments thereof with respect to substituents of Formula 2. Thus, for instance, in some embodiments of Formula 2, each instance of R¹¹ can be any group as described with respect to Formula 1, including specific embodiments in which R¹¹ is hydrogen or methyl, and each instance of R^{3a} and R^{3b} can be any group as described with respect to Formula 12, including embodiments wherein R^{3a} and R³ are methylene or ethylene. Similarly, X¹ and X² can be any group as described with respect to Formula 1, including embodiments wherein X¹ is —C(O)NR¹¹— or —C(O)O— and/or one or more (or all) X² groups can be independently a C₁-C₈ (e.g., C₁-C₆, C₁-C₄, C₁-C₃, C₂-C₈, or C₂-C₆) alkyl group.

In some embodiments, the polymer has structure of Formula 2A:

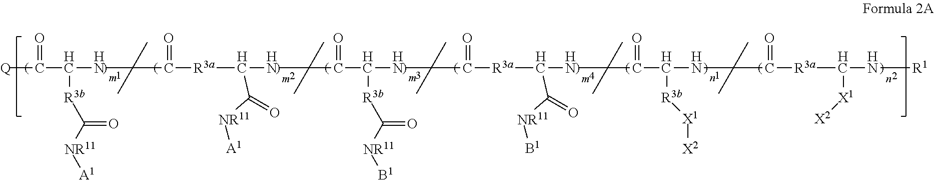

wherein

Q is of formula:

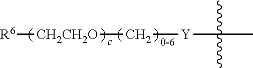

c is an integer from 0 to 50;

Y is optionally present and is a cleavable linker;

R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and

R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety.

All other aspects of Formula 2A are as described with respect to Formula 2, above, including any and all embodiments thereof.

In some embodiments, the polymer has structure of Formula 2B:

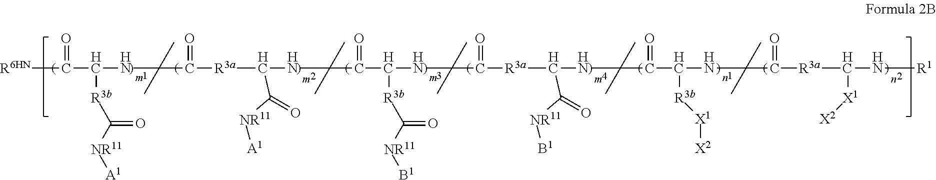

wherein

R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and

R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety.

In some embodiments, R¹ and/or R⁶ is a C₁-C₁₂ alkyl (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group), which can be linear or branched, optionally substituted with one or more substituents. In certain embodiments, the heteroalkyl or alkyl group comprises or is substituted with one or more amines, for instance, from 2 to 8 (i.e., 2, 3, 4, 5, 6, 7, or 8) tertiary amines. The tertiary amines can be a part of the heteroalkyl backbone chain or pendant substituents.

All other aspects of Formula 2B are as described with respect to Formulas 2 and 2A, above, including any and all embodiments thereof.

In some embodiments, the polymer has structure Formula 2C:

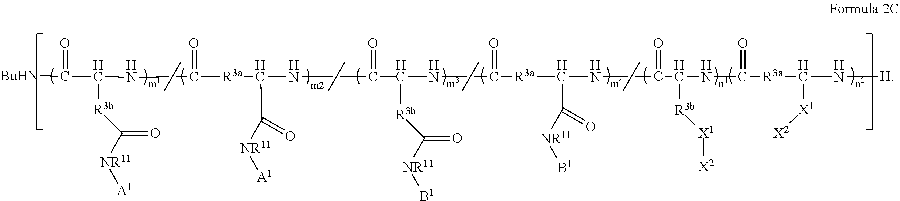

All other aspects of Formula 2C are as described with respect to Formula 2, 2A, and 2B, above, including any and all embodiments thereof.

According to Formula 2 (or Formulas 2A-2C), each of m¹, m², m³, and m⁴ is an integer from 0 to 1000 (e.g., 0 to 500, 0 to 200, 0 to 100, or 0 to 50), provided that the sum of m¹+m²+m³+m⁴ is greater than 2, such as 2-5000, 2-2000, 2-1000, 2-500, 2-100, or 2-50. In some embodiments, the sum of m¹+m²+m³+m⁴ is greater than 5 or greater than 10 (e.g., 5-5000, 5-2000, 5-1000, 5-500, 5-100, or 5-50; or 10-5000, 10-2000, 10-1000, 10-500, 10-100, or 10-50). In some embodiments, m¹+m²+m³+m⁴ is about 20-50 or 20-40. Furthermore, each of n¹ and n² is an integer from 0 to 1000 (e.g., 0 to 500, 0 to 200, 0 to 100, 0 to 50, or 0 to 25), provided that the sum of n¹+n² is greater than 2 (e.g., 2-2000, 2-1000, 2-500, 2-200, 2-100, 2-50, or 2-25). In some embodiments, the sum of n¹+n² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25). In some embodiments, the sum of n¹+n² is about 20-50 or 20-40. In particular embodiments, m¹+m²+m³+m⁴ is about 20-50 or 20-40, and the sum of n¹+n² is about 20-50 or 20-40.

In other words, the polymer comprises at least some monomeric units comprising groups A¹ and/or B¹, herein referred to collectively as the “A monomers” and “B monomers,” respectively. Similarly, the polymer comprises at least some monomeric units comprising groups X¹ and/or X², herein referred to collectively as the “X monomers.” In some embodiments, m¹ and m² are zero, such that the polymer comprises no A¹ groups. In some embodiments, m³ and m⁴ are zero, such that the polymer comprises no B¹ groups. In other embodiments, the polymer has at least some A¹ groups and at least some B¹ groups (i.e., the sum of m¹+m² is 1 or more, and the sum of m³+m⁴ is 1 or more).

The polymer according to Formula 1 can have any suitable degree of polymerization (e.g., sum of m¹+m²+n¹+n²). In some instances, the polymer has a degree of polymerization of about 20 or more (e.g., about 20 to about 1000, or about 20 to about 100, or about 20 to about 65, or about 20 to 64). In some instances, the polymer has a degree of polymerization of about 50 or more (e.g., about 50 to about 1000, or about 50 to about 500, or 66 to about 200).

The polymer can comprise any suitable ratio of A and B monomers to X monomers. In some embodiments, the polymer comprises a ratio of A and B monomers to X monomers (e.g., the ratio of (m¹+m²+m³+m⁴)/(n¹+n²)) of about 25 or less (e.g., about 10 or less, about 5 or less, about 3 or less, or about 2 or less) and, optionally, about 0.2 or more (e.g., about 0.3 or more, 0.4 or more, 0.5 or more, 0.6 or more, 0.7 or more, or about 1 or more). For example, the ratio of A and B monomers to X monomers can be about 0.3 to about 25, from about 0.3 to about 20, from about 0.3 to about 10, from about 0.3 to about 5, from about 0.3 to about 3, from about 0.3 to about 2.

In embodiments where the polymer comprises both A monomers and B monomers, the polymer can comprise any suitable ratio of A monomers to B monomers. In some embodiments, the ratio of A monomers to B monomers (e.g., (m¹+m²)/(m³+m⁴)) can be about 20 or less (e.g., about 10 or less, about 5 or less, about 2 or less, or even about 1 or less, such as about 0.5 or less or 0.2 or less). In some embodiments, the ratio of (m¹+m²)/(m³+m⁴) is about 0.1 or more, or about 0.2 or more, such as about 0.5 or more.

The polymers described herein (e.g., Formulas 1, 1A-1C, 2, or 2A-2C) can exist as any suitable structure type. For example, the polymer can exist as an alternating polymer, random polymer, block polymer, graft polymer, linear polymer, branched polymer, cyclic polymer, or a combination thereof. In certain embodiments, the polymer is a random polymer, block polymer, graft polymer, or a combination thereof.

Thus, in the structure of Formula 1 or Formula 2 (or Formulas 1A-1C or 2A-2C), the monomers (which can be referred to by their respective side chains A¹, B¹, and X¹ and/or X²) can be arranged randomly or in any order. The integers m¹, m², m³, m⁴, n¹, and n² (as applicable) merely denote the number of the respective monomers that appear in the chain overall, and do not necessarily imply or represent any particular order or blocks of those monomers, although blocks or stretches of a given monomer might be present in some embodiments. For instance, the structure of Formula 2 can comprise the monomers in the order -A¹-A¹-B¹-B¹—, —B¹-A¹-B¹-A¹-, -A¹-B¹-B¹—B¹—, etc. Furthermore, the polymer can comprise blocks of A and/or B polymers (e.g., [A monomers]ₘ₁₊ₘ₂−[B monomers]ₘ₃₊ₘ₄) in any order). The polymer can comprise individual X monomers interspersed with the A and B monomers (e.g., -A-X—B—, -A-B—X—, —B—X-A, etc.), or the polymer can be “capped” with one or more X monomers (e.g., a block of X monomers) at one or both ends of the polymer. Likewise, when the polymer comprises blocks of A and/or B monomers, the polymer can comprise blocks of X monomers interspersed between blocks of A and/or B monomers, or the polymer can be “capped” with one or more X monomers (e.g., a block of X monomers) at one or both ends of the polymer. In some embodiments, the polypeptide (e.g., polyaspartamide) backbone will be arranged in an alpha/beta configuration, such that the “m₁-like” or “m₃-like” and “m₂-like” or “m₄-like” monomers will alternate, wherein the polymer is capped with X monomers or the X monomers are interspersed throughout. However, the “A” and “B” sidechains (e.g., A¹ and B¹) can be dispersed randomly throughout the polymer backbone.

Each A¹ group is independently selected and, therefore, can be the same or different from one another. Similarly, each B¹ group (when present) is independently selected and can be the same or different from one another, and each X² group is independently selected and can be the same or different from one another. However, in some embodiments, all A¹ groups are the same, all B¹ groups are the same, and/or all X² groups are the same.

Any of the polymers described herein can further comprise monomer units with a side chain comprising a polyalkylene oxide, polyglycolic acid, polylactic acid, or combination thereof, can be linked to the polymer backbone in any suitable manner, such as directly or via a linkage comprising, for instance, an ester, an amide, or an ether group, optionally further comprising an alkylene linker (e.g., a methylene or ethylene linker). In some embodiments, the side chain comprises at least one polyethylene glycol group (a.k.a. polyethylene oxide group) having a sum total of from 2 to 200 ethylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polypropylene oxide group having a sum total of from 2 to 200 propylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polylactic acid and/or polyglycolic acid group having a sum total of from 2 to 200 polylactic acid and/or polyglycolic acid units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In certain embodiments, the side chain comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units. In embodiments where the side chain comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units, the side chain can exist as any suitable structure type. For example, the side chain can comprise the ethylene glycol and/or propylene oxide units and the polylactic acid and/or polyglycolic acid units as an alternating polymer, random polymer, block polymer, graft polymer, linear polymer, branched polymer, cyclic polymer, or a combination thereof. In certain embodiments, the side chain comprises polyalkylene oxide units (e.g., ethylene oxide units, propylene oxide units, or both) and optionally further comprises polylactic acid or polyglycolic acid units. For example, the side chain of at least some of the monomers can be derived from PLURONIC® F65 or PLURONIC® F127.

For instance, the polymer (e.g., the first polymer) can comprise monomer units with a side chain comprising a polyalkylene oxide, polyglycolic acid, polylactic acid, or combination thereof, which comprise a structure of Formula 1:

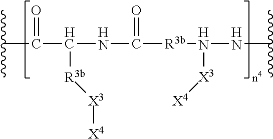

wherein:

each of n³ and n⁴ is an integer from 0 to 1000, provided that the sum of n³+n⁴ is greater than 1;

the symbol “/” indicates that the units separated thereby are linked randomly or in any order;

each instance of R^{3a} is independently a methylene or ethylene group;

each instance of R^{3b} is independently a methylene or ethylene group;

each X³ independently is —C(O)O—, —C(O)NR¹¹—, —C(O)—, —S(O)(O)—, or a bond;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X⁴ comprises polyalkylene oxide, polyglycolic acid, polylactic acid, or a combination thereof; and

each instance of p is independently an integer from 2 to 200.

The polymer can be any suitable polymer, provided the polymer comprises the foregoing polymer structure. In some embodiments, the polymer is a block copolymer comprising a polymer block having the structure of Formula 1 or Formula 2 and one or more other polymer blocks (e.g., an ethylene oxide subunit, or a propylene oxide subunit). In other embodiments, the structure of Formula 2 is the only polymeric unit of the polymer, which can comprise any suitable end groups. In certain embodiments, the polymer further comprises a substituent comprising a tissue-specific or cell-specific targeting moiety.

Non-limiting examples of the polymers provided herein include, for instance:

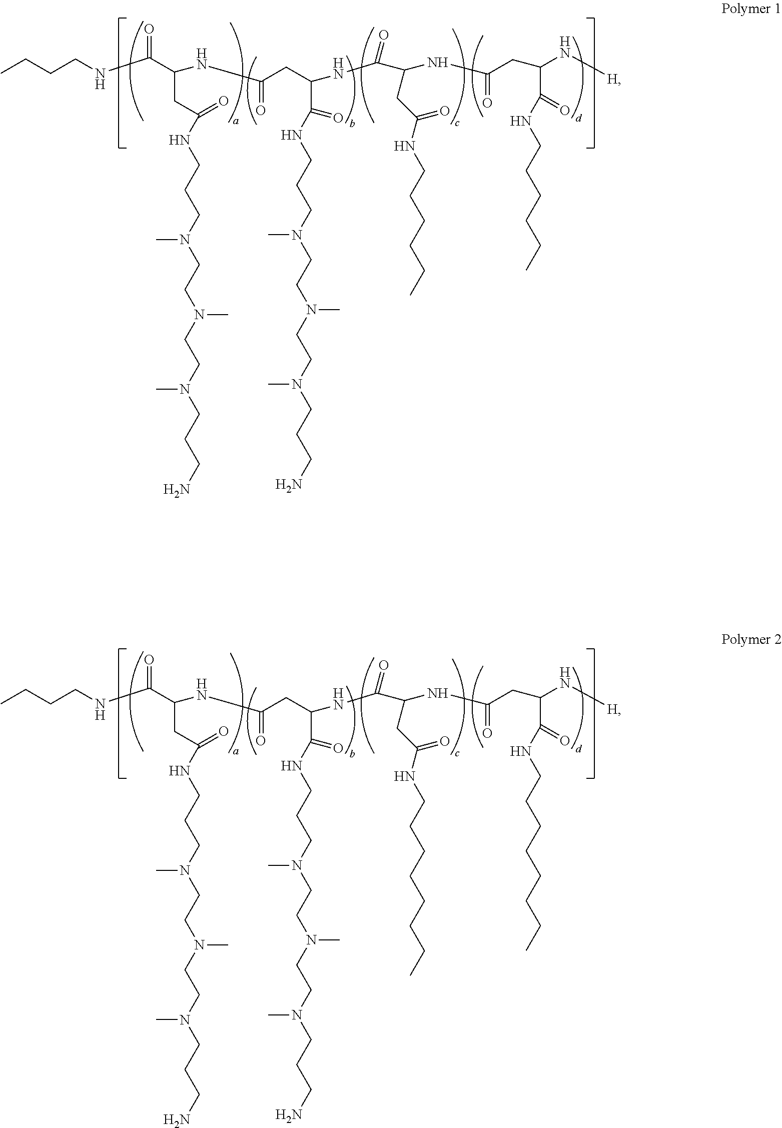

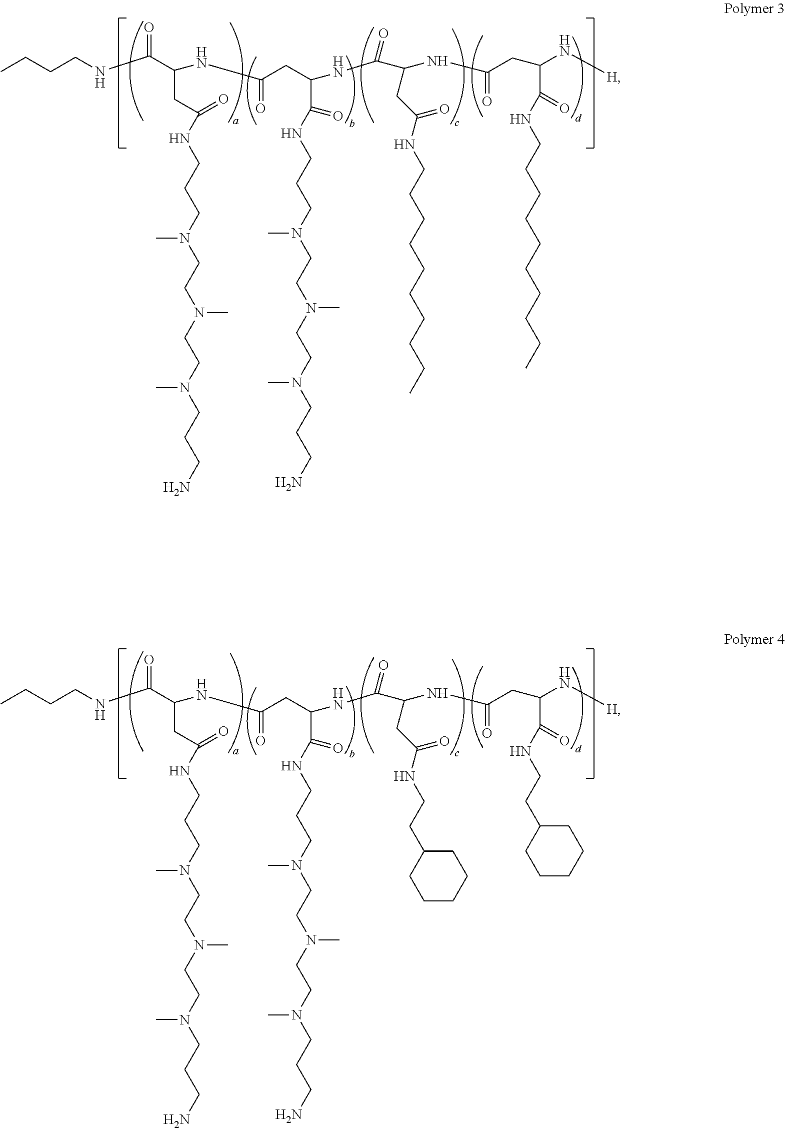

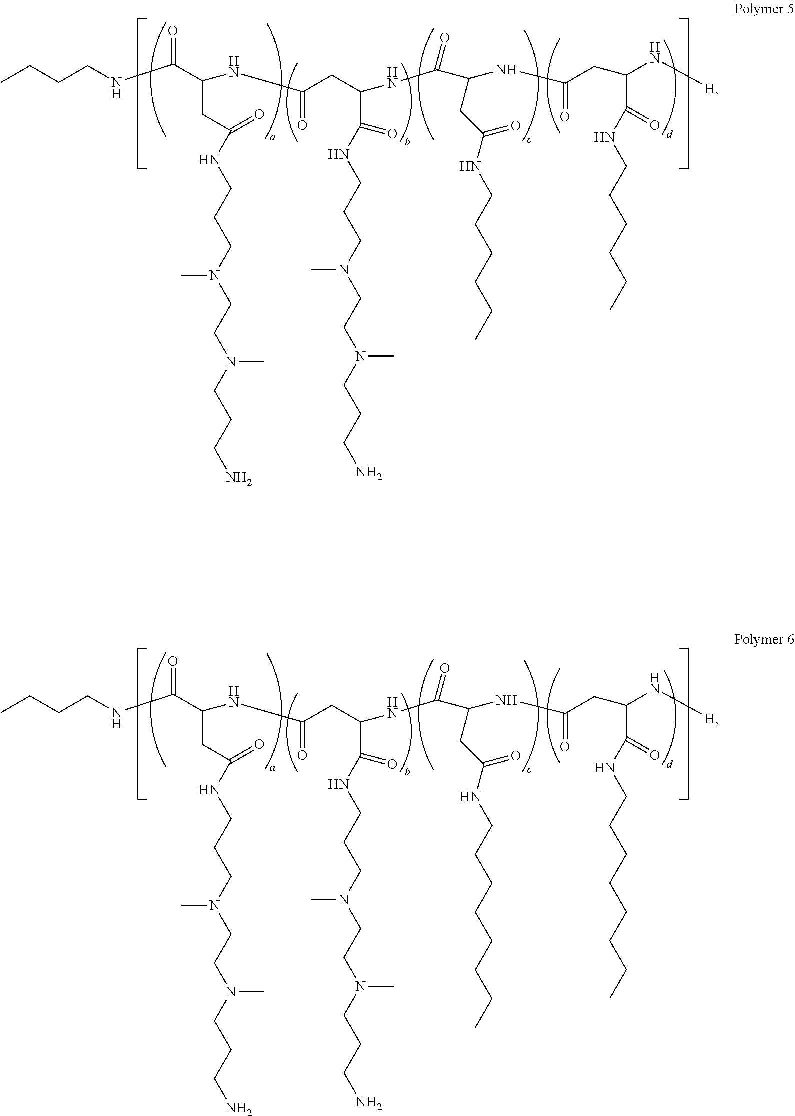

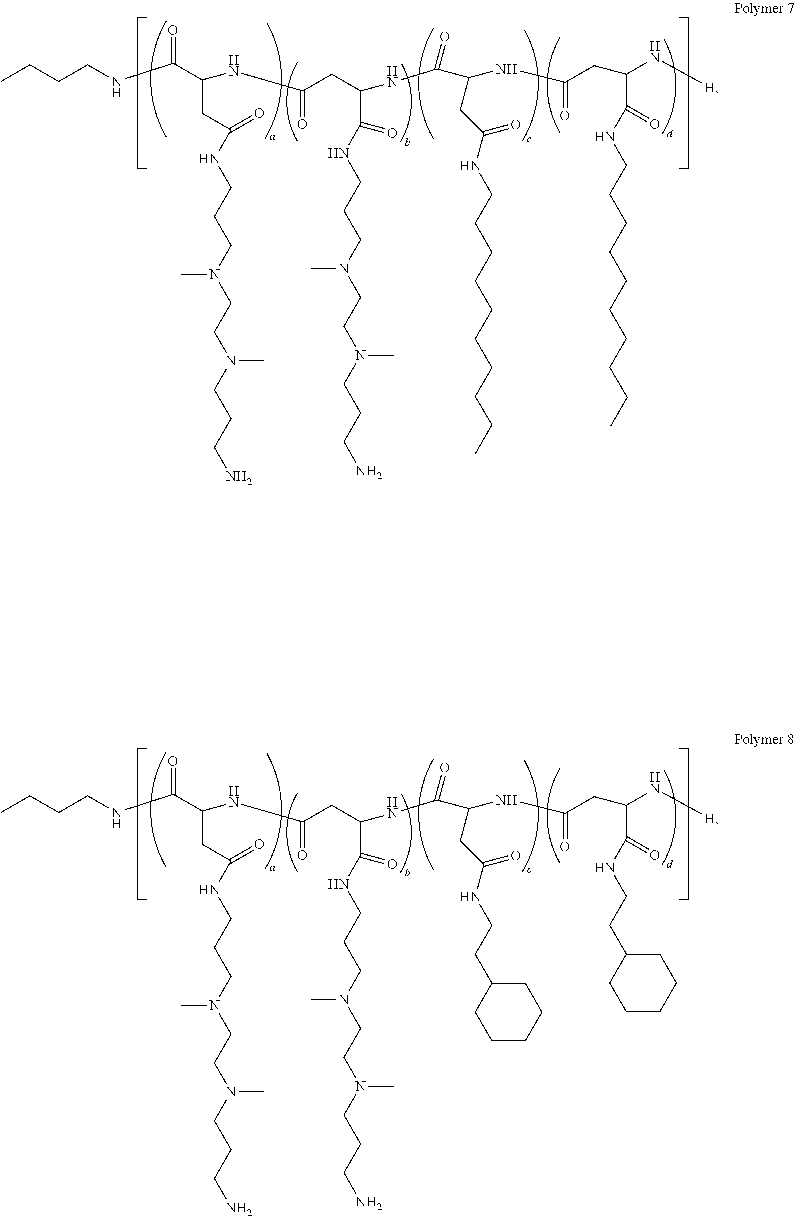

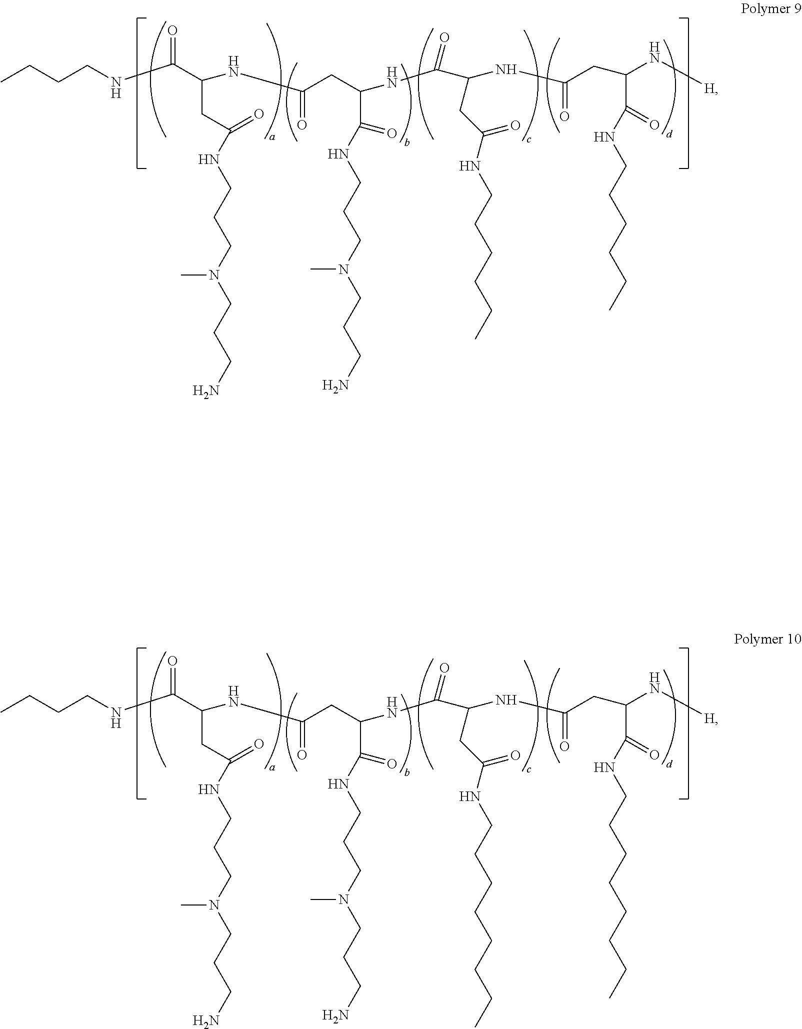

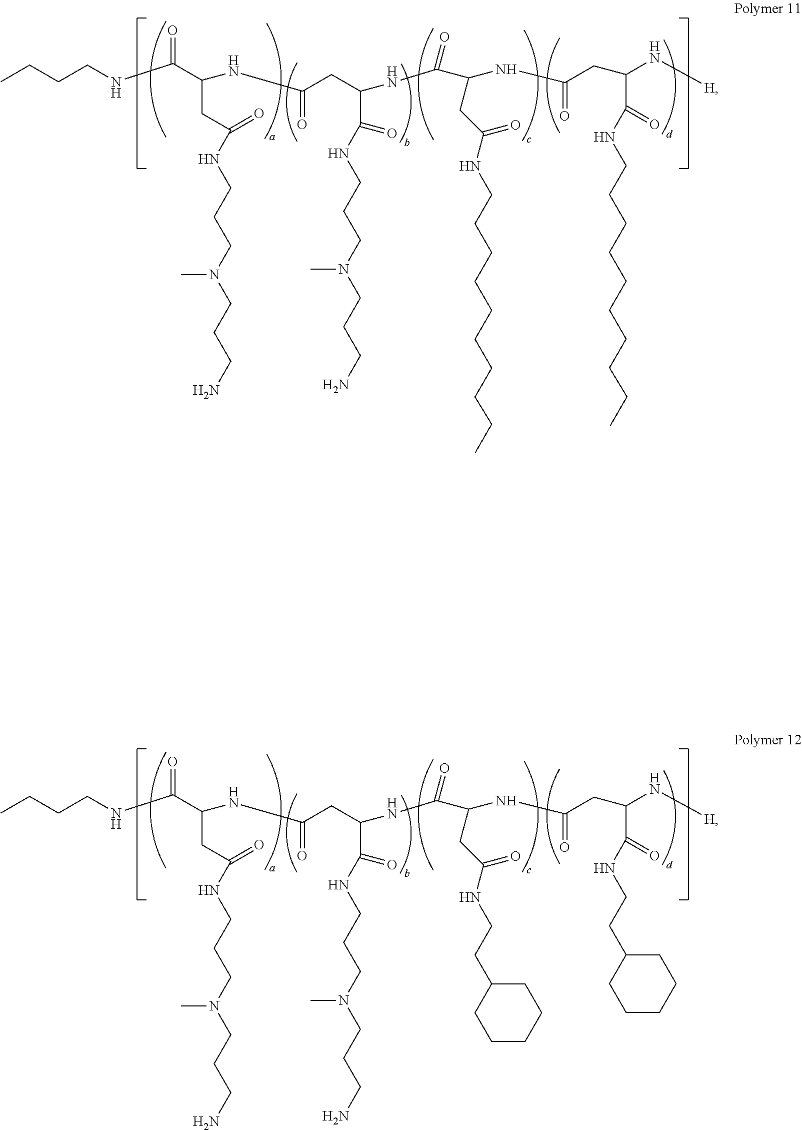

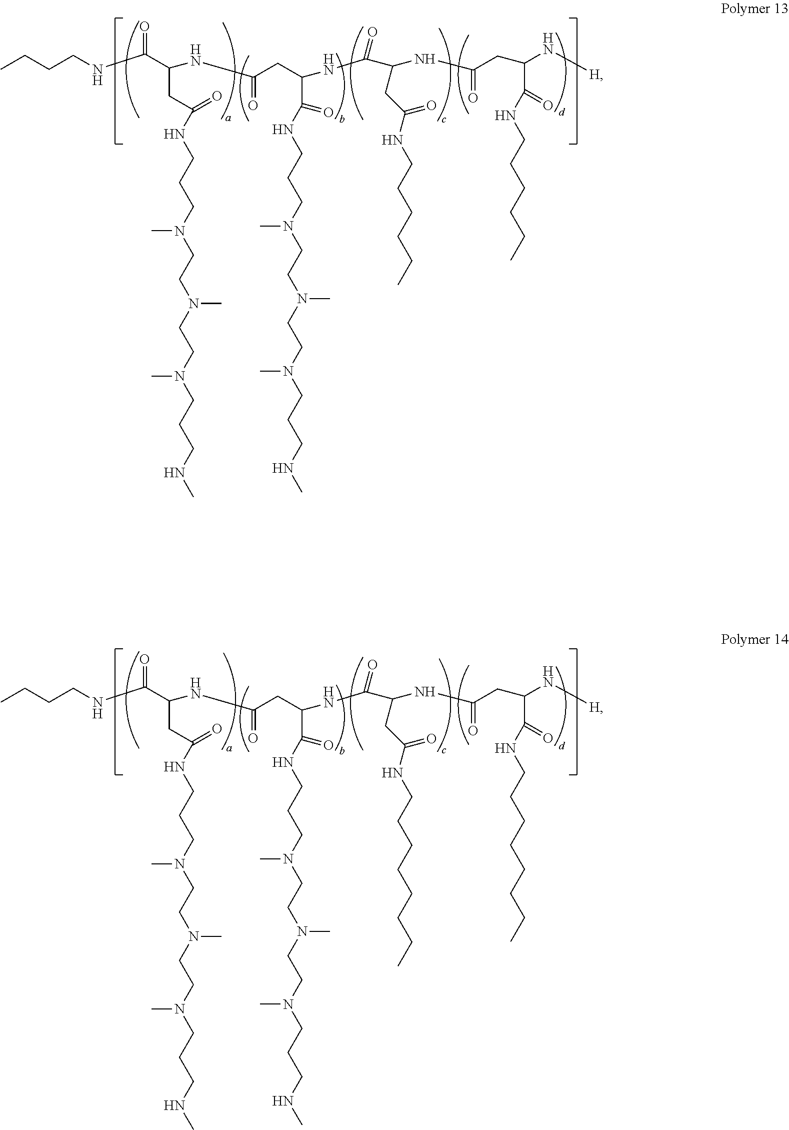

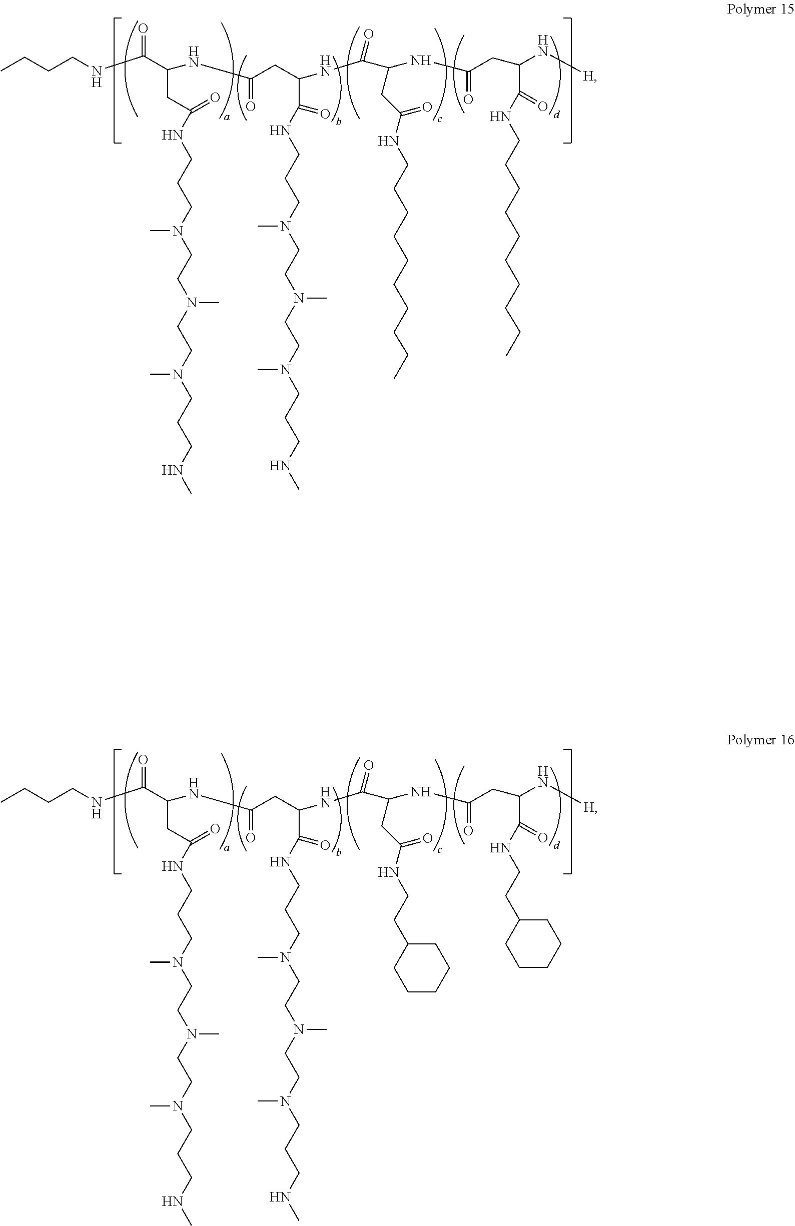

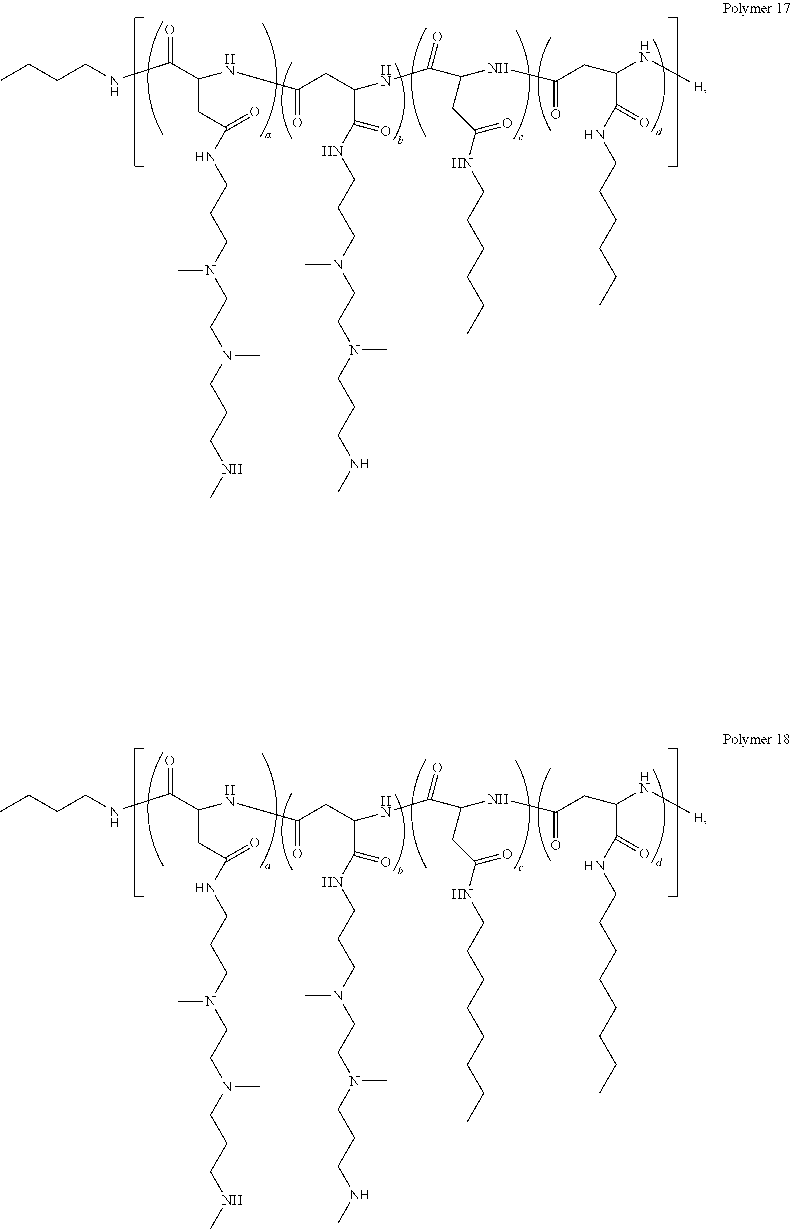

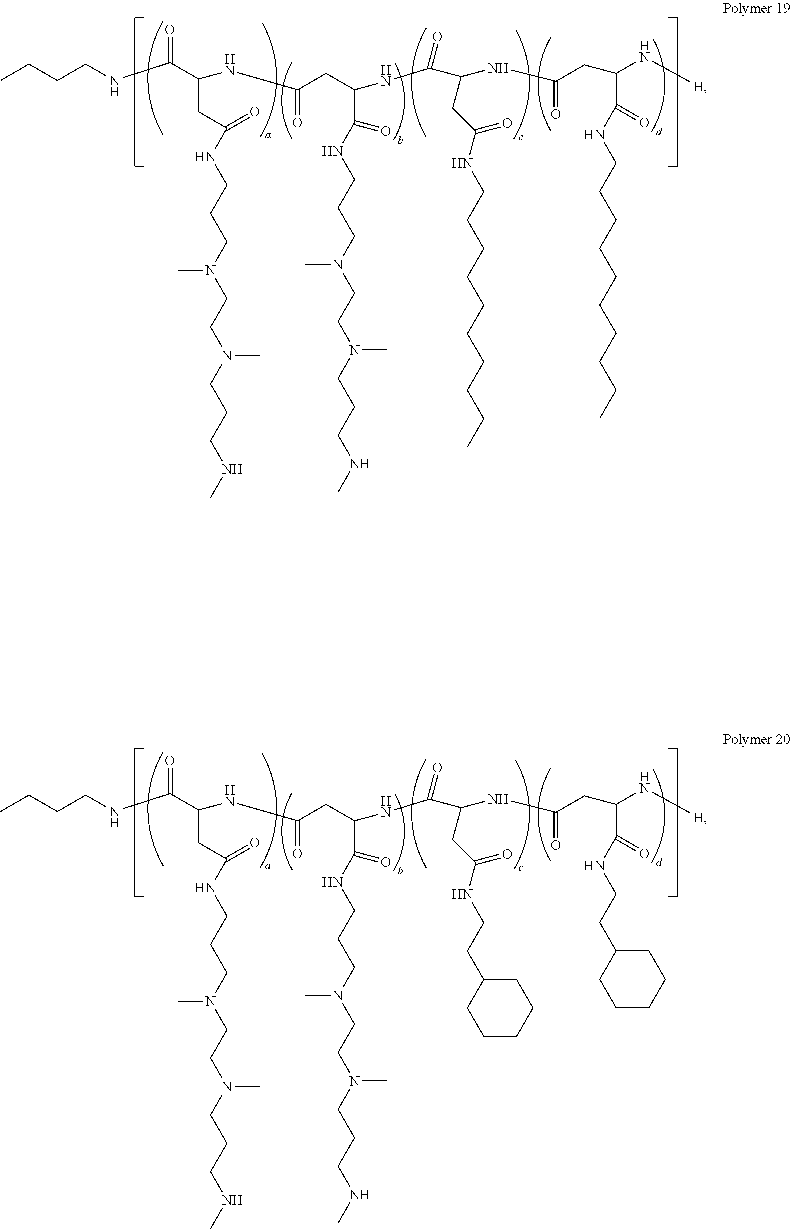

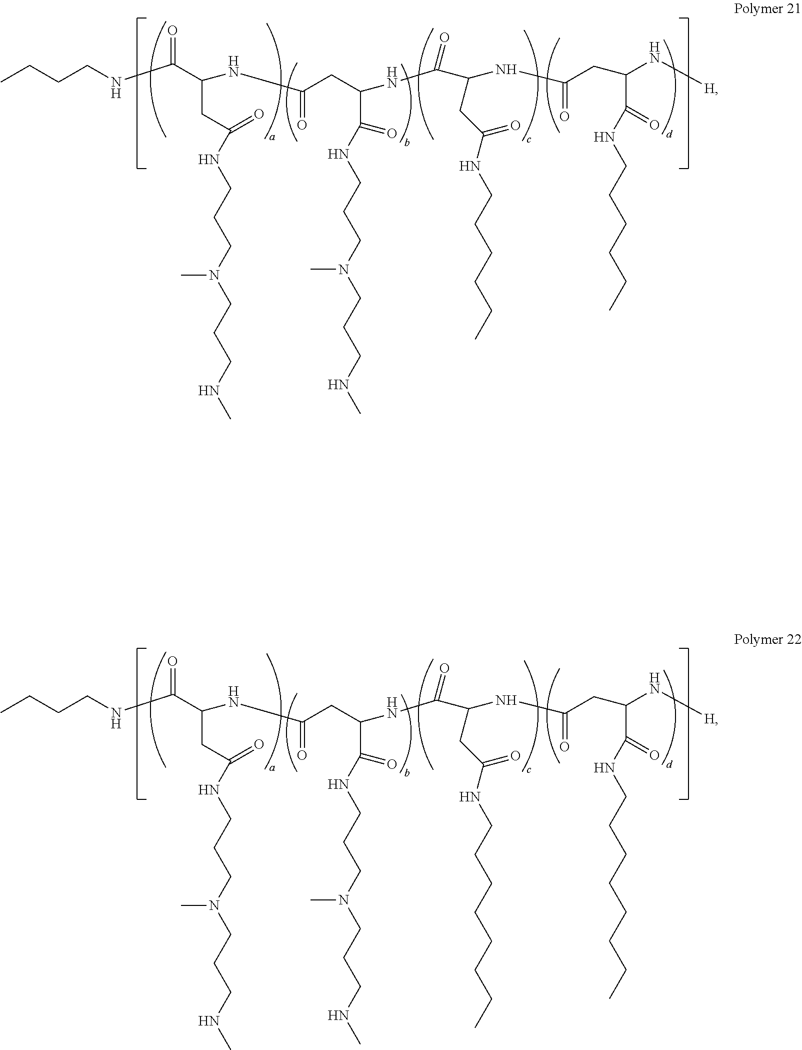

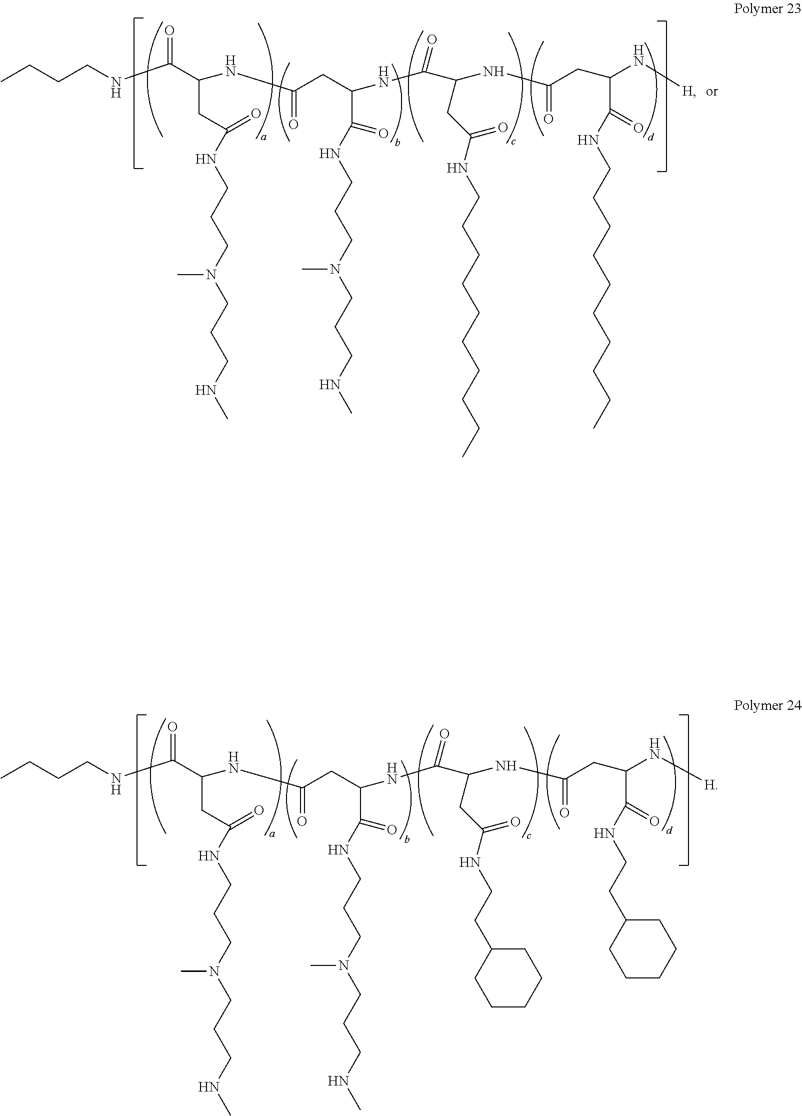

In any of the foregoing polymers, (a+b) is from about 5 to about 65 (e.g., about 5 to about 50, about 5 to about 40, about 5 to about 30, about 5 to about 20, or about 5 to about 10) and (c+d) is from about 2 to about 60 (e.g., about 2 to about 50, about 2 to about 40, about 2 to about 30, about 2 to about 20, or about 2 to about 10). In certain embodiments, (a+b) is about 55 and (c+d) is about 10. In other embodiments, (a+b) is about 45 and (c+d) is about 20. In certain embodiments, (a+b+c+d) is about 10-500, such as about 10-400, about 10-200, or about 10-100 (e.g., about 25-100, 25-64, 50-75, or 66-100). Again, the indication of the number of units (“a”, “b”, “c”, and “d”) in these exemplary polymers does not imply a block co-polymer structure; rather, these numbers indicate the number of units overall, which units can be randomly arranged as indicated by the “/” symbols in the formulas

The polymer can contain any suitable proportion of (a+b) to (c+d). In other embodiments, (a+b) ranges from 10-95% (e.g., 10-75%, 10-65%, 10-50%, 20-95%, 20-75%, 20-65%, 20-50%, 30-95%, 30-75%, 30-65%, or 30-50%) of the total number of polymer units (a+b+c+d). In other embodiments, (c+d) ranges from 5-90% of the total number of polymer units (a+b+c+d) (e.g., 5-75% 5-65%, 5-50%, 5-40%, 5-30%, 10-90%, 10-75%, 10-65%, 10-50%, 10-40%, or 10-30%). In still other embodiments, the ratio of (a+b):(c+d) can be about 1 to about 25, from about 1 to about 20, from about 1 to about 10, from about 1 to about 5, from about 5 to about 25, from about 10 to about 25, or from about 15 to about 25.

Further non-limiting examples of the polymers provided herein include, for instance:

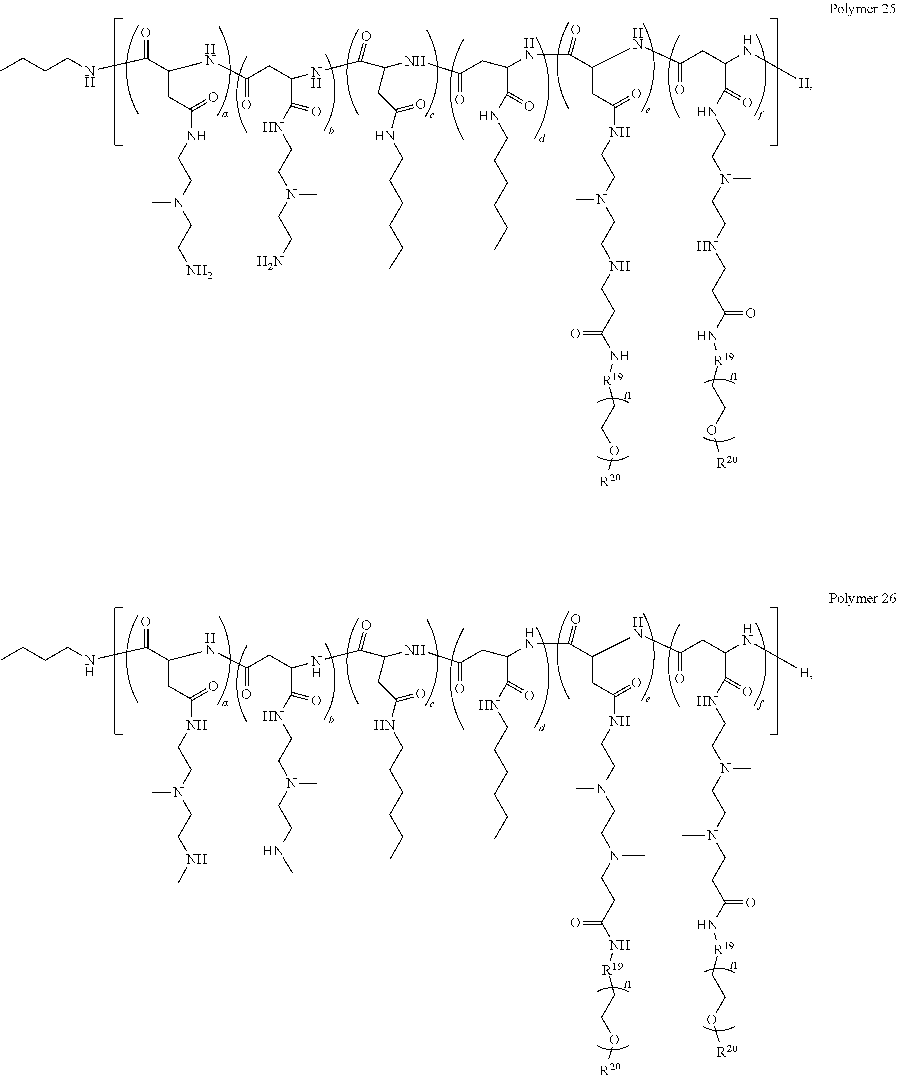

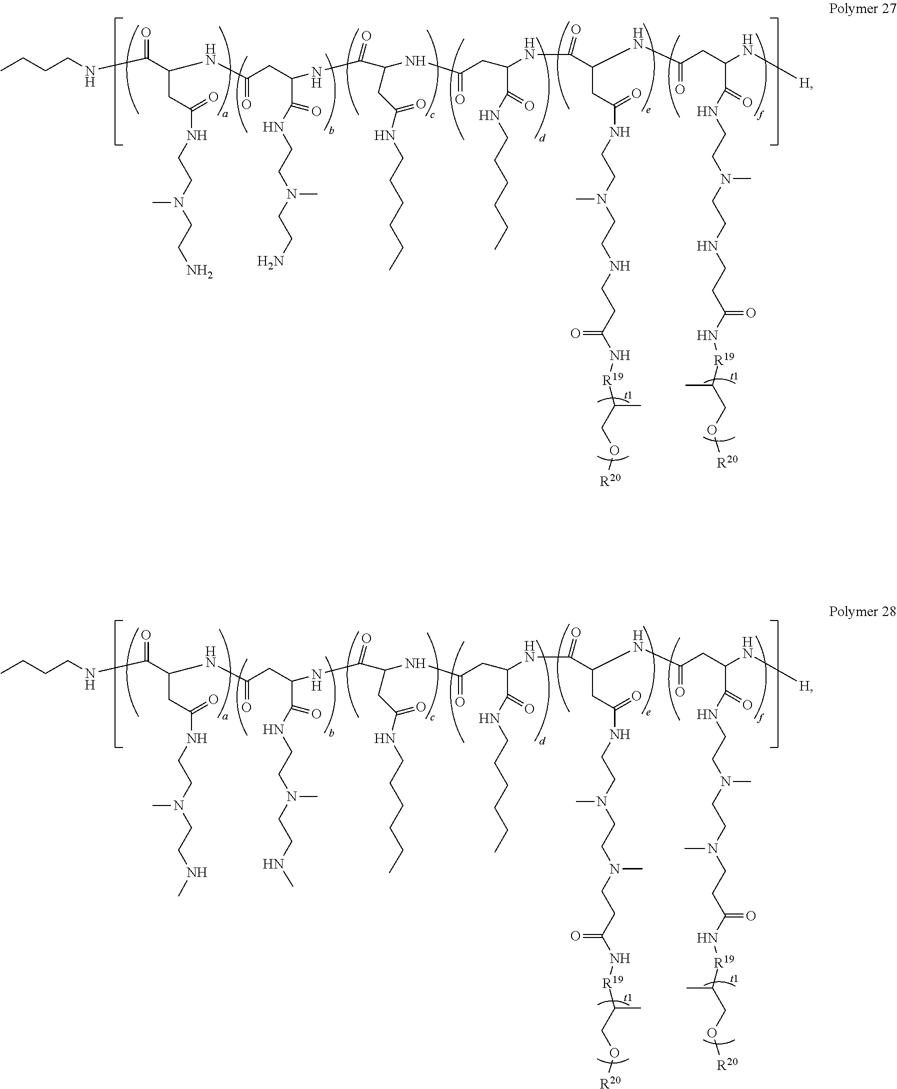

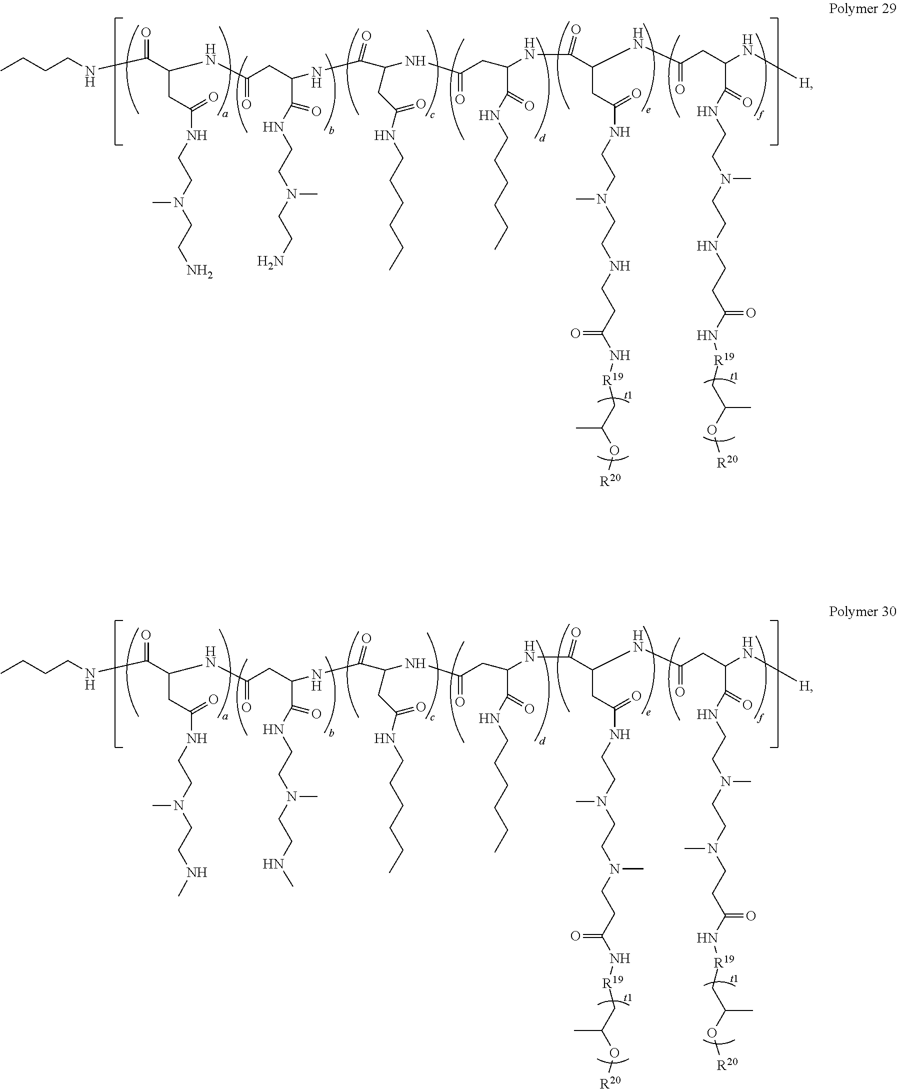

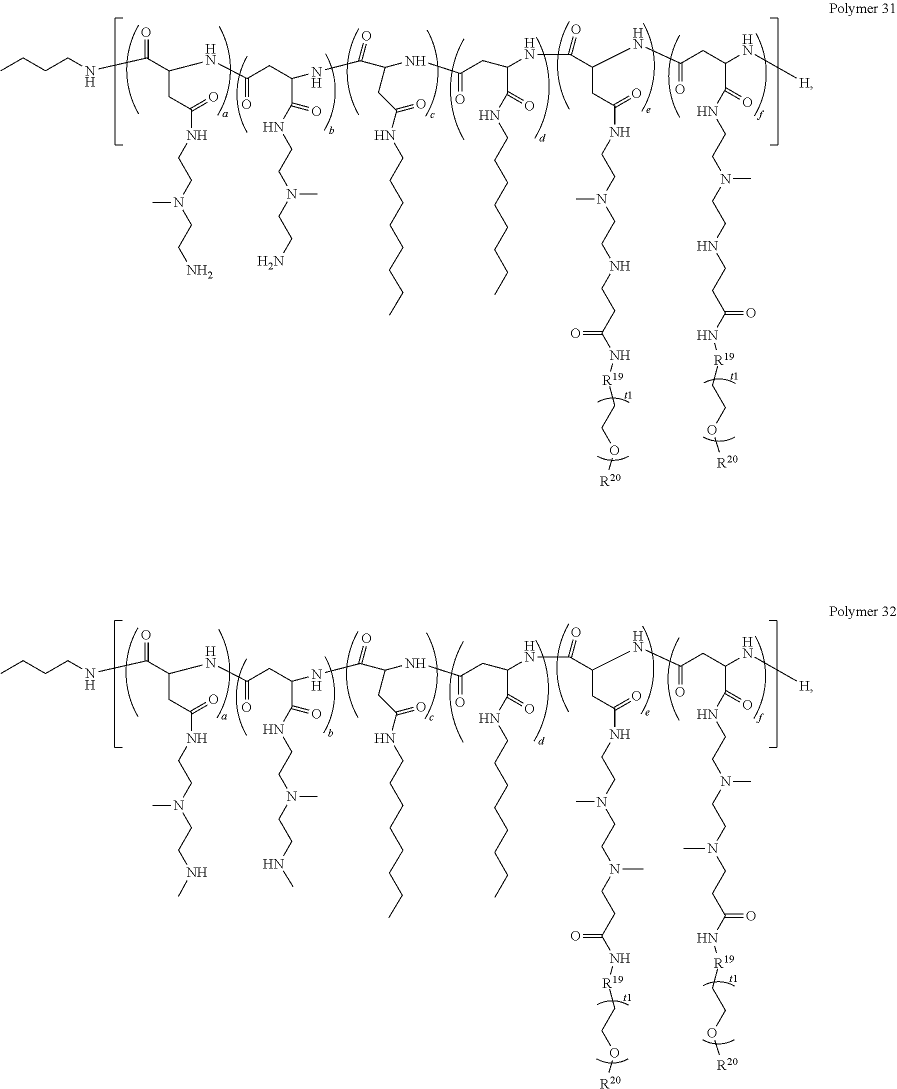

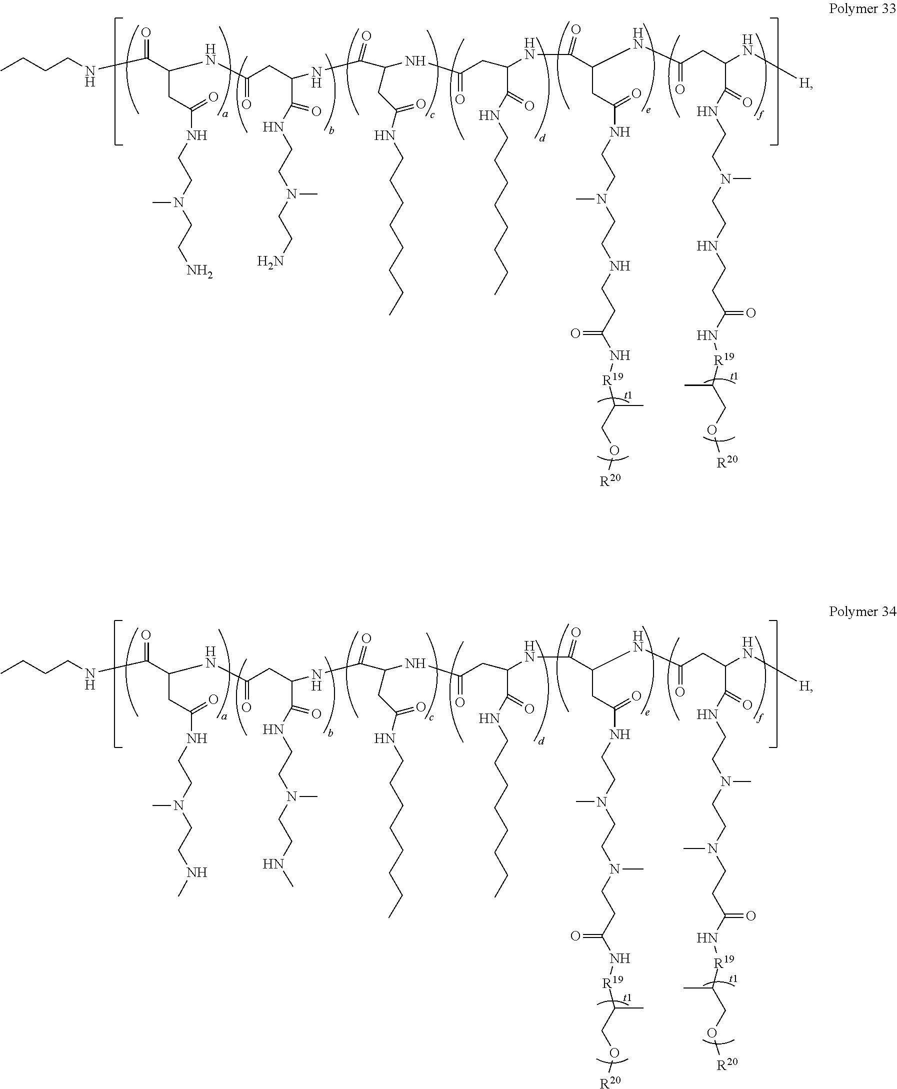

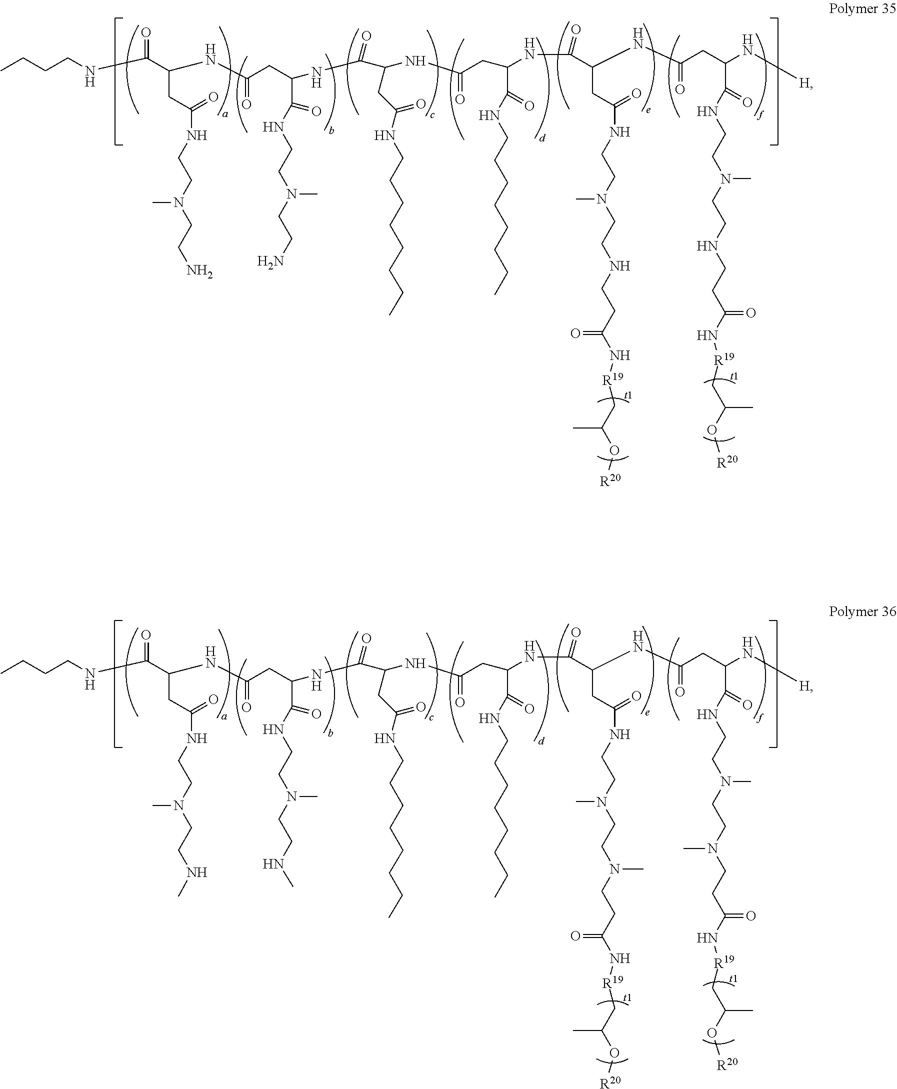

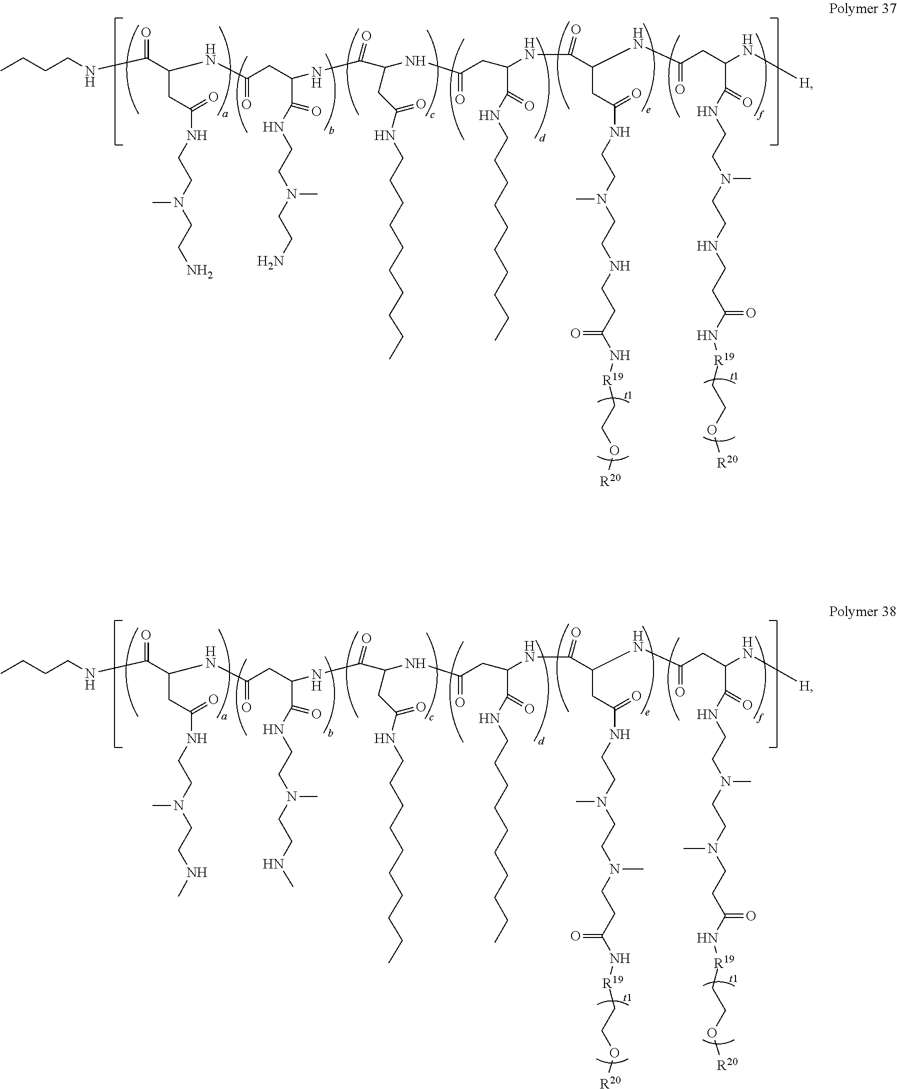

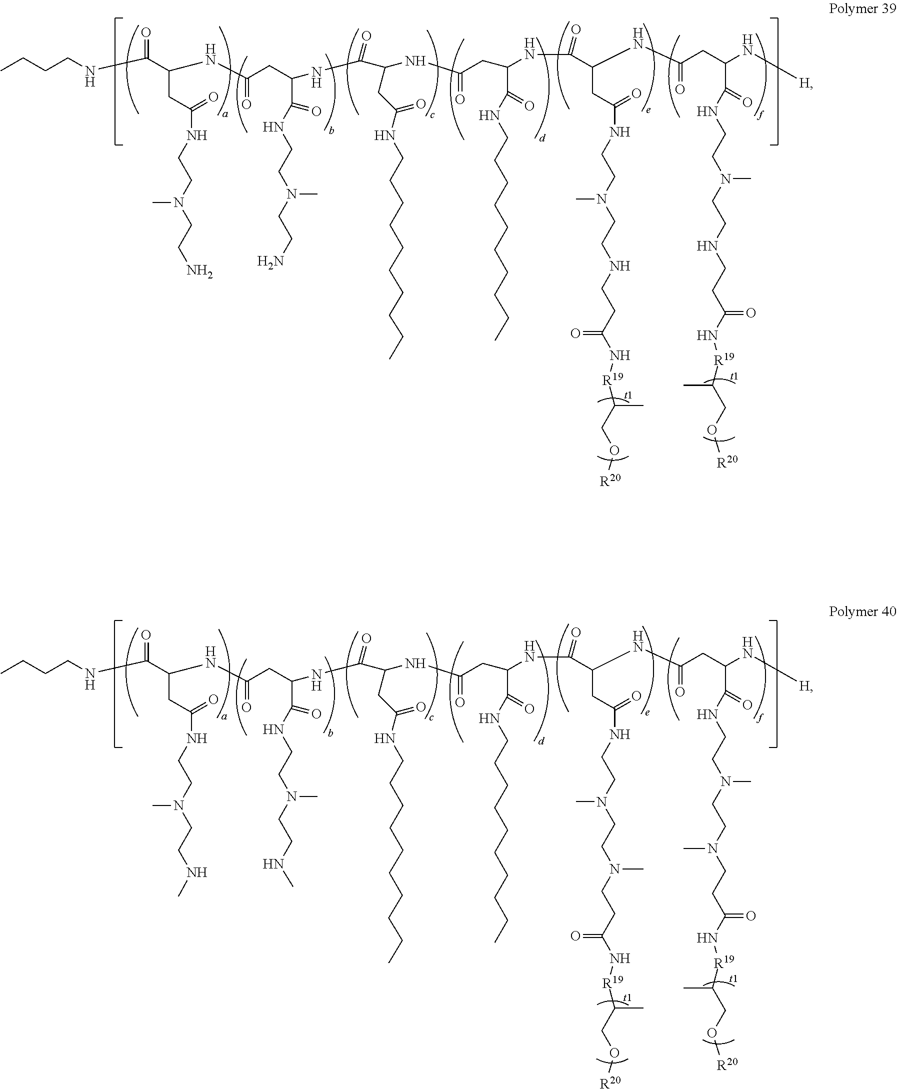

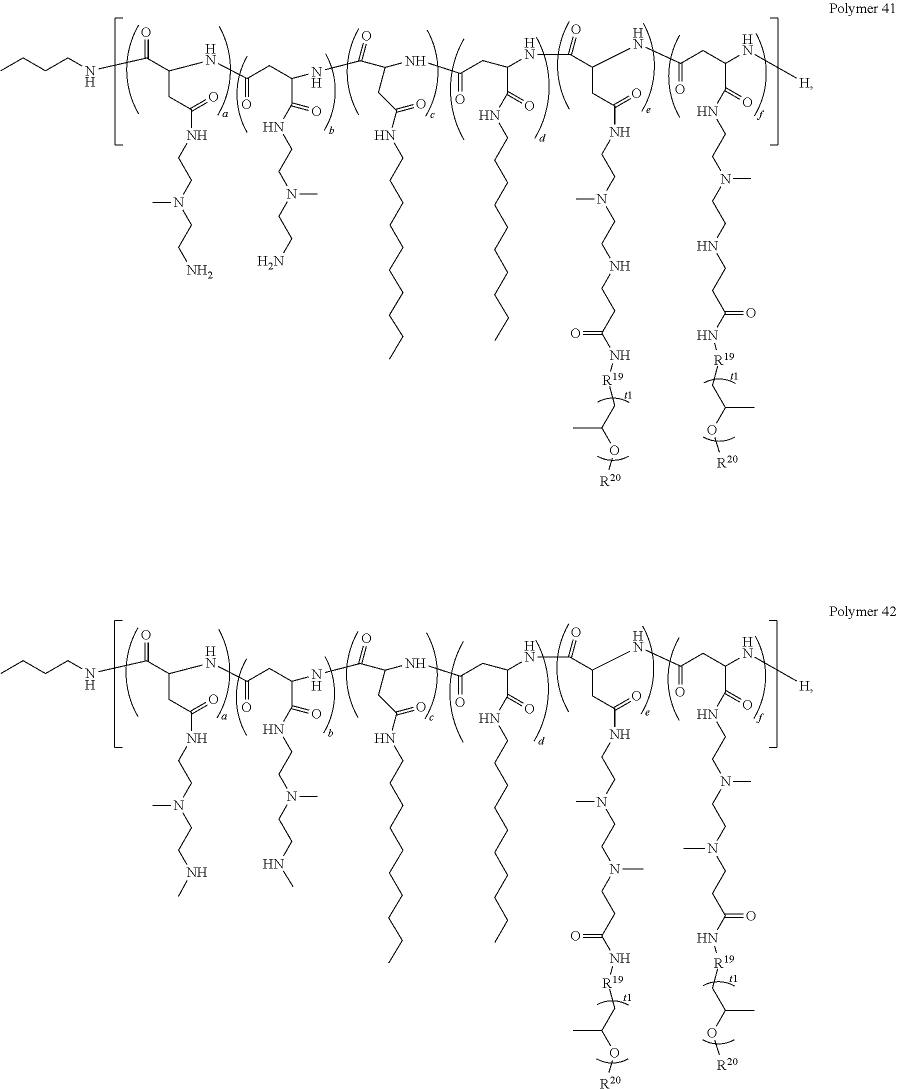

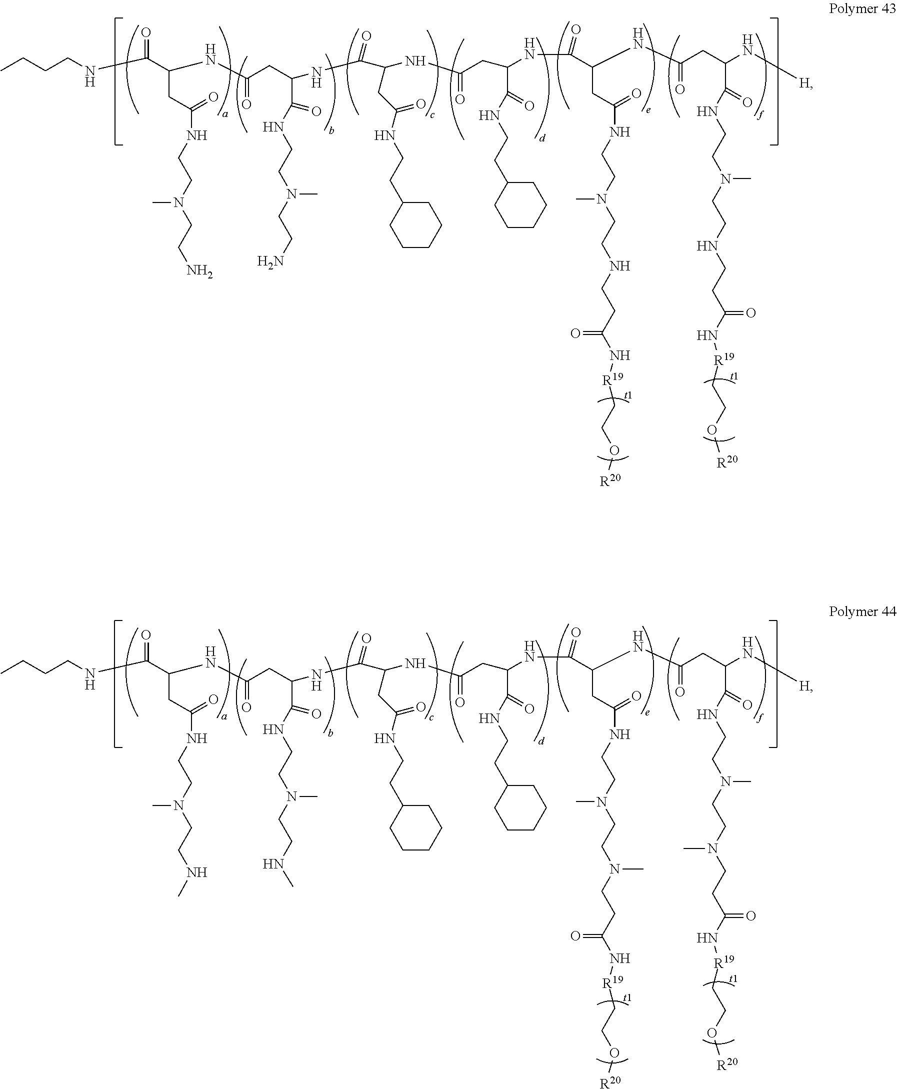

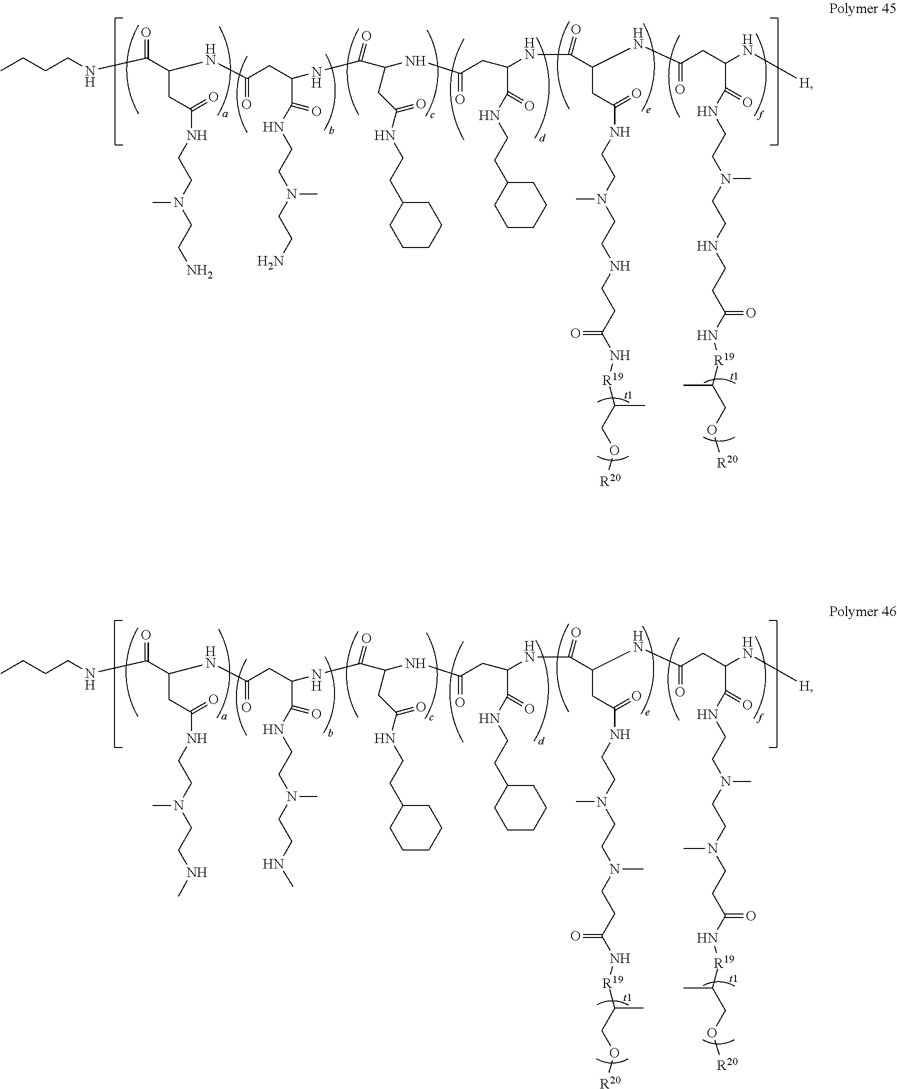

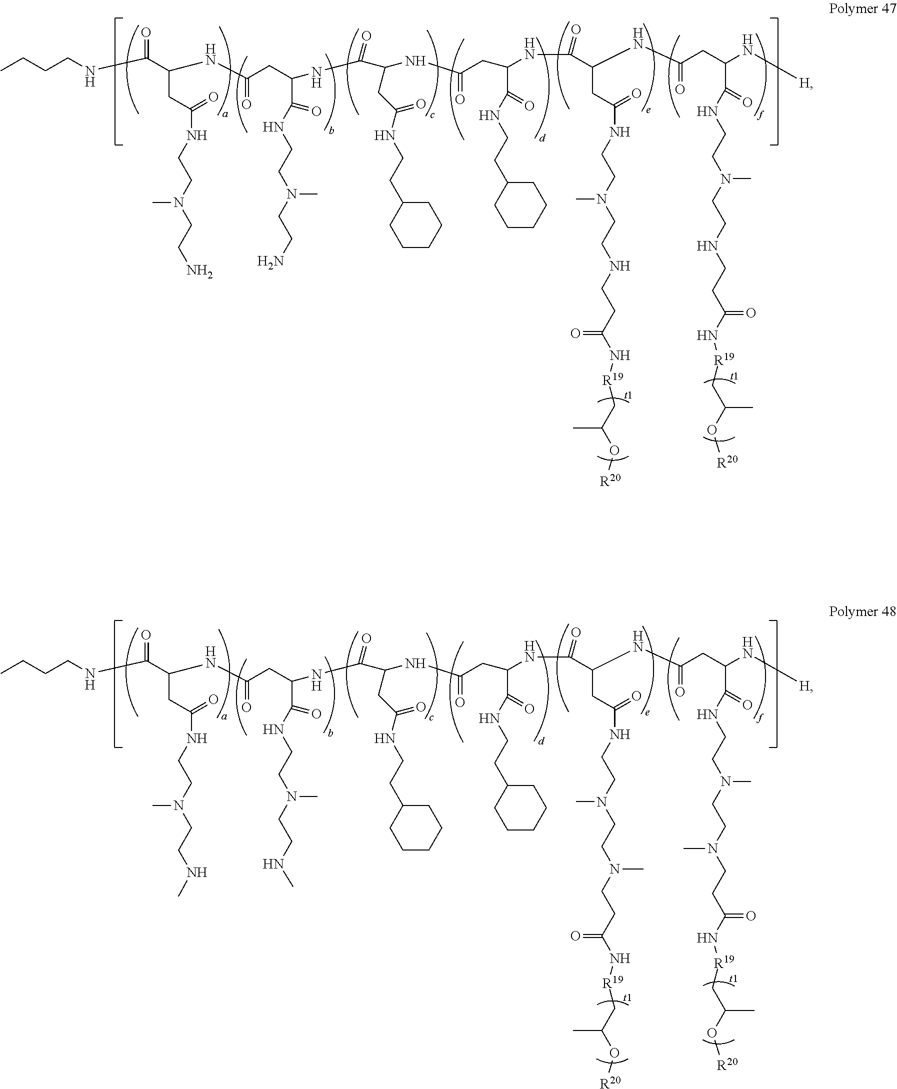

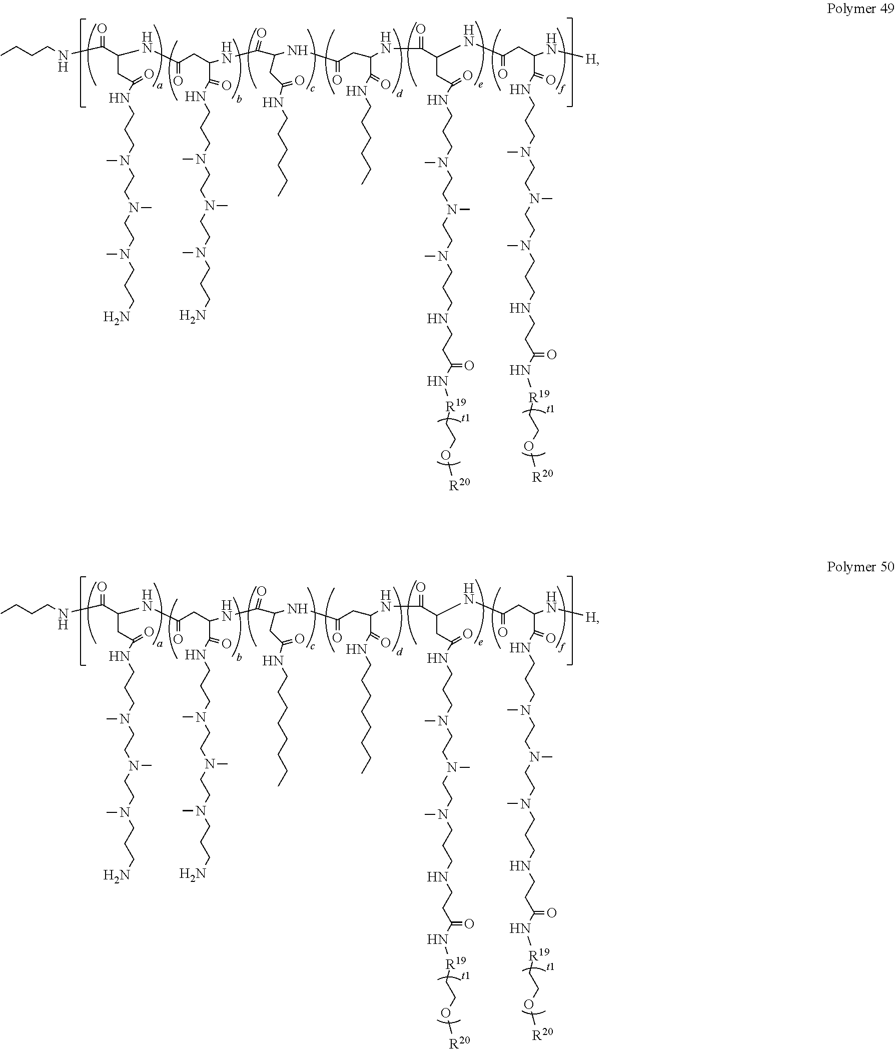

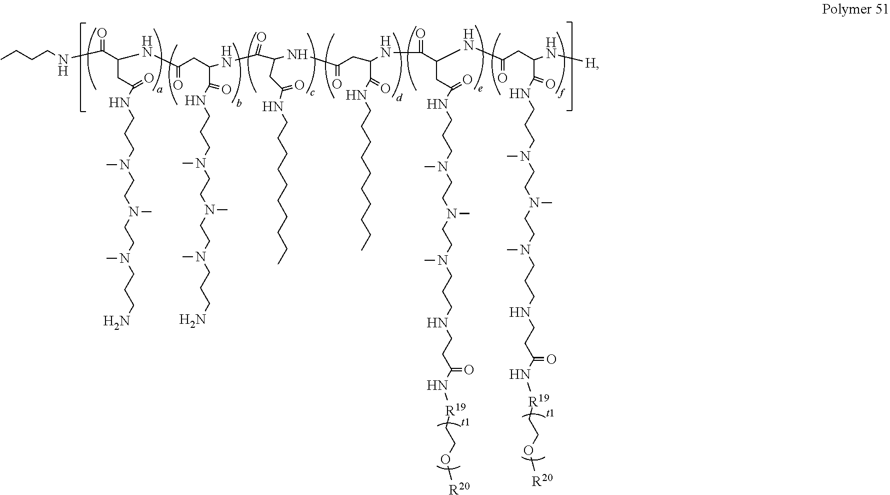

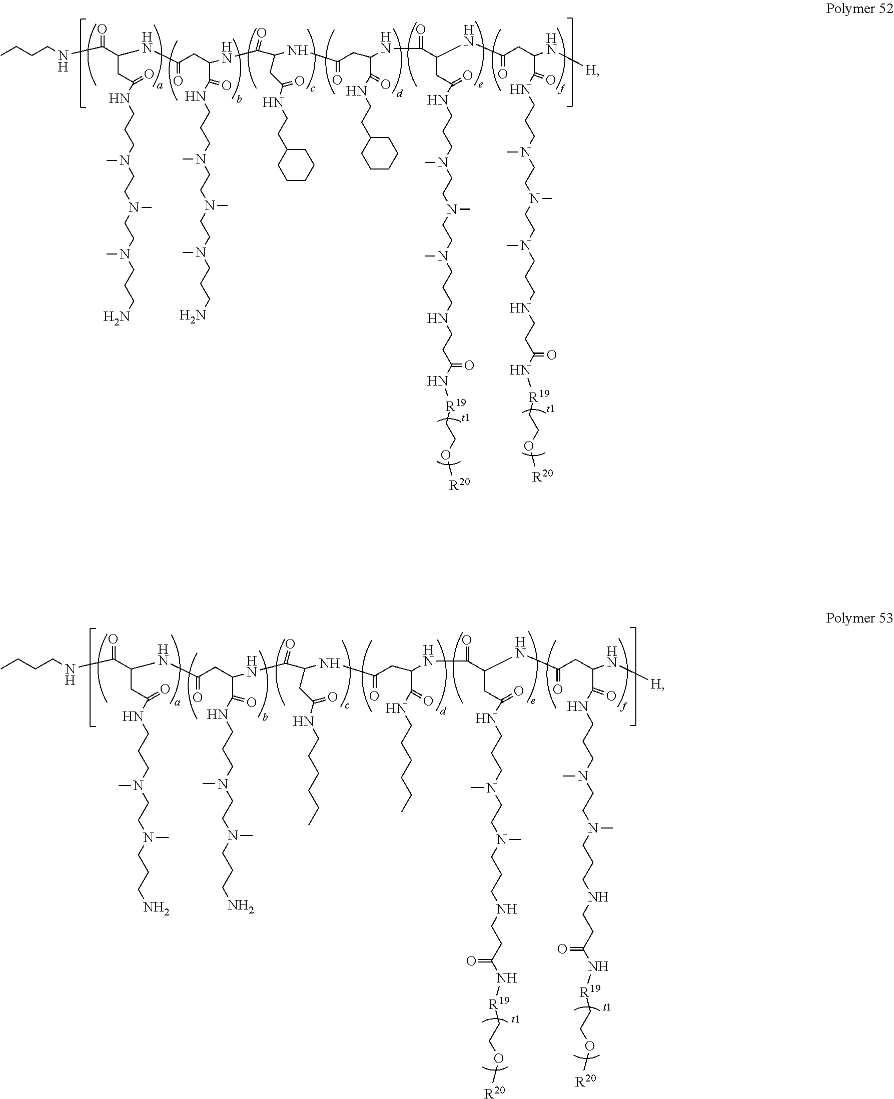

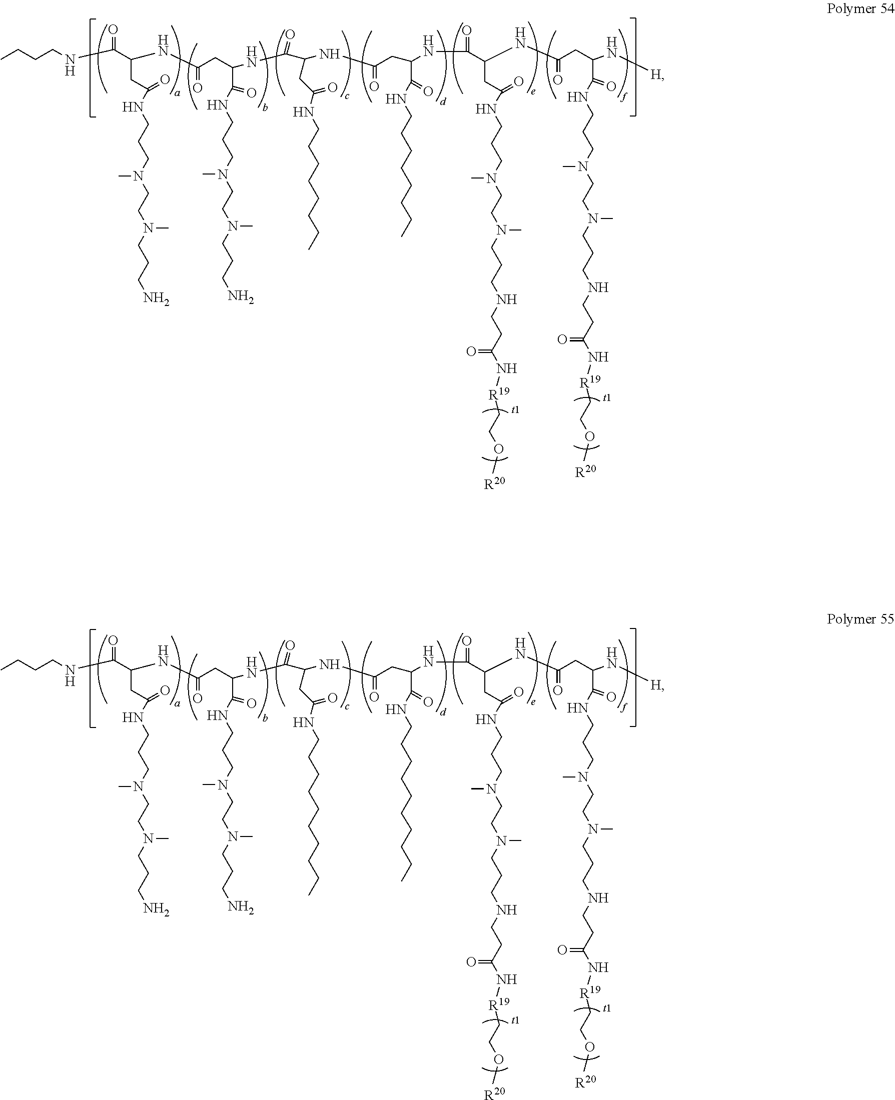

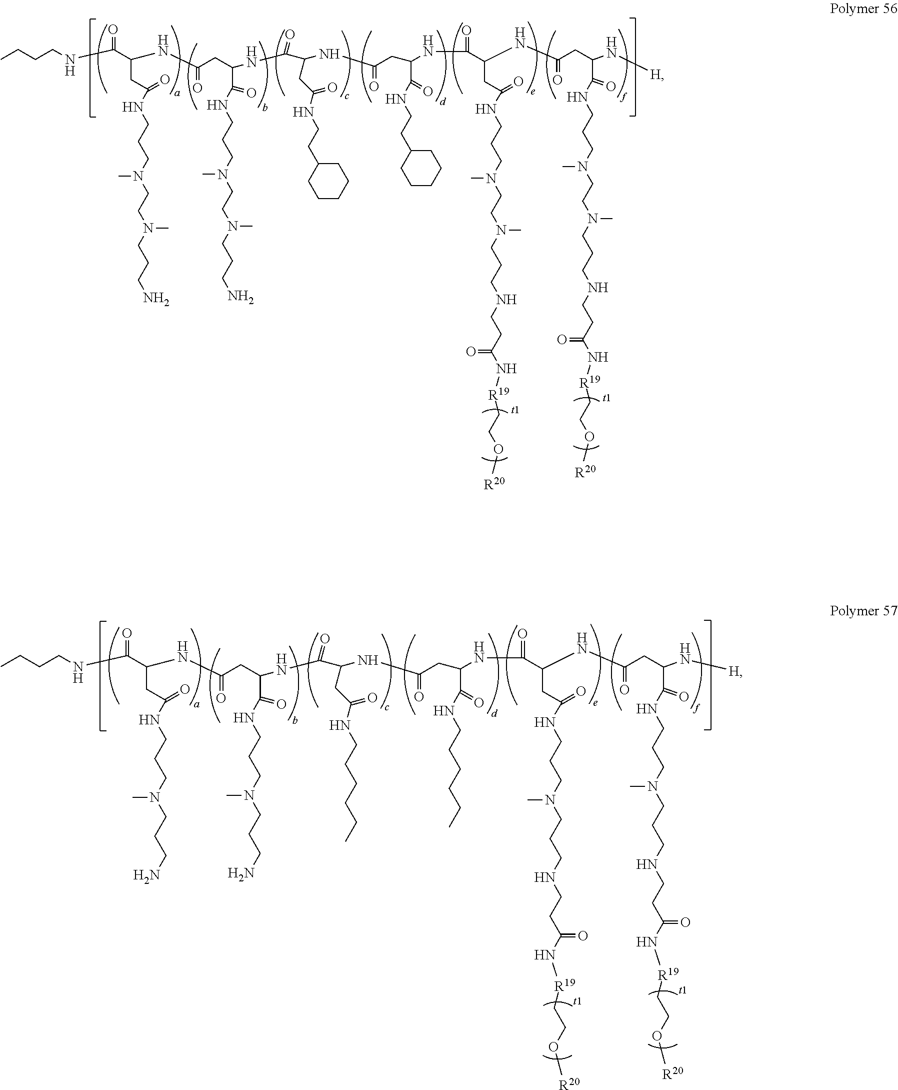

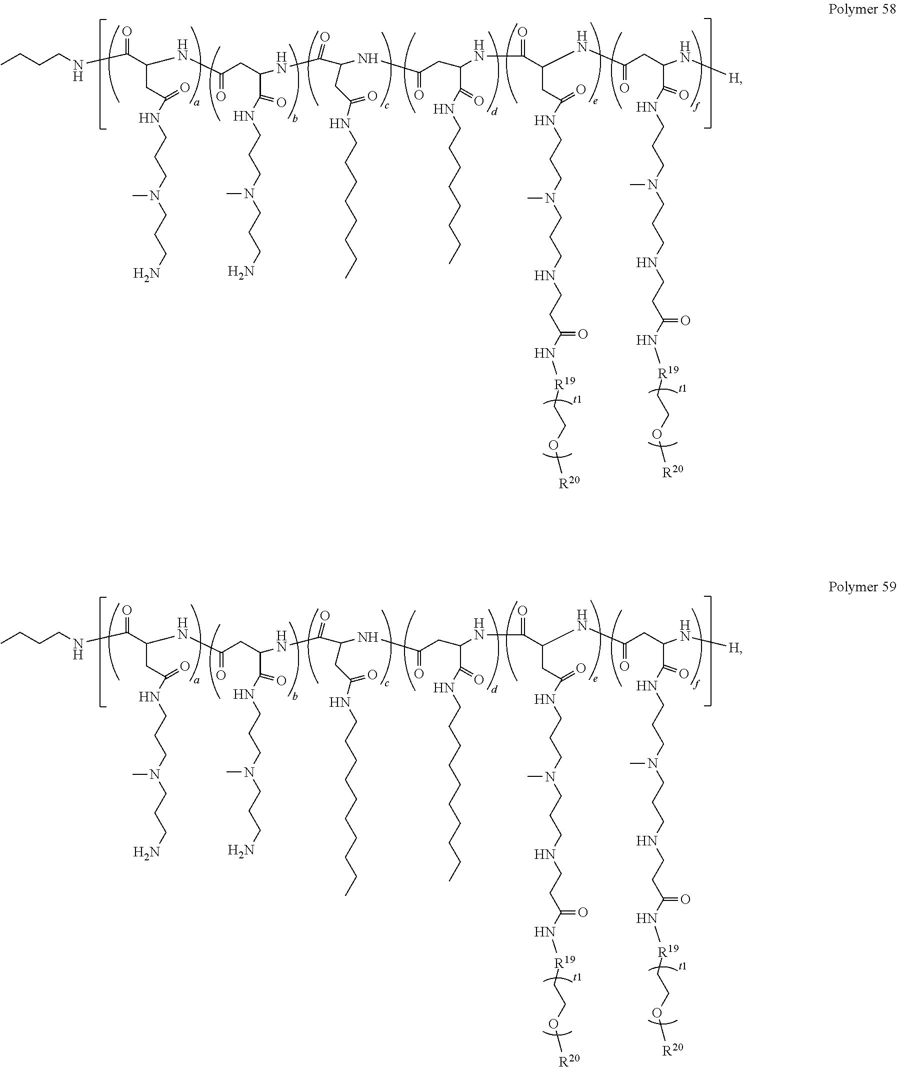

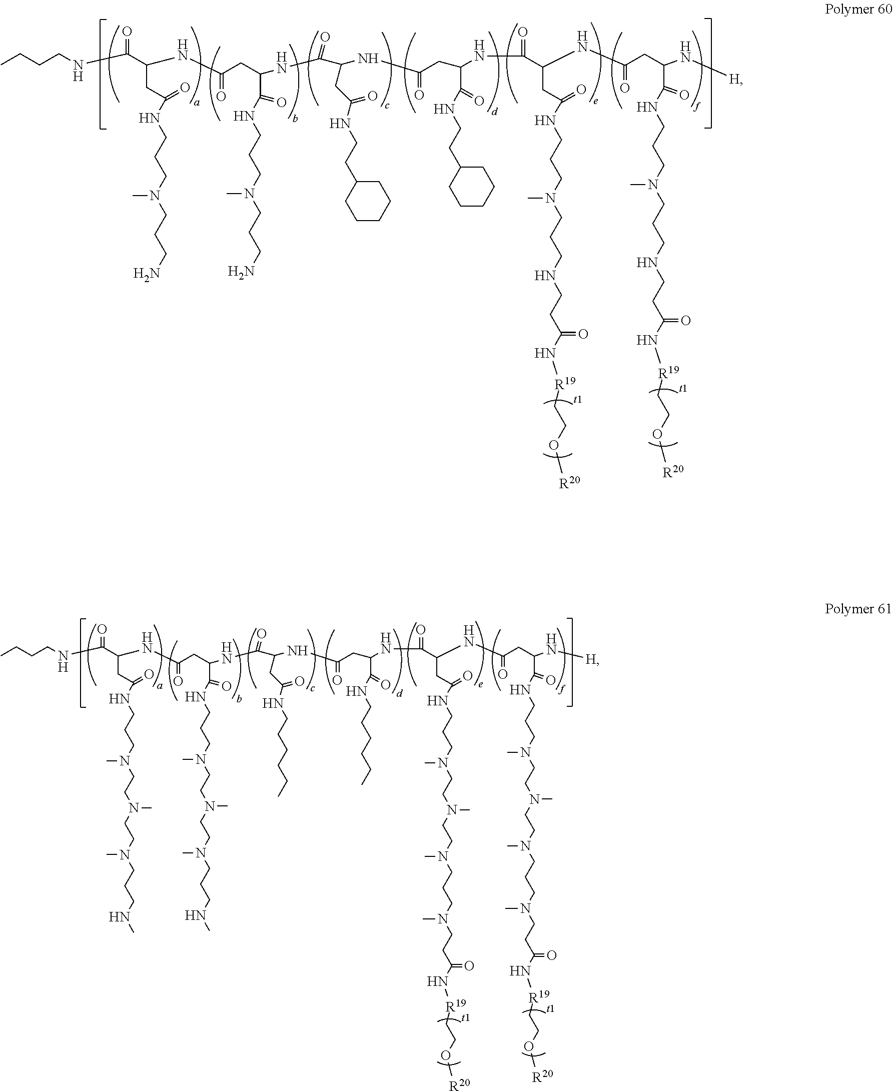

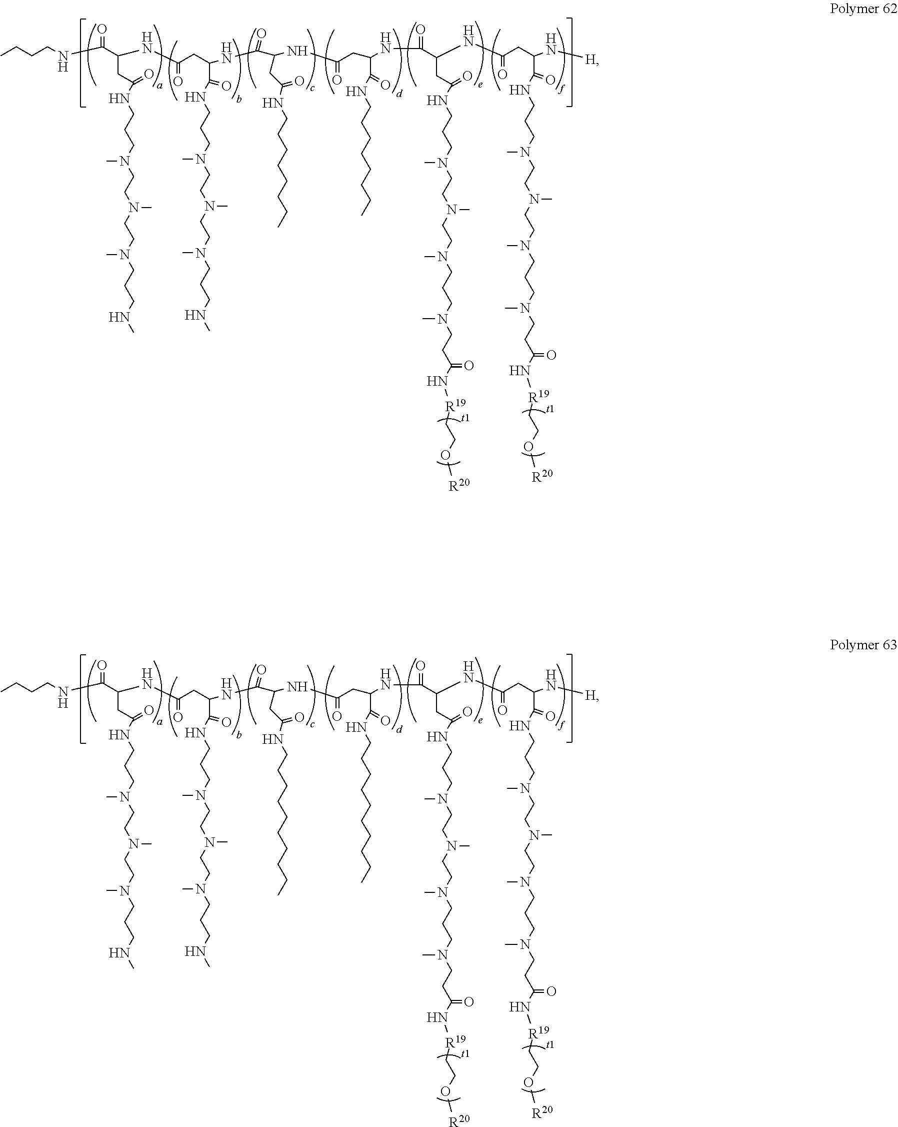

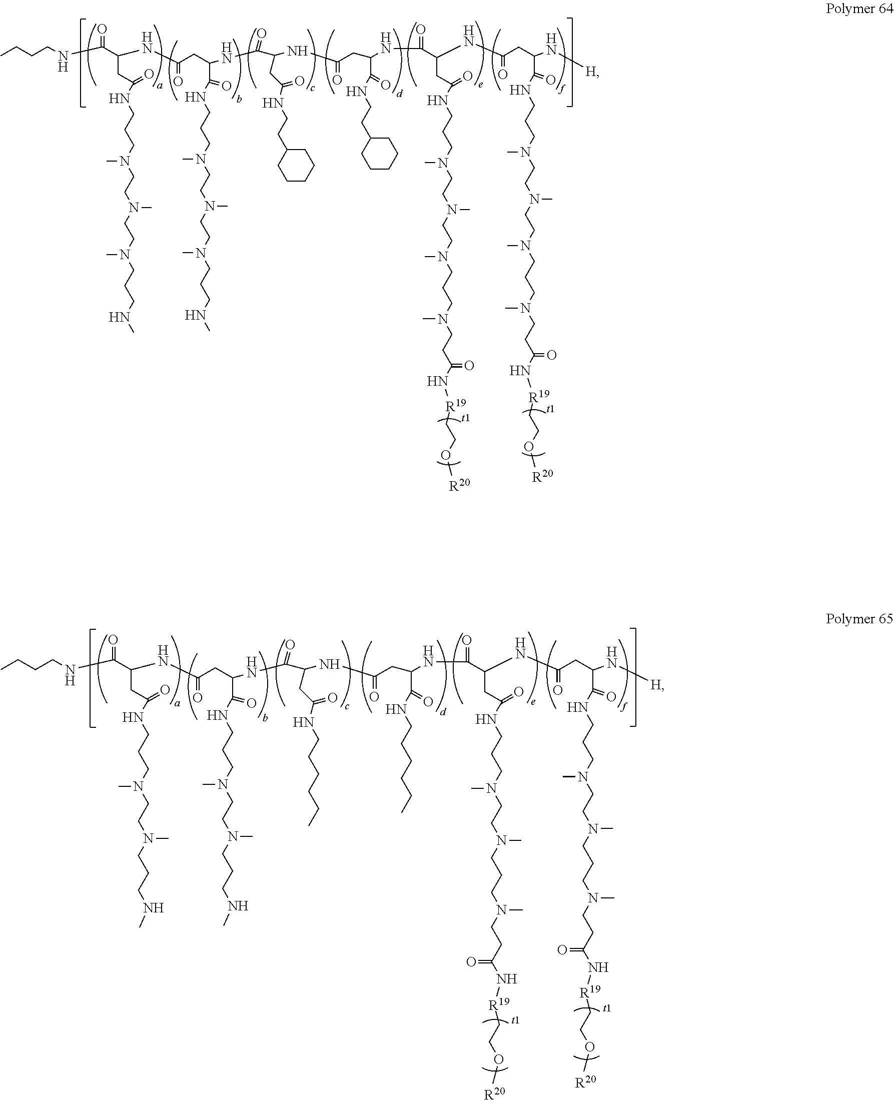

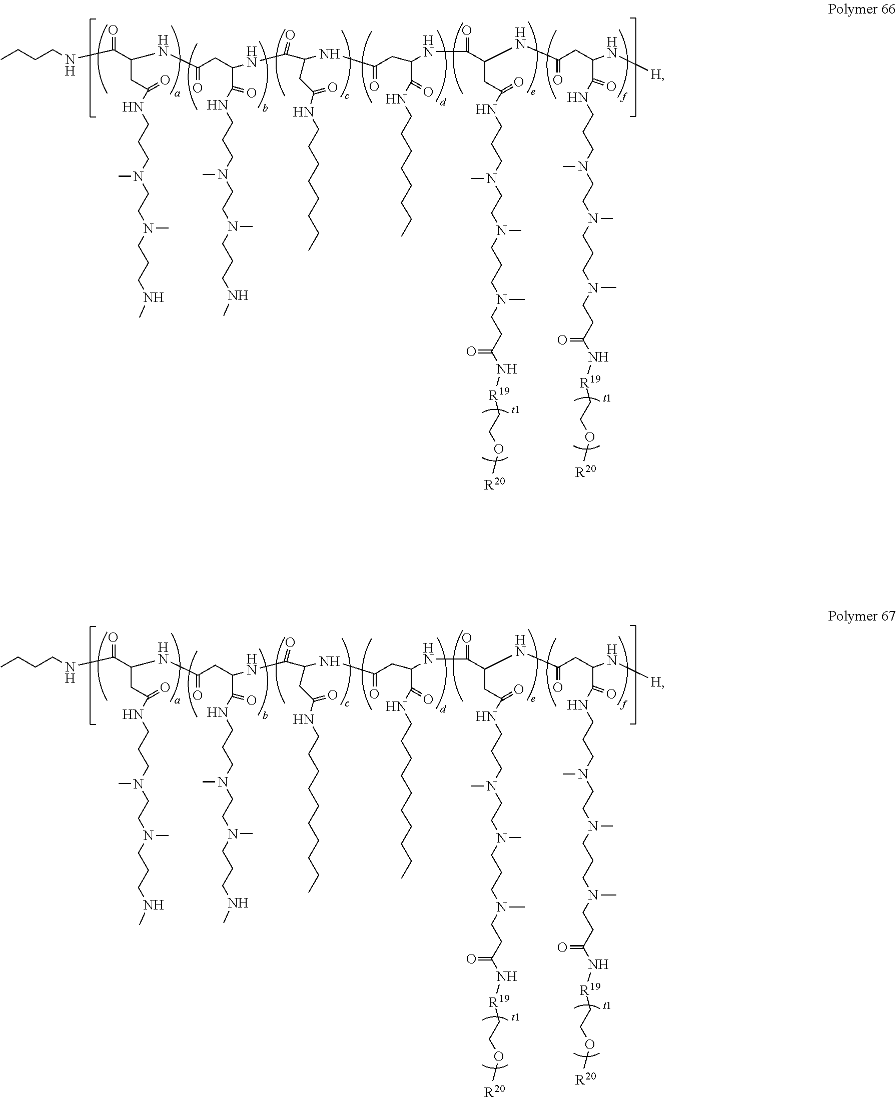

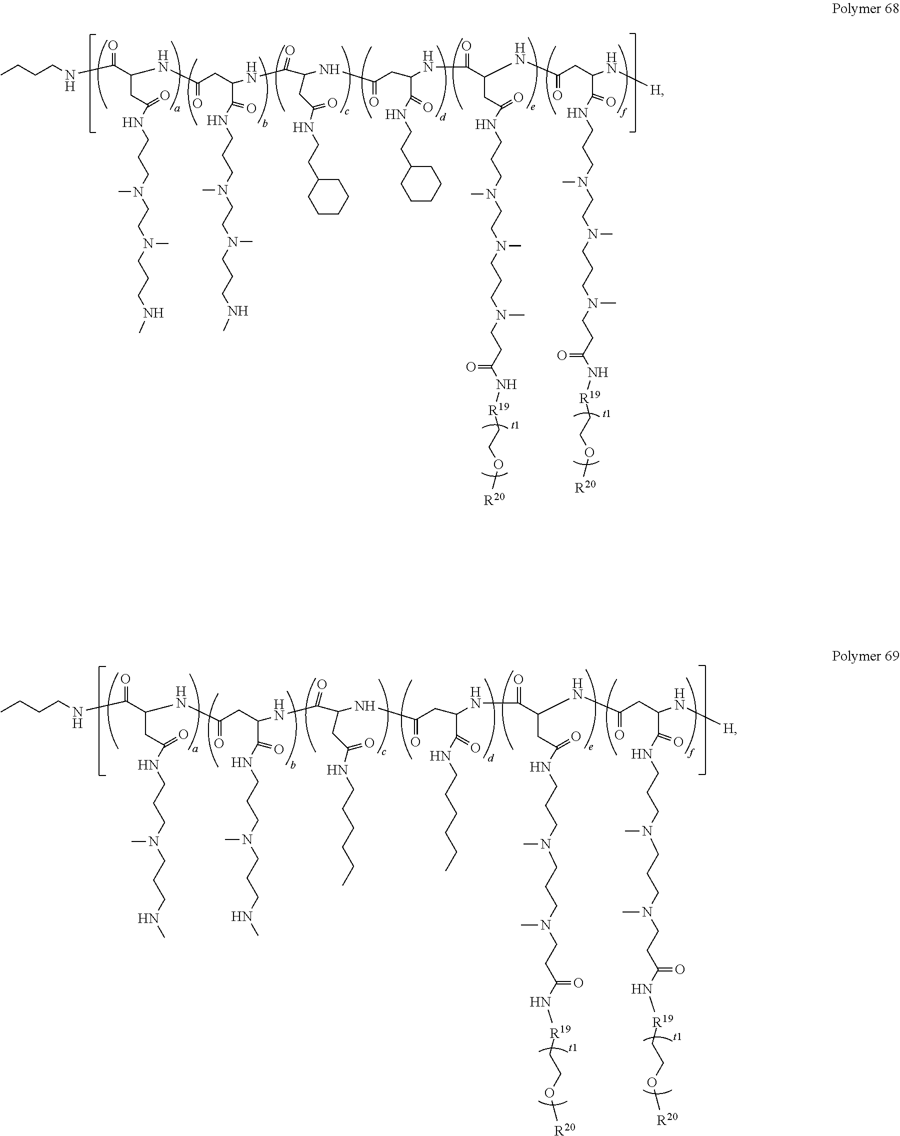

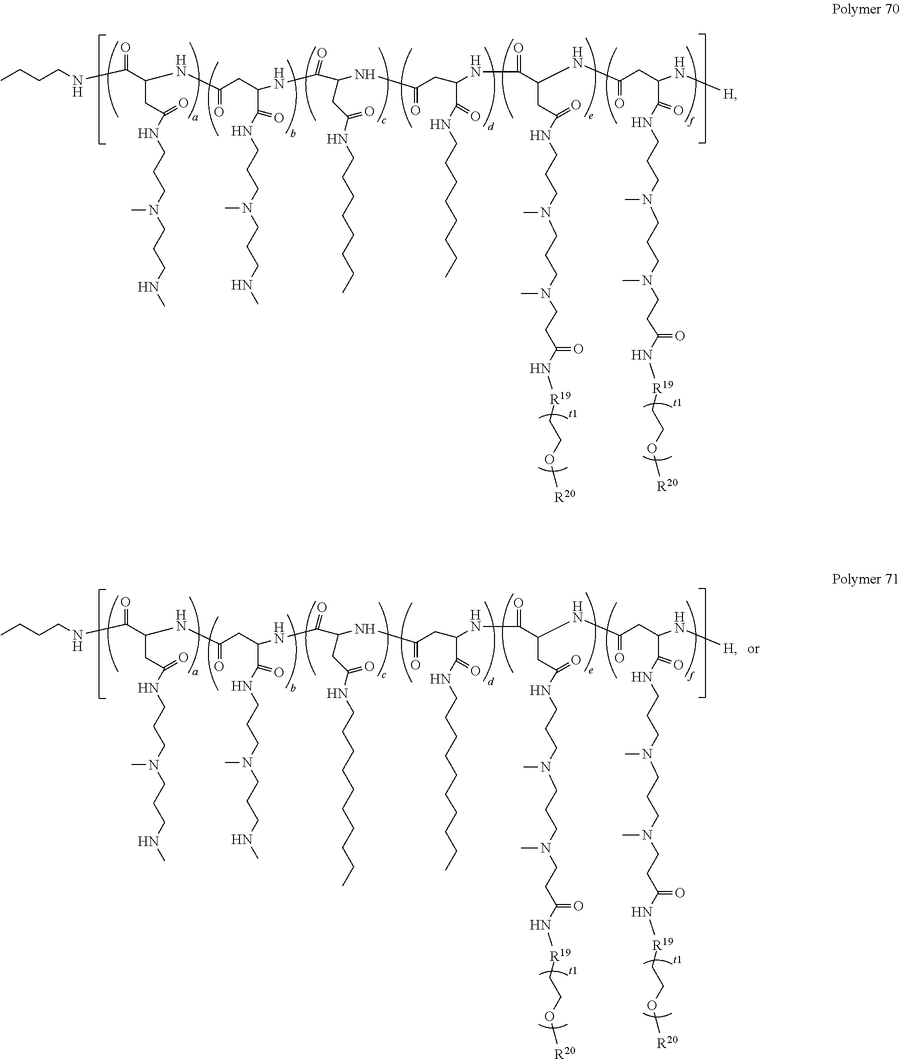

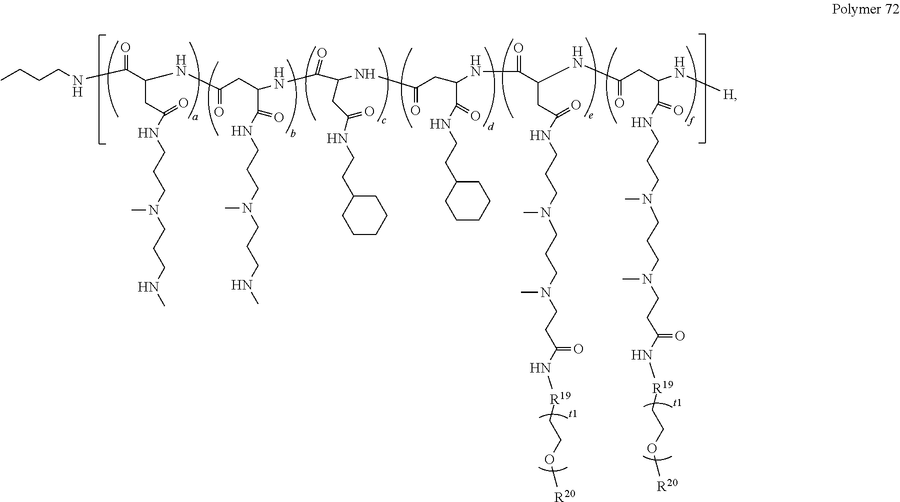

wherein R¹⁹ is a bond or a methylene, ethylene, or propylene group; R²⁰ is hydrogen, an aryl group (e.g., a C₆-C₁₂ alkyl group), a heterocyclic group (e.g., a C₂-C₁₂ heterocyclic group), a alkyl group (e.g., a C₁-C₁₂ alkyl group), alkenyl group (e.g., C₂-C₁₂ alkenyl group), cycloalkyl group (e.g., a C₃-C₁₂ cycloalkyl group), or cycloalkenyl group (e.g., a C₃-C₁₂ cycloalkenyl group), any of which can be optionally substituted with one or more substituents, optionally hydrogen or a C₁-C₃ alkyl group; and t1 is an integer from 2 to 200 (e.g., from 2 to 150, from 2 to 100, from 2 to 50, from 10 to 200, from 10 to 150, from 10 to 100, from 10 to 50, from 25 to 200, from 25 to 150, from 25 to 100, from 25 to 50, from 50 to 200, from 50 to 150, or from 50 to 100).

The indication of the number of units (“a”, “b”, “c”, “d”, “e”, and “f”) in these exemplary polymers does not imply a block co-polymer structure; rather, these numbers indicate the number of particular monomer units overall, which units can be arranged in any order, including blocks of monomers or monomers randomly arranged throughout the polymer. In some instances, but not all, this is additionally indicated by the “/” symbols in the formulas; however, the absence of a “/” should not be taken to mean that the polymers are joined in a particular order. In some embodiments of the foregoing Polymers 25-72, the monomers designated by parenthesis and an integer (“a”, “b”, “c”, “d”, “e”, or “f”) are randomly arranged or dispersed throughout the polymer.

In any of the foregoing polymers, (a+b+e+f) is from about 5 to about 65 (e.g., about 5 to about 50, about 5 to about 40, about 5 to about 30, about 5 to about 20, or about 5 to about 10) and (c+d) is from about 2 to about 60 (e.g., about 2 to about 50, about 2 to about 40, about 2 to about 30, about 2 to about 20, or about 2 to about 10). In certain embodiments, (a+b+e+f) is about 55 and (c+d) is about 10. In other embodiments, (a+b+e+f) is about 45 and (c+d) is about 20. In certain embodiments, (a+b+c+d+e+f) is about 10-500, such as about 10-400, about 10-200, or about 10-100 (e.g., about 25-100 or about 50-75). Again, the indication of the number of units (“a”, “b”, “c”, “d,” “e,” and “f”) in these exemplary polymers does not imply a block co-polymer structure; rather, these numbers indicate the number of units overall, which units can be randomly arranged.

The polymer can contain any suitable proportion of (a+b+e+f) to (c+d). In other embodiments, (a+b+e+f) ranges from 10-95% (e.g., 10-75%, 10-65%, 10-50%, 20-95%, 20-75%, 20-65%, 20-50%, 30-95%, 30-75%, 30-65%, or 30-50%) of the total number of polymer units (a+b+c+d+e+f). In other embodiments, (c+d) ranges from 5-90% of the total number of polymer units (a+b+c+d+e+f) (e.g., 5-75%, 5-65%, 5-50%, 5-40%, 5-30%, 10-90%, 10-75%, 10-65%, 10-50%, 10-40%, or 10-30%). In still other embodiments, the ratio of (a+b+e+f):(c+d) can be about 0.1 to about 1, about 0.1 to about 0.5, about 0.5 to about 1, about 1 to about 25, from about 1 to about 20, from about 1 to about 10, from about 1 to about 5, from about 5 to about 25, from about 10 to about 25, or from about 15 to about 25.

Some of the above particular examples of polymers provided by the disclosure are depicted with specific terminal groups (e.g., alkylamino or hydrogen); however, any of the foregoing particular structures can comprise different terminal groups. For example, any of the foregoing structures comprise a group of R¹, R⁶, or Q as described herein at either or both termini of the polymer backbone.

Any of the forgoing polymers can comprise a polyalkylene oxide group, a polyglycolic acid group, a polylactic acid group, a tissue-specific targeting moiety, a cell-specific targeting moiety, or a combination thereof at a position indicated in the described formulas, or the polymers can be otherwise modified to include a polyalkylene oxide group, a polyglycolic acid group, a polylactic acid group, a tissue-specific targeting moiety, a cell-specific targeting moiety, or a combination thereof. For example, the moiety can be added to a terminus of the polymer, or a terminal amine of A¹ groups can be modified (e.g., by a Michael addition reaction, an epoxide opening, a displacement reaction, or any other suitable technique) to attach the polyalkylene oxide group, a polyglycolic acid group, a polylactic acid group, a tissue-specific targeting moiety, a cell-specific targeting moiety, or a combination thereof.

Typically, the polymer is cationic (i.e., positively charged at pH 7 and 23° C.). As used herein, “cationic” polymers refer to polymers having an overall net positive charge, whether the polymer comprises only cationic monomer units or a combination of cationic monomer units and non-ionic or anionic monomer units.

In certain embodiments, the polymer has a weight average molecular weight of from about 5 kDa to about 2,000 kDa. The polymer can have a weight average molecular weight of about 2,000 kDa or less, for example, about 1,800 kDa or less, about 1,600 kDa or less, about 1,400 kDa or less, about 1,200 kDa or less, about 1,000 kDa or less, about 900 kDa, or less, about 800 kDa, or less, about 700 kDa or less, about 600 kDa or less, about 500 kDa or less, about 100 kDa or less, or about 50 kDa or less. Alternatively, or in addition, the polymer can have a weight average molecular weight of about 10 kDa or more, for example, about 50 kDa or more, about 100 kDa or more, about 200 kDa or more, about 300 kDa or more, or about 400 kDa or more. Thus, the polymer can have a weight average molecular weight bounded by any two of the aforementioned endpoints. For example, the polymer can have a weight average molecular weight of from about 10 kDa to about 50 kDa, from about 10 kDa to about 100 kDa, from about 10 kDa to about 500 kDa, from about 50 kDa to about 500 kDa, from about 100 kDa to about 500 kDa, from about 200 kDa to about 500 kDa, from about 300 kDa to about 500 kDa, from about 400 kDa to about 500 kDa, from about 400 kDa to about 600 kDa, from about 400 kDa to about 700 kDa, from about 400 kDa to about 800 kDa, from about 400 kDa to about 900 kDa, from about 400 kDa to about 1,000 kDa, from about 400 kDa to about 1,200 kDa, from about 400 kDa to about 1,400 kDa, from about 400 kDa to about 1,600 kDa, from about 400 kDa to about 1,800 kDa, from about 400 kDa to about 2,000 kDa, from about 200 kDa to about 2,000 kDa, from about 500 kDa to about 2,000 kDa, or from about 800 kDa to about 2,000 kDa.

The weight average molecular weight can be determined by any suitable technique. Generally, the weight average molecular weight is determined using size exclusion chromatography equipped with a column, selected from TSKgel Guard, GMPW, GMPW, G1000PW, and a Waters 2414 (Waters Corporation, Milford, Mass.) refractive index detector. Moreover, the weight average molecular weight is determined from calibration with polyethylene oxide/polyethylene glycol standards ranging from 150-875,000 Daltons.

In certain specific aspects, the polymer provided herein has a formula of:

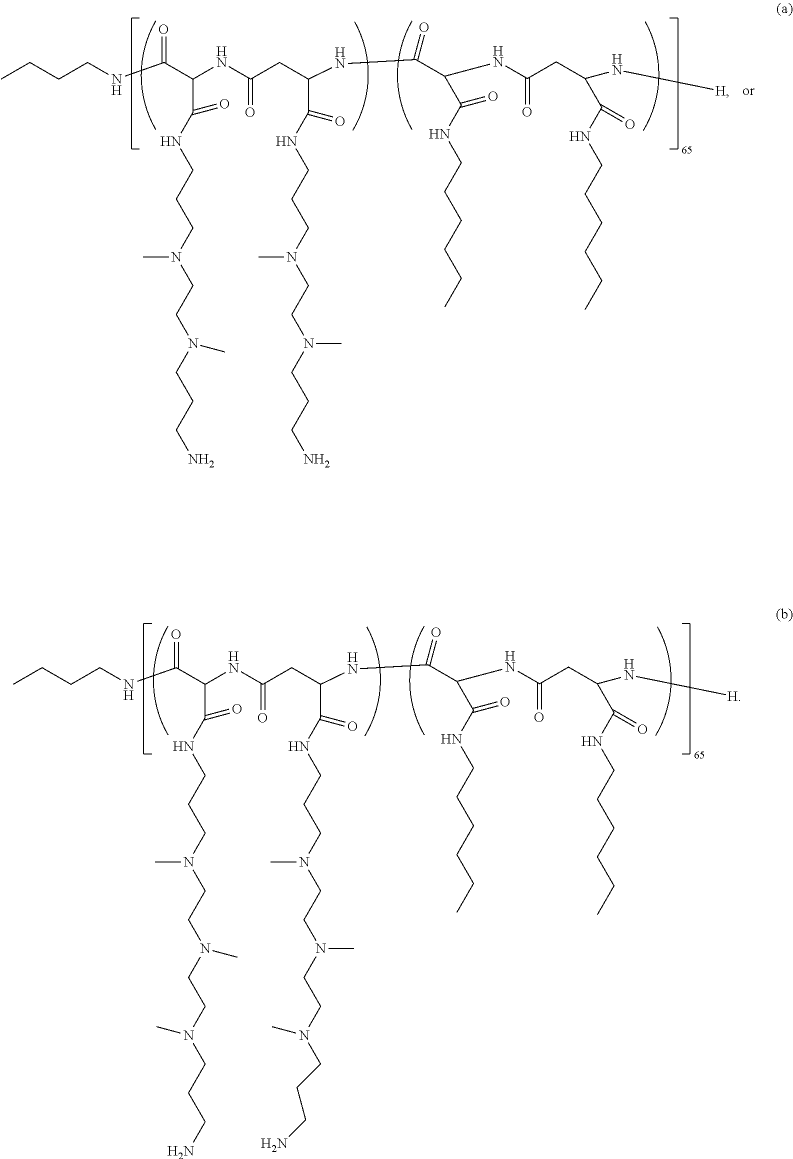

In another particular aspect, the polymer provided by any of the foregoing embodiments (e.g., a polymer of any of Formulas 1, 1A-C, 2, 2A-C, or any other described polymer) is not a polymer of (a) or (b), above. Thus, according to this aspects of the present disclosure, there is provided a polymer according to any of the foregoing embodiments, provided the polymer is not (a) or (b).

### Methods of Preparation

The invention also provides a method of preparing a polymer described herein. In some embodiments, the method comprises preparing a polymer of Formula 1:

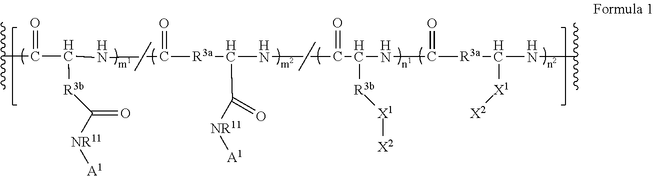

as described herein, the method comprising:

(I) reacting a polymer of Formula 3:

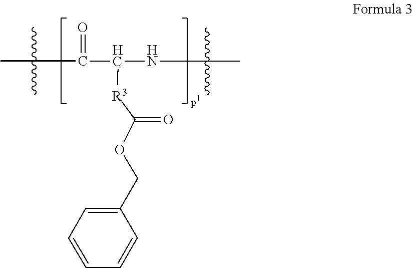

with (a) a compound of the formula HNR¹¹A¹; and (b) a compound of formula H₂NX² or HOX², simultaneously or in any sequential order; or

(II) reacting a polymer of Formula 4

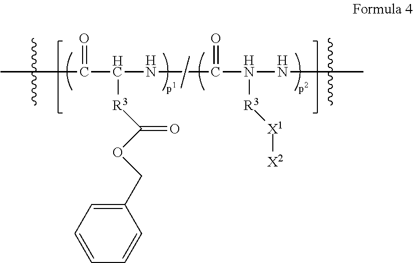

with a compound of the formula HNR¹¹A¹;
wherein,

p¹ is an integer from 1 to 2000 (e.g., from 1 to 1000, from 1 to 500, from 1 to 200, from 1 to 100, from 5 to 2000, from 5 to 1000, from 5 to 500, from 5 to 200, or from 5 to 100);

p² is an integer from 1 to 2000 (e.g., from 1 to 1000, from 1 to 500, from 1 to 200, from 1 to 100, from 2 to 2000, from 2 to 1000, from 2 to 500, from 2 to 200, or from 2 to 100);

each R³ is independently a methylene or ethylene group;

and m¹, m², n¹, n², R^{3a}, R^{3b}, R¹¹, X¹, X², and A¹ are as previously described with respect to Formulas 1 and 2. Thus, for instance, each X¹ independently is —C(O)O—, —C(O)NR¹¹—, —C(O)—, —S(O)(O)—, or a bond; and each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally substituted with one or more substituents. All other embodiments of X¹ and X² as previously described with respect to Formulas 1 and 2 also apply to X¹ and X² of Formulas 3 and 4.

The method comprises combining the structure of Formula 3 or Formula 4 with a compound of formula HNR¹¹A¹, and optionally a compound of formula H₂NX² or HOX². More specifically, the structure of Formula 3 can be combined (reacted) with (a) a compound of formula HNR¹¹A¹, and (b) a compound of formula H₂NX² or HOX², simultaneously or sequentially in any order, to provide the compound of Formula 1. Similarly, the compound of Formula 4, which already includes an X² group, can be combined (reacted) with a compound of formula HNR¹¹A¹ to provide the compound of Formula 1.

In the compound of HNR¹¹A¹, each instance of R¹¹ is as previously described with respect to the polymers of Formulas 1 and 2, including any and all embodiments thereof. Thus, for instance, each instance of R¹¹ can be independently hydrogen, an aryl group, a heterocyclic group, an alkyl group, alkenyl group, cycloalkyl group, or cycloalkenyl group, any of which can be optionally substituted with one or more substituents.

Similarly, A¹ is as previously described with respect to the polymers of Formulas 1 and 2. Thus, for instance, each instance of A¹ is independently a group of formula

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5; r1 is an integer of 0 to 5; and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In certain embodiments of A¹, p1, p3, or both are integers of 3 to 5.

Group X² of the compound of formula H₂NX² or HOX² is as described with respect to Formulas 1 and 2, including any and all embodiments thereof

All other substituents and aspects of Formulas 3 and 4, are as described herein with respect to the polymers of the invention (e.g., Formulas 1 and 2), including any and all embodiments thereof.

The compounds of HNR¹¹A¹ and of formula H₂NX² or HOX² can be added to the compound of Formula 3 or 4 in any suitable manner and amount depending upon the desired degree of substitution. In some embodiments, about 1-400 equivalents (e.g., about 1-350, 1-300, 1-250, 1-200, 1-150, 1-100, 1-50, 10-400, 10-350, 10-300, 10-250, 10-200, 10-150, 10-100, 10-50, 20-400, 20-350, 20-300, 20-250, 20-200, 20-150, 20-100, 20-50, 30-400, 30-350, 30-300, 30-250, 30-200, 30-150, 30-100, 30-50, 40-400, 40-350, 40-300, 40-250, 40-200, 40-150, 40-100, 40-50, 50-400, 50-350, 50-300, 50-250, 50-200, 50-150, or 50-100 equivalents) of the compound of formula H₂NX² or HOX² is added to polymer of Formula 3. Also, in some embodiments, about 1-400 equivalents (e.g., about 1-350, 1-300, 1-250, 1-200, 1-150, 1-100, 1-50, 10-400, 10-350, 10-300, 10-250, 10-200, 10-150, 10-100, 10-50, 20-400, 20-350, 20-300, 20-250, 20-200, 20-150, 20-100, 20-50, 30-400, 30-350, 30-300, 30-250, 30-200, 30-150, 30-100, 30-50, 40-400, 40-350, 40-300, 40-250, 40-200, 40-150, 40-100, 40-50, 50-400, 50-350, 50-300, 50-250, 50-200, 50-150, or 50-100 equivalents) of the compound of formula HNR¹¹A¹ is added to the polymer of Formula 3 or Formula 4.

In embodiments where the method comprises adding a compound of formula HNR¹¹A¹ and a compound of formula H₂NX² or HOX² to the polymer of Formula 3, the compound of formula HNR¹¹A¹ and the compound of formula H₂NX² or HOX² can be present in the reaction mixture in any suitable ratio. For example, the compound of formula HNR¹¹A¹ and the compound of formula H₂NX² or HOX² can be present in a molar ratio of about 150:1 to about 1:150. In some embodiments, a ratio of about 150:1 to about 1:1, such as about 50:1 to about 1:1 (e.g., about 25:1 to about 1:1, about 10:1 to about 1:1, about 5:1 to about 1:1, or about 2.5:1 to about 1:1) is used. In other embodiments, the ratio is about 1:150 to about 1:1, such as about 1:50 to about 1:1 (e.g., about 1:25 to about 1:1, about 1:10 to about 1:1, about 1:5 to about 1:1, or about 1:2.5 to about 1:1). In still other embodiments, the ratio is about 1:10 to about 1:150, about 1:40 to about 1:150, or about 1:80 to about 1:150.

In some embodiments, the polymer comprising a structure of Formula 3 or Formula 4 is a polymer of Formula 3A or Formula 4A, respectively:

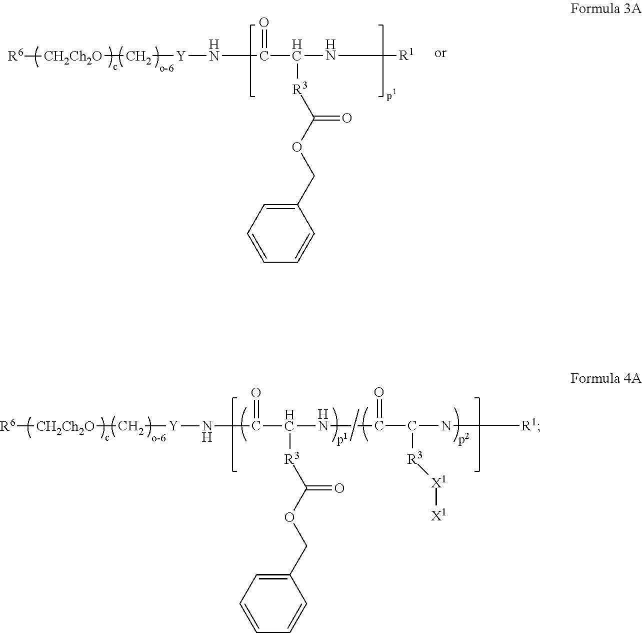

wherein c, Y, R¹, and R⁶ are as previously described with respect to the polymers of Formulas 1A and 2A, including any and all embodiments thereof; and p¹, p², R³, X¹, and X², are as described above with respect to Formulas 3 and 4. Thus, for instance:

p¹ is an integer from 1 to 2000;

p² is an integer from 1 to 2000;

each R³ is independently a methylene or ethylene group;

each X¹ independently is —C(O)O—, —C(O)NR¹—, —C(O)—, —S(O)(O)—, or a bond;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally comprising one or more primary, secondary, or tertiary amines; any of which can be substituted with one or more substituents;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents, optionally hydrogen or a C₁-C₃ alkyl group;

c is an integer from 0 to 50;

Y is optionally present and is a cleavable linker;

R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents;

R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety; and

the symbol “/” indicates that the units separated thereby are linked randomly or in any order. All aspects of Formulas 3A and 4A are otherwise as described herein with respect to the polymers of the invention (e.g., Formulas 1, 1A-1C, 2, 2A-2C, 3, and 4).

In certain embodiments, the polymer comprising a structure of Formula 3 or Formula 4 is a polymer of Formula 3B or Formula 4B, respectively:

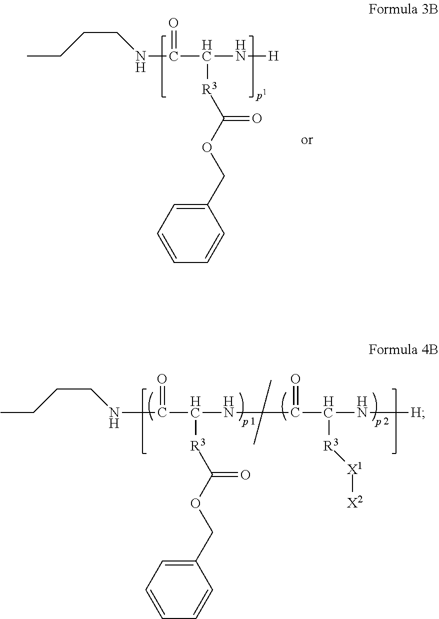

wherein p¹, p², R³, X¹, and X², are as described above with respect to Formulas 3, 3A, 4, and 4A. Thus, for instance:

p¹ is an integer from 1 to 2000;

p² is an integer from 1 to 2000;

each R³ is independently a methylene or ethylene group;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents, optionally hydrogen or a C₁-C₃ alkyl group

each X¹ independently is —C(O)O—, —C(O)NR¹—, —C(O)—, —S(O)(O)—, or a bond;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, optionally comprising one or more primary, secondary, or tertiary amines; any of which can be substituted with one or more substituents, or any other embodiments of X¹ and X² as previously described with respect to Formulas 1, 1A-1C, 2, 2A-2C, 3, and 4; and

the symbol “/” indicates that the units separated thereby are linked randomly or in any order. All aspects of Formulas 3B and 4B, are otherwise as described herein with respect to the polymers of the invention (e.g., Formulas 1, 1A-1C, 2, 2A-2C, 3, 3A, 4, and 4A).

In some embodiments, the method also provides a method of preparing a polymer of Formula 2, which comprises:

(a) providing a polymer of Formula 1 as defined herein:

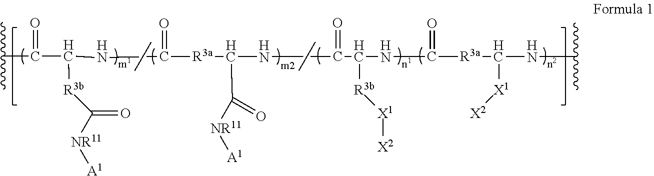

and

(b) modifying a portion of the A¹ groups of the polymer of Formula 1 to provide the polymer of Formula 2:

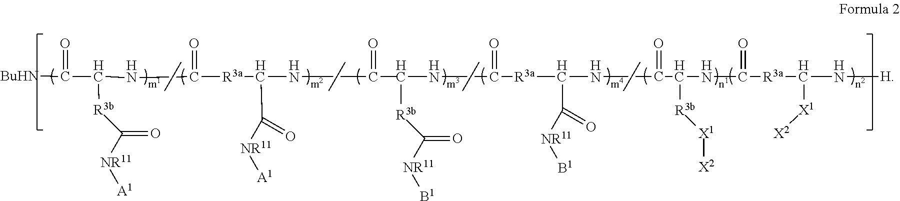

All aspects of the polymers of Formula 1 and 2 are as previously disclosed herein. Thus, for instance:

each of m¹, m², m³, and m⁴ is an integer from 0 to 1000, provided that the sum of m¹+m²+m³+m⁴ is greater than 2;

each of n¹ and n² is an integer from 0 to 1000, provided that the sum of n¹+n² is greater than 2;

the symbol “/” indicates that the units separated thereby are linked randomly or in any order;

each instance of R^{3a} is independently a methylene or ethylene group;

each instance of R^{3b} is independently a methylene or ethylene group;

each X¹ independently is —C(O)O—, —C(O)NR¹¹—, —C(O)—, —S(O)(O)—, or a bond;

each instance of R¹¹ is independently hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents;

each instance of A¹ is independently:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

each instance of B¹ is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴-R⁵,

wherein each of p¹ to p³ independently is an integer of 1 to 5, optionally wherein p¹, p³, or both are greater than p², and/or wherein p¹, p³, or both are integers of 3 to 5; r1 is an integer of 0 to 5; s1 is an integer from 1 to 5; each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof. All aspects of Formulas 1 and 2, are otherwise as described herein with respect to the polymers of the invention, including any and all embodiments of the structures of Formulas 1, 1A-1C, 2, 2A-2C, 3, 3A, 4, and 4A described herein.

The polymer comprising a structure of Formula 1 or Formula 2 can be any polymer described herein, including Formulas 1A, 1B, 1C, 2A, 2B, and 2C, as well as any and all embodiments thereof as described with respect to the polymer of the invention.

The groups designated A¹ of the polymer of Formula 1 can be modified by any suitable means to produce groups designated B¹ of the Polymer of Formula 2. For example, the groups designated A¹ can be modified by a Michael addition reaction, an epoxide opening, or a displacement reaction. In preferred embodiments, the groups designated A¹ are modified by a Michael addition reaction.

In one embodiment, A¹ groups of the polymer comprising a structure of Formula 1 are modified by a Michael addition reaction between the polymer comprising the structure of Formula 1 and α,β-unsaturated carbonyl compound. As used herein, the term “Michael addition” refers to a nucleophilic addition of a nucleophile of the polymer (e.g., a carbanion, an oxygen anion, a nitrogen anion, an oxygen atom, a nitrogen atom, or a combination thereof) to an α,β-unsaturated carbonyl compound. Accordingly, the Michael addition reaction is between the polymer comprising the structure of Formula 1 and an α,β-unsaturated carbonyl compound. In some embodiments, the nucleophile of the polymer is a nitrogen anion, a nitrogen atom, or a combination thereof.

The α,β-unsaturated carbonyl compound can be any α,β-unsaturated carbonyl compound capable of accepting a Michael addition from a nucleophile. In some embodiments, the α,β-unsaturated carbonyl compound is an acrylate, an acrylamide, a vinyl sulfone, or a combination thereof. Accordingly, the Michael addition reaction can be between the polymer comprising the structure of Formula 1 and an acrylate, an acrylamide, a vinyl sulfone, or a combination thereof. Thus, in some embodiments, the method comprises contacting the polymer comprising the structure of Formula 1 and an acrylate; contacting the polymer comprising the structure of Formula 1 and an acrylamide; or contacting the polymer comprising the structure of Formula 1 and a vinyl sulfone.

In embodiments where the groups designated A¹ are modified by a Michael addition reaction, they produce groups designated B¹ of the formula:

—(CH₂)ₚ₁—[NR²—(CH₂)ₚ₂—]ᵣ₁NR²—(CH₂)ₚ₃—NH—(CH₂)ₛ₁—R⁴-R⁵ or

—(CH₂)ₚ₁—[NR²—(CH₂)ₚ₂—]ᵣ₁NR²—(CH₂)ₚ₃—NR²—(CH₂)ₛ₁—R⁴-R⁵;

wherein each of p1 to p3 independently is an integer of 1 to 5, optionally wherein p1, p3, or both are greater than p2, and/or wherein p1, p3, or both are integers of 3 to 5; r1 is an integer of 0 to 5; s1 is an integer from 1 to 5; each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof.

Examples of acrylates, acrylamides, and vinyl sulfones suitable for use include an acrylate of the formula:

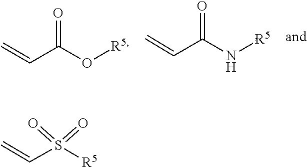

wherein R⁵ is as described with respect to any of Formulas 2 and 2A-2C.

In some embodiments, the Michael addition reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the Michael addition reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the Michael addition reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the Michael addition reaction is performed in a solvent. The solvent can be any suitable solvent, or mixture of solvents, capable of solubilizing the polymer and the α,β-unsaturated carbonyl compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, A¹ groups of the polymer are modified by an epoxide opening reaction between the polymer and an epoxide compound. As used herein, the term “epoxide opening” refers to a nucleophilic addition of a nucleophile of the polymer (e.g., a carbanion, an oxygen anion, a nitrogen anion, an oxygen atom, a nitrogen atom, or a combination thereof) to an epoxide compound, thereby opening the epoxide. Accordingly, the epoxide opening reaction is between the polymer and an epoxide compound. In some embodiments, the nucleophile of the polymer is a nitrogen anion, a nitrogen atom, or a combination thereof.

In embodiments where the groups designated A¹ are modified by an epoxide opening reaction, they produce groups designated B¹ of the formula:

—(CH₂)ₚ₁—[NR²—(CH₂)ₚ₂—]ᵣ₁NR²—(CH₂)ₚ₃—NH—CH₂—CHOH—R⁵ or

—(CH₂)ₚ₁—[NR²—(CH₂)ₚ₂—]ᵣ₁NR²—(CH₂)ₚ₃—NR²—CH₂—CHOH—R⁵;

wherein each of p1 to p3 independently is an integer of 1 to 5, optionally wherein p1, p3, or both are greater than p2, and/or wherein p1, p3, or both are integers of 3 to 5; r1 is an integer of 0 to 5; s1 is an integer from 1 to 5; each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof.

Examples of epoxides suitable for use include epoxides of the formula:

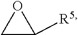

wherein R⁵ is as described with respect to any of Formulas 2 and 2A-2C.

In some embodiments, the epoxide opening reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the epoxide opening reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the epoxide opening reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the epoxide opening reaction is performed in a solvent. The solvent can be any suitable solvent, or mixture of solvents, capable of solubilizing the polymer and the epoxide compound to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In one embodiment, A¹ groups of the polymer are modified by a displacement reaction between the polymer and a compound comprising a leaving group (e.g., chloride atom, bromide atom, iodide atom, tosylate, triflate, mesylate, etc.). As used herein, the term “displacement” refers to a nucleophilic addition of a nucleophile of the polymer (e.g., a carbanion, an oxygen anion, a nitrogen anion, an oxygen atom, a nitrogen atom, or a combination thereof) to a compound comprising a leaving group. Accordingly, the displacement reaction is between the polymer and a compound comprising a leaving group. In some embodiments, the nucleophile of the polymer is a nitrogen anion, a nitrogen atom, or a combination thereof.

In embodiments where the groups designated A¹ are modified by a displacement reaction, they produce groups designated B¹ of the formula:

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁵;

wherein each of p1 to p3 independently is an integer of 1 to 5, optionally wherein p1, p3, or both are greater than p2, and/or wherein p1, p3, or both are integers of 3 to 5; each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof.

Examples of compounds containing a leaving group suitable for use include compound of formula:

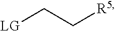

wherein LG is a leaving group (e.g., chloride atom, bromide atom, iodide atom, tosylate, triflate, mesylate, etc.) and R⁵ is as described with respect to any of Formulas 2 and 2A-2C.

In some embodiments, the displacement reaction is facilitated by an acid and/or base. The acid and/or base can be any suitable acid and/or base with any suitable pKa. The acid and/or base can be an organic acid (e.g., p-toluenesulfonic acid), organic base (e.g., triethylamine), inorganic acid (e.g., titanium tetrachloride), inorganic base (e.g., potassium carbonate), or a combination thereof.

In some embodiments, the displacement reaction is facilitated by an acid. The acid can be a Brønsted acid or a Lewis acid. In embodiments where the acid is a Brønsted acid, the acid can be a weak acid (i.e., a pKa of from about 4 to about 7) or a strong acid (i.e., a pKa of from about −2 to about 4). Typically, the acid is a weak acid. In certain embodiments, the acid is a Lewis acid. For example, the acid can be bis(trifluoromethanesulfon)imide or p-toluenesulfonic acid.

In some embodiments, the displacement reaction is facilitated by a base. The base can be a weak base (i.e., a pKa of from about 7 to about 12) or a strong base (i.e., a pKa of from about 12 to about 50). Typically, the base is a weak base. For example, the base can be triethylamine, diisopropylethylamine, pyridine, N-methyl morpholine, or N,N-dimethyl-piperazine, or derivatives thereof.

In some embodiments, the displacement reaction is performed in a solvent. The solvent can be any suitable solvent, or mixture of solvents, capable of solubilizing the polymer and the compound comprising a leaving group to be reacted. For example, the solvent can include water, protic organic solvents, and/or aprotic organic solvents. An exemplary list of solvents includes water, dichloromethane, diethyl ether, dimethyl sulfoxide, acetonitrile, methanol, and ethanol.

In some embodiments, the method further comprises isolating the polymer comprising the structure of Formula 2. The polymer comprising the structure of Formula 2 can be isolated by any suitable means. For example, the polymer comprising the structure of Formula 2 can be isolated by extraction, crystallization, recrystallization, column chromatography, filtration, or any combination thereof.

### Additional Polymer

The polymer provided herein can be used in conjunction with additional polymers. The polymers can be combined in any suitable manner (e.g., mixed) to provide a polymer nanoparticle. In one embodiment, there is provided a composition comprising a first polymer and a second polymer, wherein the first polymer is as described herein, e.g., a polymer comprising a hydrolysable polymer backbone, the polymer backbone comprising (i) monomer units comprising a hydrophobic side chain; and (ii) monomer units comprising a cationic side chain; wherein the cationic side chain comprises a polyamine with at least one tertiary amine and only a single nucleophilic center, optionally at the terminus of the polyamine, and optionally (iii) monomer units comprising a cationic side chain comprising a polyamine with at least one tertiary amine and a tissue-specific or cell-specific targeting moiety and/or a group comprising polyalkylene oxide, polyglycolic acid, or polylactic acid linked to the terminus of the polyamine. All aspects and embodiments of the first polymer of the composition are as described above.

The second polymer, according to this embodiment, comprises a hydrolysable polymer backbone, the polymer backbone comprising: (a) monomer units with a side chain comprising a hydrophobic group; (b) monomer units with a cationic side chain comprising a polyamine; and (c) monomer units with a side chain comprising a polyalkylene oxide, polyglycolic acid, polylactic acid, or combination thereof. While both the first and second polymer can comprise monomers with side chains comprising a polyalkylene oxide, polyglycolic acid, polylactic acid, or combination thereof, in some embodiments only the second polymer comprises monomers with a side chain comprising a polyalkylene oxide, polyglycolic acid, polylactic acid, or combination thereof.

The composition can comprise any suitable amount of the first polymer and second polymer. For example, the composition can comprise a ratio (by weight) of the second polymer to the first polymer from about 1:99 to 99:1. In some embodiments, the composition comprises a ratio of the second polymer to the first polymer of about 1:1 to about 1:20 (e.g., about 1:1 to about 1:15, or about 1:1 to about 1:10) by weight. The relative amounts also can be expressed as percent composition by weight. In some embodiments, the composition comprises about 1 wt. % or more (e.g., about 5 wt. % or more, about 10 wt. % or more, about 20 wt. % or more, about 30 wt. % or more, or about 40 wt. % or more) of the second polymer based on the total weight of the first and second polymers combined. Also, in some embodiments, the composition comprises about 60 wt. % or less (e.g., about 50 wt. % or less) of the second polymer based on the total weight of the first and second polymers combined. The foregoing percent compositions can also be stated as ranges. Thus, for instance, in some embodiments, the composition comprises from about 1 wt. % to about 60 wt. % (e.g., about 5 wt. % to about 60 wt. %, about 10 wt. % to about 60 wt. %, about 5 wt. % to about 50 wt. %, about 10 wt. % to about 50 wt. %, etc.) of the second polymer based on a sum total weight of the first polymer and the second polymer.

In the second polymer, the hydrolysable backbone is as described for the first polymer, and can be the same or different type of backbone structure that of the first polymer. Thus, for example, the hydrolysable polymer backbone can comprise a polyamide, poly-N-alkylamide, polyester, polycarbonate, polycarbamate, or a combination thereof. In certain embodiments, the hydrolysable polymer backbone comprises a polyamide.

The monomer units with a side chain comprising a hydrophobic group can be any as described with respect to the first polymer, and can be the same or different from those monomer groups of the first polymer. Thus, the monomer units with a side chain comprising a hydrophobic group, can comprise any hydrophobic group, and can be linked to the polymer backbone in any suitable manner, such as directly or via a linkage comprising, for instance, an ester, an amide, or an ether group, optionally further comprising an alkylene linker (e.g., a methylene or ethylene linker). Examples of hydrophobic groups include, for instance, a C₁-C₁₂ (e.g., C₂-C₁₂, C₂-C₁₀, C₂-C₈, C₂-C₆, C₃-C₁₂, C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅-C₁₀,) alkyl group, a C₂-C₁₂ (e.g., C₂-C₆, C₃-C₁₂, C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅- C₁₀,) alkenyl group, or a C₃-C₁₂ (C₃-C₁₀, C₃-C₈, C₃-C₆, C₄-C₁₂, C₄-C₁₀, C₄-C₈, C₄-C₆, C₆-C₁₂, C₆-C₈, C₅-C₁₂, C₅-C₁₀,) cycloalkyl group or cycloalkenyl group. In certain embodiments, the hydrophobic group comprises a C₄-C₁₂ alkyl group, alkenyl group, cycloalkyl group, or cycloalkenyl group. In some embodiments, the hydrophobic group comprises fewer than 8 carbons or fewer than 6 carbons. For example, the hydrophobic group can comprise a C₂-C₈ or C₂-C₆ (e.g., C₃-C₈ or C₃-C₆) alkyl group. The alkyl or alkenyl groups can be branched or straight-chain. The alkyl or alkenyl groups can be substituted, provided the substituent groups do not negate the hydrophobicity of the overall hydrophobic side-chain (e.g., does not render the side-chain hydrophilic). For instance, the alkyl or alkenyl group can be substituted with hydroxyl groups (e.g., 1 or 2 hydroxyl groups) or halogen groups (e.g., substituted with one or more fluorine, chlorine, bromine, or iodine atoms). In any of the foregoing embodiments, the hydrophobic group can be linked to the polymer backbone directly or via a linkage comprising, for instance, an ester, an amide, or an ether group, optionally further comprising an alkylene linker (e.g., a methylene or ethylene linker).

The polymer also comprises monomer units with a cationic side chain comprising an polyamine (e.g., a chemical moiety comprising two or three or more amine groups (e.g., oligoamines) or larger polyamines comprising four or more (e.g., 4, 5, 6, 7, 8, 9, 10, 11, 12, 13, 14, 15, 16, or more) amine groups). Examples of polyamines include, for instance, polyalkylamines. The polyamines can be linear or branched. In some embodiments, the polyamines are linear. The amine groups can be primary amine groups, secondary amine groups, tertiary amine groups, or any combination thereof.

The monomers with a cationic side chain comprising a polyamine of the second polymer can be the same as or different from the monomers comprising a cationic side chain of the first polymer. In some embodiments, the polyamine side chain of the second polymer is of the formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²-]r₂R²};

—(CH₂)ₚ₄—N{—(CH₂)_{q5}—N[—(CH₂)_{q6}—NR²₂]₂}₂,

—(CH₂)ₚ₁—[NR²—(CH₂)_{q1}—]ᵣ₁NR²—(CH₂)ₛ₁—R⁴-R⁵;

—(CH₂)ₚ₂—N[—(CH₂)_{q2}—NR²—(CH₂)ₛ₂—R⁴-R⁵]₂;

—(CH₂)ₚ₃—N{[—(CH₂)_{q3}—NR²₂][—(CH₂)_{q4}—NR²—]ᵣ₂(CH₂)ₛ₃—R⁴-R⁵};

—(CH₂)ₚ₄—N{—(CH₂)_{q5}—N[—(CH₂)_{q6}—NR²—(CH₂)ₛ₄—R⁴-R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R⁵;

—(CH₂)ₚ₃—N{[—(CH₂)_{q3}—NR²₂][—(CH₂)_{q4}—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N{(CH₂)ₛ₁—R⁴-R⁵}—(CH₂)_{q1}—]ᵣ₁NR²₂;

—(CH₂)ₚ₁—[N{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴-R⁵,

wherein p1 to p4, q1 to q6, r1 and r2, and s1 to s4 are each independently an integer of 1 to 5;

each instance of R² is independently hydrogen or a C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group;

each instance of R⁴ is independently —C(O)O—, —C(O)NH—, —O—C(O)O—, or —S(O)(O)—; and

each instance of R⁵ is independently an alkyl group, cycloalkyl group, alkenyl group, cycloalkenyl group, aryl group, heteroalkyl group, heterocyclic group, or combination thereof optionally comprising from 2 to 8 tertiary amines or a substituent comprising a tissue-specific or cell-specific targeting moiety.

In some embodiments, the polyamine side chain of the second polymer is of the formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]ᵣ₂R²}; or

—(CH₂)ₚ₄—N{—(CH₂)_{q5}—N[—(CH₂)_{q6}—NR²₂]₂}₂,

wherein p1 to p4, q1 to q6, and r1 and r2 are each independently an integer of 1 to 5 (e.g., 1, 2, or 3); and

each instance of R² is independently hydrogen or a C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. It is understood that the alkenyl groups must have at least 2 carbons (e.g., C₂-C₁₂, C₂-C₆, etc.) and the cycloalkyl and cycloalkenyl groups must have at least 3 carbons (e.g., C₃-C₁₂, C₃-C₆, etc.). In some embodiments, the polyamine is —(CH₂)ₚ₁—[NR²—(CH₂)_{q1}—]ᵣ₁NR²₂, optionally wherein R² is independently hydrogen or a C₁-C₃ alkyl (e.g., methyl or ethyl).

In some embodiments, the polyamine side chain of the second polymer has the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,

wherein each of p1 to p3 independently is an integer of 1 to 5 (e.g., 1, 2, 3, 4, or 5); r1 is an integer of 0 to 5 (e.g., 1, 2, 3, 4, or 5); and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In some embodiments, p1, p3, or both are greater than p2 and/or p1, p3, or both are integers of 3 to 5 (e.g., 3, 4, or 5) and, optionally, p² is an integer of 1 or 2. Each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid or combination thereof; a tissue-specific targeting moiety, a cell-specific targeting moiety, or a combination thereof.

In some embodiments, the monomer units comprise a cationic side chain of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵;

optionally wherein at least some portion (or all) comprise a side chain of the formula:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵.

In some of any of the foregoing embodiments, p1, p3, or both are greater than p2, and/or p1, p3, or both are integers of 3 to 5 (e.g., 3, 4, or 5), optionally wherein p2 is 1 or 2. In some embodiments, R² is a C₁-C₆ alkyl group, such as a C₁-C₃ alkyl group (e.g., methyl or ethyl).

In still additional embodiments, some (or all) of the monomers of the second polymer with a cationic side chain comprise a polyamine of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5 (e.g., 1, 2, 3, 4, or 5); r1 is an integer of 0 to 5 (e.g., 1, 2, 3, 4, or 5); and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In some embodiments, p1, p3, or both are greater than p2 and/or p1, p3, or both are integers of 3 to 5 (e.g., 3, 4, or 5) and, optionally, p2 is an integer of 1 or 2.

In some embodiments, the polyamine is —(CH₂)ₚ₁—[NR²—(CH₂)ₚ₂—]ᵣ₁NR²—(CH₂)ₚ₃—NH₂, optionally wherein R² is independently a C₁-C₆ alkyl group (e.g., methyl or ethyl). In some embodiments, the polyamine is —(CH₂)ₚ₁—[NR²—(CH₂)ₚ₂—]ᵣ₁NR²—(CH₂)ₚ₃—NHR², optionally wherein R² is independently a C₁-C₆ alkyl group (e.g., methyl or ethyl) group. In further embodiments comprising any of these features, p1, p3, or both can be greater than p2 and/or p1, p3, or both can be integers of 3 to 5 (e.g., 3, 4, or 5).

Specific non-limiting examples of polyamine side chains of the second polymer include, for instance,

—CH₂—CH₂—N(CH₃)—CH₂—CH₂—N(CH₃)₂;

—CH₂—CH₂—N(CH₃)—CH₂—CH₂—N(CH₃)₂;

—CH₂—CH₂—N(CH₃)—CH₂—CH₂—N(CH₃)—CH₂—CH₂—N(CH₃)₂;

—CH₂—CH₂—N(CH₃)—CH₂—CH₂—N(CH₃)—CH₂—CH₂—N(CH₃)₂;

—CH₂—CH₂—N(CH₃)—CH₂—CH₂—NH(CH₃);

—CH₂—CH₂—N(CH₃)—CH₂—CH₂—NH(CH₃);

—CH₂—CH₂—N(CH₃)—CH₂—CH₂—N(CH₃)—CH₂—CH₂—NH(CH₃);

—CH₂—CH₂—N(CH₃)—CH₂—CH₂—N(CH₃)—CH₂—CH₂—NH(CH₃);

—CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—CH₂—NH₂; or

—CH₂—CH₂—CH₂—N(CH₃)—CH₂—CH₂—CH₂—NHCH₃.

The second polymer also comprises monomer units with a side chain comprising a polyalkylene oxide, polyglycolic acid, polylactic acid, or combination thereof, can be linked to the polymer backbone in any suitable manner, such as directly or via a linkage comprising, for instance, an ester, an amide, or an ether group, optionally further comprising an alkylene linker (e.g., a methylene or ethylene linker). In some embodiments, the side chain comprises at least one polyethylene glycol group (a.k.a. polyethylene oxide group) having a sum total of from 2 to 200 ethylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polypropylene oxide group having a sum total of from 2 to 200 propylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, the side chain comprises at least one polylactic acid and/or polyglycolic acid group having a sum total of from 2 to 200 polylactic acid and/or polyglycolic acid units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In certain embodiments, the side chain comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units. In embodiments where the side chain comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units, the side chain can exist as any suitable structure type. For example, the side chain can comprise the ethylene glycol and/or propylene oxide units and the polylactic acid and/or polyglycolic acid units as an alternating polymer, random polymer, block polymer, graft polymer, linear polymer, branched polymer, cyclic polymer, or a combination thereof. In certain embodiments, the side chain comprises polyalkylene oxide units (e.g., ethylene oxide units, propylene oxide units, or both) and optionally further comprises polylactic acid or polyglycolic acid units. For example, the side chain of at least some of the monomers can be derived from PLURONIC® F65 or PLURONIC® F127.

For instance, the second polymer can comprise monomer units with a side chain comprising a polyalkylene oxide, polyglycolic acid, polylactic acid, or combination thereof, which comprise a structure of the following formula:

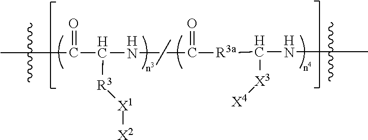

wherein:

each of n³ and n⁴ is an integer from 0 to 1000, provided that the sum of n³+n⁴ is greater than 1;

the symbol “/” indicates that the units separated thereby are linked randomly or in any order;

each instance of R^{3a} is independently a methylene or ethylene group;

each instance of R^{3b} is independently a methylene or ethylene group;

each X³ independently is —C(O)O—, —C(O)NR¹¹—, —C(O)—, —S(O)(O)—, or a bond;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X⁴ comprises polyalkylene oxide, polyglycolic acid, polylactic acid, or a combination thereof; and

each instance of p is independently an integer from 2 to 200.

Just as for the first polymer, the different monomer units of the second polymer can be arranged in any order, including blocks of monomers or monomers randomly arranged throughout the polymer. Furthermore, the polymer can comprise any suitable number or amount (e.g., weight or number percent composition) of the monomer units with a side chain comprising a hydrophobic group, the monomer units with a side chain comprising an oligoamine or polyamine, and the monomer units with a side chain comprising a polyalkylene oxide, polyglycolic acid, polylactic acid, or combination thereof. In some embodiments, the polymer comprises about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having a hydrophobic group. In some embodiment, the polymer comprises about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having an oligoamine or polyamine. In some embodiments, the polymer comprises about 1 to about 80 mol % (e.g., about 5 to about 80 mol %, about 10 to about 80 mol %, about 20 to about 80 mol %, about 40 to about 80 mol %, about 1 to about 60 mol %, about 1 to about 40 mol %, about 1 to about 20 mol %, or about 1 to about 10 mol %) of the monomer units having a polyalkylene oxide, polyglycolic acid, polylactic acid, or combination thereof.

In some embodiments, the second polymer has a polyamide backbone. For instance, the polymer can have the structure of Formula P2:

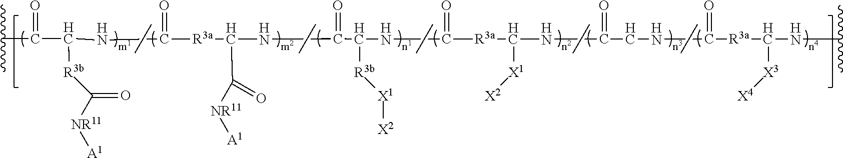

wherein

each of m¹ and m² is an integer from 0 to 1000, provided that the sum of m¹+m² is greater than 1;

each of n¹ and n² is an integer from 0 to 1000, provided that the sum of n¹+n² is greater than 1;

each of n³ and n⁴ is an integer from 0 to 1000, provided that the sum of n³+n⁴ is greater than 1;

the symbol “/” indicates that the units separated thereby are linked randomly or in any order;

each instance of R^{3a} is independently a methylene or ethylene group;

each instance of R^{3b} is independently a methylene or ethylene group;

each of X¹ and X³ are independently-C(O)O—, —C(O)NR¹¹—, —C(O)—, —S(O)(O)—, or a bond; optionally wherein X¹ and X³ are —C(O)NR¹¹—, or wherein X¹ and X³ are —C(O)O—;

each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents;

each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof; any of which are optionally substituted with one or more substituents;

each instance of X⁴ comprises polyalkylene oxide, polyglycolic acid, polylactic acid, or a combination thereof; and

each of E¹ and E² are cationic side chains comprising a polyamine.

In certain embodiments, each of E¹ and E² are independently a group of formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₁—N{[—(CH₂)_{q3}—NR²₂][—(CH₂)_{q4}—NR²—]ᵣ₂R²}; or

—(CH₂)ₚ₄—N{—(CH₂)_{q5}—N[—(CH₂)_{q6}—NR²₂]₂}₂,

—(CH₂)ₚ₁—[NR²—(CH₂)_{q1}—]ᵣ₁NR²—(CH₂)ₛ₁—R⁴-R⁵;

—(CH₂)ₚ₂—N[—(CH₂)_{q2}—NR²—(CH₂)ₛ₂—R⁴-R⁵]₂;

—(CH₂)ₚ₃—N{[—(CH₂)_{q3}—NR²₂][—(CH₂)_{q4}—NR²—]ᵣ₂(CH₂)ₛ₃—R⁴-R⁵};

—(CH₂)ₚ₄—N{—(CH₂)_{q5}—N[—(CH₂)_{q6}—NR²—(CH₂)ₛ₄—R⁴-R⁵]₂}₂;

—(CH₂)ₚ₁—[NR²—(CH₂)_{q1}—]ᵣ₁NR²—CH₂—CHOH—R⁵;

—(CH₂)ₚ₂—N[—(CH₂)_{q2}—NR²—CH₂—CHOH—R⁵;

—(CH₂)ₚ₃—N{[—(CH₂)_{q3}—NR²₂][—(CH₂)_{q4}—NR²—]ᵣ₂—CH₂—CHOH—R⁵;

—(CH₂)ₚ₄—N{—(CH₂)_{q5}—N[—(CH₂)_{q6}—NR²—CH₂—CHOH—R⁵]₂}₂;

—(CH₂)ₚ₁—[NR²—(CH₂)_{q1}—]ᵣ₁NR²—(CH₂)ₛ₁—R⁵;

—(CH₂)ₚ₂—N[—(CH₂)_{q2}—NR²—(CH₂)ₛ₂—R⁵]₂;

—(CH₂)ₚ₃—N{[—(CH₂)_{q3}—NR²₂][—(CH₂)_{q4}—NR²—]ᵣ₂(CH₂)ₛ₃—R⁵};

—(CH₂)ₚ₄—N{—(CH₂)_{q5}—N[—(CH₂)_{q6}—NR²—(CH₂)ₛ₄—R⁵]₂}₂;

—(CH₂)ₚ₁—[N—{(CH₂)ₛ₁—R⁴-R⁵}—(CH₂)_{q1}—]ᵣ₁NR²₂;

—(CH₂)ₚ₁—[N—{(CH₂)ₛ₁—R⁵}—(CH₂)_{q1}—]ᵣ₁NR²₂,

—(CH₂)ₚ₁—[NR²—(CH₂)_{q1}—]ᵣ₁NR²—CH(CONH₂)—(CH₂)ₛ₁—R⁵; or

—(CH₂)ₚ₁—[NR²—(CH₂)_{q1}—]ᵣ₁NR²—CH(CONH₂)—(CH₂)ₛ₁—R⁴-R⁵,

wherein p1 to p4, q1 to q6, r1 and r2, and s1 to s4 are each independently an integer of 1 to 5 (1, 2, 3, 4, or 5), optionally 1-3 (1, 2, or 3), or 1 or 2;

each instance of R² is independently hydrogen or a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, any of which are optionally substituted with one or more substituents, or R² is combined with a second R² so as to form a heterocyclic group;

each instance of R⁴ is independently —C(O)O—, —C(O)NH—, —O—C(O)O—, or —S(O)(O)—; optionally —C(O)O— or —C(O)NH—; and

each instance of R⁵ is independently a C₁-C₁₂ alkyl group, C₂-C₁₂ alkenyl group, C₃-C₁₂ cycloalkyl group, or C₃-C₁₂ cycloalkenyl, aryl group, C₁-C₁₂ heteroalkyl group, C₃-C₁₂ heterocyclic group, or combination thereof optionally comprising from 2 to 8 tertiary amines or a substituent comprising a tissue-specific or cell-specific targeting moiety.

In some embodiments, each of E¹ and E² are each independently a group of formula:

—(CH₂)ₚ₁—[NR²—(CH₂)_{q1}—]ᵣ₁NR²₂;

—(CH₂)ₚ₂—N[—(CH₂)_{q2}—NR²₂]₂;

—(CH₂)ₚ₃—N{[—(CH₂)_{q3}—NR²₂][—(CH₂)_{q4}—NR²—]ᵣ₂R²}; or

—(CH₂)ₚ₄—N{—(CH₂)_{q5}—N[—(CH₂)_{q6}—NR²₂]₂}₂,

In some embodiments, the polyamine is —(CH₂)ₚ₁—[NR²—(CH₂)_{q1}—]ᵣ₁NR²₂, optionally wherein R² is independently hydrogen or a C₁-C₃ alkyl (e.g., methyl or ethyl).

Alternatively, each of E¹ and E² are each independently a group of formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,

wherein each of p¹ to p³ independently is an integer of 1 to 5 (e.g., 1, 2, 3, 4, or 5); r1 is an integer of 0 to 5 (e.g., 1, 2, 3, 4, or 5); and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In some embodiments, p¹, p³, or both are greater than p² and/or p¹, p³, or both are integers of 3 to 5 (e.g., 3, 4, or 5) and, optionally, p² is an integer of 1 or 2. Each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid or combination thereof; a tissue-specific targeting moiety, a cell-specific targeting moiety, or a combination thereof.

In some such embodiments, E¹ and E² are each independently a group of formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵;

optionally wherein at least some portion (or all) comprise a side chain of the formula:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵.

In some of any of the foregoing embodiments, p1, p3, or both are greater than p², and/or p1, p3, or both are integers of 3 to 5 (e.g., 3, 4, or 5), optionally wherein p2 is 1 or 2. In some embodiments, R² is a C₁-C₆ alkyl group, such as a C₁-C₃ alkyl group (e.g., methyl or ethyl).

In still additional embodiments, some (or all) instances of E¹ and E² are of the formula:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

wherein each of p1 to p3 independently is an integer of 1 to 5 (e.g., 1, 2, 3, 4, or 5); r1 is an integer of 0 to 5 (e.g., 1, 2, 3, 4, or 5); and each instance of R² is independently C₁-C₁₂ (e.g., C₁-C₆, C₁-C₃, C₂, or C₁) alkyl group, C₂-C₁₂ (e.g., C₂-C₆, or C₂-C₃) alkenyl group, C₃-C₁₂ (e.g., C₃-C₈ or C₃-C₆) cycloalkyl group, or C₃-C₁₂ (e.g., C₃-C₈ or C₃-C₆) cycloalkenyl group, or R² is combined with a second R² so as to form a heterocyclic group. In some embodiments, p1, p3, or both are greater than p2 and/or p1, p3, or both are integers of 3 to 5 (e.g., 3, 4, or 5) and, optionally, p2 is an integer of 1 or 2.

In some embodiments, E¹ and E² are —(CH₂)ₚ₁—[NR²—(CH₂)ₚ₂—]ᵣ₁NR²—(CH₂)ₚ₃—NH₂, optionally wherein R² is independently a C₁-C₆ alkyl group (e.g., methyl or ethyl). In some embodiments, E¹ and E² are —(CH₂)ₚ₁—[NR²—(CH₂)ₚ₂—]ᵣ₁NR²—(CH₂)ₚ₃—NHR², optionally wherein R² is independently a C₁-C₆ alkyl group (e.g., methyl or ethyl) group. In further embodiments comprising any of these features, p1, p3, or both can be greater than p2 and/or p1, p3, or both can be integers of 3 to 5 (e.g., 3, 4, or 5).

Specific non-limiting examples of E¹ and E² include, for instance,

—CH₂—CH₂—N(CH₃)—CH₂—CH₂—N(CH₃)₂;

—CH₂—CH₂—N(CH₃)—CH₂—CH₂—N(CH₃)₂;

—CH₂—CH₂—N(CH₃)—CH₂—CH₂—N(CH₃)—CH₂—CH₂—N(CH₃)₂;

—CH₂—CH₂—N(CH₃)—CH₂—CH₂—N(CH₃)—CH₂—CH₂—N(CH₃)₂;

—CH₂—CH₂—N(CH₃)—CH₂—CH₂—NH(CH₃);

—CH₂—CH₂—N(CH₃)—CH₂—CH₂—NH(CH₃);

—CH₂—CH₂—N(CH₃)—CH₂—CH₂—N(CH₃)—CH₂—CH₂—NH(CH₃);

—CH₂—CH₂—N(CH₃)—CH₂—CH₂—N(CH₃)—CH₂—CH₂—NH(CH₃);

—CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—N(CH₃)—CH₂—CH₂—CH₂—NH₂;

—CH₂—CH₂—CH₂—N(CH₃)—CH₂—CH₂—N(CH₃)—CH₂—CH₂—CH₂—NHCH₃;

—CH₂—CH₂—CH₂—N(CH₃)—CH₂—CH₂—CH₂—NH₂; or

—CH₂—CH₂—CH₂—N(CH₃)—CH₂—CH₂—CH₂—NHCH₃.

As used herein, terms such as “alkyl,” “alkylene,” “aryl,” “heterocyclyl,” “heterocyclic,” “substituted,” have the same meaning as used in describing the first polymer.

According to Formula P2, each of m¹, m², n¹, n², n³, and n⁴ is an integer from 0 to 1000 (e.g., 0 to 500, 0 to 200, 0 to 100, or 0 to 50), provided that the sum of m¹+m²+n¹+n²+n³+n⁴ is greater than 5, such as 5-5000, 5-2000, 5-1000, 5-500, 5-100, or 5-50. In some embodiments, the sum of m¹+m²+n¹+n²+n³+n⁴ is greater than 10 or greater than 20 (e.g., 10-5000, 10-2000, 10-1000, 10-500, 10-100, or 10-50; or 20-5000, 20-2000, 20-1000, 20-500, 20-100, or 20-50). In some embodiments, each of m¹ and m² is an integer from 0 to 1000 (e.g., 0 to 500, 0 to 200, 0 to 100, 0 to 50, or 0 to 25), provided that the sum of m¹+m² is greater than 1 (e.g., 1-2000, 1-1000, 1-500, 1-200, 1-100, 1-50, or 1-25). In some embodiments, the sum of m¹+m² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25). In certain embodiments, each of n¹ and n² is an integer from 0 to 1000 (e.g., 0 to 500, 0 to 200, 0 to 100, 0 to 50, or 0 to 25), provided that the sum of n¹+n² is greater than 1 (e.g., 1-2000, 1-1000, 1-500, 1-200, 1-100, 1-50, or 1-25). In some embodiments, the sum of n¹+n² is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25). In some embodiments, each of n³ and n⁴ is an integer from 0 to 1000 (e.g., 0 to 500, 0 to 200, 0 to 100, 0 to 50, or 0 to 25), provided that the sum of n³+n⁴ is greater than 1 (e.g., 1-2000, 1-1000, 1-500, 1-200, 1-100, 1-50, or 1-25). In some embodiments, the sum of n³+n⁴ is greater than 5 or greater than 10 (e.g., 5-2000, 5-1000, 5-500, 5-200, 5-100, 5-50, or 5-25; or 10-2000, 10-1000, 10-500, 10-200, 10-100, 10-50, or 10-25).

In other words, the second polymer comprises at least some n³ and/or n⁴ monomer units, i.e., at least some monomer units comprising X⁴ groups, which comprise a polyalkylene oxide, polyglycolic acid, polylactic acid, or a combination thereof (referred to collectively as “X⁴” monomers). The second polymer also comprises at least some m¹ and/or m² monomeric units, i.e., monomer units comprising variables E¹ or E² comprising polyamine groups (referred to collectively as “E” monomers). In addition, the second polymer comprises at least some monomeric units comprising groups n¹ and/or n² monomer units, i.e., monomer units comprising X² groups which include a hydrophobic group (referred to collectively as “X²” monomers).

The second polymer can comprise any suitable ratio of E monomers, X² monomers, and X⁴ monomers. In some embodiments, the polymer comprises a number ratio of E monomers to the sum of X² and X⁴ monomers (i.e., (m¹+m²)/(n¹+n²+n³+n⁴)) can be about 0.5 to about 500 (e.g., about 0.5 to about 250, about 0.5 to about 100, about 0.5 to about 50, about 0.5 to about 10, about 0.5 to about 5, about 0.5 to about 2, about 0.5 to about 1.5, about 0.5 to about 1, about 1 to about 500, about 5 to about 500, about 10 to about 500, about 50 to about 500, about 100 to about 500, or about 250 to about 500). Alternatively, or in addition, the ratio of X² monomers to the sum of X⁴ monomers and E monomers (i.e., (n¹+n²)/(m¹+m²+n³+n⁴)) in some embodiments can be about 0.002 to about 500 (e.g., about 0.002 to about 250, about 0.002 to about 100, about 0.002 to about 50, about 0.002 to about 10, about 0.002 to about 5, about 0.002 to about 1, about 0.5 to about 250, about 0.5 to about 100, about 0.5 to about 50, about 0.5 to about 10, about 0.5 to about 5, about 0.5 to about 2, about 0.5 to about 1.5, about 0.5 to about 1, about 1 to about 500, about 5 to about 500, about 10 to about 500, about 50 to about 500, about 100 to about 500, or about 250 to about 500). Alternatively, or in addition, in some embodiments, the ratio of X⁴ monomers to the sum of X² and E monomers (i.e., (n³+n⁴)/(m¹+m²+n¹+n²)) can be about 0.002 to about 500 (e.g., about 0.002 to about 250, about 0.002 to about 100, about 0.002 to about 50, about 0.002 to about 10, about 0.002 to about 5, about 0.002 to about 1, about 0.002 to about 0.5, about 0.002 to about 0.25), about 0.01 to about 500 (e.g., about 0.01 to about 250, about 0.01 to about 100, about 0.01 to about 50, about 0.01 to about 10, about 0.01 to about 5, about 0.01 to about 1, about 0.01 to about 0.5, about 0.01 to about 0.25), about 0.05 to about 500 (e.g., about 0.05 to about 250, about 0.05 to about 100, about 0.05 to about 50, about 0.05 to about 10, about 0.05 to about 5, about 0.05 to about 1, about 0.05 to about 0.5, about 0.05 to about 0.25), about 0.1 to about 500 (e.g., about 0.1 to about 250, about 0.1 to about 100, about 0.1 to about 50, about 0.1 to about 10, about 0.1 to about 5, about 0.1 to about 1, about 0.1 to about 0.5, about 0.1 to about 0.25), about 0.5 to about 500 (e.g., about 0.5 to about 250, about 0.5 to about 100, about 0.5 to about 50, about 0.5 to about 10, about 0.5 to about 5, about 0.5 to about 1), about 1 to about 500 (e.g., about 1 to about 250, about 1 to about 100, about 1 to about 50, about 1 to about 10, about 1 to about 5, about 1 to about 2). The polymer can have any suitable type of structure. For example, the second polymer can be an alternating polymer wherein the different monomers alternate according to some sequence or pattern; a block polymer wherein multiple monomers of a given type (e.g., “E” monomers, “X²” monomers, or “X⁴” monomers) are arranged in blocks; or random or grafted polymer wherein the various monomer types (e.g., “E” monomers, “X²” monomers, or “X⁴” monomers) are arranged in any order. The second polymer can be structured as a linear polymer, branched polymer, cyclic polymer, or a combination thereof.

By way of further illustration, the monomers (which can be referred to by reference to their E¹, E², X², and X⁴ side chains according to Formula P2) can be arranged randomly or in any order. Thus, m¹, m², n¹, n², n³, and n⁴, merely denote the number of the respective monomers that appear in the chain overall, and do not describe or imply blocks of those monomers, although blocks or stretches of a given monomer might be present in some embodiments. For instance, the structure of Formula P2 can comprise the monomers in the order -E¹-E²-X²-X⁴—, -E²-E¹-X²-X⁴—, -E¹-X²-E²-X⁴—, etc. Furthermore, the polymer can comprise blocks of E¹, E², X², and/or X⁴ monomers in any order. In some embodiments, the E¹, E², X², and X⁴ monomers are randomly distributed throughout the polymer. In other embodiments, the polymer comprises one or more segments in which the E¹, E², and X² monomers are randomly distributed, and a segment comprising a block of X⁴ monomers. In some embodiments, the segment comprising a block of X⁴ monomers can be sandwiched between segments comprising E¹, E², and X² monomers. In other embodiments, the segment comprising a block of X⁴ monomers cap the polymer at one or both ends. In some embodiments, at least some portion (or the entirety) of the polymer (e.g., polyaspartamide) backbone will comprising monomers arranged in an alpha/beta configuration, such that monomers comprising R^{3a} and R^{3b} (referring to Formula P2) will alternate along the backbone.

In the second polymer, each instance of R¹¹ is independently hydrogen or a C₁-C₁₂ (e.g., C₁-C₈, C₁-C₆, or C₁-C₃) alkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, aryl group, or heterocyclic group (e.g., 3-12, 3-10, 3-8, or 3-6 membered heterocyclic group comprising one, two, or three heteroatoms), any of which can be substituted with one or more substituents. In some embodiments, R¹¹ is a C₁-C₁₂ alkyl group (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group) which can be linear or branched. In some embodiments, R¹¹ is a C₁-C₄ alkyl group, a C₁-C₃ alkyl group, or a C₁ or C₂ alkyl group. In certain embodiments, each R¹¹ is methyl or hydrogen. In some embodiments, R¹¹ is methyl; in other embodiments, R¹¹ is hydrogen. Each R¹¹ is independently chosen and can be the same or different; however, in some embodiments, each R¹¹ is the same (e.g., all are methyl or all are hydrogen).

In the second polymer, each instance of X² is independently C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, C₃-C₁₂ (e.g., C₃-C₈, C₃-C₆, or C₃-C₅) cycloalkenyl group, aryl group, or heterocyclic group (e.g., 3-12, 3-10, 3-8, or 3-6 membered heterocyclic group comprising one, two, or three heteroatoms) or combination thereof, any of which can be substituted with one or more substituents. In some embodiments, X² comprises a cyclic or fused ring hydrophobic group (e.g., cycloalkyl, cycloalkenyl, heterocyclyl, or aryl group having 4 to 12 members (e.g., C₄-C₁₂). In some embodiments, X² optionally can comprise one or more primary, secondary, or tertiary amines. In other embodiments, the alkyl or alkenyl group can be substituted with one or more hydroxyl groups (e.g., 1 or 2 hydroxyl groups) or halogen groups (e.g., substituted with one or more fluorine, chlorine, bromine, or iodine atoms). In embodiments, X² is hydrophobic, and any substituents should not negate the hydrophobicity of the X² group (e.g., does not render the group hydrophilic). Each X² is independently selected and, therefore, can be the same or different from one another. In some embodiments, one or more (or all) X² groups can be independently a C₂-C₁₂ (e.g., C₃-C₁₂, C₃-C₈, C₃-C₆, C₄-C₁₂, C₄-C₆, C₆-C₁₂, or C₅-C₁₂) alkyl group or alkenyl group, or C₃-C₁₂ (e.g., C₃-C₈, C₃-C₆, C₄-C₁₂, C₄-C₆, C₆-C₁₂, or C₈-C₁₂) cycloalkenyl group, optionally substituted with one or more hydroxyl groups (e.g., 1 or 2 hydroxyl groups) or halogen groups (e.g., substituted with one or more fluorine, chlorine, bromine, or iodine atoms). In other embodiments, one or more (or all) X² groups can be independently C₁-C₈ (e.g., C₁-C₆, C₁-C₄, C₁-C₃, C₂-C₈, C₂-C₆, C₃-C₈, or C₃-C₆) alkyl groups, optionally substituted with one or more hydroxyl groups (e.g., 1 or 2 hydroxyl groups) or halogen groups (e.g., substituted with one or more fluorine, chlorine, bromine, or iodine atoms). Any of the foregoing alkyl or alkenyl groups can be linear or branched.

In the second polymer, each instance of X⁴ is a polyalkylene oxide, polyglycolic acid, polylactic acid, or a combination thereof. In some embodiments, each instance of X⁴ comprises at least one polyethylene glycol group (a.k.a. polyethylene oxide group) having a sum total of from 2 to 200 ethylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, X⁴ comprises at least one polypropylene oxide group having a sum total of from 2 to 200 propylene oxide units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In some embodiments, X⁴ comprises at least one polylactic acid and/or polyglycolic acid group having a sum total of from 2 to 200 polylactic acid and/or polyglycolic acid units (e.g., from 2 to 150 units, from 2 to 100 units, from 2 to 50 units, from 10 to 200 units, from 10 to 150 units, from 10 to 100 units, from 10 to 50 units, from 25 to 200 units, from 25 to 150 units, from 25 to 100 units, from 25 to 50 units, from 50 to 200 units, from 50 to 150 units, or from 50 to 100 units). In certain embodiments, X⁴ comprises at least one polyethylene glycol/polypropylene oxide group having a sum total of from 2 to 200 ethylene glycol and/or propylene oxide units and at least one polylactic acid and/or polyglycolic acid group having a sum total of from 2 to 200 polylactic acid and/or polyglycolic acid units. In some embodiments, each instance of X⁴ comprises

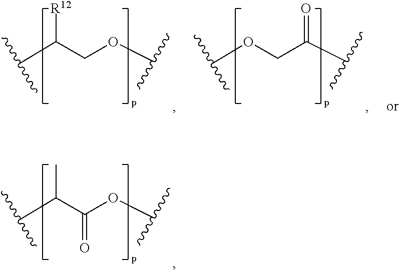

or some combination thereof, wherein p is independently an integer from 2 to 200 (e.g., from 2 to 150, from 2 to 100, from 2 to 50, from 10 to 200, from 10 to 150, from 10 to 100, from 10 to 50, from 25 to 200, from 25 to 150, from 25 to 100, from 25 to 50, from 50 to 200, from 50 to 150, or from 50 to 100), and each instance of R¹² is independently hydrogen or methyl. The X⁴ group can further be terminated with an R¹¹ group as defined herein. In some embodiments, X⁴ is

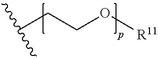

wherein p and R¹¹ are as previously defined.

Each instance of R⁴ is independently —C(O)O—, —C(O)NH—, or —S(O)(O)—. In some embodiments, each instance of R⁴ is independently —C(O)O— or —C(O)NH—. In certain embodiments, each instance of R⁴ is —C(O)O—. In certain embodiments, each instance of R⁴ is —C(O)NH—.

Each instance of R⁵ is independently an alkyl group, cycloalkyl group, alkenyl group, cycloalkenyl group, aryl group, heteroalkyl group, heterocyclic group, or combination thereof, optionally comprising from 2 to 8 tertiary amines or a substituent comprising a tissue-specific or cell-specific targeting moiety. R⁵ can comprise from about 2 to about 50 carbon atoms (e.g., from about 2 to about 40 carbon atoms, from about 2 to about 30 carbon atoms, from about 2 to about 20 carbon atoms, from about 2 to about 16 carbon atoms, from about 2 to about 12 carbon atoms, from about 2 to about 10 carbon atoms, or from about 2 to about 8 carbon atoms). In some embodiments, R⁵ is a heteroalkyl group comprising from 2 to 8 (i.e., 2, 3, 4, 5, 6, 7, or 8) tertiary amines. The tertiary amines can be part of the heteroalkyl backbone (i.e., the longest continuous chain of atoms in the heteroalkyl group, or a pendant substituent. Thus, for instance, the heteroalkyl group comprising the tertiary amines can provide an alkylamino group, amino alkyl group, alkylaminoalkyl group, aminoalkylamino group, or the like comprising 2 to 8 tertiary amines.

In some embodiments, each R⁵ is independently selected from:

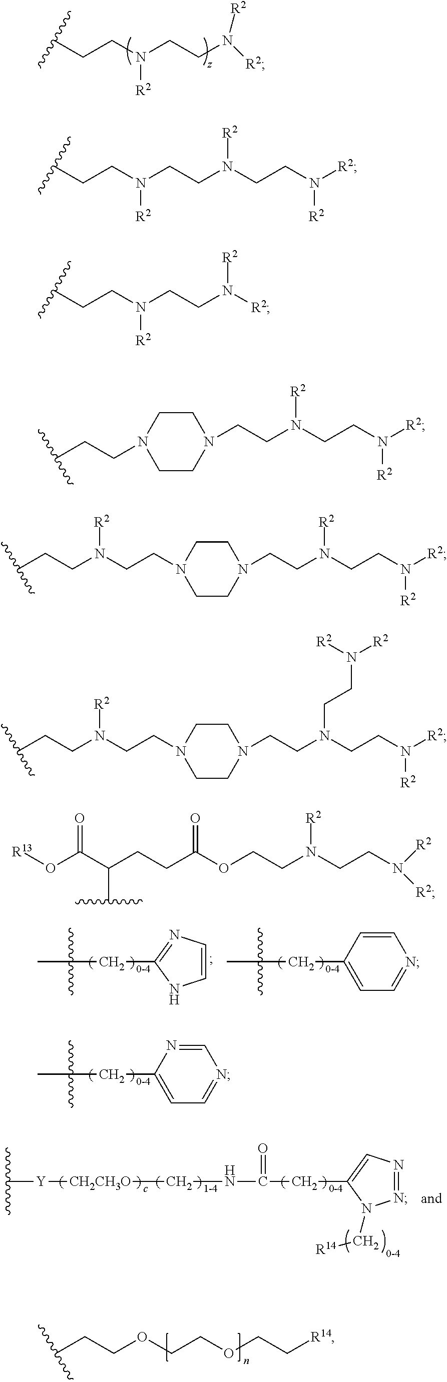

wherein

each instance of R² is as described above;

R¹³ is a C₁-C₅₀ alkyl group, alkenyl group, cycloalkyl group, or cycloalkenyl group optionally substituted with one or more amines;

z is an integer from 1 to 5;

c is an integer from 0 to 50;

Y is optionally present and is a cleavable linker;

n is an integer from 0 to 50; and

R¹⁴ is a tissue-specific or cell-specific targeting moiety. C₁-C₁₂ alkyl group, alkenyl group, cycloalkyl group, or cycloalkenyl group.

R¹³ can be a C₁-C₅₀ (e.g., C₁-C₄₀, C₁-C₃₀, C₁-C₂₀, C₁-C₁₀, C₄-C₁₂, or C₆-C₈) alkyl group, alkenyl group, cycloalkyl group, or cycloalkenyl group optionally substituted with one or more amines. In some embodiments, R¹³ is a C₄-C₁₂, such as a C₆-C₈, alkyl group, alkenyl group, cycloalkyl group, or cycloalkenyl group optionally substituted with one or more amines. In some embodiments, R¹³ is substituted with one or more amines. In certain embodiments, R¹³ is substituted with 2 to 8 (i.e., 2, 3, 4, 5, 6, 7, or 8) tertiary amines. The tertiary amines can be a part of the alkyl group (i.e., encompassed in the alkyl group backbone) or a pendant substituent.

Each instance of Y is optionally present. As used herein, the phrase “optionally present” means that a substituent designated as optionally present can be present or not present, and when that substituent is not present, the adjoining substituents are bound directly to each other. When Y is present, Y is a cleavable linker. As used herein, the phrase “cleavable linker” refers to any chemical element that connects two species that can be cleaved as to separate the two species. For example, the cleavable linker can be cleaved by a hydrolytic process, photochemical process, radical process, enzymatic process, electrochemical process, or a combination thereof. Exemplary cleavable linkers include, but are not limited to:

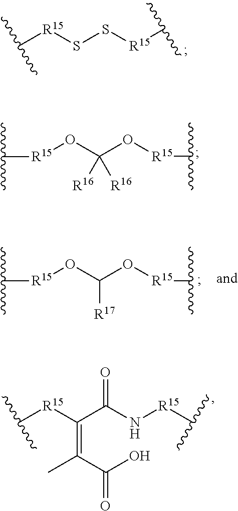

wherein each occasion of R¹⁵ independently is a C₁-C₄ alkyl group, each occasion of R¹⁶ independently is hydrogen, an aryl group, a heterocyclic group (e.g., aromatic or non-aromatic), a C₁-C₁₂ alkyl group, alkenyl group, cycloalkyl group, or cycloalkenyl group, and R¹⁷ is a six-membered aromatic or heteroaromatic group optionally substituted with one or more —OCH₃, —NHCH₃, —N(CH₃)₂, —SCH₃, —OH, or a combination thereof.

In some embodiments of the foregoing polymers, E¹ and E² are —(CH₂)ₚ₁—(NR²—(CH₂)_{q1}—)ᵣ₁NR²₂, optionally wherein R² is independently hydrogen or a C₁-C₃ alkyl (e.g., methyl or ethyl), and p¹, q1, and r1 are each independently an integer of 1 to 5 (e.g., 1, 2, or 3); for instance, E¹ and E² can be —(CH₂)₂—NR²—(CH₂)₂—NR²₂ or —(CH₂)₂—NR²—(CH₂)₂—NHR²; X² is a C₁-C₈ (e.g., C₁-C₆, C₁-C₄, C₁-C₃, C₂-C₈, C₂-C₆, C₃-C₈, or C₃-C₆) linear or branched alkyl group, optionally substituted with one or more hydroxyl groups (e.g., 1 or 2 hydroxyl groups) or halogen groups (e.g., substituted with one or more fluorine, chlorine, bromine, or iodine atoms); and X⁴ is group comprising a polyalkylene oxide, polyglycolic acid, polylactic acid, or a combination thereof (e.g., a group comprising a polyalkylene oxide). Also, in such embodiments, R^{3a} and R^{3b} can both be methylene, X¹ and X³ can both be —C(O)NR¹¹—, and each R¹¹ can be methyl or hydrogen.

The polymer can have any suitable terminal groups. In some embodiments, the polymer has a structure of Formula P2A:

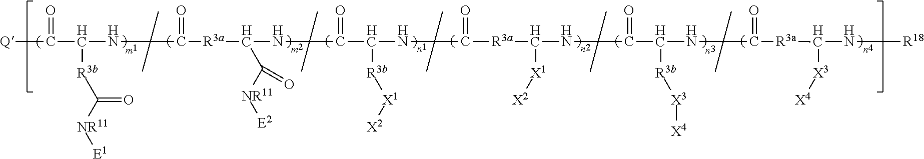

wherein

Q′ is of formula:

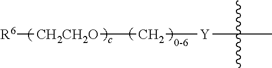

c is an integer from 0 to 50 (e.g., 0), or c is an integer from 2 to 200 (e.g., 2 to 150, 2 to 100, 2 to 50, 10 to 200, 10 to 150, 10 to 100, 10 to 50, 25 to 200, 25 to 150, 25 to 100, 25 to 50, 50 to 200, 50 to 150, or 50 to 100);

Y is optionally present and is a cleavable linker;

R¹⁸ is hydrogen, an aryl group, a heterocyclic group, a C₁-C₁₂ (e.g., C₁-C₈, C₁-C₆, or C₁-C₃) alkyl or heteroalkyl group, C₂-C₁₂ (e.g., C₂-C₈, C₂-C₆, or C₂-C₃) alkenyl group, C₃-C₁₂ (e.g., C₃-C₈, C₃-C₆, or C₃-C₅) cycloalkyl group, or C₃-C₁₂ (e.g., C₃-C₈, C₃-C₆, or C₃-C₅) cycloalkenyl group, optionally substituted with one or more substituents; and

R⁶ is hydrogen, an amino group, an aryl group, a heterocyclic group, a C₁-C₁₂ (e.g., C₁-C₈, C₁-C₆, or C₁-C₃) alkyl or heteroalkyl group, a C₂-C₁₂ (e.g., C₂-C₈, C₂-C₆, or C₂-C₃) alkenyl group, a C₃-C₁₂ (e.g., C₃-C₈, C₃-C₆, or C₃-C₅) cycloalkyl group, or a C₃-C₁₂ (e.g., C₃-C₈, C₃-C₆, or C₃-C₅) cycloalkenyl group, optionally substituted with one or more amines; or a tissue-specific or cell-specific targeting moiety; and

all other variables and substituents of Formula P2A are as defined with respect to Formula P2, including any and all embodiments thereof. In some embodiments, R¹⁸ and/or R⁶ is a C₁-C₁₂ alkyl (e.g., a C₁-C₁₀ alkyl group; a C₁-C₈ alkyl group; a C₁-C₆ alkyl group; a C₁-C₄ alkyl group, a C₁-C₃ alkyl group, or a C₁ or C₂ alkyl group), which can be linear or branched, optionally substituted with one or more substituents. In certain embodiments, the heteroalkyl or alkyl group comprises or is substituted with one or more amines, for instance, from 2 to 8 (i.e., 2, 3, 4, 5, 6, 7, or 8) tertiary amines. The tertiary amines can be a part of the heteroalkyl backbone chain or pendant substituents.

Non-limiting examples of the second polymer provided herein include, for instance:

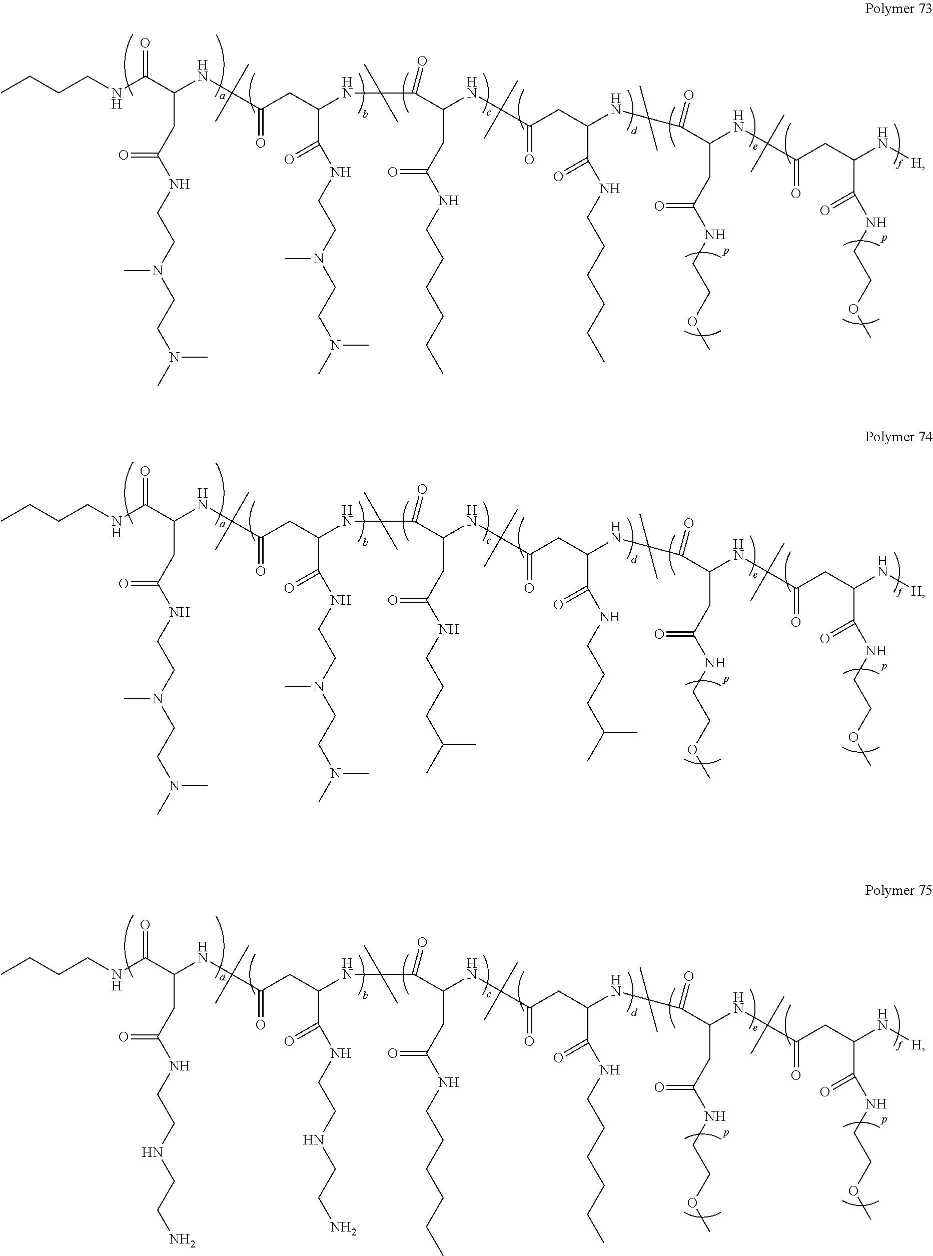

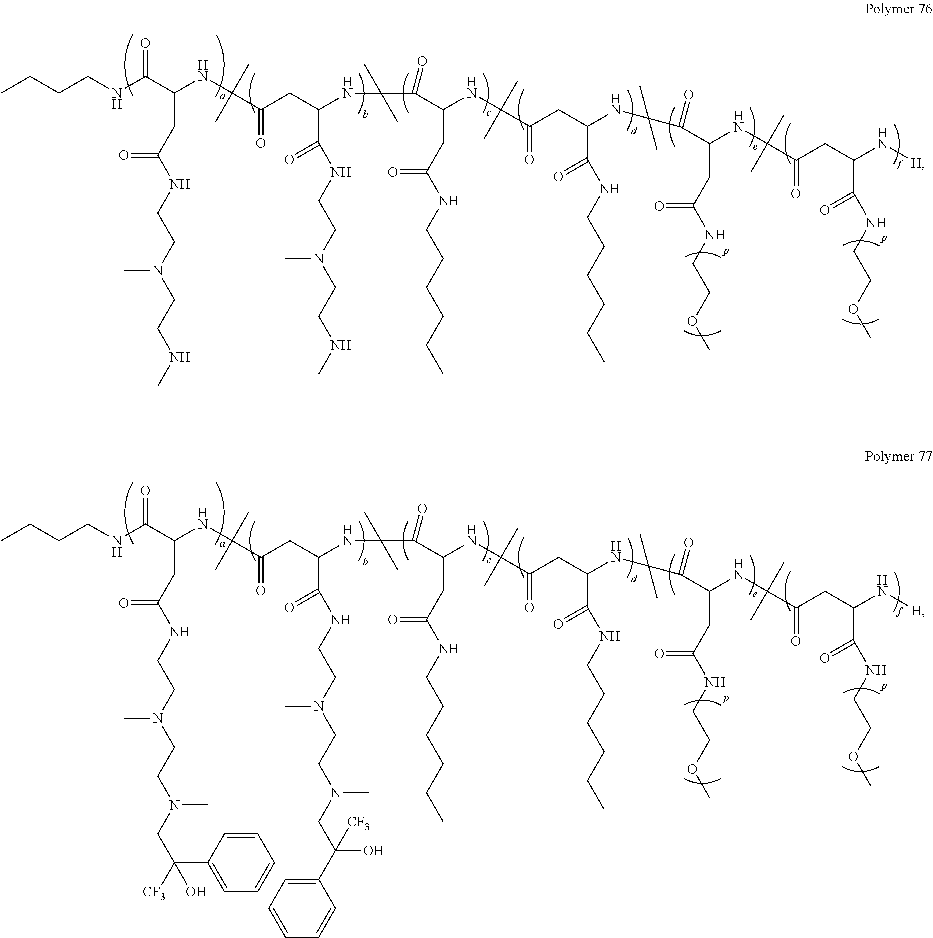

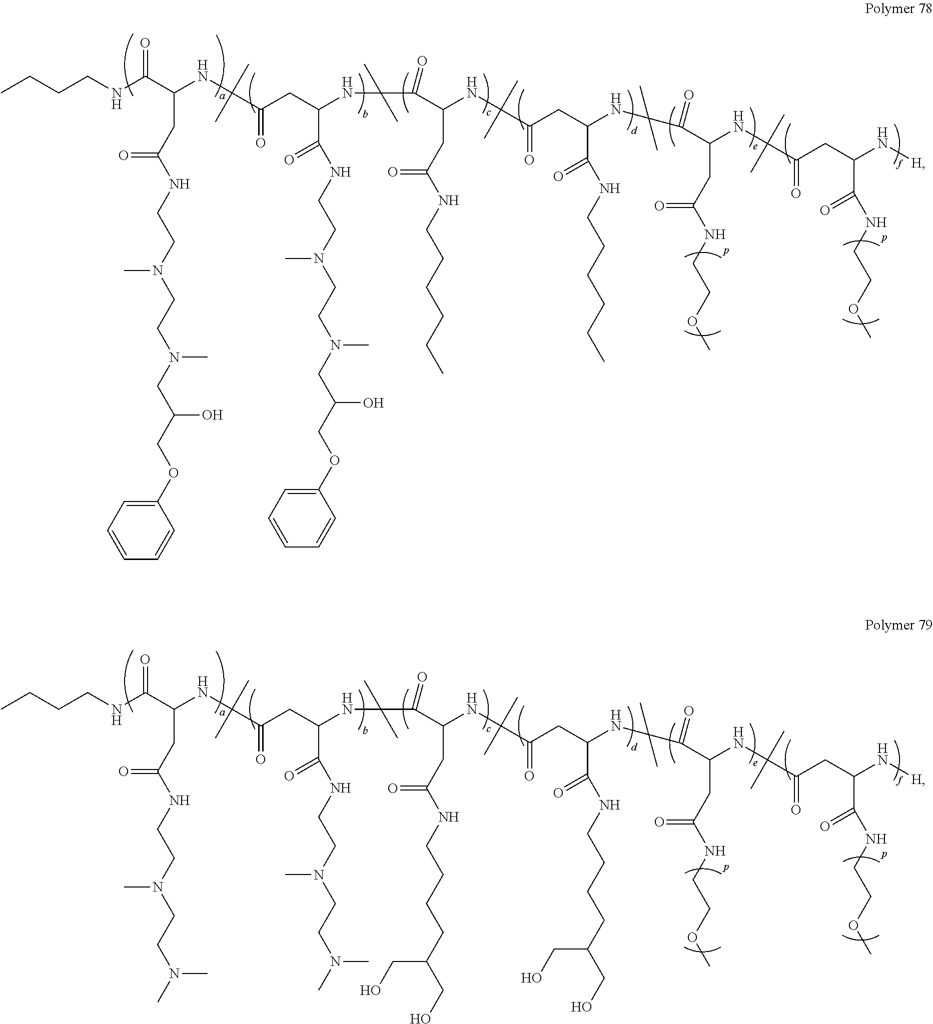

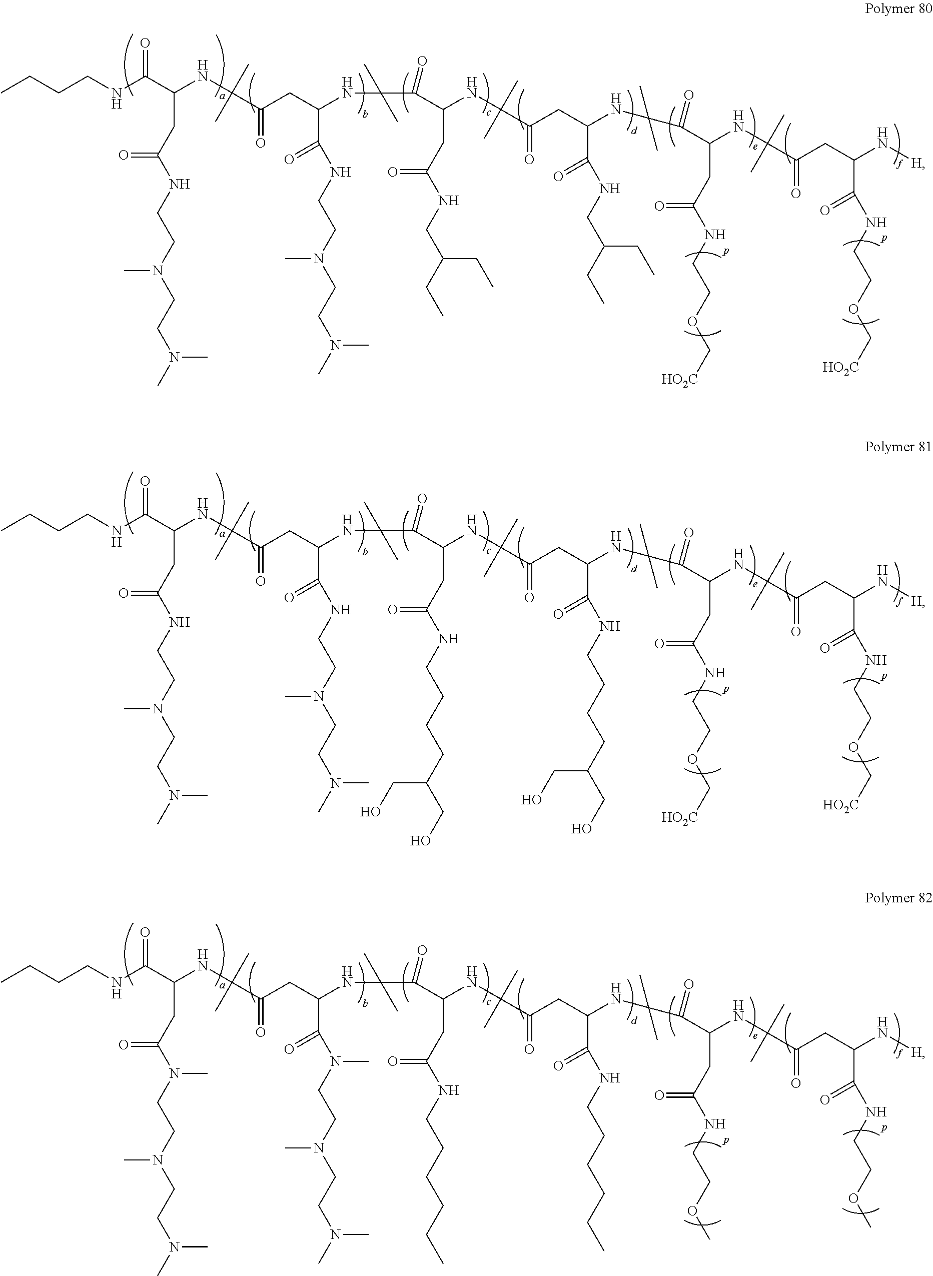

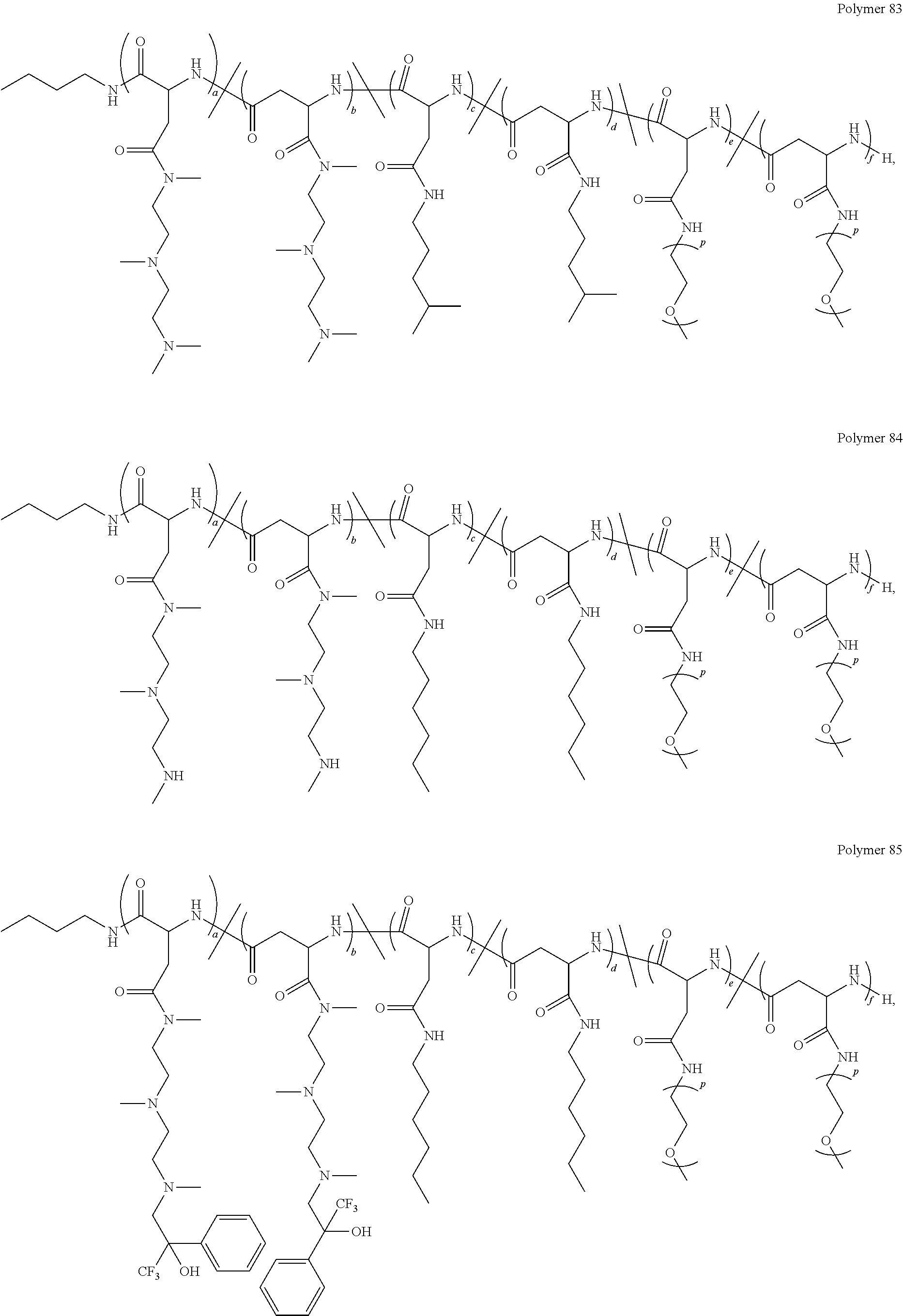

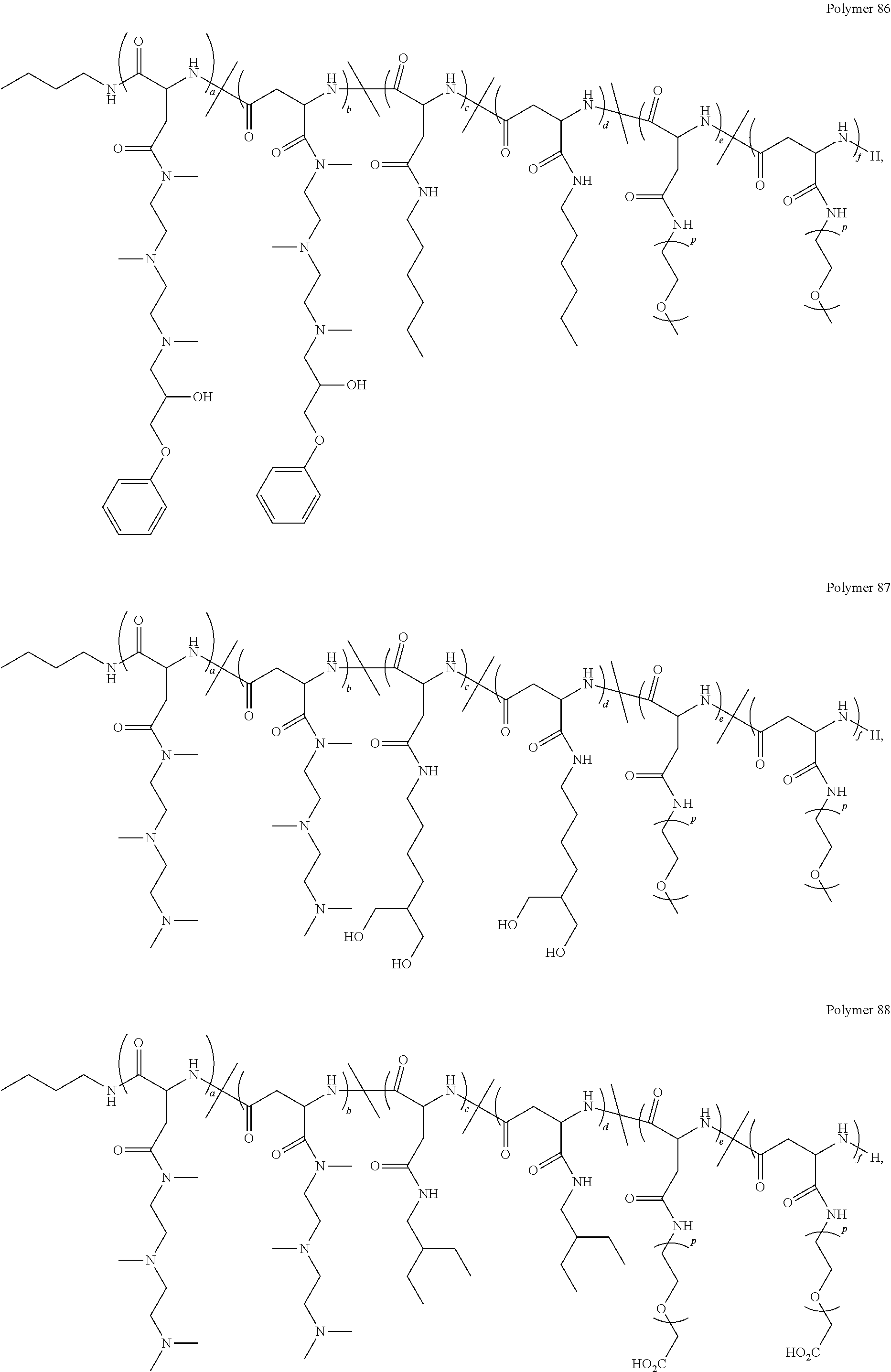

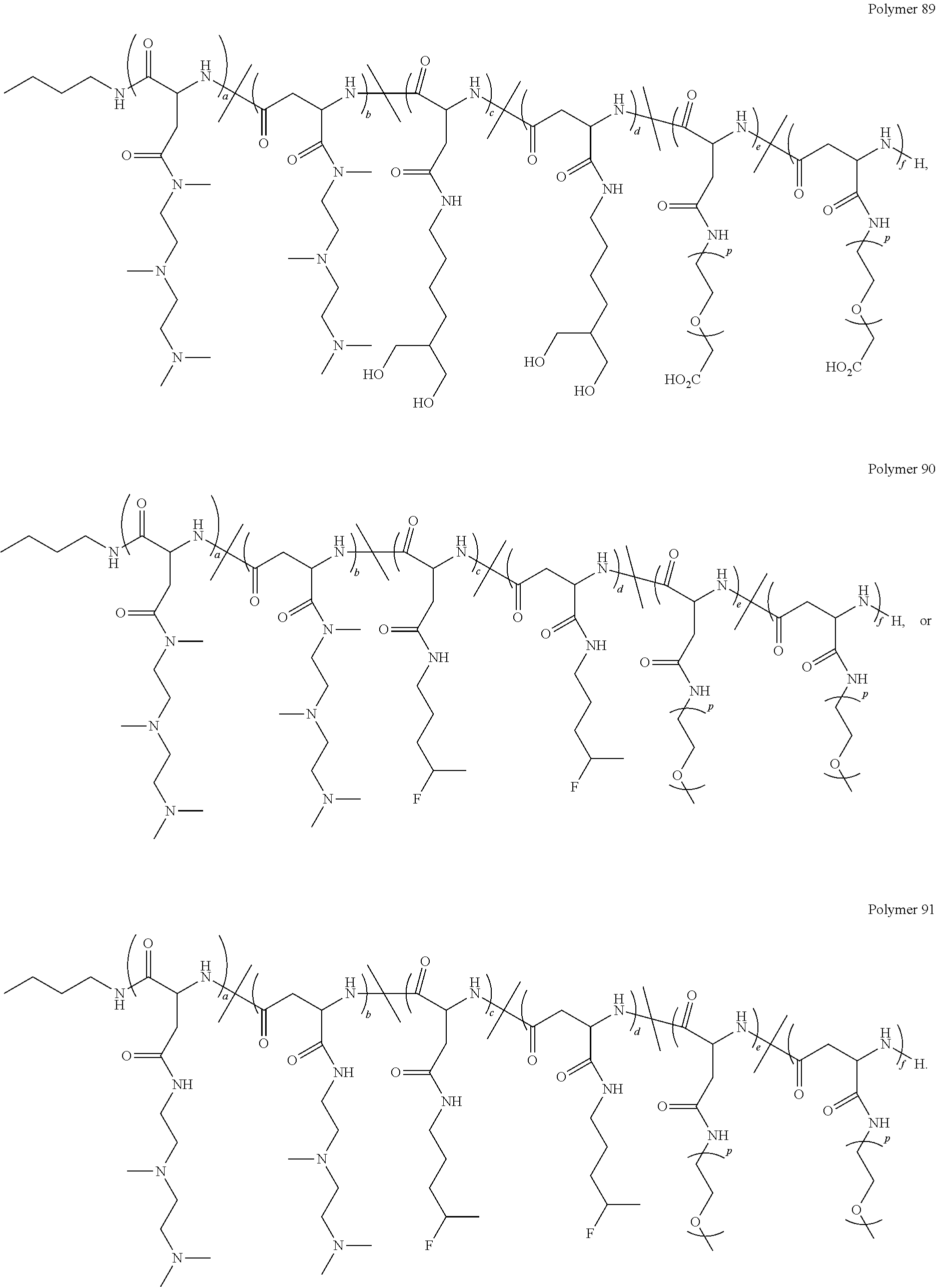

The indication of the number of units (“a”, “b”, “c”, “d”, “e”, and “f”) in these examples of polymers does not imply a block co-polymer structure; rather, these numbers indicate the number of particular monomer units overall, which units can be arranged in any order, including blocks of monomers or monomers randomly arranged throughout the polymer. In some instances, this is additionally indicated by the “/” symbols in the formulas; however, the absence of a “/” should not be taken to mean that the polymers are joined in a particular order. In some embodiments of the foregoing polymers, the monomers designated by parenthesis and an integer (“a”, “b”, “c”, “d”, “e”, and “f” as applicable) are randomly arranged or dispersed throughout the polymer.

In some embodiments of the foregoing polymers, (a+b) is from about 5 to about 65 (e.g., about 5 to about 50, about 5 to about 40, about 5 to about 30, about 5 to about 20, or about 5 to about 10), (c+d) is from about 2 to about 60 (e.g., about 2 to about 50, about 2 to about 40, about 2 to about 30, about 2 to about 20, or about 2 to about 10), (e+f) is from about 2 to about 60 (e.g., about 2 to about 50, about 2 to about 40, about 2 to about 30, about 2 to about 20, or about 2 to about 10), and each instance of p is independently an integer from 2 to 200 (e.g., 2 to 150, 2 to 100, 2 to 50, 6 to 36, 6 to 30, 6 to 24, 6 to 18, 8 to 36, 8 to 30, 8 to 26, 8 to 18, 10 to 200, 10 to 150, 10 to 100, 10 to 50, 25 to 200, 25 to 150, 25 to 100, 25 to 50, 50 to 200, 50 to 150, or 50 to 100).

In a particular embodiment, the second polymer has the structure of any one of polymers 73-91, or has the structure:

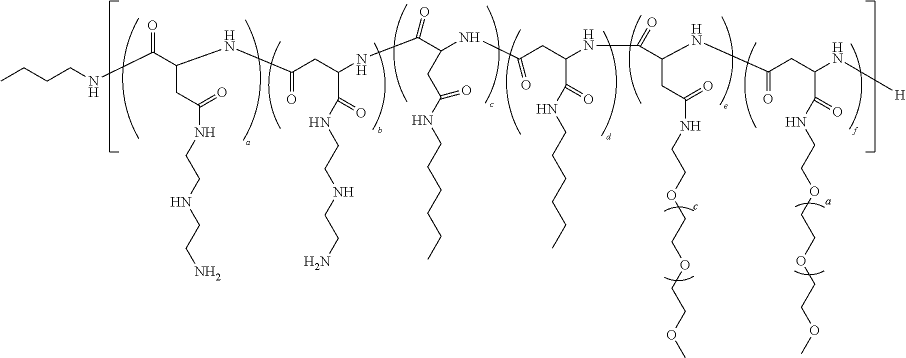

wherein n is 6-24, or more (e.g., 24-60 or 40-50), and a, b, c, d, e, and f are as described above.

Some of the above particular examples of polymers provided by the disclosure are depicted with specific terminal groups (e.g., alkylamino, hydrogen, etc.); however, any of the foregoing particular structures can comprise different terminal groups. For example, any of the foregoing structures comprise a group of R¹, R⁶, or Q as described herein at either or both termini of the polymer backbone.

Typically, the polymer is cationic (i.e., positively charged at pH 7 and 23° C.). As used herein, “cationic” polymers refer to polymers having an overall net positive charge, whether the polymer comprises only cationic monomer units or a combination of cationic monomer units and non-ionic or anionic monomer units.

In certain embodiments, the second polymer described herein has a weight average molecular weight of from about 5 kDa to about 2,000 kDa. The polymer can have a weight average molecular weight of about 2,000 kDa or less, for example, about 1,800 kDa or less, about 1,600 kDa or less, about 1,400 kDa or less, about 1,200 kDa or less, about 1,000 kDa or less, about 900 kDa, or less, about 800 kDa, or less, about 700 kDa or less, about 600 kDa or less, about 500 kDa or less, about 100 kDa or less, or about 50 kDa or less. Alternatively, or in addition, the polymer can have a weight average molecular weight of about 10 kDa or more, for example, about 50 kDa or more, about 100 kDa or more, about 200 kDa or more, about 300 kDa or more, or about 400 kDa or more. Thus, the polymer can have a weight average molecular weight bounded by any two of the aforementioned endpoints. For example, the polymer can have a weight average molecular weight of from about 10 kDa to about 50 kDa, from about, from about 10 kDa to about 100 kDa, from about 10 kDa to about 500 kDa, from about 50 kDa to about 500 kDa, from about 100 kDa to about 500 kDa, from about 200 kDa to about 500 kDa, from about 300 kDa to about 500 kDa, from about 400 kDa to about 500 kDa, from about 400 kDa to about 600 kDa, from about 400 kDa to about 700 kDa, from about 400 kDa to about 800 kDa, from about 400 kDa to about 900 kDa, from about 400 kDa to about 1,000 kDa, from about 400 kDa to about 1,200 kDa, from about 400 kDa to about 1,400 kDa, from about 400 kDa to about 1,600 kDa, from about 400 kDa to about 1,800 kDa, from about 400 kDa to about 2,000 kDa, from about 200 kDa to about 2,000 kDa, from about 500 kDa to about 2,000 kDa, or from about 800 kDa to about 2,000 kDa. The weight average molecular weight can be determined by any suitable technique. Generally, the weight average molecular weight is determined using size exclusion chromatography equipped with a column, selected from TSKgel Guard, GMPW, GMPW, G1000PW, and a Waters 2414 (Waters Corporation, Milford, Mass.) refractive index detector. Moreover, the weight average molecular weight is determined from calibration with polyethylene oxide/polyethylene glycol standards ranging from 150-875,000 Daltons.

### Method of Preparing Second Polymer

The second polymer can be prepared by any suitable method, examples of which are described herein. In some embodiments, the method comprises preparing a polymer comprising a structure of Formula P2:

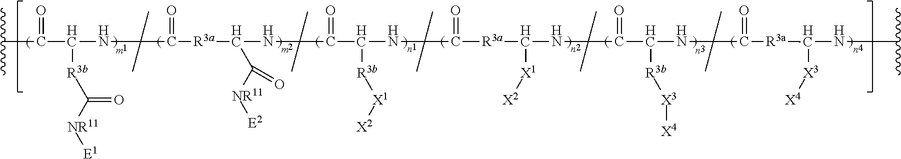

as described herein from a polymer comprising a structure of Formula 6 or Formula 7:

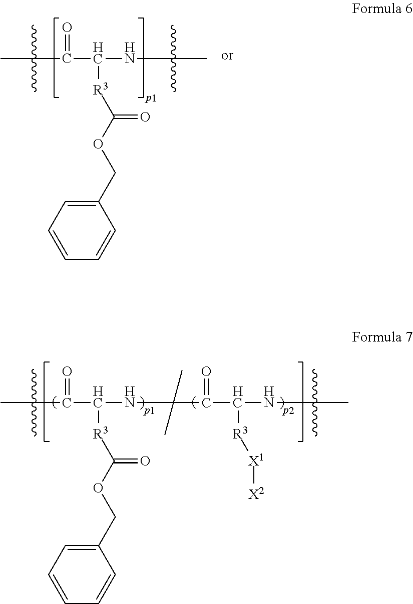

wherein,

p¹ is an integer from 1 to 2000 (e.g., from 1 to 1000, from 1 to 500, from 1 to 200, from 1 to 100, from 5 to 2000, from 5 to 1000, from 5 to 500, from 5 to 200, or from 5 to 100);

p² is an integer from 1 to 2000 (e.g., from 1 to 1000, from 1 to 500, from 1 to 200, from 1 to 100, from 2 to 2000, from 2 to 1000, from 2 to 500, from 2 to 200, or from 2 to 100);

each R³ is independently a methylene or ethylene group;

and all other substituents and variables are as previously described with respect to Formula P2 and P2A, including any and all embodiments thereof. According to one aspect of the method, the structure of Formula 6 can be combined (reacted) with (a) a compound of the formula HNR¹¹E¹ and/or HNR¹E²; (b) a compound of formula H₂NX⁴ or HOX⁴; and (c) a compound of formula H₂NX² or HOX², simultaneously or in any sequential order, to a polymer comprising a structure of Formula P2. According to another aspect, the compound of Formula 7 (which already includes an X² group) can be combined (reacted) with (a) a compound of the formula HNR¹E¹ and/or HNR¹¹E² and (b) a compound of formula H₂NX⁴ or HOX⁴, simultaneously or in any sequential order, to a polymer comprising a structure of Formula P2.

In the compound of formula HNR¹¹E¹, HNR¹¹E², H₂NX², HOX², H₂NX⁴, or HOX⁴ each instance of R¹, E¹, E², X², and X⁴ is as previously described with respect to the polymers of Formula P2 and P2A, including any and all embodiments thereof. Furthermore, The polymer of Formula 6 or 7 can have any suitable terminal groups as desired in the resulting polymer of Formula P2 or P2A. Where the second polymer (e.g., according to Formula P2) is to have groups E¹ and E² that include R⁵ or R⁴-R⁵ groups, such polymers can be provided directly using the above method, or by preparing a polymer in which groups E¹ and E² are polyamines that do not include R⁵ or R⁴-R⁵ groups and subsequently modifying the terminus of the polyamine (e.g., via a Michael addition reaction, an epoxide opening, a displacement reaction, or other suitable technique) to a R⁵ or R⁴-R⁵ groups using a suitable compound, such as a compound having the formula:

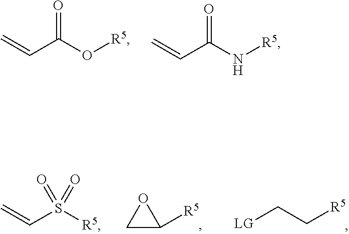

wherein LG is a leaving group (e.g., chloride atom, bromide atom, iodide atom, tosylate, triflate, mesylate, etc.) and R⁵ is as described herein with respect to the second polymer (e.g., of Formula P2).

### Compositions

The polymers provided herein can be used for any purpose. However, it is believed the polymers are particularly useful for delivering nucleic acids and/or polypeptides (e.g., protein) to cells. Thus, provided herein is a composition comprising a polymer as described herein and a nucleic acid and/or polypeptide (e.g., protein).

In some embodiments, the composition comprises a nucleic acid. Any nucleic acid can be used. An exemplary list of nucleic acids includes guide and/or donor nucleic acids of CRISPR systems, siRNA, microRNA, interfering RNA or RNAi, dsRNA, mRNA, DNA vector, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

The composition also can comprise any protein for delivery, in addition to or instead of a nucleic acid. The polypeptide can be any suitable polypeptide. For example, the polypeptide can be a zinc finger nuclease, a transcription activator-like effector nuclease (“TALEN”), a recombinase, a deaminase, an endonuclease, or a combination thereof. In some embodiments, the polypeptide is an RNA-guided endonuclease (e.g., a Cas9 polypeptide, a Cpf1 polypeptide, or variants thereof) or a DNA recombinase (e.g., a Cre polypeptide).

It is believed the polymers provided herein are particularly useful for delivering one or more components of a CRISPR system. Thus, in some embodiments, the composition comprises a guide RNA, an RNA-guided endonuclease or nucleic acid encoding same, and/or a donor nucleic acid. The composition can comprise one, two, or all three components together with the polymer described herein. Furthermore, the composition can comprise a plurality of guide RNAs, RNA-guided endonucleases or nucleic acids encoding same, and/or donor nucleic acids. For instance, multiple different guide RNAs for different target sites could be included, optionally with multiple different donor nucleic acids and even multiple different RNA guided endonucleases or nucleic acids encoding same.

Furthermore, the components of the CRISPR system can be combined with one another (when multiple components are present) and the polymer in any particular manner or order. In some embodiments, the guide RNA is complexed with an RNA endonuclease prior to combining with the polymer. In addition, or instead, the guide RNA can be linked (covalently or non-covalently) to a donor nucleic acid prior to combining with the polymer.

The compositions are not limited with respect to any particular CRISPR system (i.e., any particular guide RNA, RNA-guided endonuclease, or donor nucleic acid), many of which are known. Nevertheless, for the sake of further illustration, the components of some such systems are described below.

Donor Nucleic Acid

The donor nucleic acid (or “donor sequence” or “donor polynucleotide” or “donor DNA”) is a nucleic acid sequence to be inserted at the cleavage site induced by an RNA-directed endonuclease (e.g., a Cas9 polypeptide or a Cpf1 polypeptide). The donor polynucleotide will contain sufficient homology to a target genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some embodiments may be used for other purposes (e.g., to signify expression at the targeted genomic locus). In some embodiments, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Amplification procedures such as rolling circle amplification can also be advantageously employed, as exemplified herein. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or polymer, or can be delivered by viruses (e.g., adenovirus, AAV), as described herein for nucleic acids encoding a Cas9 guide RNA and/or a Cas9 fusion polypeptide and/or donor polynucleotide.

Guide Nucleic Acid

In some embodiments, the composition comprises guide nucleic acid. Guide nucleic acids suitable for inclusion in a composition of the present disclosure include single-molecule guide RNAs (“single-guide RNA”/“sgRNA”) and dual-molecule guide nucleic acids (“dual-guide RNA”/“dgRNA”).

A guide nucleic acid (e.g., guide RNA) suitable for inclusion in a complex of the present disclosure directs the activities of an RNA-guided endonuclease (e.g., a Cas9 or Cpf1 polypeptide) to a specific target sequence within a target nucleic acid. A guide nucleic acid (e.g., guide RNA) comprises: a first segment (also referred to herein as a “nucleic acid targeting segment”, or simply a “targeting segment”); and a second segment (also referred to herein as a “protein-binding segment”). The terms “first” and “second” do not imply the order in which the segments occur in the guide RNA. The order of the elements relative to one another depends upon the particular RNA-guided polypeptide to be used. For instance, guide RNA for Cas9 typically has the protein-binding segment located 3′ of the targeting segment, whereas guide RNA for Cpf1 typically has the protein-binding segment located 5′ of the targeting segment.

The guide RNA may be introduced into a cell in linear or circular form. If introduced in linear form, the ends of the guide RNA may be protected (e.g., from exonucleolytic degradation) by methods known to those of skill in the art. Amplification procedures such as rolling circle amplification can also be advantageously employed, as exemplified herein.

First Segment: Targeting Segment

The first segment of a guide nucleic acid (e.g., guide RNA) includes a nucleotide sequence that is complementary to a sequence (a target site) in a target nucleic acid. In other words, the targeting segment of a guide nucleic acid (e.g., guide RNA) can interact with a target nucleic acid (e.g., an RNA, a DNA, a double-stranded DNA) in a sequence-specific manner via hybridization (i.e., base pairing). As such, the nucleotide sequence of the targeting segment may vary and can determine the location within the target nucleic acid that the guide nucleic acid (e.g., guide RNA) and the target nucleic acid will interact. The targeting segment of a guide nucleic acid (e.g., guide RNA) can be modified (e.g., by genetic engineering) to hybridize to any desired sequence (target site) within a target nucleic acid.

The targeting segment can have a length of from 12 nucleotides to 100 nucleotides. The nucleotide sequence (the targeting sequence, also referred to as a guide sequence) of the targeting segment that is complementary to a nucleotide sequence (target site) of the target nucleic acid can have a length of 12 nt or more. For example, the targeting sequence of the targeting segment that is complementary to a target site of the target nucleic acid can have a length of 12 nt or more, 15 nt or more, 17 nt or more, 18 nt or more, 19 nt or more, 20 nt or more, 25 nt or more, 30 nt or more, 35 nt or more or 40 nt.

The percent complementarity between the targeting sequence (i.e., guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some embodiments, the percent complementarity between the targeting sequence of the targeting segment and the target site of the target nucleic acid is 100% over the seven contiguous 5′-most nucleotides of the target site of the target nucleic acid. In some embodiments, the percent complementarity between the targeting sequence of the targeting segment and the target site of the target nucleic acid is 60% or more over 20 contiguous nucleotides. In some embodiments, the percent complementarity between the targeting sequence of the targeting segment and the target site of the target nucleic acid is 100% over the seventeen, eighteen, nineteen or twenty contiguous 5′-most nucleotides of the target site of the target nucleic acid and as low as 0% or more over the remainder. In such a case, the targeting sequence can be considered to be 17, 18, 19 or 20 nucleotides in length, respectively.

Second Segment: Protein-Binding Segment

The protein-binding segment of a guide nucleic acid (e.g., guide RNA) interacts with (binds) an RNA-guided endonuclease. The guide nucleic acid (e.g., guide RNA) guides the bound endonuclease to a specific nucleotide sequence within target nucleic acid (the target site) via the above mentioned targeting segment/targeting sequence/guide sequence. The protein-binding segment of a guide nucleic acid (e.g., guide RNA) comprises two stretches of nucleotides that are complementary to one another. The complementary nucleotides of the protein-binding segment hybridize to form a double stranded RNA duplex (dsRNA).

Single and Dual Guide Nucleic Acids

A dual guide nucleic acid (e.g., guide RNA) comprises two separate nucleic acid molecules. Each of the two molecules of a subject dual guide nucleic acid (e.g., guide RNA) comprises a stretch of nucleotides that are complementary to one another such that the complementary nucleotides of the two molecules hybridize to form the double stranded RNA duplex of the protein-binding segment.

In some embodiments, the duplex-forming segment of the activator is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, the duplex-forming segment of the targeter is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

A dual guide nucleic acid (e.g., guide RNA) can be designed to allow for controlled (i.e., conditional) binding of a targeter with an activator. Because a dual guide nucleic acid (e.g., guide RNA) is not functional unless both the activator and the targeter are bound in a functional complex with Cas9, a dual guide nucleic acid (e.g., guide RNA) can be inducible (e.g., drug inducible) by rendering the binding between the activator and the targeter to be inducible. As one non-limiting example, RNA aptamers can be used to regulate (i.e., control) the binding of the activator with the targeter. Accordingly, the activator and/or the targeter can include an RNA aptamer sequence.

Aptamers (e.g., RNA aptamers) are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the nucleic acid molecule (e.g., RNA, DNA/RNA hybrid, etc.) of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator with an aptamer may not be able to bind to the cognate targeter unless the aptamer is bound by the appropriate drug; (ii) a targeter with an aptamer may not be able to bind to the cognate activator unless the aptamer is bound by the appropriate drug; and (iii) a targeter and an activator, each comprising a different aptamer that binds a different drug, may not be able to bind to each other unless both drugs are present. As illustrated by these examples, a dual guide nucleic acid (e.g., guide RNA) can be designed to be inducible.

Examples of aptamers and riboswitches can be found, for example, in: Nakamura et al., Genes Cells. 2012 May; 17(5):344-64; Vavalle et al., Future Cardiol. 2012 May; 8(3):371-82; Citartan et al., Biosens Bioelectron. 2012 Apr. 15; 34(1):1-11; and Liberman et al., Wiley Interdiscip Rev RNA. 2012 May-June; 3(3):369-84; all of which are herein incorporated by reference in their entirety.

Non-limiting examples of nucleotide sequences that can be included in a dual guide nucleic acid (e.g., guide RNA) included in International Patent Application Nos. PCT/US2016/052690 and PCT/US2017/062617, or complements thereof that can hybridize to form a protein binding segment.

A subject single guide nucleic acid (e.g., guide RNA) comprises two stretches of nucleotides (much like a “targeter” and an “activator” of a dual guide nucleic acid) that are complementary to one another, hybridize to form the double stranded RNA duplex (dsRNA duplex) of the protein-binding segment (thus resulting in a stem-loop structure), and are covalently linked by intervening nucleotides (“linkers” or “linker nucleotides”). Thus, a subject single guide nucleic acid (e.g., a single guide RNA) can comprise a targeter and an activator, each having a duplex-forming segment, where the duplex-forming segments of the targeter and the activator hybridize with one another to form a dsRNA duplex. The targeter and the activator can be covalently linked via the 3′ end of the targeter and the 5′ end of the activator. Alternatively, targeter and the activator can be covalently linked via the 5′ end of the targeter and the 3′ end of the activator.

The linker of a single guide nucleic acid can have a length of from 3 nucleotides to 100 nucleotides. In some embodiments, the linker of a single guide nucleic acid (e.g., guide RNA) is 4 nt.

An exemplary single guide nucleic acid (e.g., guide RNA) comprises two complementary stretches of nucleotides that hybridize to form a dsRNA duplex. In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the activator (tracrRNA) molecules set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

In some embodiments, one of the two complementary stretches of nucleotides of the single guide nucleic acid (e.g., guide RNA) (or the DNA encoding the stretch) is 60%, 65%, 70%, 75%, 80%, 85%, 90%, 95%, 98%, 99% or more identical or 100% identical to one of the targeter (crRNA) sequences or activator (tracrRNA) sequences set forth in International Patent Application Nos. PCT/US2016/052690 and PCT/US2017/062617, or a complement thereof, over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides).

Appropriate cognate pairs of targeters and activators can be routinely determined by taking into account the species name and base-pairing (for the dsRNA duplex of the protein-binding domain) Any activator/targeter pair can be used as part of dual guide nucleic acid (e.g., guide RNA) or as part of a single guide nucleic acid (e.g., guide RNA).

In some embodiments, an activator (e.g., a trRNA, trRNA-like molecule, etc.) of a dual guide nucleic acid (e.g., guide RNA) (e.g., a dual guide RNA) or a single guide nucleic acid (e.g., guide RNA) (e.g., a single guide RNA) includes a stretch of nucleotides with 60%, 65%, 70%, 75%, 80%, 85%, 90%, 95%, 98%, 99% or more, or 100% sequence identity with an activator (tracrRNA) molecule set forth in International Patent Application Nos. PCT/US2016/052690 and PCT/US2017/062617, or a complement thereof.

In some embodiments, an activator (e.g., a trRNA, trRNA-like molecule, etc.) of a dual guide nucleic acid (e.g., a dual guide RNA) or a single guide nucleic acid (e.g., a single guide RNA) includes 30 or more nucleotides (nt) (e.g., 40 or more, 50 or more, 60 or more, 70 or more, 75 or more nt). In some embodiments, an activator (e.g., a trRNA, trRNA-like molecule, etc.) of a dual guide nucleic acid (e.g., a dual guide RNA) or a single guide nucleic acid (e.g., a single guide RNA) has a length in a range of from 30 to 200 nucleotides (nt).

The protein-binding segment can have a length of from 10 nucleotides to 100 nucleotides.

Also with regard to both a subject single guide nucleic acid (e.g., single guide RNA) and to a subject dual guide nucleic acid (e.g., dual guide RNA), the dsRNA duplex of the protein-binding segment can have a length from 6 base pairs (bp) to 50 bp. The percent complementarity between the nucleotide sequences that hybridize to form the dsRNA duplex of the protein-binding segment can be 60% or more. For example, the percent complementarity between the nucleotide sequences that hybridize to form the dsRNA duplex of the protein-binding segment can be 65% or more, 70% or more, 75% or more, 80% or more, 85% or more, 90% or more, 95% or more, 98% or more, or 99% or more (e.g., in some embodiments, there are some nucleotides that do not hybridize and therefore create a bulge within the dsRNA duplex. In some embodiments, the percent complementarity between the nucleotide sequences that hybridize to form the dsRNA duplex of the protein-binding segment is 100%.

Hybrid Guide Nucleic Acids

In some embodiments, a guide nucleic acid is two RNA molecules (dual guide RNA). In some embodiments, a guide nucleic acid is one RNA molecule (single guide RNA). In some embodiments, a guide nucleic acid is a DNA/RNA hybrid molecule. In such embodiments, the protein-binding segment of the guide nucleic acid is RNA and forms an RNA duplex. Thus, the duplex-forming segments of the activator and the targeter is RNA. However, the targeting segment of a guide nucleic acid can be DNA. Thus, if a DNA/RNA hybrid guide nucleic acid is a dual guide nucleic acid, the “targeter” molecule and be a hybrid molecule (e.g., the targeting segment can be DNA and the duplex-forming segment can be RNA). In such embodiments, the duplex-forming segment of the “activator” molecule can be RNA (e.g., in order to form an RNA-duplex with the duplex-forming segment of the targeter molecule), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment of the single guide nucleic acid) can be RNA, and nucleotides outside of the targeting and duplex-forming segments can be RNA or DNA.

A DNA/RNA hybrid guide nucleic can be useful in some embodiments, for example, when a target nucleic acid is an RNA. Cas9 normally associates with a guide RNA that hybridizes with a target DNA, thus forming a DNA-RNA duplex at the target site. Therefore, when the target nucleic acid is an RNA, it is sometimes advantageous to recapitulate a DNA-RNA duplex at the target site by using a targeting segment (of the guide nucleic acid) that is DNA instead of RNA. However, because the protein-binding segment of a guide nucleic acid is an RNA-duplex, the targeter molecule is DNA in the targeting segment and RNA in the duplex-forming segment. Hybrid guide nucleic acids can bias Cas9 binding to single stranded target nucleic acids relative to double stranded target nucleic acids.

Exemplary Guide Nucleic Acids

Any guide nucleic acid can be used. Many different types of guide nucleic acids are known in the art. The guide nucleic selected will be appropriately paired to the particular CRISPR system being used (e.g., the particular RNA guided endonuclease being used). Thus, the guide nucleic acid can be, for instance, a guide nucleic acid corresponding to any RNA guided endonuclease described herein or known in the art. Guide nucleic acids and RNA guided endonucleases are described, for example, in International Patent Application Nos. PCT/US2016/052690 and PCT/US2017/062617

In some embodiments, a suitable guide nucleic acid includes two separate RNA polynucleotide molecules. In some embodiments, the first of the two separate RNA polynucleotide molecules (the activator) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof. In some embodiments, the second of the two separate RNA polynucleotide molecules (the targeter) comprises a nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or a complement thereof.

In some embodiments, a suitable guide nucleic acid is a single RNA polynucleotide and comprises first and second nucleotide sequence having 60% or more (e.g., 65% or more, 70% or more, 75% or more, 80% or more, 85% or more, 90% or more, 95% or more, 98% or more, 99% or more, or 100%) nucleotide sequence identity over a stretch of 8 or more contiguous nucleotides (e.g., 8 or more contiguous nucleotides, 10 or more contiguous nucleotides, 12 or more contiguous nucleotides, 15 or more contiguous nucleotides, or 20 or more contiguous nucleotides) to any one of the nucleotide sequences set forth in International Patent Application Nos. PCT/US2016/052690 and PCT/US2017/062617, or complements thereof.

In some embodiments, the guide RNA is a Cpf1 and/or Cas9 guide RNA. A Cpf1 and/or Cas9 guide RNA can have a total length of from 30 nucleotides (nt) to 100 nt, e.g., from 30 nt to 40 nt, from 40 nt to 45 nt, from 45 nt to 50 nt, from 50 nt to 60 nt, from 60 nt to 70 nt, from 70 nt to 80 nt, from 80 nt to 90 nt, or from 90 nt to 100 nt. In some embodiments, a Cpf1 and/or Cas9 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt. A Cpf1 and/or Cas9 guide RNA can include a target nucleic acid-binding segment and a duplex-forming segment.

The target nucleic acid-binding segment of a Cpf1 and/or Cas9 guide RNA can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some embodiments, the target nucleic acid-binding segment has a length of 23 nt. In some embodiments, the target nucleic acid-binding segment has a length of 24 nt. In some embodiments, the target nucleic acid-binding segment has a length of 25 nt.

The target nucleic acid-binding segment of a Cpf1 and/or Cas9 guide RNA can have 100% complementarity with a corresponding length of target nucleic acid sequence. The targeting segment can have less than 100% complementarity with a corresponding length of target nucleic acid sequence. For example, the target nucleic acid binding segment of a Cpf1 and/or Cas9 guide RNA can have 1, 2, 3, 4, or 5 nucleotides that are not complementary to the target nucleic acid sequence. For example, in some embodiments, where a target nucleic acid-binding segment has a length of 25 nucleotides, and the target nucleic acid sequence has a length of 25 nucleotides, in some embodiments, the target nucleic acid-binding segment has 100% complementarity to the target nucleic acid sequence. As another example, in some embodiments, where a target nucleic acid-binding segment has a length of 25 nucleotides, and the target nucleic acid sequence has a length of 25 nucleotides, in some embodiments, the target nucleic acid-binding segment has 1 non-complementary nucleotide and 24 complementary nucleotides with the target nucleic acid sequence.

The duplex-forming segment of a Cpf1 and/or Cas9 guide RNA can have a length of from 15 nt to 25 nt, e.g., 15 nt, 16 nt, 17 nt, 18 nt, 19 nt, 20 nt, 21 nt, 22 nt, 23 nt, 24 nt, or 25 nt.

In some embodiments, the duplex-forming segment of a Cpf1 guide RNA can comprise the nucleotide sequence 5′-AAUUUCUACUGUUGUAGAU-3′.

Additional Elements

In some embodiments, a guide nucleic acid (e.g., guide RNA) includes an additional segment or segments (in some embodiments at the 5′ end, in some embodiments the 3′ end, in some embodiments at either the 5′ or 3′ end, in some embodiments embedded within the sequence (i.e., not at the 5′ and/or 3′ end), in some embodiments at both the 5′ end and the 3′ end, in some embodiments embedded and at the 5′ end and/or the 3′ end, etc.). For example, a suitable additional segment can include a 5′ cap (e.g., a 7-methylguanylate cap (m⁷G)); a 3′ polyadenylated tail (i.e., a 3′ poly(A) tail); a ribozyme sequence (e.g. to allow for self-cleavage of a guide nucleic acid or component of a guide nucleic acid, e.g., a targeter, an activator, etc.); a riboswitch sequence (e.g., to allow for regulated stability and/or regulated accessibility by proteins and protein complexes); a sequence that forms a dsRNA duplex (i.e., a hairpin)); a sequence that targets an RNA to a subcellular location (e.g., nucleus, mitochondria, chloroplasts, and the like); a modification or sequence that provides for tracking (e.g., a label such as a fluorescent molecule (i.e., fluorescent dye), a sequence or other moiety that facilitates fluorescent detection; a sequence or other modification that provides a binding site for proteins (e.g., proteins that act on DNA, including transcriptional activators, transcriptional repressors, DNA methyltransferases, DNA demethylases, histone acetyltransferases, histone deacetylases, proteins that bind RNA (e.g., RNA aptamers), labeled proteins, fluorescently labeled proteins, and the like); a modification or sequence that provides for increased, decreased, and/or controllable stability; and combinations thereof.

RNA-Guided Endonuclease

In addition to, or instead of, a guide nucleic acid, the composition can comprise an RNA-guided endonuclease protein or nucleic acid (e.g., mRNA or vector) encoding same. Any RNA-guided endonuclease can be used. The selection of the RNA guided endonuclease used will depend, at least in part, to the intended end-use of the CRISPR system employed.

In some embodiments, the polypeptide is a Cas 9 polypeptide. Suitable Cas9 polypeptides for inclusion in a composition of the present disclosure include a naturally-occurring Cas9 polypeptide (e.g., naturally occurs in bacterial and/or archaeal cells), or a non-naturally-occurring Cas9 polypeptide (e.g., the Cas9 polypeptide is a variant Cas9 polypeptide, a chimeric polypeptide as discussed below, and the like), as described below. In some embodiments, one skilled in the art can appreciate that the Cas9 polypeptide disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity. As such, a Cas9 polypeptide that is suitable for inclusion in a composition of the present disclosure can be an enzymatically active Cas9 polypeptide, e.g., can make single- or double-stranded breaks in a target nucleic acid, or alternatively can have reduced enzymatic activity compared to a wild-type Cas9 polypeptide.

Naturally occurring Cas9 polypeptides bind a guide nucleic acid, are thereby directed to a specific sequence within a target nucleic acid (a target site), and cleave the target nucleic acid (e.g., cleave dsDNA to generate a double strand break, cleave ssDNA, cleave ssRNA, etc.). A subject Cas9 polypeptide comprises two portions, an RNA-binding portion and an activity portion. The RNA-binding portion interacts with a subject guide nucleic acid, and an activity portion exhibits site-directed enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc. In some embodiments the activity portion exhibits reduced nuclease activity relative to the corresponding portion of a wild type Cas9 polypeptide. In some embodiments, the activity portion is enzymatically inactive.

Assays to determine whether a protein has an RNA-binding portion that interacts with a subject guide nucleic acid can be any convenient binding assay that tests for binding between a protein and a nucleic acid. Exemplary binding assays include binding assays (e.g., gel shift assays) that involve adding a guide nucleic acid and a Cas9 polypeptide to a target nucleic acid.

Assays to determine whether a protein has an activity portion (e.g., to determine if the polypeptide has nuclease activity that cleave a target nucleic acid) can be any convenient nucleic acid cleavage assay that tests for nucleic acid cleavage. Exemplary cleavage assays that include adding a guide nucleic acid and a Cas9 polypeptide to a target nucleic acid.

In some embodiments, a suitable Cas9 polypeptide for inclusion in a composition of the present disclosure has enzymatic activity that modifies target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other embodiments, a suitable Cas9 polypeptide for inclusion in a composition of the present disclosure has enzymatic activity that modifies a polypeptide (e.g., a histone) associated with target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Many Cas9 orthologues from a wide variety of species have been identified and in some embodiments, the proteins share only a few identical amino acids. All identified Cas9 orthologues have the same domain architecture with a central HNH endonuclease domain and a split RuvC/RNaseH domain. Cas9 proteins share 4 key motifs with a conserved architecture. Motifs 1, 2, and 4 are RuvC like motifs while motif 3 is an HNH-motif.

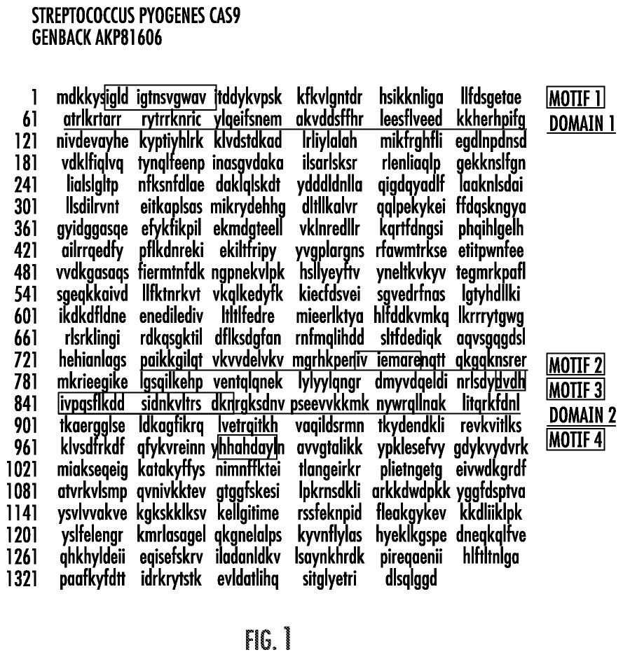

In some embodiments, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to the Cas9 amino acid sequence depicted in FIG. 1 (SEQ ID NO:1); or alternatively to motifs 1-4 of the Cas9 amino acid sequence depicted in Table 1 below; or alternatively to amino acids 7-166 or 731-1003 of the Cas9 amino acid sequence depicted in FIG. 1 (SEQ ID NO:1)

In some embodiments, a Cas9 polypeptide comprises an amino acid sequence having 75% or more, 80% or more, 85% or more, 90% or more, 95% or more, or 98%, amino acid sequence identity to the amino acid sequence depicted in FIG. 1 and set forth in SEQ ID NO:1; and comprises amino acid substitutions of N497, R661, Q695, and Q926 relative to the amino acid sequence set forth in SEQ ID NO:1; or comprises an amino acid substitution of K855 relative to the amino acid sequence set forth in SEQ ID NO:1; or comprises amino acid substitutions of K810, K1003, and R1060 relative to the amino acid sequence set forth in SEQ ID NO:1; or comprises amino acid substitutions of K848, K1003, and R1060 relative to the amino acid sequence set forth in SEQ ID NO:1.

As used herein, the term “Cas9 polypeptide” encompasses the term “variant Cas9 polypeptide”; and the term “variant Cas9 polypeptide” encompasses the term “chimeric Cas9 polypeptide.”

Variant Cas9 Polypeptides

A suitable Cas9 polypeptides for inclusion in a composition of the present disclosure includes a variant Cas9 polypeptide. A variant Cas9 polypeptide has an amino acid sequence that is different by one amino acid (e.g., has a deletion, insertion, substitution, fusion) (i.e., different by at least one amino acid) when compared to the amino acid sequence of a wild type Cas9 polypeptide (e.g., a naturally occurring Cas9 polypeptide, as described above). In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 polypeptide. In some embodiments, the variant Cas9 polypeptide has no substantial nuclease activity. When a Cas9 polypeptide is a variant Cas9 polypeptide that has no substantial nuclease activity, it can be referred to as “dCas9.”

In some embodiments, a variant Cas9 polypeptide has reduced nuclease activity. For example, a variant Cas9 polypeptide suitable for use in a binding method of the present disclosure exhibits less than about 20%, less than about 15%, less than about 10%, less than about 5%, less than about 1%, or less than about 0.1%, of the endonuclease activity of a wild-type Cas9 polypeptide, e.g., a wild-type Cas9 polypeptide comprising an amino acid sequence as depicted in FIG. 1 (SEQ ID NO:1).

In some embodiments, a variant Cas9 polypeptide can cleave the complementary strand of a target nucleic acid but has reduced ability to cleave the non-complementary strand of a double stranded target nucleic acid. For example, the variant Cas9 polypeptide can have a mutation (amino acid substitution) that reduces the function of the RuvC domain (e.g., “domain 1” of FIG. 1). As a non-limiting example, in some embodiments, a variant Cas9 polypeptide has a D10A mutation (e.g., aspartate to alanine at an amino acid position corresponding to position 10 of SEQ ID NO:1) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 polypeptide cleaves a double stranded target nucleic acid) (see, for example, Jinek et al., Science. 2012 Aug. 17; 337(6096):816-21).

In some embodiments, a variant Cas9 polypeptide can cleave the non-complementary strand of a double stranded target nucleic acid but has reduced ability to cleave the complementary strand of the target nucleic acid. For example, the variant Cas9 polypeptide can have a mutation (amino acid substitution) that reduces the function of the HNH domain (RuvC/HNH/RuvC domain motifs, “domain 2” of FIG. 1). As a non-limiting example, in some embodiments, the variant Cas9 polypeptide can have an H840A mutation (e.g., histidine to alanine at an amino acid position corresponding to position 840 of SEQ ID NO:1) (FIG. 1) and can therefore cleave the non-complementary strand of the target nucleic acid but has reduced ability to cleave the complementary strand of the target nucleic acid (thus resulting in a SSB instead of a DSB when the variant Cas9 polypeptide cleaves a double stranded target nucleic acid). Such a Cas9 polypeptide has a reduced ability to cleave a target nucleic acid (e.g., a single stranded target nucleic acid) but retains the ability to bind a target nucleic acid (e.g., a single-stranded or a double-stranded target nucleic acid).

In some embodiments, a variant Cas9 polypeptide has a reduced ability to cleave both the complementary and the non-complementary strands of a double stranded target nucleic acid. As a non-limiting example, in some embodiments, the variant Cas9 polypeptide harbors both the D10A and the H840A mutations (e.g., mutations in both the RuvC domain and the HNH domain) such that the polypeptide has a reduced ability to cleave both the complementary and the non-complementary strands of a double stranded target nucleic acid. Such a Cas9 polypeptide has a reduced ability to cleave a target nucleic acid (e.g., a single-stranded target nucleic acid or a double-stranded target nucleic acid) but retains the ability to bind a target nucleic acid (e.g., a single stranded target nucleic acid or a double-stranded target nucleic acid).

As another non-limiting example, in some embodiments, the variant Cas9 polypeptide harbors W476A and W1126A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, W476A, and WI126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors H840A, D10A, W476A, and WI126A, mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

As another non-limiting example, in some embodiments, the variant Cas9 polypeptide harbors D10A, H840A, P475A, W476A, N477A, D1125A, W1126A, and D1127A mutations such that the polypeptide has a reduced ability to cleave a target nucleic acid. Such a Cas9 polypeptide has a reduced ability to cleave a target nucleic acid but retains the ability to bind a target nucleic acid.

Other residues can be mutated to achieve the above effects (i.e. inactivate one or the other nuclease portions). As non-limiting examples, residues D10, G12, G17, E762, H840, N854, N863, H982, H983, A984, D986, and/or A987 can be altered (i.e., substituted) (see Table 1 for more information regarding the conservation of Cas9 amino acid residues). Also, mutations other than alanine substitutions are suitable.

In some embodiments, a variant Cas9 polypeptide that has reduced catalytic activity (e.g., when a Cas9 protein has a D10, G12, G17, E762, H840, N854, N863, H982, H983, A984, D986, and/or a A⁹⁸⁷ mutation, e.g., D10A, G12A, G17A, E762A, H840A, N854A, N863A, H982A, H983A, A984A, and/or D986A), the variant Cas9 polypeptide can still bind to target nucleic acid in a site-specific manner (because it is still guided to a target nucleic acid sequence by a guide nucleic acid) as long as it retains the ability to interact with the guide nucleic acid.

TABLE 1
Table 1 lists 4 motifs that are present in Cas9
sequences from various species The amino
acids listed here are from the
Cas9 from S. pyogenes (SEQ ID NO: 1).

| | | | Highly |
|---|---|---|---|
| Motif | Motif | Amino acids (residue #s) | conserved |
| 1 | RuvC | IGLDIGTNSVGWAVI (7-21) | D10, G12, |
| | | (SEQ ID NO: 3) | G17 |
| 2 | RuvC | IVIEMARE (759-766) | E762 |
| | | (SEQ ID NO: 4) | |
| 3 | HNH- | DVDHIVPQSFLKDDSIDNKVLTRSDKN | H840, N854, |
| | motif | (837-863) (SEQ ID NO: 5) | N863 |
| 4 | RuvC | HHAHDAYL (982-989) | H982, H983, |
| | | (SEQ ID NO: 6) | A984, D986, |
| | | | A987 |

In addition to the above, a variant Cas9 protein can have the same parameters for sequence identity as described above for Cas9 polypeptides. Thus, in some embodiments, a suitable variant Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity of the Cas9 amino acid sequence depicted in FIG. 1 (SEQ ID NO:1), or alternatively to motifs 1-4 (motifs 1-4 of SEQ ID NO:1 are SEQ ID NOs: 3-6, respectively, as depicted in Table 1); or alternatively to amino acids 7-166 or 731-1003 of the Cas9 amino acid sequence depicted in FIG. 1 (SEQ ID NO:1). Any Cas9 protein as defined above can be used as a Cas9 polypeptide, or as part of a chimeric Cas9 polypeptide, in a composition of the present disclosure, including those specifically referenced in International Patent Application Nos. PCT/US2016/052690 and PCT/US2017/062617.

In some embodiments, a suitable variant Cas9 polypeptide comprises an amino acid sequence having 60% or more, 70% or more, 75% or more, 80% or more, 85% or more, 90% or more, 95% or more, 99% or more, or 100% amino acid sequence identity to the Cas9 amino acid sequence depicted in FIG. 1 (SEQ ID NO:1). Any Cas9 protein as defined above can be used as a variant Cas9 polypeptide or as part of a chimeric variant Cas9 polypeptide in a composition of the present disclosure, including those specifically referenced in International Patent Application Nos. PCT/US2016/052690 and PCT/US2017/062617.

Chimeric Polypeptides (Fusion Polypeptides)

In some embodiments, a variant Cas9 polypeptide is a chimeric Cas9 polypeptide (also referred to herein as a fusion polypeptide, e.g., a “Cas9 fusion polypeptide”). A Cas9 fusion polypeptide can bind and/or modify a target nucleic acid (e.g., cleave, methylate, demethylate, etc.) and/or a polypeptide associated with target nucleic acid (e.g., methylation, acetylation, etc., of, for example, a histone tail).

A Cas9 fusion polypeptide is a variant Cas9 polypeptide by virtue of differing in sequence from a wild type Cas9 polypeptide (e.g., a naturally occurring Cas9 polypeptide). A Cas9 fusion polypeptide is a Cas9 polypeptide (e.g., a wild type Cas9 polypeptide, a variant Cas9 polypeptide, a variant Cas9 polypeptide with reduced nuclease activity (as described above), and the like) fused to a covalently linked heterologous polypeptide (also referred to as a “fusion partner”). In some embodiments, a Cas9 fusion polypeptide is a variant Cas9 polypeptide with reduced nuclease activity (e.g., dCas9) fused to a covalently linked heterologous polypeptide. In some embodiments, the heterologous polypeptide exhibits (and therefore provides for) an activity (e.g., an enzymatic activity) that will also be exhibited by the Cas9 fusion polypeptide (e.g., methyltransferase activity, acetyltransferase activity, kinase activity, ubiquitinating activity, etc.). In some such embodiments, a method of binding, e.g., where the Cas9 polypeptide is a variant Cas9 polypeptide having a fusion partner (i.e., having a heterologous polypeptide) with an activity (e.g., an enzymatic activity) that modifies the target nucleic acid, the method can also be considered to be a method of modifying the target nucleic acid. In some embodiments, a method of binding a target nucleic acid (e.g., a single stranded target nucleic acid) can result in modification of the target nucleic acid. Thus, in some embodiments, a method of binding a target nucleic acid (e.g., a single stranded target nucleic acid) can be a method of modifying the target nucleic acid.

In some embodiments, the heterologous sequence provides for subcellular localization, i.e., the heterologous sequence is a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some embodiments, a variant Cas9 does not include a NLS so that the protein is not targeted to the nucleus (which can be advantageous, e.g., when the target nucleic acid is an RNA that is present in the cytosol). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the heterologous sequence can provide for increased or decreased stability (i.e., the heterologous sequence is a stability control peptide, e.g., a degron, which in some embodiments is controllable (e.g., a temperature sensitive or drug controllable degron sequence, see below). In some embodiments, the heterologous sequence can provide for increased or decreased transcription from the target nucleic acid (i.e., the heterologous sequence is a transcription modulation sequence, e.g., a transcription factor/activator or a fragment thereof, a protein or fragment thereof that recruits a transcription factor/activator, a transcription repressor or a fragment thereof, a protein or fragment thereof that recruits a transcription repressor, a small molecule/drug-responsive transcription regulator, etc.). In some embodiments, the heterologous sequence can provide a binding domain (i.e., the heterologous sequence is a protein binding sequence, e.g., to provide the ability of a Cas9 fusion polypeptide to bind to another protein of interest, e.g., a DNA or histone modifying protein, a transcription factor or transcription repressor, a recruiting protein, an RNA modification enzyme, an RNA-binding protein, a translation initiation factor, an RNA splicing factor, etc.). A heterologous nucleic acid sequence may be linked to another nucleic acid sequence (e.g., by genetic engineering) to generate a chimeric nucleotide sequence encoding a chimeric polypeptide.

A subject Cas9 fusion polypeptide (Cas9 fusion protein) can have multiple (1 or more, 2 or more, 3 or more, etc.) fusion partners in any combination of the above. As an illustrative example, a Cas9 fusion protein can have a heterologous sequence that provides an activity (e.g., for transcription modulation, target modification, modification of a protein associated with a target nucleic acid, etc.) and can also have a subcellular localization sequence. In some embodiments, such a Cas9 fusion protein might also have a tag for ease of tracking and/or purification (e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). As another illustrative example, a Cas9 protein can have one or more NLSs (e.g., two or more, three or more, four or more, five or more, 1, 2, 3, 4, or 5 NLSs). In some embodiments a fusion partner (or multiple fusion partners) (e.g., an NLS, a tag, a fusion partner providing an activity, etc.) is located at or near the C-terminus of Cas9. In some embodiments a fusion partner (or multiple fusion partners) (e.g., an NLS, a tag, a fusion partner providing an activity, etc.) is located at the N-terminus of Cas9. In some embodiments a Cas9 has a fusion partner (or multiple fusion partners)(e.g., an NLS, a tag, a fusion partner providing an activity, etc.) at both the N-terminus and C-terminus.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled in part by the degron sequence. In some embodiments, a suitable degron is constitutive such that the degron exerts its influence on protein stability independent of experimental control (i.e., the degron is not drug inducible, temperature inducible, etc.) In some embodiments, the degron provides the variant Cas9 polypeptide with controllable stability such that the variant Cas9 polypeptide can be turned “on” (i.e., stable) or “off” (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off”, degraded) above the threshold temperature. As another example, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1).: Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing Cas9 (e.g., wild type Cas9; variant Cas9; variant Cas9 with reduced nuclease activity, e.g., dCas9; and the like) to a degron sequence produces a “tunable” and “inducible” Cas9 polypeptide. Any of the fusion partners described herein can be used in any desirable combination. As one non-limiting example to illustrate this point, a Cas9 fusion protein (i.e., a chimeric Cas9 polypeptide) can comprise a YFP sequence for detection, a degron sequence for stability, and transcription activator sequence to increase transcription of the target nucleic acid. A suitable reporter protein for use as a fusion partner for a Cas9 polypeptide (e.g., wild type Cas9, variant Cas9, variant Cas9 with reduced nuclease function, etc.), includes, but is not limited to, the following exemplary proteins (or functional fragment thereof): his3, β-galactosidase, a fluorescent protein (e.g., GFP, RFP, YFP, cherry, tomato, etc., and various derivatives thereof), luciferase, β-glucuronidase, and alkaline phosphatase. Furthermore, the number of fusion partners that can be used in a Cas9 fusion protein is unlimited. In some embodiments, a Cas9 fusion protein comprises one or more (e.g. two or more, three or more, four or more, or five or more) heterologous sequences.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, any of which can be directed at modifying nucleic acid directly (e.g., methylation of DNA or RNA) or at modifying a nucleic acid-associated polypeptide (e.g., a histone, a DNA binding protein, and RNA binding protein, and the like). Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

Examples of various additional suitable fusion partners (or fragments thereof) for a subject variant Cas9 polypeptide include, but are not limited to those described in the PCT patent applications: WO2010/075303, WO2012/068627, and WO2013/155555 which are hereby incorporated by reference in their entirety.

Suitable fusion partners include, but are not limited to, a polypeptide that provides an activity that indirectly increases transcription by acting directly on the target nucleic acid or on a polypeptide (e.g., a histone, a DNA-binding protein, an RNA-binding protein, an RNA editing protein, etc.) associated with the target nucleic acid. Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

Additional suitable fusion partners include, but are not limited to, a polypeptide that directly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.).

Non-limiting examples of fusion partners to accomplish increased or decreased transcription include transcription activator and transcription repressor domains (e.g., the Krnppel associated box (KRAB or SKD); the Mad mSIN3 interaction domain (SID); the ERF repressor domain (ERD), etc.). In some such embodiments, a Cas9 fusion protein is targeted by the guide nucleic acid to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target nucleic acid or modifies a polypeptide associated with the target nucleic acid). In some embodiments, the changes are transient (e.g., transcription repression or activation). In some embodiments, the changes are inheritable (e.g., when epigenetic modifications are made to the target nucleic acid or to proteins associated with the target nucleic acid, e.g., nucleosomal histones).

Non-limiting examples of fusion partners for use when targeting ssRNA target nucleic acids include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); heliembodiments; RNA-binding proteins; and the like. It is understood that a fusion partner can include the entire protein or in some embodiments can include a fragment of the protein (e.g., a functional domain).

In some embodiments, the heterologous sequence can be fused to the C-terminus of the Cas9 polypeptide. In some embodiments, the heterologous sequence can be fused to the N-terminus of the Cas9 polypeptide. In some embodiments, the heterologous sequence can be fused to an internal portion (i.e., a portion other than the N- or C-terminus) of the Cas9 polypeptide.

In addition the fusion partner of a chimeric Cas9 polypeptide can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase I, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP S1, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI D1 and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627.

Some RNA splicing factors that can be used (in whole or as fragments thereof) as fusion partners for a Cas9 polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP A1 can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cd-elements that are located in either the core exon region or the exon extension region (i.e., between the two alternative 5′ splice sites). For more examples, see WO2010075303.

In some embodiments, a Cas9 polypeptide (e.g., a wild type Cas9, a variant Cas9, a variant Cas9 with reduced nuclease activity, etc.) can be linked to a fusion partner via a peptide spacer.

In some embodiments, a Cas9 polypeptide comprises a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD attached to another molecule facilitates entry of the molecule into the nucleus (e.g., in some embodiments, a PTD includes a nuclear localization signal (NLS)). In some embodiments, a Cas9 polypeptide comprises two or more NLSs, e.g., two or more NLSs in tandem. In some embodiments, a PTD is covalently linked to the amino terminus of a Cas9 polypeptide. In some embodiments, a PTD is covalently linked to the carboxyl terminus of a Cas9 polypeptide. In some embodiments, a PTD is covalently linked to the amino terminus and to the carboxyl terminus of a Cas9 polypeptide. In some embodiments, a PTD is covalently linked to a nucleic acid (e.g., a guide nucleic acid, a polynucleotide encoding a guide nucleic acid, a polynucleotide encoding a Cas9 polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:8); Transportan GWTLNSAGYLLGKINLKALAALAKKIL (SEQ ID NO:9); KALAWEAKLAKALAKALAKHLAKALAKALKCEA (SEQ ID NO:10); and RQIKIWFQNRRMKWKK (SEQ ID NO:11). Exemplary PTDs include but are not limited to, YGRKKRRQRRR (SEQ ID NO:12), RKKRRQRRR (SEQ ID NO:13); an arginine homopolymer of from 3 arginine residues to 50 arginine residues; Exemplary PTD domain amino acid sequences include, but are not limited to, any of the following: YGRKKRRQRRR (SEQ ID NO:14); RKKRRQRR (SEQ ID NO:15); YARAAARQARA (SEQ ID NO:16); THRLPRRRRRR (SEQ ID NO:17); and GGRRARRRRRR (SEQ ID NO:18). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R⁹”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

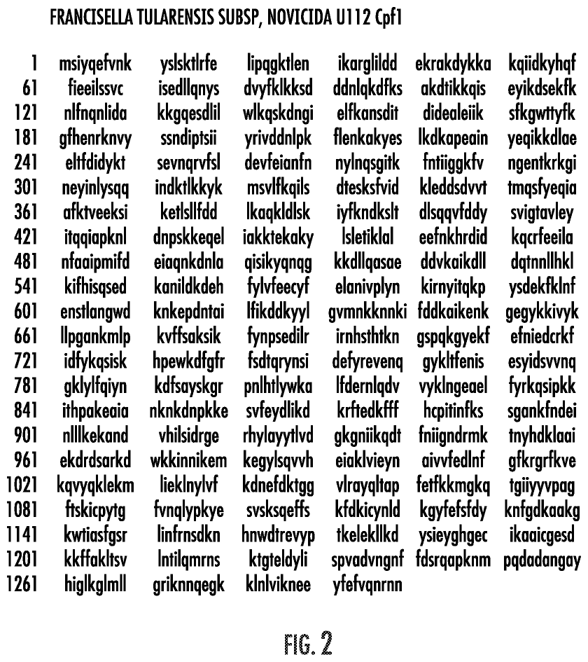

In some embodiments, the composition can comprise a Cpf1 RNA-guided endonuclease, an example of which is provided in FIG. 2, 3, or 4. Another name for the Cpf1 RNA-guided endonuclease is Cas12a. The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 bp overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

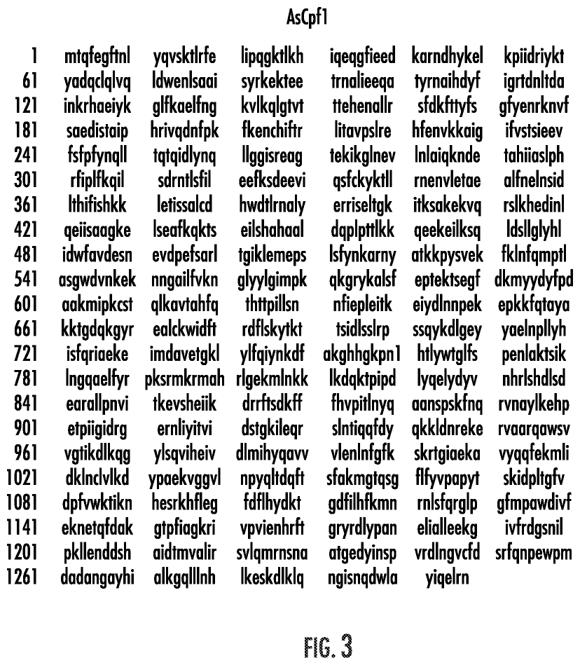

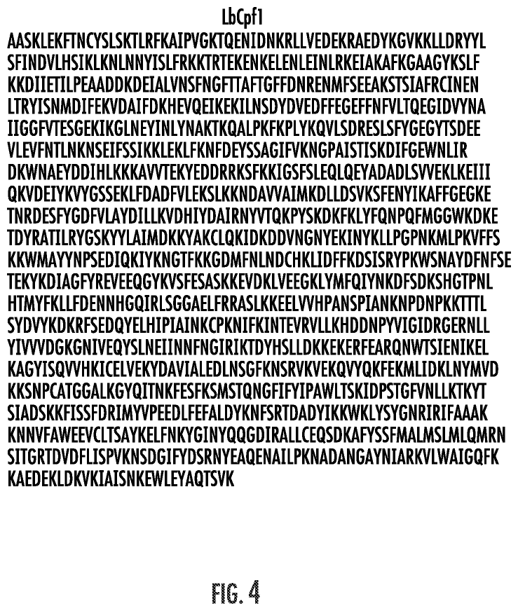

Persons skilled in the art will appreciate that the Cpf1 disclosed herein can be any variant derived or isolated from any source, many of which are known in the art. For example, in some embodiments, the Cpf1 peptide of the present disclosure can include FnCPFT (e.g., SEQ ID NO: 2) set forth in FIG. 2, AsCpf1 (e.g., FIG. 3), LbCpf1 (e.g., FIG. 4) or any other of the many known Cpf1 proteins from various other microorganism species, and synthetic variants thereof.

In some embodiments, the composition comprises a Cpf1 polypeptide. In some embodiments, the Cpf1 polypeptide is enzymatically active, e.g., the Cpf1 polypeptide, when bound to a guide RNA, cleaves a target nucleic acid. In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 3, or 4), and retains DNA binding activity.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 3, or 4. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to a contiguous stretch of from 100 amino acids to 200 amino acids (aa), from 200 aa to 400 aa, from 400 aa to 600 aa, from 600 aa to 800 aa, from 800 aa to 1000 aa, from 1000 aa to 1100 aa, from 1100 aa to 1200 aa, or from 1200 aa to 1300 aa, of the amino acid sequence depicted in FIG. 2, 3, or 4.

In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCI domain of a Cpf1 polypeptide of the amino acid sequence depicted in FIG. 2, 3, or 4. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCII domain of a Cpf1 polypeptide of the amino acid sequence depicted in FIG. 2, 3, or 4. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the RuvCIII domain of a Cpf1 polypeptide of the amino acid sequence depicted in FIG. 2, 3, or 4.

In some embodiments, the Cpf1 polypeptide exhibits reduced enzymatic activity relative to a wild-type Cpf1 polypeptide (e.g., relative to a Cpf1 polypeptide comprising the amino acid sequence depicted in FIG. 2, 3, or 4), and retains DNA binding activity. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 3, or 4; and comprises an amino acid substitution (e.g., a D→A substitution) at an amino acid residue corresponding to amino acid 917 of the amino acid sequence depicted in FIG. 2, 3, or 4. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 3, or 4; and comprises an amino acid substitution (e.g., an E→A substitution) at an amino acid residue corresponding to amino acid 1006 of the amino acid sequence depicted in FIG. 2, 3, or 4. In some embodiments, a Cpf1 polypeptide comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the amino acid sequence depicted in FIG. 2, 3, or 4; and comprises an amino acid substitution (e.g., a D→A substitution) at an amino acid residue corresponding to amino acid 1255 of the amino acid sequence depicted in FIG. 2, 3, or 4.

In some embodiments, the Cpf1 polypeptide is a fusion polypeptide, e.g., where a Cpf1 fusion polypeptide comprises: a) a Cpf1 polypeptide; and b) a heterologous fusion partner. In some embodiments, the heterologous fusion partner is fused to the N-terminus of the Cpf1 polypeptide. In some embodiments, the heterologous fusion partner is fused to the C-terminus of the Cpf1 polypeptide. In some embodiments, the heterologous fusion partner is fused to both the N-terminus and the C-terminus of the Cpf1 polypeptide. In some embodiments, the heterologous fusion partner is inserted internally within the Cpf1 polypeptide.

Suitable heterologous fusion partners include NLS, epitope tags, fluorescent polypeptides, and the like.

Linked Guide RNA and Donor Nucleic Acid

In one aspect, the invention provides a complex comprising a CRISPR system comprising an RNA-guided endonuclease (e.g. a Cas9 or Cpf1 polypeptide), a guide RNA and a donor polynucleotide, wherein the guide RNA and the donor polynucleotide are linked. As exemplified herein, the guide RNA and donor polynucleotide can be either covalently or non-covalently linked. In one embodiment, the guide RNA and donor polynucleotide are chemically ligated. In another embodiment, the guide RNA and donor polynucleotide are enzymatically ligated. In one embodiment, the guide RNA and donor polynucleotide hybridize to each other. In another embodiment, the guide RNA and donor polynucleotide both hybridize to a bridge sequence. Any number of such hybridization schemes are possible.

Deaminase

In some embodiments, the complex or composition further comprises a deaminase (e.g., an adenine base editor). As used herein, the term “deaminase” or “deaminase domain” refers to an enzyme that catalyzes the removal of an amine group from a molecule, or deamination. In some embodiments, the deaminase is a cytidine deaminase, catalyzing the hydrolytic deamination of cytidine or deoxycytidine to uridine or deoxyuridine, respectively. In some embodiments, the deaminase is a cytosine deaminase, catalyzing the hydrolytic deamination of cytosine to uracil (e.g., in RNA) or thymine (e.g., in DNA).

In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium. In some embodiments, the deaminase or deaminase domain is a variant of a naturally-occurring deaminase from an organism, such as a human, chimpanzee, gorilla, monkey, cow, dog, rat, or mouse.

In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the deaminase is APOBEC1 or a variant thereof.

The deaminase can be used in conjugation with any of the other CRISPR elements described herein (i.e., as a composition), or the deaminase can be fused to any of the other CRISPR elements (e.g., Cas9 or Cpf1) described herein (i.e., as a complex). In certain embodiments, the deaminase is fused to Cas9, Cpf1, or a variant thereof.

Other Components

The composition can further comprise any other components typically used in nucleic acid or protein delivery formulations. For instance, the composition can further comprise lipids, lipoproteins (e.g., cholesterol and derivatives), phospholipids, polymers or other components of liposomal or micellar delivery vehicles. The composition also can comprise solvent or carrier suitable for administration to cells or hosts, such as a mammal or human.

In some embodiments, the composition further comprises one or more surfactants. The surfactant can be a non-ionic surfactant and/or a zwitterionic surfactant. In some embodiments, the surfactant is a polymer or copolymer of ethylene oxide (EO), propylene oxide (PO), butylene oxide (BO), glycolic acid (GA), lactic acid (LA), or combinations thereof. For example, the surfactant can be polyethylene glycol (PEG), polypropylene glycol, polyglycolic acid (PGA), polylactic acid, or mixtures thereof. A list of exemplary surfactants includes, but is not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-6301NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether, and sorbitan esters (commonly known as the Spans), such as sorbitan trioleate (Span 85) and sorbitan monolaurate. In some embodiments, the surfactant is an anticoagulant (e.g., heparin or the like). In some embodiments, the composition further comprises one or more pharmaceutically acceptable carriers and/or excipients.

In some instances, a component (e.g., a nucleic acid component (e.g., a guide nucleic acid, etc.); a protein component (e.g., a Cas9 or Cpf1 polypeptide, a variant Cas9 or Cpf1 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels)(e.g., a fluorescent label) and indirectly detectable labels (indirect labels) (e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label)(e.g., ³H, ¹²⁵I, ³⁵S, ¹⁴C, or ³²P); an enzyme (an indirect label)(e.g., peroxidase, alkaline phosphatase, galactosidase, luciferase, glucose oxidase, and the like); a fluorescent protein (a direct label) (e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some embodiments, when two or more components are labeled, they can be labeled with label moieties that are distinguishable from one another.

Encapsulation and Nanoparticles

In some embodiments of the composition, the polymer combines with the nucleic acid and/or polypeptide and partially or completely encapsulates the nucleic acid and/or polypeptide. The composition can, in some formulations, provide a nanoparticle comprising the polymer and nucleic acid and/or polypeptide.

In some embodiments, the composition can comprise a core nanoparticle in addition to the polymer described herein and the nucleic acid or polypeptide. Any suitable nanoparticle can be used, including metal (e.g., gold) nanoparticles or polymer nanoparticles.

The polymer described herein and the nucleic acid (e.g., guide RNA, donor polynucleotide, or both) or polypeptide can be conjugated directly or indirectly to a nanoparticle surface. For example, the polymer described herein and the nucleic acid (e.g., guide RNA, donor polynucleotide, or both) or polypeptide can be conjugated directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In embodiments where the nanoparticle includes gold, a linker can be any thiol-containing molecule. Reaction of a thiol group with the gold results in a covalent sulfide (—S—) bond. Linker design and synthesis are well known in the art.

In some embodiments, the nucleic acid conjugated to the nanoparticle is a linker nucleic acid that serves to non-covalently bind one or more elements described herein (e.g., a Cas9 polypeptide, and a guide RNA, a donor polynucleotide, and a Cpf1 polypeptide) to the nanoparticle-nucleic acid conjugate. For instance, the linker nucleic acid can have a sequence that hybridizes to the guide RNA or donor polynucleotide.

The nucleic acid conjugated to the nanoparticle (e.g., a colloidal metal (e.g., gold) nanoparticle; a nanoparticle comprising a biocompatible polymer) can have any suitable length. When the nucleic acid is a guide RNA or donor polynucleotide, the length will be as suitable for such molecules, as discussed herein and known in the art. If the nucleic acid is a linker nucleic acid, it can have any suitable length for a linker, for instance, a length of from 10 nucleotides (nt) to 1000 nt, e.g., from about 1 nt to about 25 nt, from about 25 nt to about 50 nt, from about 50 nt to about 100 nt, from about 100 nt to about 250 nt, from about 250 nt to about 500 nt, or from about 500 nt to about 1000 nt. In some instances, the nucleic acid conjugated to the nanoparticle (e.g., a colloidal metal (e.g., gold) nanoparticle; a nanoparticle comprising a biocompatible polymer) nanoparticle can have a length of greater than 1000 nt.

When the nucleic acid linked (e.g., covalently linked; non-covalently linked) to a nanoparticle comprises a nucleotide sequence that hybridizes to at least a portion of the guide RNA or donor polynucleotide present in a complex of the present disclosure, it has a region with sequence identity to a region of the complement of the guide RNA or donor polynucleotide sequence sufficient to facilitate hybridization. In some embodiments, a nucleic acid linked to a nanoparticle in a complex of the present disclosure has at least 50%, at least 60%, at least 70%, at least 80%, at least 90%, at least 95%, at least 98%, at least 99%, or 100%, nucleotide sequence identity to a complement of from 10 to 50 nucleotides (e.g., from 10 nucleotides (nt) to 15 nt, from 15 nt to 20 nt, from 20 nt to 25 nt, from 25 nt to 30 nt, from 30 nt to 40 nt, or from 40 nt to 50 nt) of a guide RNA or donor polynucleotide present in the complex.

In some embodiments, a nucleic acid linked (e.g., covalently linked; non-covalently linked) to a nanoparticle is a donor polynucleotide, or has the same or substantially the same nucleotide sequence as a donor polynucleotide. In some embodiments, a nucleic acid linked (e.g., covalently linked; non-covalently linked) to a nanoparticle comprises a nucleotide sequence that is complementary to a donor DNA template.

The nanoparticle can have any suitable size so long as the nanoparticle is stable. For example, the nanoparticle can have an average diameter of from about 20 nm to about 500 nm, from about 50 nm to about 500 nm, from about 20 nm to about 400 nm, from about 20 nm to about 300 nm, from about 20 nm to about 250 nm, from about 50 nm to about 400 nm, from about 50 nm to about 300 nm, or from about 50 nm to about 250 nm. In certain embodiments, the nanoparticle has an average diameter of from about 50 nm to about 25 nm.

The nanoparticle can have any suitable polydispersity index (PDI). For example, the nanoparticle can have a polydispersity index of from 0.05 to about 0.5, from about 0.1 to about 0.5, from about 0.2 to about 0.5, from about 0.05 to about 0.4, from about 0.1 to about 0.4, from about 0.2 to about 0.4, from about 0.05 to about 0.3, from about 0.1 to about 0.3, or from about 0.2 to about 0.3. In certain embodiments, the nanoparticle has an average polydispersity index of from about 0.1 to about 0.3.

Method of Use

Also provided herein is a method of delivering a nucleic acid and/or polypeptide to a cell, wherein the cell can be in vitro or in vivo. The method comprises administering a composition comprising the polymer and nucleic acid and/or polypeptide, as described herein, to the cell or to a subject containing the cell. The method can be used with respect to any type of cell or subject, but is particularly useful for mammalian cells (e.g., human cells). In some embodiments, the polymer comprises a targeting agent, such that nucleic acid and/or polypeptide is delivered predominantly or exclusively to target cells or tissues (e.g., cells or tissues of the peripheral nervous system, the central nervous system, the eye of the subject, liver, muscle, lung, bone (e.g., hematopoietic cells), or tumor cells or tissues).

When used to deliver a protein or nucleic acid to a cell in a subject (i.e., in vivo), it is desirable that the polymer is stable in serum. Stability in serum can be assessed as a function of the efficiency by which the polymer delivers a protein or nucleic acid payload to a cell in serum (e.g., in vitro or in vivo). Thus, in some embodiments, the polymer delivers a given protein or nucleic acid to a cell in serum with an efficiency or tropism for a particular tissue type that is greater than that produced using pAsp[DET] under the same conditions.

In some embodiments, the polymers disclosed herein exhibit a particular tropism towards certain cell or tissue types. In some embodiments, the polymer disclosed herein has a tropism for lung tissue (i.e., upon administration to a host, the polymer is delivered in greatest amount or concentration to the lung as compared to other tissues), and is used to deliver a protein or nucleic acid to lung tissue (e.g., deliver a protein or nucleic acid to a cell or tissue of the lung of a mammal). The polymer can be any polymer described herein (e.g., a polymer of Formula 1, 1A-C, 2, 2A-C, or as otherwise described herein, including any and all features and embodiments thereof). In some embodiments, the polymer is a polymer of Formula 1, 1A-C, 2, 2A-C, wherein:

each instance of A¹ is independently:

—(CH₂)ₚ₁—[NR²—(CH₂)ₚ₂—]ᵣ₁NR²—(CH₂)ₚ₃—NH₂ or

—(CH₂)ₚ₁—[NR²—(CH₂)ₚ₂—]ᵣ₁NR²—(CH₂)ₚ₃—NHR²,

and each instance of B¹ (when present) is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—CH₂—CHOH—R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁵;

—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—CH₂—CHOH—R⁵;

—(CH₂)ₚ₁—[NR²—(CH₂)ₚ₂—]ᵣ₁NR²—(CH₂)ₚ₃—NR²—(CH₂)ₛ₁—R⁵;

—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or

—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵;

or wherein each instance of B1 (when present) is independently:

—(CH₂)ₚ₁—[NR²—(CH₂)ₚ₂—]ᵣ₁NR²—(CH₂)ₚ₃—NH—(CH₂)ₛ₁—R⁴-R⁵;

—(CH₂)ₚ₁—[NR²—(CH₂)ₚ₂—]ᵣ₁NR²—(CH₂)ₚ₃—NH—(CH₂)ₛ₁—R⁵;

—(CH₂)ₚ₁—[NR²—(CH₂)ₚ₂—]ᵣ₁NR²—(CH₂)ₚ₃—NR²—(CH₂)ₛ₁—R⁴-R⁵;

—(CH₂)ₚ₁—[NR²—(CH₂)ₚ₂—]ᵣ₁NR²—(CH₂)ₚ₃—NR²—(CH₂)ₛ₁—R⁵;

and wherein each of p1 to p3 independently is an integer of 1 to 5, and wherein p1, p3, or both are greater than p2, and/or wherein p1, p3, or both are integers of 3 to 5, and all other aspects of the polymer are as otherwise described herein including any and all features and embodiments thereof.

When used with a composition comprising one or more components of a CRISPR system, the method may be employed to edit a target nucleic acid or gene. In some embodiments, a method of modifying a target nucleic acid comprises homology-directed repair (HDR). In some embodiments, use of a complex of the present disclosure to carry out HDR provides an efficiency of HDR of at least 1%, at least 2%, at least 3%, at least 4%, at least 5%, at least 10%, at least 15%, at least 20%, at least 25%, or more than 25%. In some embodiments, a method of modifying a target nucleic acid comprises non-homologous end joining (NHEJ). In some embodiments, use of a complex of the present disclosure to carry out HDR provides an efficiency of NHEJ of at least 1%, at least 2%, at least 3%, at least 4%, at least 5%, at least 10%, at least 15%, at least 20%, at least 25%, or more than 25%.

The following examples further illustrate the invention but, of course, should not be construed as in any way limiting its scope.

### Example 1

This example provides guidance for the synthesis of Comparative Polymer 1 (“H27N”) for use in the following examples. The synthesis includes modifying PBLA with N¹-(2-aminoethyl)-N¹,N²,N²-trimethylethane-1,2-diamine and hexyl amine. An exemplary procedure is as follows.

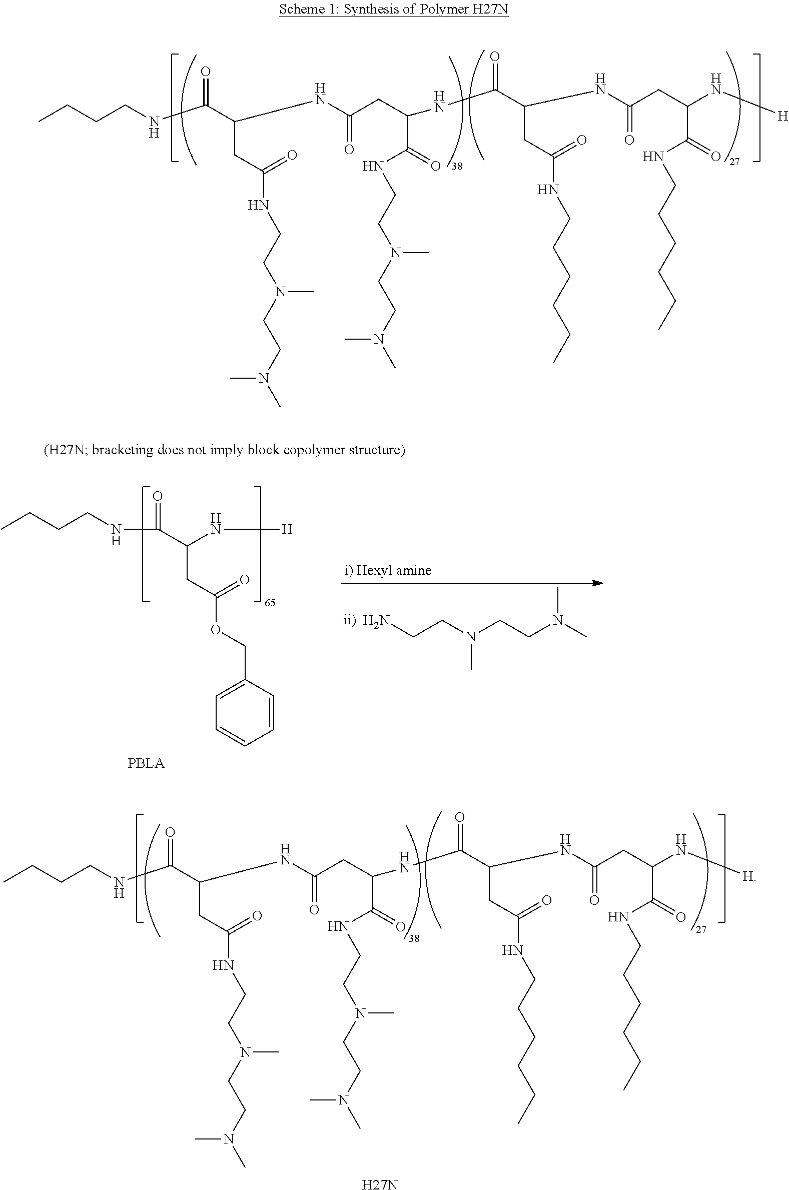

Lyophilized PBLA (50 mg, 0.0037 mmol; degree of polymerization (“DP”) 65) was placed into a flask and dissolved in tetrahy drofuran/N-methyl-2-pyrrolidine (1 mL each). To the clear solution was added n-hexylamine (160 equivalents), and the clear reaction mixture was stirred for 24 hours at room temperature. After approximately 24 hours, N¹-(2-aminoethyl)-N¹,N²,N²-trimethylethane-1,2-diamine (50 equivalents to benzyl group of PBLA segment) was added to the clear mixture under mild anhydrous conditions. After approximately 18 hours at room temperature, the reaction mixture was precipitated into diethyl ether (10-12× volume, 35 mL). The precipitate was then centrifuged, and washed twice with diethyl ether. The polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give the polymer product.

### Example 2

This example provides guidance for the synthesis of ELN10 described herein. The synthesis includes modifying PBLA with hexyl amine and amine compound 4. An exemplary procedure is as follows.

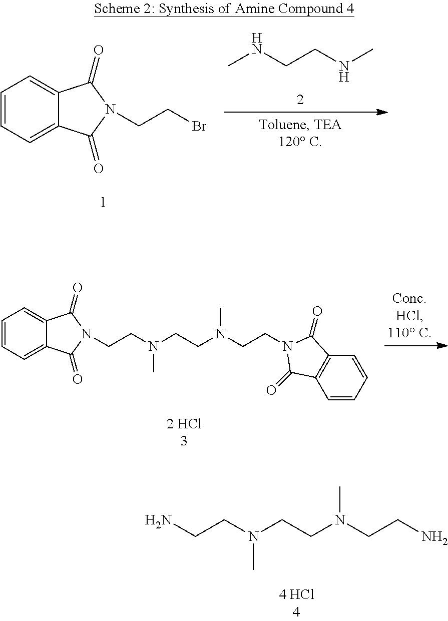

Amine compound 4 was synthesized using the protocol set forth in Scheme 2.

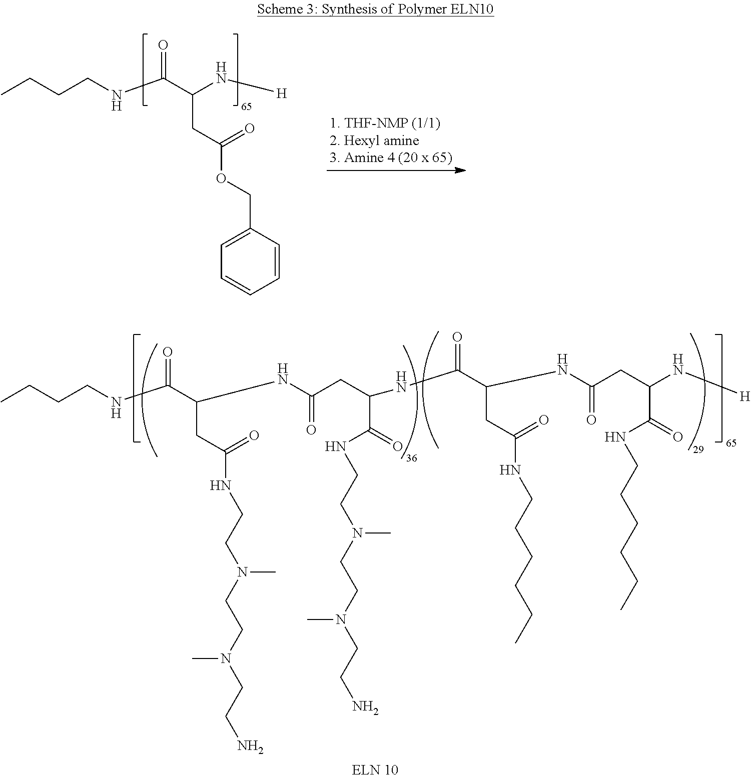

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine salt 4 (407.8 mg, 2.34 mmol) as a free amine form dissolved into 500 μL NMP, 500 μL THF and 500 μL triethylamine.

The free amine was prepared by dissolving 20 g of amine salt 4 into 30% NaOH solution (150 mL) [Caution: Exothermic reaction]. The alkaline solution was stirred at room temperature for 20 min, and then kept in an ice bath for an additional 15 min. Ethyl acetate (200 mL) was added and extracted vigorously. The organic layer was separated from the aqueous layer. The extraction was repeated a second time, and the combined organic layers dried over anhydrous sodium sulfate. A turbid solution of free amine was collected via filtration and to this solution was added 10 mL methanol, and ethyl acetate was removed to yield partially free amine. The partially free amine was resuspended into 30% NaOH solution (150 mL) and extracted with ethyl acetate two times as described above. The combined organic layers were dried over anhydrous sodium sulfate and filtered. To this turbid solution was added 10 mL methanol and the organic solvent was removed to yield 9.8 g of the free amine.

The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield ELN10 (20 mg) as a white solid.

### Example 3

This example provides guidance for the synthesis of Polymer 5 (“GenP13” and “GenP9”). The synthesis includes modifying PBLA with hexyl amine and amine compound 7. An exemplary procedure is as follows.

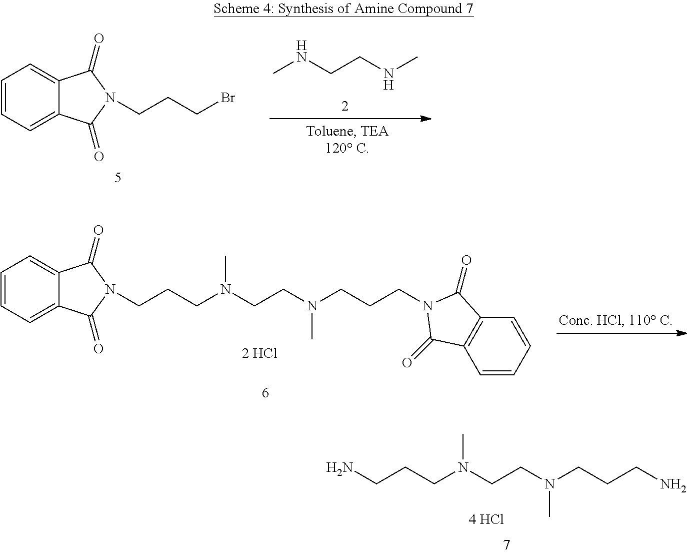

Amine compound 7 was synthesized using the protocol set forth in Scheme 4.

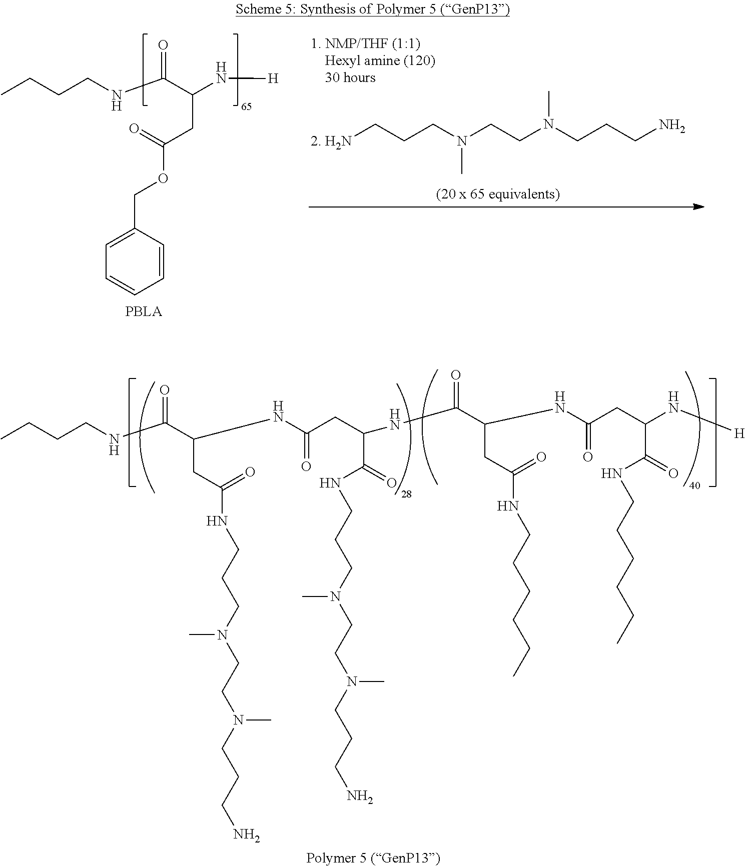

PBLA (100 mg, 0.0072 mmol) was dissolved into the mixture of 2 mL NMP and 2 mL of THF. Then to this reaction mixture was added hexyl amine (89.94 mg, 0.888 mmol) and the reaction mixture was stirred for 30 hours at room temperature. Then to this solution was added amine compound 7 (1.94 g, 9.62 mmol) as a free amine form dissolved into 500 L NMP, 500 μL THF and 500 μL triethylamine.

The free amine was prepared by dissolving 20 g of amine salt 7 into 30% NaOH solution (150 mL). The alkaline solution was stirred at room temperature for 20 min, and then kept in an ice bath for an additional 15 min. Ethyl acetate (200 mL) was added and extracted vigorously. The organic layer was separated from the aqueous layer. The extraction was repeated a second time, and the combined organic layers dried over anhydrous sodium sulfate. A turbid solution of free amine was collected via filtration and to this solution was added 10 mL methanol, and ethyl acetate was removed to yield partially free amine. The partially free amine was resuspended into 30% NaOH solution (150 mL) and extracted with ethyl acetate two times as described above. The combined organic layers were dried over anhydrous sodium sulfate and filtered. To this turbid solution was added 10 mL methanol and the organic solvent was removed to yield 9.8 g of the free amine.

The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer 5 (“GenP13”).

GenP9, having the same structure but a different ratio of hydrophobic to polyamine side chains, was synthesized using a similar procedure: PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at rt. Then to this solution was added 7 (473.38 mg, 2.34 mmol) as a free amine form dissolved into 125 μL NMP, 125 μL THF. The resulting reaction mixture was stirred at rt for 24 hours and the crude reaction mixture was precipitated into diethyl ether (40 mL) to yield crude polymer. Crude polymer was dissolved into 2 mL 1N HCl solution and was dialyzed using 20 KD cut-off membrane dialysis bag for 48 hours at 4° C. The purified polymer was lyophilized to yield Polymer 5 (“GenP9”) (20 mg) with approximately 35 amine side chains and approximately 30 C₆ sidechains as a white solid.

### Example 4

This example provides guidance for the synthesis of Polymer 6 (“GenP14”). The synthesis includes modifying PBLA with octyl amine and amine compound 7. An exemplary procedure is as follows.

Amine compound 7 was synthesized using the protocol set forth in Scheme 4 of Example 3

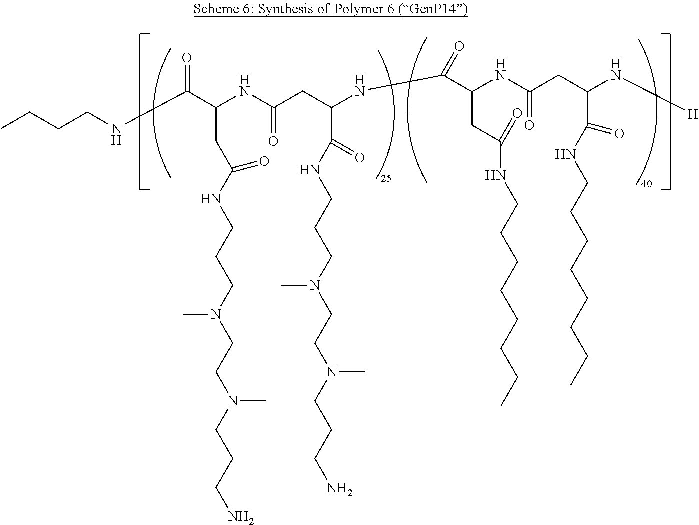

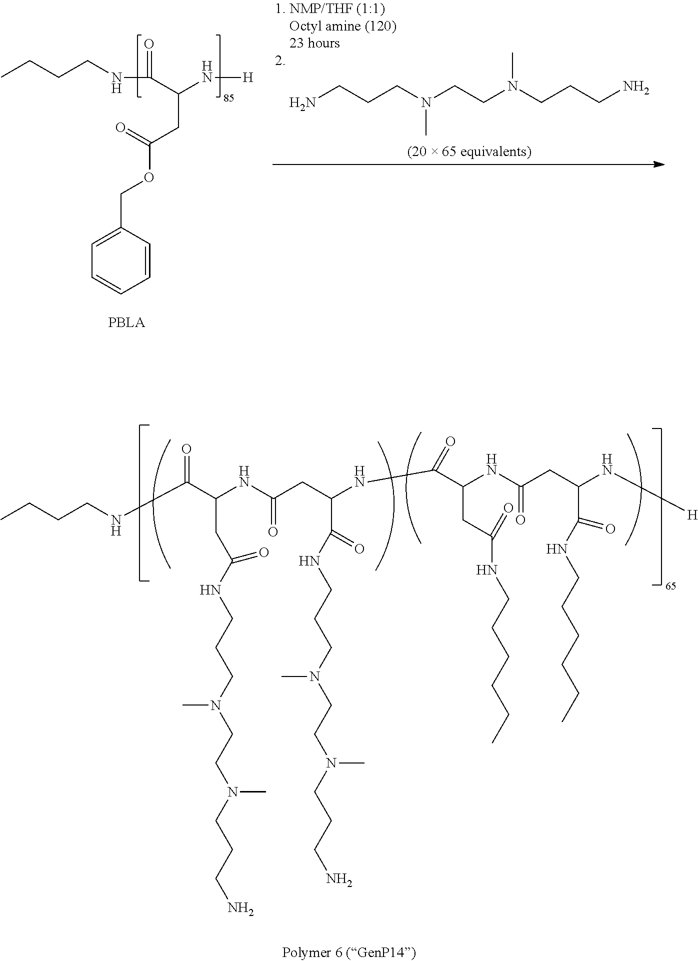

PBLA (100 mg, 0.0072 mmol) was dissolved into the mixture of 2 mL NMP and 2 mL of THF. Then to this reaction mixture was added octyl amine (115.0 mg, 0.888 mmol) and the reaction mixture was stirred for 30 hours at room temperature. Then to this solution was added amine salt 7 (1.94 g, 9.62 mmol) as a free amine form dissolved into 500 μL NMP, 500 μL THF and 500 μL triethylamine.

The free amine was prepared by dissolving 20 g of amine salt 7 into 30% NaOH solution (150 mL) [Caution: Exothermic reaction]. The alkaline solution was stirred at room temperature for 20 min, and then kept in an ice bath for an additional 15 min. Ethyl acetate (200 mL) was added and extracted vigorously. The organic layer was separated from the aqueous layer. The extraction was repeated a second time, and the combined organic layers dried over anhydrous sodium sulfate. A turbid solution of free amine was collected via filtration and to this solution was added 10 mL methanol, and ethyl acetate was removed to yield partially free amine. The partially free amine was resuspended into 30% NaOH solution (150 mL) and extracted with ethyl acetate two times as described above. The combined organic layers were dried over anhydrous sodium sulfate and filtered. To this turbid solution was added 10 mL methanol and the organic solvent was removed to yield 9.8 g of the free amine.

The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer 6 (“GenP14”).

### Example 5

This example provides guidance for the synthesis of Polymer 1 (“GenP17”, “GenP21”, “GenP27”, and “GenP29”). The synthesis includes modifying PBLA with hexyl amine and amine compound 10. An exemplary procedure is as follows.

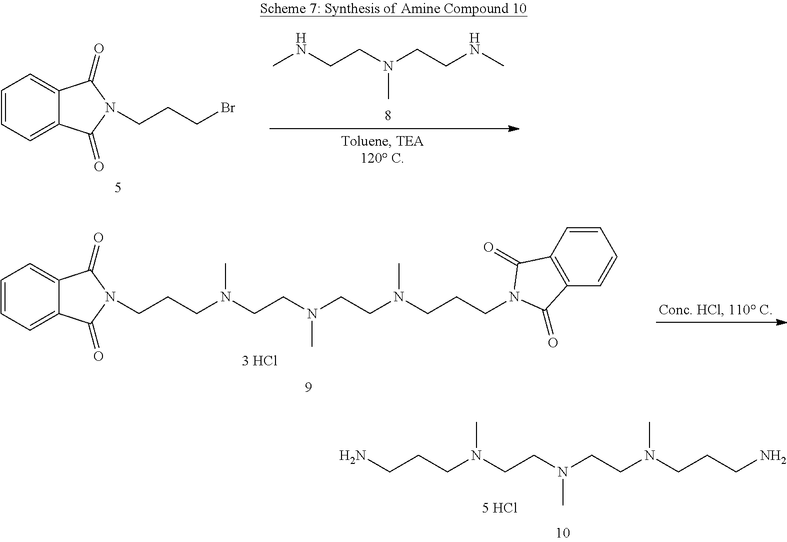

Amine compound 10 was synthesized using the protocol set forth in Scheme 7.

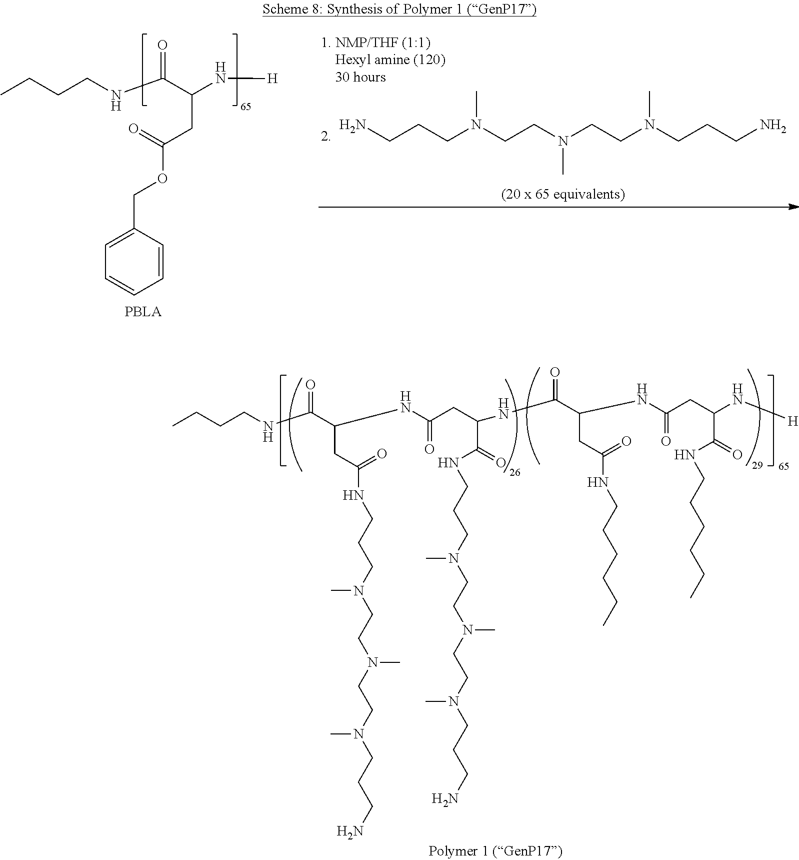

PBLA (25 mg, 0.0018 mmol) was dissolved into the mixture of 500 μL NMP and 500 μL of THF. Then to this reaction mixture was added hexyl amine (21.85 mg, 0.216 mmol) and the reaction mixture was stirred for 23 hours at room temperature. Then to this solution was added amine compound 10 (607 mg, 2.34 mmol) as a free amine form dissolved into 500 μL NMP, 500 μL THF and 500 μL triethylamine.

The free amine was prepared by dissolving 20 g of amine salt 10 into 30% NaOH solution (150 mL). The alkaline solution was stirred at room temperature for 20 min, and then kept in an ice bath for an additional 15 min. Ethyl acetate (200 mL) was added and extracted vigorously. The organic layer was separated from the aqueous layer. The extraction was repeated a second time, and the combined organic layers dried over anhydrous sodium sulfate. A turbid solution of free amine was collected via filtration and to this solution was added 10 mL methanol, and ethyl acetate was removed to yield partially free amine. The partially free amine was resuspended into 30% NaOH solution (150 mL) and extracted with ethyl acetate two times as described above. The combined organic layers were dried over anhydrous sodium sulfate and filtered. To this turbid solution was added 10 mL methanol and the organic solvent was removed to yield 9.8 g of the free amine.

The resulting reaction mixture was stirred at room temperature for 24 hours and the crude reaction mixture was precipitated into diethyl ether (40 mL) to yield crude polymer. Crude polymer was dissolved into 2 mL 1N HCl solution and was dialyzed using 3.5-5 KD cut-off membrane dialysis bag for 48 hours at 4° C. The purified polymer was lyophilized to yield Polymer 1 (“GenP17”).

GenP21 was synthesized using the same procedure except the following amounts of PBLA (50 mg, 0.0096 mmol), hexyl amine (97.13 mg, 0.96 mmol), and amine compound 10 (250 mg, 460.8 mmol) as the free amine were utilized, and the solution concentrations were modified based on scale, to form Polymer 1 (“GenP21”) with approximately 14 amine side chains and approximately 10 C₆ sidechains.

GenP27 was synthesized using the same procedure except the following amounts of PBLA (25 mg, 0.0018 mmol), hexyl amine (30 mg, 0.296 mmol), and amine compound 10 (607 mg, 2.34 mmol) as the free amine were utilized, and the solution concentrations were modified based on scale, to form Polymer 1 (“GenP27”) with approximately 20 amine side chains and approximately 45 C₆ sidechains.

GenP29 was synthesized using the same procedure except the following amounts of PBLA (700 mg, 0.0518 mmol), hexyl amine (629.62 mg, 6.22 mmol), and amine compound 10 (17.47 g, 67.34 mmol) as the free amine were utilized, and the solution concentrations were modified based on scale, to form Polymer 1 (“GenP29”) with approximately 22 amine side chains and approximately 43 C₆ sidechains.

### Example 6

The following example illustrates the effect of carbon length on the ability of the polymers ELN10, GenP9, GenP13, and GenP17 to form nanoparticles with mRNA.

Nanoparticles were formed with GFP mRNA in 20 mM HEPES (pH 7) at 5:1 weight ratio of polymer to mRNA and characterized by DLS. The particle diameter and polydispersity index were measured in triplicate and the average values are shown in Table 2.

TABLE 2
Nanoparticle Formation

| Polymer | Particle Diameter (nm) | Polydispersity Index (PDI) |
|---|---|---|
| ELN10 | 167.70 | 0.343 |
| GenP9 | 149.43 | 0.132 |
| GenP13 | 124.90 | 0.180 |
| GenP17 | 185.40 | 0.248 |

As is apparent from the results set forth in Table 2, GenP polymers GenP9, GenP13, and GenP17, having propyl units in the oligoamine/polyamine branches, have a much lower polydispersity index than polymer ELN10, having ethyl units in the oligoamine/polyamine branches, thereby demonstrating that the GenP polymers produce a more homogeneous distribution of nanoparticles than the ELN10 polymer.

### Example 7

The following example illustrates the effect of polymers ELN10, GenP9, GenP13, and GenP17 on the ability to deliver GFP mRNA to Hep3B cells.

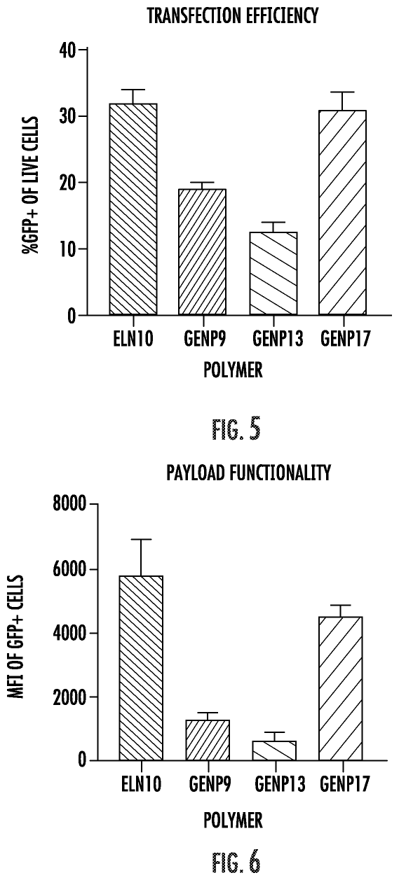

Nanoparticles were formed with GFP mRNA in 20 mM HEPES (pH 7) at 5:1 weight ratio of polymer to mRNA. Hep3B cells were treated with particles at 200 ng mRNA per well in media containing 10% FBS for 2-4 hours at 37° C. Cell viability and mRNA expression were assayed at 18-24 hours post transfection by flow cytometry. The experiments were performed in triplicate and the average transfection efficiency and payload functionality are plotted in FIGS. 5 and 6, respectively.

### Example 8

H27N polymer (as described in Example 1) nanoparticles and GenP13, GenP14, GenP17, GenP21, and GenP27 polymer (as described in Examples 3-5) nanoparticles were formulated with Cre mRNA in 20 mM HEPES using an Ignite™ microfluidic device,commercially available from Precision NanoSystems™, Vancouver, BC. The resulting particles were lyophilized in 20 mM Hepes, 1 μg/μL sucrose, and resuspended with 20 mM Hepes prior to administration.

Approximately eight-week-old LSL-luciferase mice were injected intravenously (IV) with 150-200 μL of the aforementioned particles at a dose of 2 mg/kg Cre mRNA cargo. Nanoparticle delivery of the Cre mRNA cargo to tissues was determined by whole body in vivo bioluminescence images (BLI) on day 6-7. The following day, major organs were harvested and ex vivo bioluminescence imaging was performed to verify the whole body BLI.

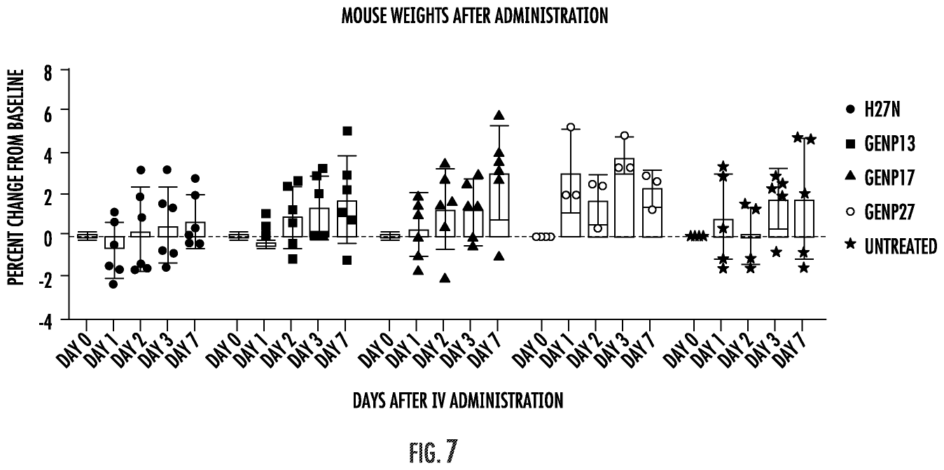

The mice were ear-tagged and randomized into treatment groups of 3-5 mice per group. The mice were then prepared and injected according to an IV injection protocol. The mice were weighed and the volume of PNP sample to deliver 2 mg/kg Cre mRNA cargo was calculated. Mice treated with AAV9 1e12 dose were used as a control. The mice were warmed using a heating pad and heat lamp and were placed in a restraint and 100-200 μL of the PNP samples was injected into the tail vein over 1-2 minutes. Each of the mice was observed for 15-30 minutes post-injection to ensure normal behavior and no complications from the injection. Body weights were determined and recorded on day 0, 1, 2, 3 and 7 as an indirect indication of systemic toxicity, and the results for the mice treated with H27N polymer nanoparticles and GenP13, GenP17, and GenP27 polymer nanoparticles are set forth in FIG. 7.

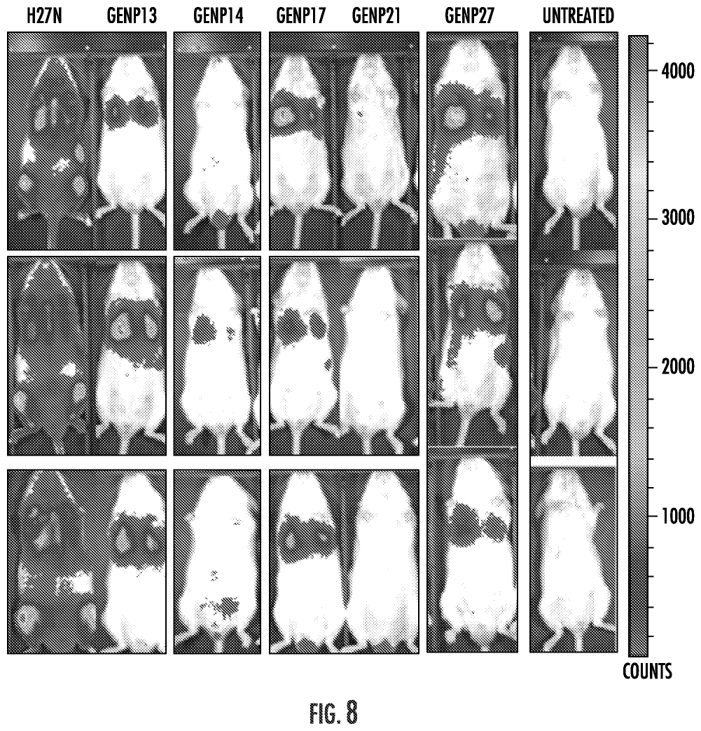

Six to seven days after injection, delivery and expression of CRE protein, resulting in excision of the transcriptional stop sequences allowing luciferase expression, was determined using a Perkin-Elmer IVIS in vivo Imaging System according to ex vivo bioluminescence imaging protocol. The treated mice were anesthetized by isoflurane inhalation and D-luciferin (300 mg/kg) was administered by intraperitoneal injection. Four to six minutes after administration of D-luciferin, the mice are placed in a supine position, belly-side up, into the IVIS instrument and images are recorded following the manufacturer's directions. The resulting images for the mice treated with H27N polymer nanoparticles and GenP13, GenP14, GenP17, GenP21, and GenP27 polymer nanoparticles are shown in FIG. 8.

As demonstrated by FIG. 8, administration of the H27N polymer nanoparticles results in mRNA delivery to many tissues including the lung, spleen, bone, and muscular tissues. In contrast, administration of the GenP13, GenP17, and GenP27 polymer nanoparticles resulted in targeted delivery to the lung, with minimal delivery to other parts of the body. Administration of the GenP14 and GenP21 polymer nanoparticles resulted in limited delivery to the lungs compared to the GenP13, GenP17, and GenP27 polymer nanoparticles.

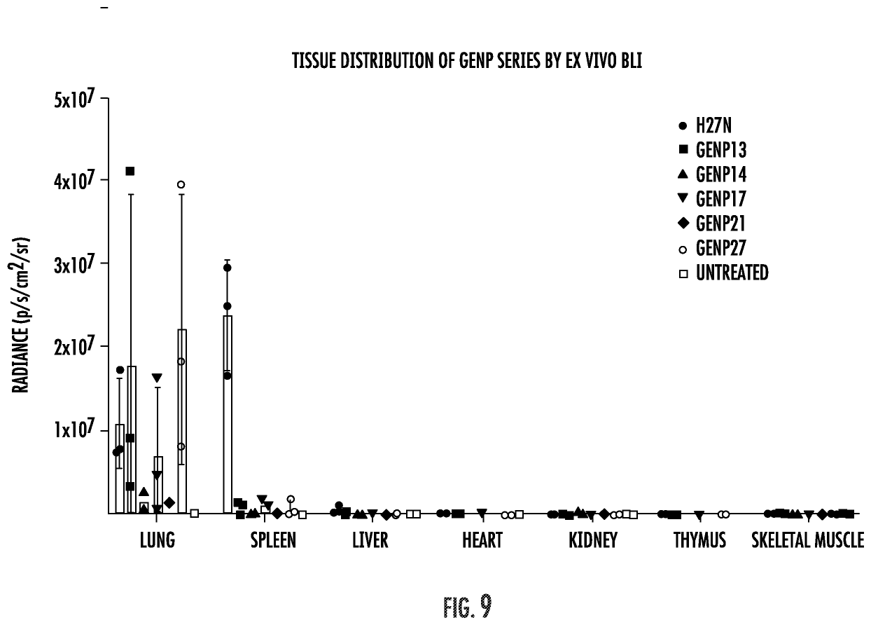

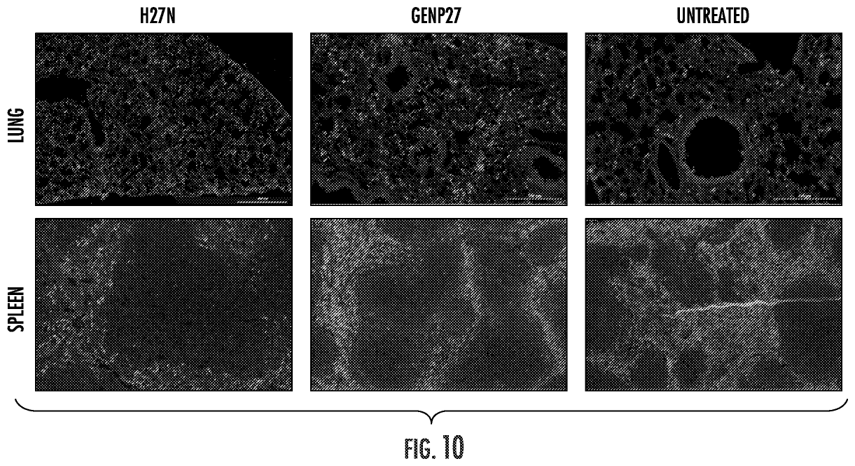

For ex vivo imaging of major organs, five minutes after administration of D-luciferin, the mice were sacrificed by cervical dislocation and necropsy was performed. Major organs (lung, liver, spleen, thymus, heart, kidneys, and gastrocnemius muscle) were harvested and quickly placed into separate wells of a 6-well plate containing ice cold 1 mg/ml solution of D-luciferin in DPBS. The plates were then place inside the IVIS instrument and were imaged immediately, and the results for the mice treated with H27N polymer nanoparticles and GenP13, GenP14, GenP17, GenP21, and/or GenP27 polymer nanoparticles are set forth in FIG. 9.

As demonstrated by FIG. 9, administration of the H27N polymer nanoparticles results in mRNA delivery to the lung and spleen. In contrast, administration of the GenP13, GenP17, and GenP27 polymer nanoparticles resulted in targeted delivery to the lung, with minimal delivery to other parts of the body. More particularly, administration of the GenP13, GenP17, and GenP27 polymer nanoparticles resulted in delivery to the liver at approximately two orders of magnitude lower than the lungs. Administration of the GenP14 and GenP21 polymer nanoparticles resulted in limited delivery to the lungs compared to the GenP13, GenP17, and GenP27 polymer nanoparticles.

### Example 9

H27N polymer (as described in Example 1) nanoparticles and GenP27 polymer (as described in Example 5) nanoparticles were formulated with Cre mRNA in 20 mM HEPES using an Ignite™ microfluidic device, commercially available from Precision NanoSystems™, Vancouver, BC. The resulting particles were lyophilized in 20 mM Hepes, 1 μg/μL sucrose, and resuspended with 20 mM Hepes prior to administration.

Ai9 mice were injected intravenously (IV) with 150-200 μL of the aforementioned particles at a dose of 2 mg/kg Cre mRNA cargo. The mice were sacrificed 10-14 days after injection and the lungs and spleen were harvested and slides were prepared for immunohistochemical analysis. Coverslips were applied with Prolong™ Gold with DAPI, and the resulting slides were imaged in a Cytation 5 microscope from BioTek™. Expression of tdTomato RFP is shown in red and CD68+ macrophages are shown in green, and representative photos are set forth in FIG. 10.

As demonstrated by FIG. 10, administration of the H27N polymer nanoparticles results in mRNA delivery to both the lungs and spleen. In contrast, administration of the GenP27 polymer nanoparticles resulted in significantly increased delivery to the lungs as compared to the spleen.

### Example 10

This example illustrates the preparation of a polymer comprising monomers with polyamine, hydrophobic, and PEG side chains, which can be used as secondary polymers in conjunction with the polymers otherwise disclosed herein to provide polymer nanoparticles.

A polymer designated P2K25 and having the following formula:

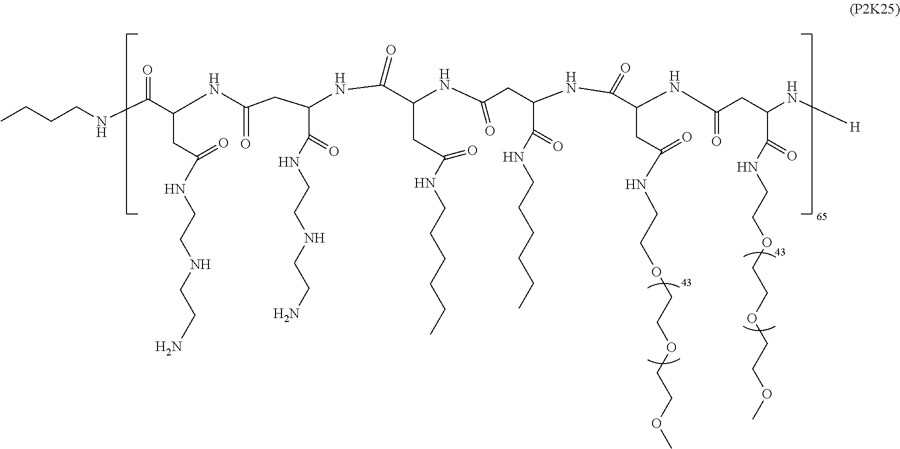

was prepared by modifying PBLA with a PEGylated amine, hexyl amine, and N-(2-aminoethyl)ethane-1,2-diamine (“DET”), as in Scheme 9.

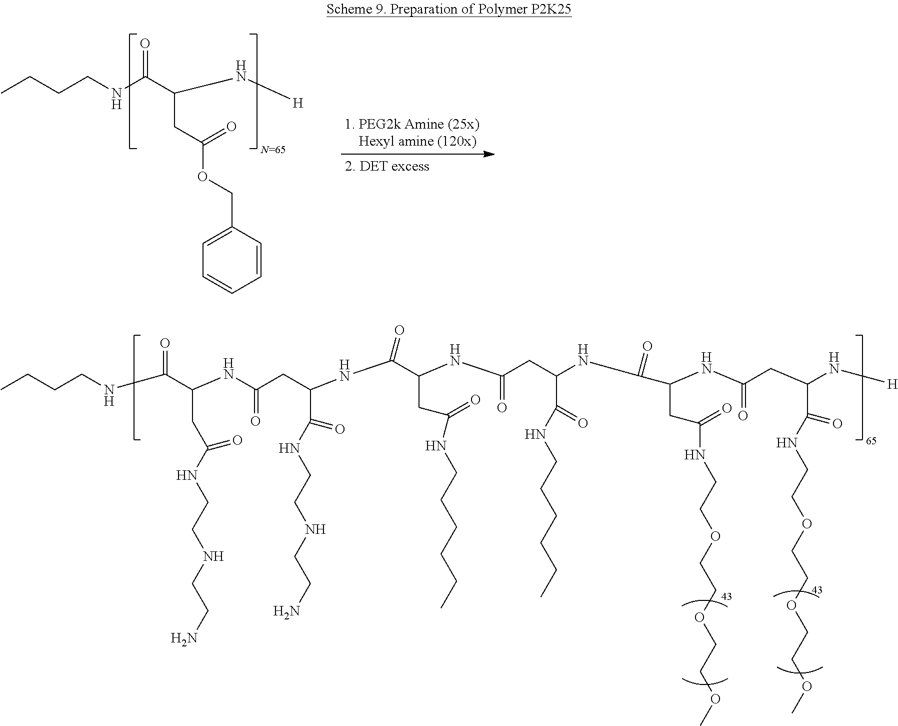

More particularly, lyophilized PBLA (50 mg, 0.0037 mMol; degree of polymerization (“DP”) 65) was placed into a flask and dissolved in tetrahydrofuran/N-methyl-2-pyrrolidine (1 mL each). To the clear solution was added n-hexylamine (58.8 μL, 0.44 mMol, 120 equivalents) and PEGylated amine (PEG2K amine; weight average MW=2000; number of ethylene oxide units (n)=43-46 (approx.)) (25 equivalents), and the clear reaction mixture was stirred for 24 hours at room temperature. After approximately 24 hours, diethylenetriamine (excess equivalents to benzyl group of PBLA segment, 1.0 g) was added to the clear mixture under mild anhydrous conditions. After approximately 18 hours at room temperature, the reaction mixture was precipitated into diethyl ether (10-12× volume, 35 mL). The precipitate was then centrifuged, and washed twice with diethyl ether. The polymer was dissolved in 1 M HCl (3 mL) and dialyzed in an excess of deionized water in a 3.5-5 KD cut-off membrane. When the pH of the solution was between 5-6, the dialysis was stopped, and the solution was lyophilized, to give the polymer product.

Similar procedures using different alkyamine and polyamine reactants in suitable proportions can be used to produce other such polymers as described herein. For instance, in Scheme 10, a polymer with a different polyamine side chain is provided by a similar reaction using a different polyamine reactant:

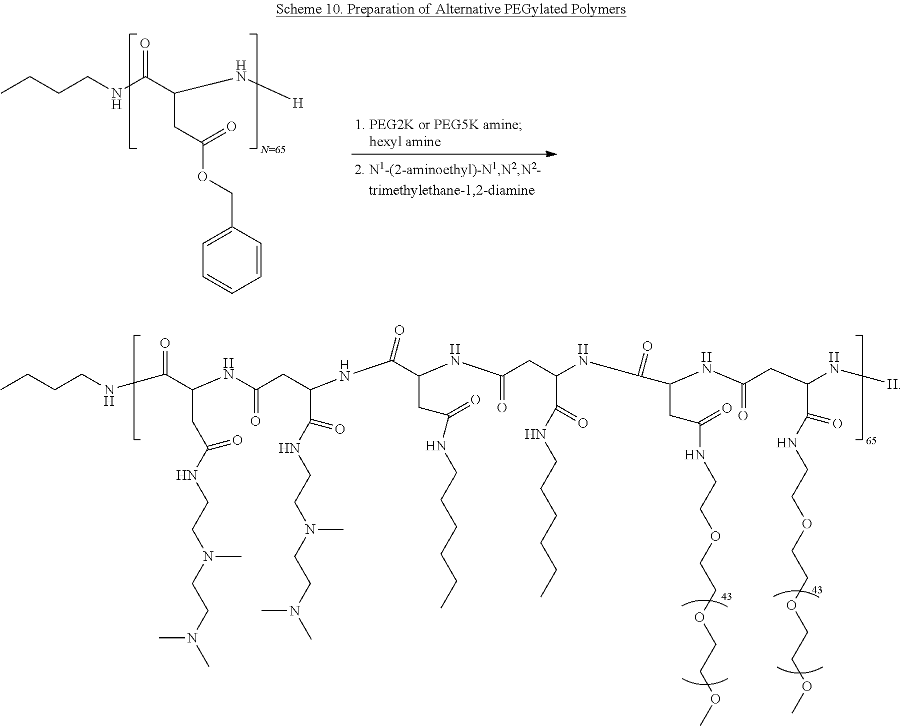

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a complex” includes a plurality of such complexes and reference to “the Cas9 polypeptide” includes reference to one or more Cas9 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

1. A polymer comprising a hydrolysable polymer backbone, the polymer backbone comprising:
(i) monomer units comprising a hydrophobic side chain; and
(ii) monomer units comprising a cationic side chain;
wherein the cationic side chain comprises a polyamine with at least one tertiary amine and only a single nucleophilic center, optionally at the terminus of the polyamine.

2. The polymer of claim 1, wherein the hydrophobic group comprises an alkyl group, alkenyl group, cycloalkyl group, cycloalkenyl group, or combinations thereof.

3. The polymer of claim 1, wherein the hydrophobic group comprises a C₃-C₁₂ linear or branched alkyl group, optionally a C₃-C₈ or C₃-C₆ linear or branched alkyl group.

4. The polymer of any of claims 1-3, wherein the polyamine comprises only one primary amine or only one secondary amine as the single nucleophilic center at the terminus of the polyamine, and all other amines in the polyamine are tertiary amines.

5. The polymer of claim 4, wherein the polyamine is a group of the formula:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
wherein each of p1 to p3 independently is an integer of 1 to 5; optionally wherein p1, p3, or both are greater than p2, or wherein p1, p3, or both are integers of 3 to 5;
r1 is an integer of 0 to 5;
and each instance of R² is independently C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group.

6. The polymer of any of claims 1-5, wherein the polymer further comprises monomers with a cationic polyamine side chain comprising a group of the formula:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,
wherein each of p1 to p3 independently is an integer of 1 to 5; optionally wherein p1, p3, or both are greater than p2, and/or wherein p1, p3, or both are integers of 3 to 5;
r1 is an integer of 0 to 5;
s1 is an integer from 1 to 5;
each instance of R² is independently C₁-C₆ alkyl group, C₂-C₆ alkenyl group, C₃-C₆ cycloalkyl group, or C₃-C₆ cycloalkenyl group, or R² is combined with a second R² so as to form a heterocyclic group;
each instance of R⁴ is independently —C(O)O—, —C(O)NH—, —C—O—C(O)—O—C—, —O—, or —S(O)(O)—; and
R⁵ is a group comprising a polyalkylene oxide, a polyglycolic acid, a polylactic acid or combination thereof, a tissue-specific or cell-specific targeting moiety; or a combination thereof.

7. The polymer of any one of claims 1-6, wherein the polymer comprises about 1 to about 80 mol % of the monomer units comprising a hydrophobic side chain and about 1 to about 80 mol % of the monomer units having a cationic side chain.

8. The polymer of any one of claims 1-7, wherein the hydrolysable polymer backbone comprises a polyamide, poly-N-alkylamide, polyester, polycarbonate, polycarbamate, or a combination thereof.

10. The polymer of claim 8, wherein the hydrolysable polymer backbone comprises a polyamide.

11. The polymer of claim 1, comprising a structure of Formula 1: 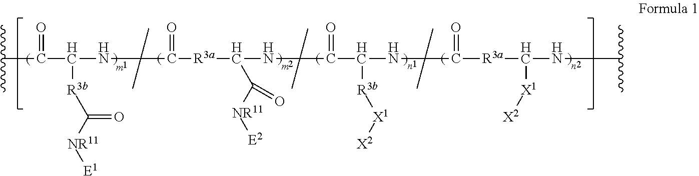
wherein:
each of m¹ and m² is an integer from 0 to 1000, provided that the sum of m¹+m² is greater than 2;
each of n¹ and n² is an integer from 0 to 1000, provided that the sum of n¹+n² is greater than 2;
the symbol “/” indicates that the units separated thereby are linked randomly or in any order;
each instance of R^{3a} is independently a methylene or ethylene group;
each instance of R^{3b} is independently a methylene or ethylene group;
each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents; optionally wherein R¹¹ is a hydrogen or a C₁-C₃ alkyl group;
each instance of X¹ independently is —C(O)O—, —C(O)NR¹—, —C(O)—, —S(O)(O)—, or a bond;
each instance of X² is a hydrophobic side chain;
and each instance of A¹ is a polyamine with at least one tertiary amine and only a single nucleophilic center, optionally at the terminus of the polyamine.

12. The polymer of claim 11, wherein:
each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents;
and each instance of A¹ is independently a group of formula
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
wherein each of p1 to p3 independently is an integer of 1 to 5, optionally wherein p1, p3, or both are greater than p2, and/or wherein p1, p3, or both are integers of 3 to 5; r1 is an integer of 0 to 5; and each instance of R² is independently C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group.

13. The polymer of any of claim 11 or 12, having the structure of Formula 1A: 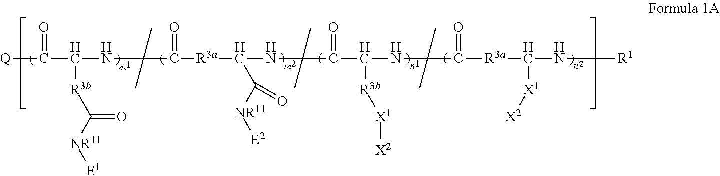
wherein
Q is of formula: 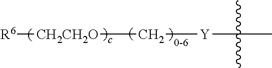
c is an integer from 0 to 50;
Y is optionally present and is a cleavable linker;
R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and
R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety;
or having the structure of Formula 1B: 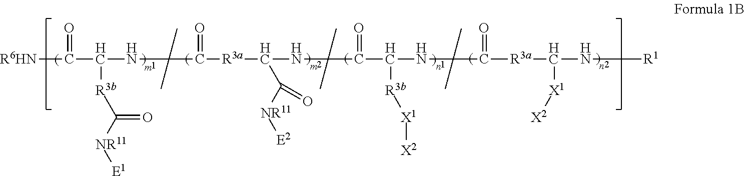
wherein
R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and
R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety;
or having the structure of Formula 1C:

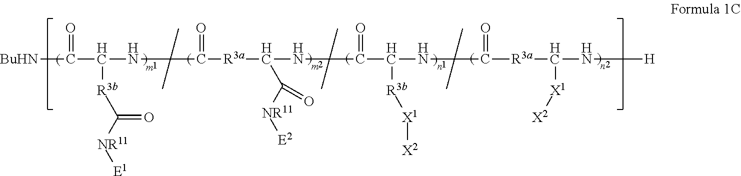

14. A polymer of any of claims 11-13, wherein the ratio of (m¹+m²)/(n¹+n²) is about 0.3 to 3.

15. A polymer comprising a hydrolysable polymer backbone, the polymer backbone comprising:
(i) monomer units comprising a hydrophobic side chain; and
(ii) monomer units comprising a cationic side chain comprising:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₁—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,
wherein:
each of p1 to p3 independently is an integer of 1 to 5; optionally wherein p1, p3, or both are greater than p2, and/or wherein p1, p3, or both are integers of 3 to 5;
r1 is an integer of 0 to 5;
s1 is an integer from 1 to 5;
each instance of R² is independently C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group;
each instance of R⁴ is independently —C(O)O—, —C(O)NH—, —C—O—C(O)—O—C—, —O—, or —S(O)(O)—; and
R⁵ is a group comprising a polyalkylene oxide, a polyglycolic acid, a polylactic acid, or combination thereof; a tissue-specific or cell-specific targeting moiety; or a combination thereof.

16. The polymer of claim 15, wherein the polymer comprises monomers with a cationic side comprising a group of the formula:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
and comprises monomers with a cationic side chain comprising monomers with a group of the formula:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵.

17. The polymer of claim 15, wherein the polymer comprises monomers with a cationic side comprising a group of the formula:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵.

18. The polymer of claim 15, wherein the polymer comprises monomers with a cationic side comprising a group of the formula:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
and comprises monomers with a cationic side chain comprising a group of the formula:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—(CH₂)ₛ₁—R⁵.

19. The polymer of claim 15, comprising a structure of Formula 2: 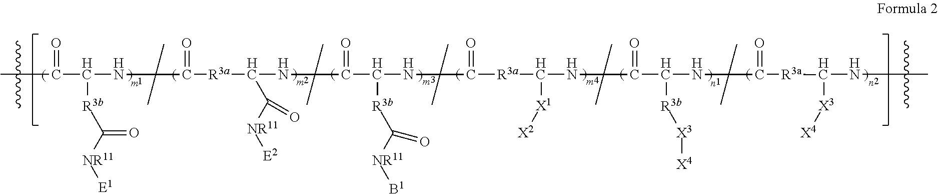
wherein:
each of m¹, m², m³, and m⁴ is an integer from 0 to 1000, provided that the sum of m¹+m²+m³+m⁴ is greater than 2;
each of n¹ and n² is an integer from 0 to 1000, provided that the sum of n¹+n² is greater than 2;
the symbol “/” indicates that the units separated thereby are linked randomly or in any order;
each instance of R^{3a} is independently a methylene or ethylene group;
each instance of R^{3b} is independently a methylene or ethylene group;
each instance of R¹¹ is independently hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group;
each X¹ independently is —C(O)O—, —C(O)NR¹—, —C(O)—, —S(O)(O)—, or a bond;
each instance of X² is a hydrophobic side chain;
each instance of A¹ is independently:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
each instance of B¹ is independently:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R⁴-R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴- R⁵,
wherein each of p1 to p3 independently is an integer of 1 to 5, optionally wherein p1, p3, or both are greater than p2, and/or wherein p1, p3, or both are integers of 3 to 5;
r1 is an integer of 0 to 5;
s1 is an integer from 1 to 5;
each instance of R² is independently C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group;
each instance of R⁴ is independently —C(O)O—, —C(O)NH—, —C—O—C(O)—O—C—, —O—, or —S(O)(O)—; and
R⁵ is a group comprising a polyalkylene oxide, a polyglycolic acid, a polylactic acid, or combination thereof; a tissue-specific or cell-specific targeting moiety; or a combination thereof.

20. The polymer of claim 18 or 19, wherein each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents.

21. The polymer of claim 19 or 20, having the structure of Formula 2A: 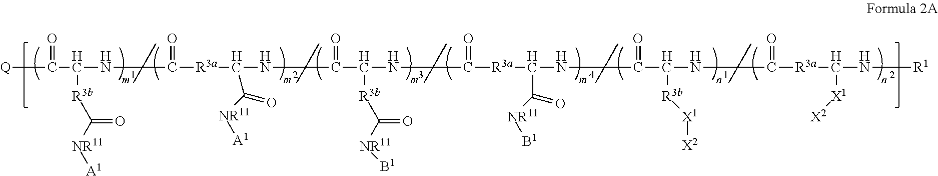
wherein
Q is of formula: 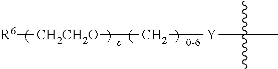
c is an integer from 0 to 50;
Y is optionally present and is a cleavable linker;
R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and
R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety;
or having the structure of Formula 2B: 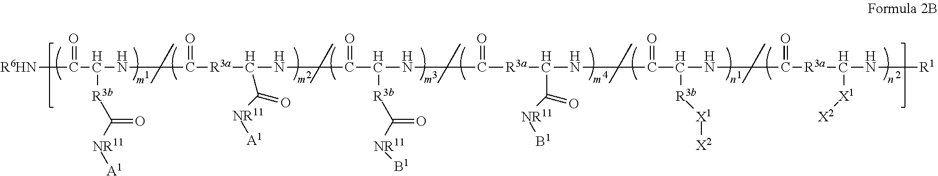
wherein
R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and
R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety;
or having the structure of Formula 2C:

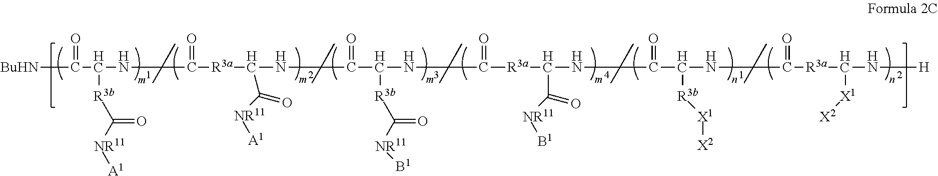

22. The polymer of any of claims 19-21, wherein each instance of A¹ is independently:
—(CH₂)ₚ₁—[NR²—(CH₂)ₚ₂—]ᵣ₁NR²—(CH₂)ₚ₃—NH₂;
and each instance of B¹ is independently:
—(CH₂)ₚ₁—[NR²—(CH₂)ₚ₂—]ᵣ₁NR²—(CH₂)ₚ₃—NH—(CH₂)ₛ₁—R⁴-R⁵.

23. The polymer of any of claims 6-22, wherein each instance of R⁵ is independently of the formula: 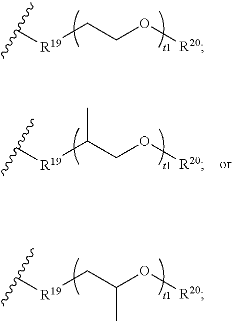
wherein:
R¹⁹ is a bond or a methylene, ethylene, or propylene group;
R²⁰ is hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group; and
t1 is an integer from 2 to 200.

24. The polymer of any of claims 19-23, wherein the ratio of (m¹+m²+m³+m⁴)/(n¹+n²) is about 0.3 to 3.

25. The polymer of any of claims 15-23, wherein at least one of p1 or p3, or both are greater than p2, and/or at least one of p1 or p3, or both are integers of 3-5.

26. The polymer of claim 25, wherein the polymer is one of polymers 1-72.

27. The polymer of any of claims 1-25, provided the polymer is not:

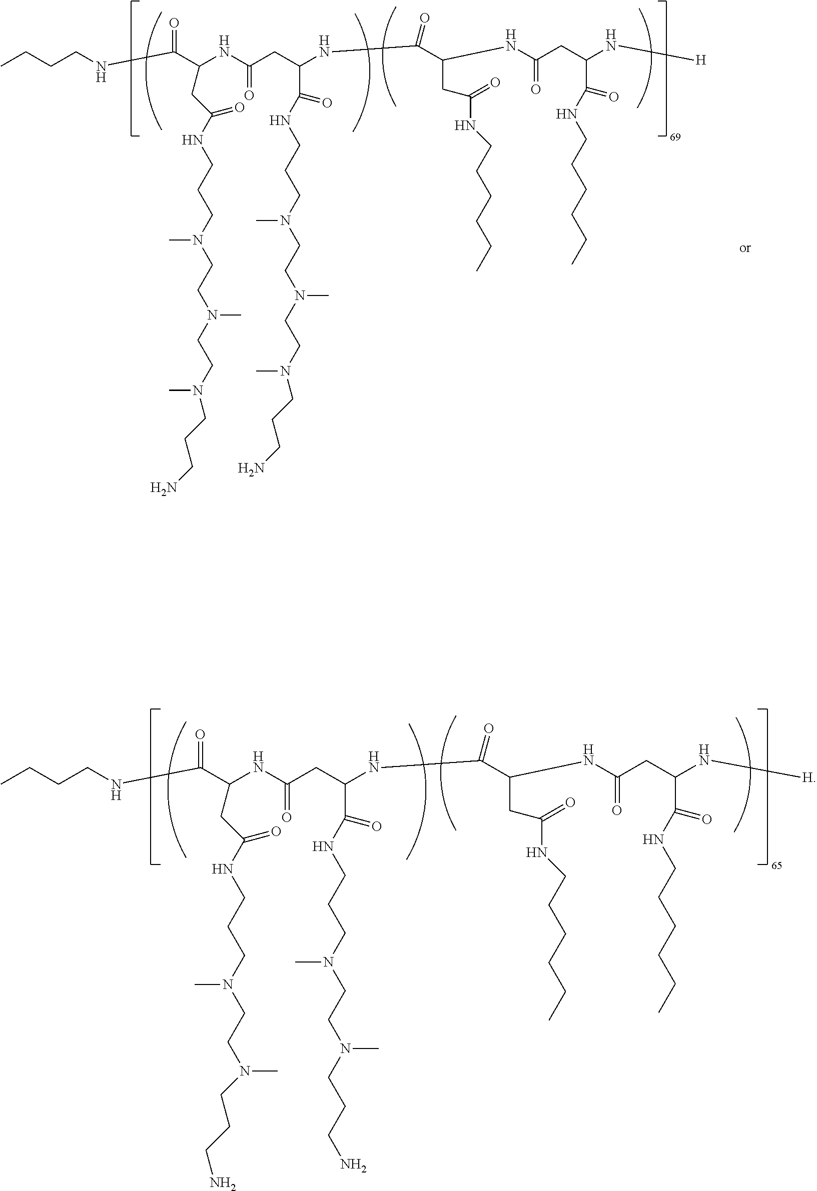

28. A polymer having the structure:

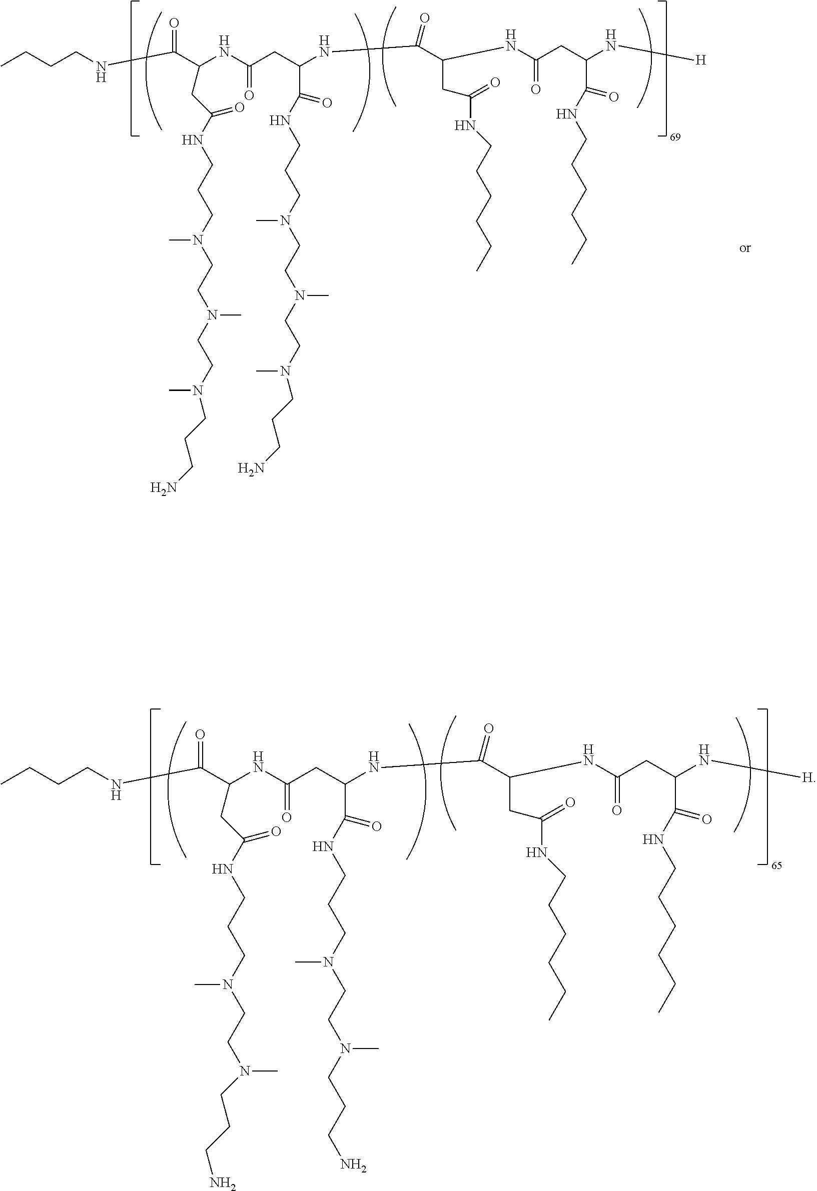

29. A method of preparing a polymer of Formula 2, the method comprising:
(a) providing a polymer of Formula 1 as defined in any of claims 11-14: 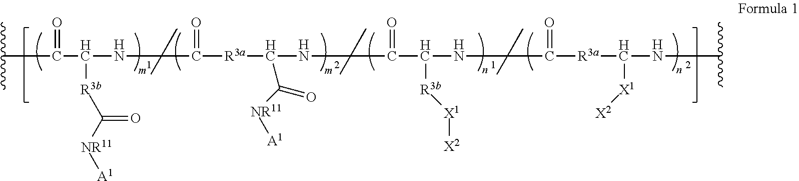
and
(b) modifying a portion of groups A¹ of the polymer of Formula 1 to provide the polymer of Formula 2: 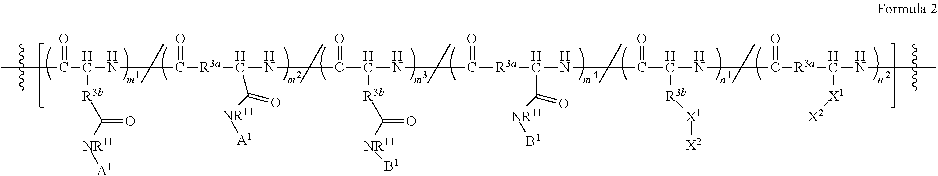
wherein:
each of m¹, m², m³, and m⁴ is an integer from 0 to 1000, provided that the sum of m¹+m²+m³+m⁴ is greater than 2;
each of n¹ and n² is an integer from 0 to 1000, provided that the sum of n¹+n² is greater than 2;
the symbol “/” indicates that the units separated thereby are linked randomly or in any order;
each instance of R^{3a} is independently a methylene or ethylene group;
each instance of R^{3b} is independently a methylene or ethylene group;
each X¹ independently is —C(O)O—, —C(O)NR¹—, —C(O)—, —S(O)(O)—, or a bond;
each instance of R¹¹ is independently hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group;
each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof, any of which can be substituted with one or more substituents;
each instance of A¹ is independently:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²,
each instance of B¹ is independently:
—(CH₂)ₚ₁—[NR²—(CH₂)ₚ₂—]ᵣ₁NR²—(CH₂)ₚ₃—NH—(CH₂)ₛ₁—R⁴-R⁵;
—(CH₂)ₚ₁—[NR²—(CH₂)ₚ₂—]ᵣ₁NR²—(CH₂)ₚ₃—NH—CH₂—CHOH—R⁵;
—(CH₂)ₚ₁—[NR²—(CH₂)ₚ₂—]ᵣ₁NR²—(CH₂)ₚ₃—NH—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁵;
—(CH₂)ₚ₁—[NR²—(CH₂)ₚ₂—]ᵣ₁NR²—(CH₂)ₚ₃—NH—CH(CONH₂)—(CH₂)ₛ₁—R⁴-R⁵,
—(CH₂)ₚ₁—[NR²—(CH₂)ₚ₂—]ᵣ₁NR²—(CH₂)ₚ₃—NR²—(CH₂)ₛ₁—R—R⁵;
—(CH₂)ₚ₁—[NR²—(CH₂)ₚ₂—]ᵣ₁NR²—(CH₂)ₚ₃—NR²—CH₂—CHOH—R⁵;
—(CH₂)ₚ₁—[NR²—(CH₂)ₚ₂—]ᵣ₁NR²—(CH₂)ₚ₃—NR²—(CH₂)ₛ₁—R⁵;
—(CH₂)ₚ₁—[NR²—(CH₂)ₚ₂—]ᵣ₁NR²—(CH₂)ₚ₃—NR²—CH(CONH₂)—(CH₂)ₛ₁—R⁵; or
—(CH₂)ₚ₁—[NR²—(CH₂)ₚ₂—]ᵣ₁NR²—(CH₂)ₚ₃—NR²—CH(CONH₂)—(CH₂)ₛ₁—R⁴-R⁵,
wherein each of p1 to p3 independently is an integer of 1 to 5, optionally wherein p1, p3, or both are greater than p2, and/or wherein p1, p3, or both are integers of 3 to 5; r1 is an integer of 0 to 5; s1 is an integer from 1 to 5; each instance of R² is independently a C₁-C₁₂ alkyl group, C₂-C₁₂ alkenyl group, C₃-C₁₂ cycloalkyl group, or C₃-C₁₂ cycloalkenyl group, or R² is combined with a second R² so as to form a heterocyclic group; each instance of R⁴ is independently —C(O)O—, —C(O)NH—, —C—O—C(O)—O—C—, —O—, or —S(O)(O)—; and R⁵ is a group comprising a polyalkylene oxide, a polyglycolic acid, a polylactic acid, or combination thereof, a tissue-specific or cell-specific targeting moiety; or a combination thereof.

30. The method of claim 29, wherein modifying a portion of groups A¹ of the polymer of Formula 1 comprises reacting a portion of the A¹ groups with a compound having the structure: 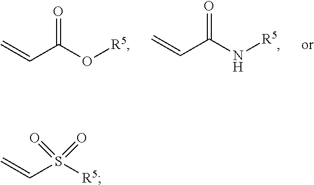
to provide the polymer of Formula 2;
wherein A¹ is:
—(CH₂)ₚ₁—[NR²—(CH₂)ₚ₂—]ᵣ₁NR²—(CH₂)ₚ₃—NH₂ or
—(CH₂)ₚ₁—[NR²—(CH₂)ₚ₂—]ᵣ₁NR²—(CH₂)ₚ₃—NHR²
and B¹ is:
—(CH₂)ₚ₁—[NR²—(CH₂)ₚ₂—]ᵣ₁NR²—(CH₂)ₚ₃—NH—(CH₂)ₛ₁—R⁴-R⁵ or
—(CH₂)ₚ₁—[NR²—(CH₂)ₚ₂—]ᵣ₁NR²—(CH₂)ₚ₃—NR²—(CH₂)ₛ₁—R⁴-R⁵.

31. The method of claim 29 or 30, wherein A¹ is:
—(CH₂)ₚ₁—[NR²—(CH₂)ₚ₂—]ᵣ₁NR²—(CH₂)ₚ₃—NH₂
and B¹ is:
—(CH₂)ₚ₁—[NR²—(CH₂)ₚ₂—]ᵣ₁NR²—(CH₂)ₚ₃—NH—(CH₂)ₛ₁—R⁴-R⁵.

32. A method of preparing a polymer of Formula 1 according to any of claims 11-14: 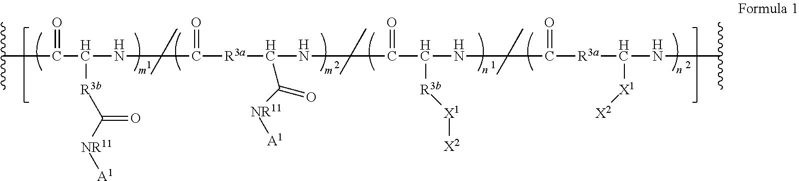
the method comprising:
(I) reacting a polymer of Formula 3: 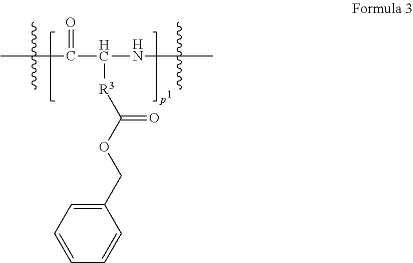
with (a) a compound of the formula HNR¹A; and (b) a compound of formula H₂NX² or HOX², simultaneously or in any sequential order;
or
(II) reacting a polymer of Formula 4 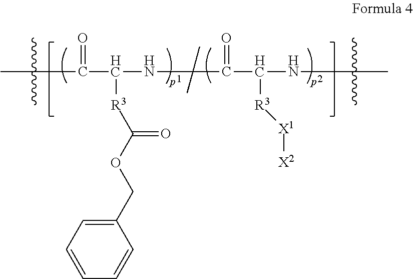
with a compound of the formula HNR¹¹A¹;
wherein,
p¹ is an integer from 1 to 2000;
p² is an integer from 1 to 2000;
each R³ is independently a methylene or ethylene group;
and m¹, m², n¹, n², R^{3a}, R^{3b}, R¹¹, X¹, X², and A¹ are as defined in any of claims 11-14.

33. The method of claim 32, wherein the polymer comprising the structure of Formula 3 or Formula 4 is a polymer of Formula 3A or Formula 4A, respectively: 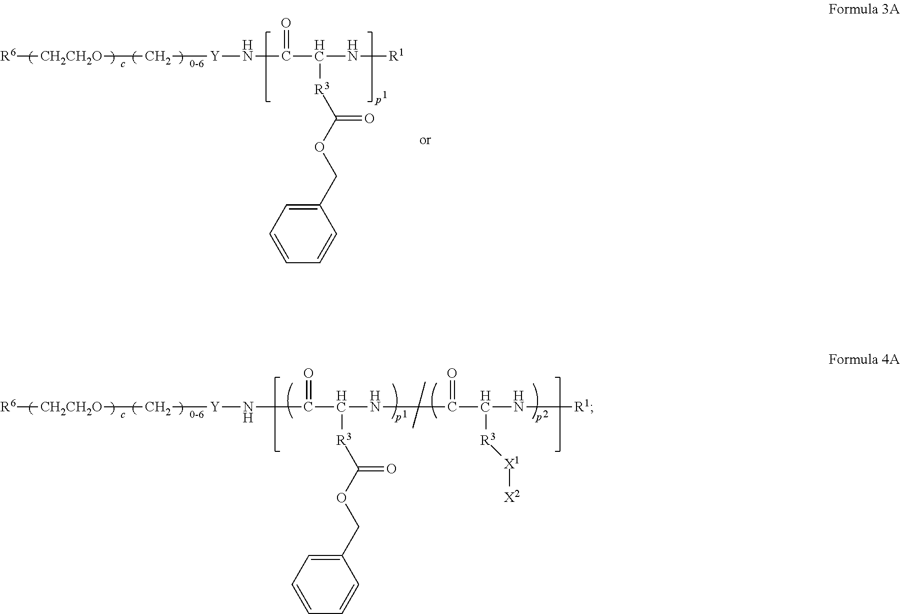
wherein,
p¹ is an integer from 1 to 2000;
p² is an integer from 1 to 2000;
each R³ is independently a methylene or ethylene group;
each X¹ independently is —C(O)O—, —C(O)NR¹¹—, —C(O)—, —S(O)(O)—, or a bond;
each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally comprising one or more primary, secondary, or tertiary amines; any of which can be substituted with one or more substituents;
each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents, optionally hydrogen or a C₁-C₃ alkyl group;
c is an integer from 0 to 50;
Y is optionally present and is a cleavable linker;
R¹ is hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which are optionally substituted with one or more substituents; and
R⁶ is hydrogen, an amino group, an aryl group, a heterocyclic group, a C₁-C₁₂ alkyl group, a C₁-C₁₂ heteroalkyl group, C₂-C₁₂ alkenyl group, C₃-C₁₂ cycloalkyl group, or C₃-C₁₂ cycloalkenyl group, any of which are optionally substituted with one or more substituents or a tissue-specific or cell-specific targeting moiety.

34. The method of claim 32, wherein the polymer comprising the structure of Formula 3 or Formula 4 is a polymer of Formula 3B or Formula 4B, respectively: 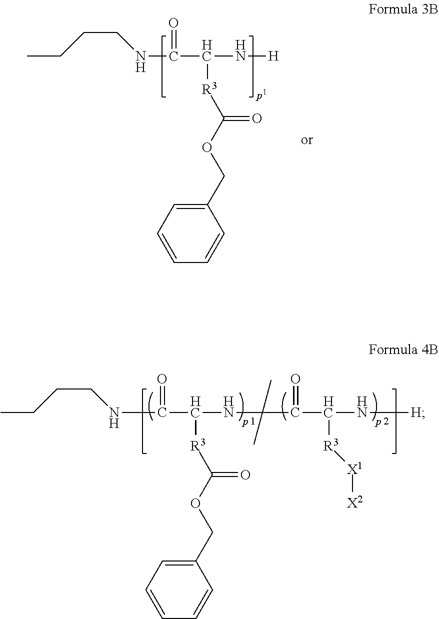
wherein,
p is an integer from 1 to 2000;
p² is an integer from 1 to 2000;
each R³ is independently a methylene or ethylene group;
each X¹ independently is —C(O)O—, —C(O)NR¹¹—, —C(O)—, —S(O)(O)—, or a bond;
each instance of X² is independently a C₁-C₁₂ alkyl or heteroalkyl group, C₃-C₁₂ cycloalkyl group, C₂-C₁₂ alkenyl group, C₃-C₁₂ cycloalkenyl group, aryl group, heterocyclic group, or combination thereof optionally comprising one or more primary, secondary, or tertiary amines; any of which can be substituted with one or more substituents; and
each instance of R¹¹ is independently hydrogen, an aryl group, a heterocyclic group, a C₁-C₁₂ alkyl group, C₂-C₁₂ alkenyl group, C₃-C₁₂ cycloalkyl group, or C₃-C₁₂ cycloalkenyl group, any of which can be optionally substituted with one or more substituents, optionally hydrogen or a C₁-C₃ alkyl group.

35. The method of claim 32 comprising combining a compound of Formula 3 with (a) a compound of HNR¹A, and (b) compound of formula H₂NX² or HOX², wherein (a) and (b) are present in a molar ratio of about 1:10 to about 1:150, optionally about 1:40 to about 1:150, or about 1:80 to about 1:150.

36. The method of any of claims 29-35, wherein each instance of R⁵ is independently of the formula: 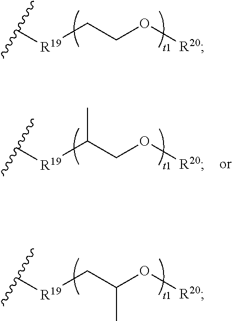
wherein:
R¹⁹ is a bond or a methylene, ethylene, or propylene group;
R²⁰ is hydrogen, an aryl group, a heterocyclic group, a C₁-C₁₂ alkyl group, alkenyl group, cycloalkyl group, or cycloalkenyl group, any of which can be optionally substituted with one or more substituents, optionally hydrogen or a C₁-C₃ alkyl group; and
t1 is an integer from 2 to 200.

37. A composition comprising a polymer of any one of claims 1-28 and a nucleic acid and/or polypeptide.

38. The composition of claim 37, wherein the composition comprises a guide nucleic acid and/or donor nucleic acid.

39. The composition of claim 36 or 37, wherein the composition comprises an endonuclease.

40. The composition of claim 39, wherein the composition comprises an RNA-guided endonuclease or nucleic acid encoding same.

41. The composition of claim 40, wherein the RNA-guided endonuclease is Cas9, Cpf1, or a combination thereof.

42. The composition of any one of claims 37-41, wherein the composition comprises a DNA recombinase.

43. The composition of claim 42, wherein the DNA recombinase is Cre recombinase.

44. The composition of any one of claims 37-43, wherein the composition comprises a zinc finger nuclease.

45. The composition of any one of claims 37-44, wherein the composition comprises a transcription activator-like effector nuclease.

46. The composition of any one of claims 37-45, wherein the composition comprises a nanoparticle comprising the polymer of any of claims 1-23 and the nucleic acid or polypeptide.

47. The composition of any one of claims 37-46, wherein the composition comprises a second polymer that comprises a polyalkylene oxide, polyglycolic acid, polylactic acid, or combination thereof.

48. A method of delivering a nucleic acid and/or polypeptide to a cell, the method comprising administering the composition of any one of claims 37-47 to the cell.

49. The method of claim 48, wherein the cell is in a subject and the composition of any one of claims 37-48 is administered to the subject.

50. The method of claim 48 or 49, wherein the method is for delivery of the nucleic acid and/or polypeptide to lung tissue.

51. The method of claim 48 or 49, wherein the method is for delivery of the nucleic acid and/or polypeptide to muscle tissue.

52. The method of claim 50, wherein the polymer comprises a targeting moiety that preferentially binds to tumor cells.

53. The method of any of claims 48-52, wherein the composition comprises one or more of an RNA guided endonuclease or nucleic acid encoding same, a guide nucleic acid, and a donor nucleic acid, and the composition facilitates editing of a target gene in the cell.

54. The method of any of claims 48-53, wherein the cell is in a host, optionally a human, and the composition is delivered to the cell by administering the composition to the host.

55. The polymer of any of claims 1-28 or the composition of any one of claims 37-47 for delivering a nucleic acid and/or polypeptide to a cell, optionally in accordance with the methods of claims 48-54.

56. Use of the polymer of any of claims 1-28 or the composition of any one of claims 37-47 for preparing a medicament for delivering a nucleic acid and/or polypeptide to a cell, optionally in accordance with the methods of claims 48-54.
